(19) United States
(12) Patent Application Publication
(10) Pub. No.: US 2023/0357754 A1
(43) Pub. Date: Nov. 9, 2023

(54) RAPID EXTRACELLULAR ANTIBODY PROFILING (REAP) FOR THE DISCOVERY AND USE OF SAID ANTIBODIES

(71) Applicant: YALE UNIVERSITY, New Haven, CT (US)

(72) Inventors: Connor Rosen, New Haven, CT (US); Eric Wang, New Haven, CT (US); Aaron Ring, New Haven, CT (US); Yile Dai, New Haven, CT (US)

(21) Appl. No.: 17/906,759

(22) PCT Filed: Mar. 22, 2021
(86) PCT No.: PCT/US21/23521

§ 371 Date: Sep. 20, 2022

Related U.S. Application Data
(60) Provisional application No. 62/992,484, filed on Mar. 20, 2020.

(51) Int. Cl. C12N 15/10

(52) U.S. Cl. CPC C12N 15/1037; C12N 15/1065

(57) ABSTRACT

The present invention relates to methods for a sensitive and high-throughput detection of various antibodies and targets thereof. For example, in one aspect, methods of the present invention can successfully detect autoantibodies against extracellular and secreted proteins. In various embodiments, the present invention provides methods of diagnosing, assessing prognosis, preventing, and treating diseases or disorders associated with antibodies or targets thereof detected via the high-throughput detection methods of the present invention.

### CROSS REFERENCE TO RELATED APPLICATIONS

This application claims priority to U.S. Provisional Application No. 62/992,484, filed Mar. 20, 2020 which is hereby incorporated by reference herein in its entirety.

### STATEMENT REGARDING FEDERALLY SPONSORED RESEARCH OR DEVELOPMENT

This invention was made with government support under CA196530 awarded by National Institutes of Health. The government has certain rights in the invention.

### BACKGROUND OF THE INVENTION

Antibodies are natural products of the immune system that normally mediate host-defense against foreign pathogens. Auto-reactive antibodies that recognize against self-antigens play a major role in numerous facets of normal health and disease. For instance, autoantibodies underlie a wide range of autoimmune diseases, but they also contribute to anti-tumor immune responses against cancer. The precise targets of autoantibodies have been shown in many cases to determine the pathophysiology of disease, in both exacerbating and mitigating mechanisms. In some cases, autoantibodies of particular specificity may be diagnostic. In others, if the autoantibodies are functional and can exert immunomodulatory effects, they can drive disease pathogenesis or attenuate disease severity. Hence, identifying the precise molecular specificities of autoantibodies is critical for understanding the molecular basis for numerous diseases. Furthermore, knowledge of autoantibody reactivities may reveal new therapeutic disease targets, for instance by revealing anti-cancer antibody targets (e.g., endogenous anti-HER2 responses seen in breast cancer and anti-MUC1 in carcinoma) or immunosuppressive targets in autoimmune disease (e.g., endogenous anti-IFN-α in less severe cases of systemic lupus erythematosus). Autoantibodies themselves may represent therapeutic agents, given that they are fully human, recognize a native human antigen, and exert a desired therapeutic activity that can be inferred from clinical outcomes associated with the seroreactivity.

One major barrier in the identification of autoantibodies is limitations in modern autoantibody discovery methods. On one hand, current autoantibody detection methods that maximize sensitivity are limited in throughput, which forces autoantibody discovery to be done in a deductive process on the basis of well-known protein targets. On the other hand, current high-throughput autoantibody discovery methods that enable unbiased autoantibody detection, such as protein microarray or phage-based peptide display methods, do not effectively detect antibodies against extracellular and secreted proteins (the “exoproteome”) due to the conformational nature of these antigens. This is a major limitation because the “exoproteome” contains the very proteins that reside topologically outside the cell and are actually accessible to circulating autoantibodies. As such, extracellular proteins constitute the most likely targets of functional autoantibodies.

Thus, there is a need in the art for a sensitive and high-throughput detection method of antibodies and targets thereof that can successfully detect autoantibodies against extracellular and secreted proteins. The present invention addresses this need.

### BRIEF SUMMARY OF THE INVENTION

In one embodiment, the invention provides a method of identifying at least one polypeptide which binds to at least one antibody, wherein the method comprises:
(a) contacting a library of display cells or particles with a sample comprising at least one antibody, wherein the library of display cells comprises a plurality of cells or particles wherein together the plurality of cells or particles comprises nucleic acid molecules for expression of a plurality of extracellular proteins, secreted proteins or a combination thereof,
wherein each cell or particle of the plurality of cells or particles comprises a barcoded nucleic acid molecule, wherein each nucleic acid molecule comprises
   i) a nucleotide sequence encoding a polypeptide of interest for display on the surface of the cell or particle; and
   ii) a unique nucleotide barcode sequence;
(b) isolating one or more antibody-bound cell or particle;
(c) isolating at least one barcoded nucleic acid molecule from at least one cell or particle of step (b); and
(d) identifying the barcoded nucleic acid molecule, thereby identifying the associated encoded polypeptide as an antigen for binding by at least one antibody in the sample.

In one embodiment, the method of isolating one or more antibody-bound cell or particle comprises high-throughput magnetic separation.

In one embodiment, the method further comprises the step of:
(b′) expanding the one or more isolated antibody-bound cell or particle.

In one embodiment, the method of identifying the barcoded nucleic acid molecule comprises at least one selected from the group consisting of amplifying the barcoded nucleic acid molecule and sequencing the barcoded nucleic acid molecule.

In one embodiment, the method comprises:
in step (b), isolating multiple antibody bound cells,
in step (c), isolating the barcoded nucleic acid molecules from the cells of step (b), and
in step (d), sequencing the isolated barcoded nucleic acid molecules, and identifying the associated encoded polypeptide as an antigen for binding by the antibody based on an enrichment of the number of reads of the associated barcode in the sequencing data as compared to a threshold level.

In one embodiment, the threshold level is selected from the group consisting of a predetermined threshold level, a statistically determined threshold, and a threshold level determined using z-scores.

In one embodiment, the library of display cells or particles comprises a library of barcoded nucleic acid molecules encoding at least one selected from an extracellular domain of a protein, an extracellular protein, and a secreted protein.

In one embodiment, the library of barcoded nucleic acid molecules comprises a plurality of nucleic acid molecules which together encode the human exoproteome.

In one embodiment, the library of barcoded nucleic acid molecules comprises at least one nucleic acid molecule encoding at least one polypeptide sequence selected from SEQ ID NO:1-3092.

In one embodiment, the library of barcoded nucleic acid molecules comprises a plurality of nucleic acid molecules which together encode each of SEQ ID NO:1-3092.

In one embodiment, the library of barcoded nucleic acid molecules comprises at least one nucleic acid molecule comprising a nucleotide sequence selected from SEQ ID NO:3093-6185.

In one embodiment, the library of barcoded nucleic acid molecules comprises a plurality of nucleic acid molecules which together comprise each of SEQ ID NO:3093-6185.

In one embodiment, the sample comprises a biological sample selected from the group consisting of a body fluid, blood, serum, plasma, cerebrospinal fluid, tissue, and any combination thereof.

In one embodiment, the sample comprises at least one antibody purified from a biological sample selected from the group consisting of a body fluid, blood, serum, plasma, cerebrospinal fluid, tissue, and any combination thereof.

In one embodiment, the at least one antibody is purified from a biological sample by at least one selected from the group consisting of:
(a) affinity purification for a specific antibody isotype of interest, and
(b) contacting the sample with a control cell or particle comprising an empty expression plasmid.

In one embodiment, the sample is from a subject diagnosed as having a disease or disorder, and whereby the antigen for binding by at least one antibody is a disease-associated antigen.

In one embodiment, the antibody is an autoantibody.

In one embodiment, the antibody is associated with an autoimmune disease or disorder, cancer, inflammatory disease or disorder, metabolic disease or disorder, neurodegenerative disease or disorder, organ tissue rejection, organ transplant rejection, or any combination thereof.

In one embodiment, the invention relates to a method of preventing or treating a disease or disorder in a subject in need thereof; the method comprising administering a therapeutic agent to the subject, wherein the therapeutic agent comprises an agent for modifying the level or reactivity of at least one antibody which interacts with at least one antigen selected from the group consisting of the antigens as set forth in SEQ ID NO:1-3092.

In one embodiment, the antigen is identified as a target for at least one antibody according to a method comprising:
(a) contacting a library of display cells or particles with a sample comprising at least one antibody, wherein the library of display cells comprises a plurality of cells or particles wherein together the plurality of cells or particles comprises nucleic acid molecules for expression of a plurality of extracellular proteins, secreted proteins or a combination thereof,
wherein each cell or particle of the plurality of cells or particles comprises a barcoded nucleic acid molecule, wherein each nucleic acid molecule comprises
   i) a nucleotide sequence encoding a polypeptide of interest for display on the surface of the cell or particle; and
   ii) a unique nucleotide barcode sequence;
(b) isolating one or more antibody-bound cell or particle;
(c) isolating at least one barcoded nucleic acid molecule from at least one cell or particle of step (b); and
(d) identifying the barcoded nucleic acid molecule, thereby identifying the associated encoded polypeptide as an antigen for binding by at least one antibody in the sample

In one embodiment, the at least one antigen is selected from the group consisting of an antigen as set forth in Table 3, and further wherein the disease or disorder is the disease or disorder associated with the antigen as set forth in Table 3.

In one embodiment, the therapeutic agent comprises an agent for decreasing the level or reactivity of at least one antibody with at least one disease-associated antigen selected from the group consisting of the antigens as set forth in Table 3.

In one embodiment, the at least one antigen is selected from the group consisting of an antigen as set forth in Table 6, and further wherein the disease or disorder is the disease or disorder associated with the antigen as set forth in Table 6.

In one embodiment, the therapeutic agent comprises a therapeutically effective amount of at least agent that reduces or eliminates at least one antibody.

In one embodiment, the therapeutic agent comprises a composition comprising an antigen selected from the group consisiting of an antigen as set forth in SEQ

ID NO:1-3092 linked to a domain for endocytosis and degradation.

In one embodiment, the therapeutic agent comprises a composition comprising an antigen selected from the group consisiting of an antigen as set forth in Table 6 linked to a domain for endocytosis and degradation.

In one embodiment, the domain for endocytosis and degradation comprises an asialoglycoprotein receptor binding domain.

In one embodiment, the agent that reduces or eliminates at least one antibody comprises a molecule for targeting and destruction of at least one antibody-expressing cell.

In one embodiment, the agent comprises a chimeric antigen receptor (CAR) T cell expressing an antigen selected from the group consisting of an antigen as set forth in SEQ ID NO:1-3092, or a fragment thereof.

In one embodiment, the CAR T cell expresses an antigen selected from the group consisting of an antigen as set forth in Table 6.

In one embodiment, the therapeutic agent comprises an agent for increasing the level or reactivity of at least one antibody with at least one disease-associated antigen selected from the group consisting of the antigens as set forth in Table 3.

In one embodiment, the at least one antigen is selected from the group consisting of an antigen as set forth in Table 5, and further wherein the disease or disorder is the disease or disorder associated with the antigen as set forth in Table 5.

In one embodiment, the therapeutic agent comprises a therapeutically effective amount of at least one antibody, or fragment thereof, wherein the antibody specifically binds to a disease-associated antigen.

In one embodiment, the disease or disorder is selected from the group consisting of an autoimmune disease or disorder, cancer, inflammatory disease or disorder, metabolic disease or disorder, neurodegenerative disease or disorder, organ tissue rejection, organ transplant rejection, or any combination thereof.

In one embodiment, the disease or disorder is selected from the group consisting of antineutrophil cytoplasmic antibody (ANCA)-associated vasculitis, autoimmune polyendocrinopathy candidiasis ecto-dermal dystrophy, antiphospholipid antibody syndrome, chronic inflammatory demyelinating polyradiculoneuropathy, cutaneous lupus erythematosus, COVID-19, drug-induced lupus, dermatomyositis, glomerulonephritis, a disease or disorder associated with kidney transplant, malaria, mixed connective tissue disease, myasthenia gravis, malignant melanoma, neuromyelitis optica, non-small cell lung cancer, pediatric autoimmune neuropsychiatric disorders associated with streptococcal infections, systemic lupus erythematosus, sjogren's syndrome, scleroderma, susac syndrome, undifferentiated connective tissue disease, and any combination thereof.

In one embodiment, the invention relates to a method of diagnosing, assessing the prognosis, or assessing the effectiveness of treatment of a disease or disorder in a subject in need thereof, the method comprising assessing the level or reactivity of at least one antibody which interacts with at least one antigen selected from the group consisting of an antigen as set forth in SEQ ID NO:1-3092.

In one embodiment, the at least one antigen is selected from the group consisting of an antigen as set forth in Table 3, and further wherein the disease or disorder is the disease or disorder associated with the antigen as set forth in Table 3.

In one embodiment, the at least one antigen is selected from the group consisting of an antigen as set forth in Table 4, and further wherein the disease or disorder is the disease or disorder associated with the antigen as set forth in Table 4.

In one embodiment, the disease or disorder is selected from the group consisting of an autoimmune disease or disorder, cancer, inflammatory disease or disorder, metabolic disease or disorder, neurodegenerative disease or disorder, organ tissue rejection, organ transplant rejection, or any combination thereof.

In one embodiment, the disease or disorder is selected from the group consisting of antineutrophil cytoplasmic antibody (ANCA)-associated vasculitis, autoimmune polyendocrinopathy candidiasis ecto-dermal dystrophy, antiphospholipid antibody syndrome, chronic inflammatory demyelinating polyradiculoneuropathy, cutaneous lupus erythematosus, COVID-19, drug-induced lupus, dermatomyositis, glomerulonephritis, a disease or disorder associated with kidney transplant, malaria, mixed connective tissue disease, myasthenia gravis, malignant melanoma, neuromyelitis optica, non-small cell lung cancer, pediatric autoimmune neuropsychiatric disorders associated with streptococcal infections, systemic lupus erythematosus, sjogren's syndrome, scleroderma, susac syndrome, undifferentiated connective tissue disease, and any combination thereof.

In one embodiment, the invention relates to a composition comprising an antigen selected from the group consisting of an antigen as set forth in SEQ ID NO:1-3092, or a fragment thereof, linked to a domain for endocytosis, degradation, or a combination thereof.

In one embodiment, the composition comprises an antigen selected from the group consisting of an antigen as set forth in Table 6 linked to a domain for endocytosis, degradation, or a combination thereof.

In one embodiment, the domain for endocytosis, degradation, or a combination thereof comprises an asialoglycoprotein receptor binding domain.

In one embodiment, the invention relates to a composition for targeting and destruction of at least one antibody-expressing cell comprising an antigen selected from the group consisting of an antigen as set forth in SEQ ID NO:1-3092, or a fragment thereof.

In one embodiment, the agent comprises a chimeric antigen receptor (CAR)

T cell expressing an antigen as set forth in SEQ ID NO:1-3092, or a fragment thereof. In one embodiment, the CAR T cell expresses an antigen selected from the group consisting of an antigen as set forth in Table 6.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts a REAP schematic. Simplified schematic of REAP. Antibodies are incubated with a genetically-barcoded yeast library displaying members of the exoproteome in 96-well microtiter plates. Antibody bound yeast are enriched by magnetic column-based sorting and enrichment is quantified by next-generation sequencing.

FIG. 2A and FIG. 2B depict exemplary experimental data demonstrating that REAP detects known targets of monoclonal antibodies. A panel of nine monoclonal antibodies were screened using REAP. FIG. 2A depicts a heatmap of results from REAP screen of nine monoclonal antibodies. Only relevant monoclonal antibody targets (gene names) are displayed. FIG. 2B depicts a representative sample from the screen. Monoclonal antibody target is highlighted in red and labelled. Background subtraction was performed by subtracting the score of a selection performed with beads and secondary alone. Scores below the average background level are not shown.

FIG. 3 depicts exemplary experimental data demonstrating a REAP screen of APECED patient samples. Reactivities uncovered in a REAP screen of 77 APECED patients and 20 healthy controls. Heatmap of REAP scores is depicted. Antigen groups were manually categorized.

FIG. 4 depicts exemplary experimental data demonstrating the concordance of REAP results and clinical anti-GIF autoantibody tests in APECED patients. Violin plot of GIF REAP scores in APECED samples stratified by intrinsic factor clinical autoantibody test results.

FIG. 5A and FIG. 5B depict exemplary experimental data demonstrating a REAP screen with serial dilutions of APECED 19 sample. REAP screen conducted with half log serial dilutions of APECED 19 IgG. Results are composed of technical duplicates. Only results from known autoantibody targets in APECED are depicted. Results are depicted as (FIG. 5A) the uncapped score of reactivities at various concentrations of APECED IgG and as (FIG. 5B) normalized, dose-response curves of reactivities where reactivities are measured by log 2 fold enrichment rather than score. Curves were fit using a sigmoidal 4 parameter logistic curve. Error bars represent standard deviation.

FIG. 6A and FIG. 6B depict exemplary experimental data demonstrating that REAP sensitivity can exceed that of ELISA. REAP (FIG. 6A) versus ELISA (FIG. 6B) dose-response curve comparison for APECED 19 autoantibodies against four proteins. Results are the averages of technical duplicates. Curves were fit using a sigmoidal 4 parameter logistic curve. Error bars represent standard deviation.

FIG. 7 depicts exemplary data demonstrating that REAP exhibits high reproducibility. Box plot of Log 2[fold enrichment] R2 coefficient of determination values between technical replicates of APECED patients screened in FIG. 3.

FIG. 8 depicts exemplary data demonstrating a REAP screen of SLE patient samples. Reactivities uncovered in a REAP screen of a cohort of 106 unique SLE patients spanning 155 samples and 20 healthy controls. Heatmap of REAP scores is depicted where each column is a unique patient. For patients with longitudinal samples, the maximum REAP score for each given reactivity is shown. Antigen groups were manually categorized. Patients are ordered from left to right by increasing SLEDAI score. White stars symbolize detection of a therapeutic antibody. Score was artificially capped at 7 to aid visualization.

FIG. 9A through FIG. 9E depict exemplary data demonstrating the biochemical and functional validation of novel SLE autoantibodies. FIG. 9A depicts an anti-PD-L2 pan-IgG ELISAs conducted with serial dilutions of SLE or control serum. FIG. 9D depicts an anti-IL-33 pan-IgG ELISAs conducted with serial dilutions of SLE or control serum. FIG. 9B depicts a schematic and FIG. 9C depicts results of PD-L2 blocking assay conducted with serial dilutions of serum from a control and the SLE patient in FIG. 9A. FIG. 9E depicts a schematic and FIG. 9F depicts results of IL-33 neutralization assay conducted with serial dilutions of IgG from a control and the SLE patient in FIG. 9D. All error bars in this figure represent standard deviation.

FIG. 10 depicts exemplary data demonstrating a REAP screen of immunotherapy-treated NSCLC patients. Reactivities uncovered in a REAP screen of 63 immunotherapy-treated non-small cell lung cancer (NSCLC) patients and 16 healthy donors. Of the 63 patients, longitudinal samples for 57 patients were available. Results are composed of technical duplicates. Longitudinal reactivities for each patient were collapsed and each reactivity was classified as increased, decreased, constant, therapeutic. The maximum reactivity for each protein in the healthy donor group is shown. Only proteins reactivities that developed or regressed in at least one patient are shown. Maximum score is defined as the maximum score of the protein at any time point. Score was not artificially capped. Increased responses are defined as those where the score of the protein increased by 2 or more at any time point after the first screened time point. Decreased responses are defined as those where the maximum score of the protein after the first screened time point was decreased by 2 or more from the initial score. Therapeutic responses are those where the patient was known to be receiving a therapeutic antibody against that protein. Patients are grouped by response to immunotherapy treatment.

FIG. 11 depicts exemplary data demonstrating that REAP scores can accurately reflect longitudinal changes in autoantibodies. Single point anti-OX40 isotype specific ELISAs conducted with serum from patient 3 at all available time points. REAP reactivity scores are depicted below with score artificially capped at 5. 1:100 serum dilutions were used. Results are averages of technical duplicates.

FIG. 12 depicts exemplary data demonstrating that unique sample clusters can be identified from REAP data. UMAP analysis of scores from previously described REAP screens of NSCLC, SLE, and UCTD patients. Each dot on the plot represents one patient sample at one time point. UMAP analysis was performed and visualized using a custom R script.

FIG. 13 depicts a REAP screen of scleroderma patients. Reactivities uncovered in a REAP screen of limited cutaneous systemic sclerosis, diffuse cutaneous systemic sclerosis patients, and healthy controls. Heatmap of REAP scores is depicted where each column is a unique patient. Antigen groups were manually categorized. Patient modified Rodnan skin score (mRSS), disease duration in months, and age in years is displayed below the heatmap.

FIG. 14 depicts immune-targeting autoantibody reactivities uncovered in COVID-19 patients. Heatmap of REAP scores for autoantibodies against immune-related antigens uncovered in a REAP screen of 194 COVID-19 patients. Antigen groups were manually categorized. Patients were stratified by disease severity. The negative group consists of control samples from uninfected healthcare workers. Abbreviations are as follows: asym: asymptomatic. Score was artificially capped at 7 to aid visualization.

FIG. 15 depicts tissue-targeting autoantibody reactivities uncovered in COVID-19 patients. Heatmap of REAP scores for autoantibodies against tissue-associated antigens uncovered in a REAP screen of COVID-19 patients. Antigen groups were manually categorized. Patients were stratified by disease severity. The negative group consists of control samples from uninfected healthcare workers. Abbreviations are as follows: asym—asymptomatic. Score was artificially capped at 7 to aid visualization.

FIG. 16 depicts a REAP screen of immunotherapy-treated melanoma patients. Heatmap of REAP score for autoantibodies identified in a screen of 222 CPI-treated melanoma patients and 62 healthy control samples. Score was artificially capped at 7 to aid visualization.

FIG. 17 depicts a REAP screen of kidney transplant patients. Heatmap of REAP score for immune-related autoantibodies identified in a screen of 108 kidney transplant patients with pre and post transplantation serum samples. Longitudinal reactivities for each patient were collapsed and each reactivity was classified as increased, decreased, stable. Patients are grouped by rejection and infection status after transplantation.

FIG. 18 depicts representative ELISA and LIPS validation data. FIG. 18A depicts an anti-OX40 autoantibody enzyme-linked immunosorbent assay (ELISA) titrations of NSCLC patient 3 serum at different time points. Reactivities were considered validated if average optical density (OD) at 1:100 serum dilution was at least 3 healthy donor standard deviations above the average 1:100 healthy donor serum dilution OD. Results are averages of technical duplicates. Error bars represent standard deviation. FIG. 18B depicts an anti-VEGFB autoantibody single-point luciferase immunoprecipitation systems (LIPS) with various NSCLC patient serum and healthy donor serum. 1:100 serum dilutions were used. Reactivities were considered validated if average relative light units (RLU) was at least 3 healthy donor standard deviations above the average healthy donor RLU.

FIG. 19 depicts an analysis of the sensitivity and specificity of REAP. An ROC curve based on orthogonal validation data of APECED and SLE screen reactivities is shown. Orthogonal validation was performed with LIPS or ELISA. For ELISA and LIPS, valid reactivities were defined as those 3 standard deviations above the healthy donor average for a given protein in each assay. ROC analysis was performed using 247 test pairs across 25 different proteins.

FIG. 20 depicts a schematic for targeted degradation of autoantigen-specific antibodies. Autoantigens are conjugated with a degradation moiety (e.g., a binding partner of the asialoglycoprotein receptor or other endocytosis promoting receptor). Once pathogenic autoantibodies bind to their respective autoantigen, they will be removed from circulation by endocytosis and degradation in the lysosome or other intracellular compartment.

FIG. 21 depicts a schematic for removal of autoantigen-specific B/plasma cells. CAR-T or CAR-NK cells are designed such that instead of an scFv targeting domain, instead, an autoantigen identified via REAP is used to direct CAR activity. Once CAR-T/NK cells bind to autoreactive B cells (that present B cell receptors/immunoglobulin on their plasma membrane), the CAR-T/NK cells will initiate cytotoxic programs that kill the corresponding autoreactive B/plasma cell.

FIG. 22 depicts schematic for autoantigen engineering to remove unwanted interaction with endogenous binding partners. To avoid unwanted interaction with their native binding partners, autoantigens are engineered to maintain autoantibody binding, but avoid interaction with their native binding partners. For example, a type I interferon engineered with decreasing binding to its receptors IFNAR1 and IFNAR2, but with maintained interaction with anti-interferon autoantibodies. The engineered autoantigens can subsequently be used for targeted autoantibody degradation (FIG. 20) or targeted B cell removal (FIG. 21).

FIG. 23 depicts a summary of validation data. ELISA or LIPS validation data for reactivities identified in REAP.

### DETAILED DESCRIPTION

The present invention relates to methods for the sensitive and high-throughput detection of various antibodies and targets thereof. For example, in one aspect, methods of the present invention identify target extracellular, secreted, and/or transmembrane proteins that specifically bind to various antibodies of interest. In another aspect, the present invention provides methods of preventing or treating diseases or disorders associated with antibodies and/or targets thereof detected via the high-throughput detection methods of the present invention. In various embodiments, the present invention provides methods of diagnosing, assessing prognosis, and assessing the effectiveness of treatments of diseases or disorders associated with antibodies detected via the high-throughput detection methods of the present invention. In another aspect, the present invention provides methods of predicting a response to a therapy. In another aspect, the present invention provides methods of alleviating toxicity of a cancer treatment.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “antibody,” as used herein, refers to an immunoglobulin molecule which is able to specifically bind to a specific epitope of an antigen. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab, Fab′, F(ab)2 and F(ab′)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen.

By the term “synthetic antibody” as used herein, is meant an antibody which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see for example EP-A-0239400 and EP-A-054951).

A “chimeric antibody” refers to a type of engineered antibody which contains a naturally-occurring variable region (light chain and heavy chains) derived from a donor antibody in association with light and heavy chain constant regions derived from an acceptor antibody.

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

By the term “recombinant antibody” as used herein, is meant an antibody which is generated using recombinant DNA technology, such as, for example, an antibody expressed by a bacteriophage or yeast cell expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

As used herein, “antigen-binding domain” means that part of the antibody, recombinant molecule, the fusion protein, or the immunoconjugate of the invention which recognizes the target or portions thereof.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequence or a partial nucleotide sequence encoding a protein that elicits an adaptive immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, tumor sample, cell, biological fluid, body fluid, blood, serum, plasma, tissue, or any combination thereof.

As used herein, the terms “targeting domain”, “targeting moiety”, or “targeting group” are used interchangeably and refer to all molecules capable of specifically binding to a particular target molecule and forming a bound complex as described above. Thus, the ligand and its corresponding target molecule form a specific binding pair.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The term “adjuvant” as used herein is defined as any molecule to enhance an antigen-specific adaptive immune response.

“Immunogen” refers to any substance introduced into the body in order to generate an immune response. That substance can a physical molecule, such as a protein, or can be encoded by a vector, such as DNA, mRNA, or a virus.

“Immune response,” as the term is used herein, means a process involving the activation and/or induction of an effector function in, by way of non-limiting examples, a T cell, B cell, natural killer (NK) cell, and/or an antigen-presenting cell (APC). Thus, an immune response, as would be understood by the skilled artisan, includes, but is not limited to, any detectable antigen-specific activation and/or induction of a helper T cell or cytotoxic T cell activity or response, production of antibodies, antigen presenting cell activity or infiltration, macrophage activity or infiltration, neutrophil activity or infiltration, and the like.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “DNA” as used herein is defined as deoxyribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

The term “RNA” as used herein is defined as ribonucleic acid.

The term “recombinant RNA” as used herein is defined as RNA produced by joining pieces of RNA from different sources.

As used herein, “conjugated” refers to covalent attachment of one molecule to a second molecule.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis. In various embodiments, the variant sequence is at least 99%, at least 98%, at least 97%, at least 96%, at least 95%, at least 94%, at least 93%, at least 92%, at least 91%, at least 90%, at least 89%, at least 88%, at least 87%, at least 86%, at least 85% identical to the reference sequence.

As used herein, the term “identical” refers to two or more sequences or subsequences which are the same.

In addition, the term “substantially identical,” as used herein, refers to two or more sequences which have a percentage of sequential units which are the same when compared and aligned for maximum correspondence over a comparison window, or designated region as measured using a comparison algorithm or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence.

As used herein, “fragment” is defined as at least a portion of a sequence. For example, in one embodiment, the term “fragment” refers to a portion of the variable region of the immunoglobulin molecule which binds to its target, i.e. the antigen binding region. Some of the constant region of the immunoglobulin may be included.

In the context of the present invention, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

In some instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In addition, the nucleotide sequence may contain modified nucleosides that are capable of being translated by translational machinery in a cell. Exemplary modified nucleosides are described elsewhere herein. For example, an mRNA where some or all of the uridines have been replaced with pseudouridine, 1-methyl psuedouridine, or another modified nucleoside, such as those described elsewhere herein. In some embodiments, the nucleotide sequence may contain a sequence where some or all cytodines are replaced with methylated cytidine, or another modified nucleoside, such as those described elsewhere herein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

The term “promoter” as used herein is defined as a DNA sequence recognized by the synthetic machinery of the cell, or introduced synthetic machinery, required to initiate the specific transcription of a polynucleotide sequence. By way of one non-limiting example, a promoter that is recognized by bacteriophage RNA polymerase and is used to generate the mRNA by in vitro transcription.

The terms “patient,” “subject,” “individual,” and the like are used interchangeably herein, and refer to any animal, or cells thereof whether in vitro or in situ, amenable to the methods described herein. In some non-limiting embodiments, the patient, subject or individual is a human. In various embodiments, the subject is a human subject, and may be of any race, sex, and age.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including, but not limited to, the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), soft tissues (e.g., muscle, fat, etc.), and hematopoietic system (e.g., lymphoma, myeloma, leukemia, acute lymphocytic leukemia, chronic lymphocytic leukemia, acute myeloid leukemia, chronic myeloid leukemia, etc.).

A disease or disorder is “alleviated” if the severity of at least one sign or symptom of the disease or disorder, the frequency with which such a sign or symptom is experienced by a patient, or both, is reduced.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as, a human.

The term “inhibit,” as used herein, means to suppress or block an activity or function by at least about ten percent relative to a control value. In various embodiments, the activity is suppressed or blocked by at least 50% compared to a comparator value, or by at least 55%, or by at least 60%, or by at least 65%, or by at least 70%, or by at least 75%, or by at least 80%, or by at least 85%, or by at least 90%, or by at least 95%.

As used herein, the term “diagnosis” refers to the determination of the presence of a disease or disorder. In various embodiments of the present invention, methods for making a diagnosis are provided which permit determination of the presence of a particular disease or disorder.

To “treat” a disease as the term is used herein, means to reduce the frequency and/or severity of at least one sign or symptom of a disease or disorder experienced by a subject.

An “effective amount” as used herein, means an amount which provides a therapeutic or prophylactic benefit.

The term “therapeutic” as used herein means a treatment and/or prophylaxis. A therapeutic effect is obtained by suppression, diminution, remission, prevention, or eradication of at least one sign or symptom of a disease or disorder.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

The term “solvate” in accordance with this invention should be understood as meaning any form of the active compound in accordance with the invention in which said compound is bonded by a non-covalent bond to another molecule (normally a polar solvent), including especially hydrates and alcoholates.

As used herein, an “immunoassay” refers to any binding assay that uses an antibody capable of binding specifically to a target molecule to detect and quantify the target molecule.

The term “amplification” refers to the operation by which the number of copies of a target nucleotide sequence present in a sample is multiplied.

The term “next generation sequencing” herein refers to sequencing methods that allow for massively parallel sequencing of clonally amplified molecules and of single nucleic acid molecules. Next generation sequencing is synonymous with “massively parallel sequencing” for most purposes. Non-limiting examples of next generation sequencing include sequencing-by-synthesis using reversible dye terminators, and sequencing-by-ligation.

Assays for amplification of the known sequence are also disclosed. For example primers for PCR may be designed to amplify regions of the sequence. For RNA, a first reverse transcriptase step may be used to generate double stranded DNA from the single stranded RNA. The array may be designed to detect sequences from an entire genome; or one or more regions of a genome, for example, selected regions of a genome such as those coding for a protein or RNA of interest; or a conserved region from multiple genomes; or multiple genomes, arrays and methods of genetic analysis using arrays is described in Cutler, et al., 2001, Genome Res. 11(11): 1913-1925 and Warrington, et al., 2002, Hum Mutat 19:402-409 and in US Patent Pub No 20030124539, each of which is incorporated herein by reference in its entirety.

“Instructional material,” as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains one or more components of the invention or be shipped together with a container that contains the one or more components of the invention. Alternatively, the instructional material may be shipped separately from the container with the intention that the recipient uses the instructional material and the components cooperatively.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to methods of detecting various antibodies and targets thereof. In one aspect, the present invention provides methods of identifying a target extracellular, secreted, and/or transmembrane protein that specifically binds to an antibody of interest. In another aspect, the present invention provides methods of preventing or treating diseases or disorders associated with antibodies and/or a targets thereof identified via the methods of the present invention. In another aspect, the present invention provides methods of diagnosing, assessing prognosis, or assessing the effectiveness of treatments of diseases or disorders associated with antibodies and/or a targets thereof identified via the methods of the present invention. In another aspect, the present invention provides methods of predicting a response to a therapy. In another aspect, the present invention provides methods of alleviating toxicity of a cancer treatment.

### Methods of Identifying Antibodies and Targets Thereof

The present invention relates, in part, to methods of identifying antibodies or binding partners thereof. In one aspect, the method comprises identifying an antigenic polypeptide that specifically binds to an antibody of interest. In one aspect, the method comprises identifying novel antibody-antigen interactions.

In one embodiment, the invention relates to a screening method for antigen antibody interactions, wherein the method comprises generating a display library of polypeptides that are then screened for interactions with at least one antibody. Therefore, in one embodiment, the invention relates to a polypeptide display library and methods of use thereof for screening for antigen-antibody interactions.

### Display Library

In various embodiments, the invention relates to methods of screening using a cellular display library. In some embodiments, the cellular display library comprises a plurality of cells, wherein together the plurality of cells displays at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000 or more than 10,000 different polypeptides on the surface of the cells. In one embodiment, the plurality of cells of the display library display proteins or polypeptides of the secretome, representing a plurality of secreted proteins, the exoproteome, representing a plurality of extracellular proteins, or a combination thereof. In one embodiment, the plurality of cells of the display library display a combination of at least 10, 20, 30, 40, 50, 60, 70, 80, 90, 100, 200, 300, 400, 500, 600, 700, 800, 900, 1000, 2000, 3000, 4000, 5000, 6000, or more than 6,000 extracellular and secreted polypeptides or proteins. In one embodiment, together the plurality of cells in the display library, display each of the polypeptide amino acid sequences set forth in SEQ ID NO:1-3092.

In some embodiments, the polypetides for display are fusion proteins with polypeptides that allow expression and exposure on a cell or particle surface. In one embodiment, nucleic acids encoding the molecules can be cloned into a display vector. The vector is designed to express the fusion molecules and display the encoded antigen on the outer surface of a display cell or partilce containing the vector. For example, antigens can be expressed as fusion proteins with a phage coat protein from the outer surface of the phage. In some embodiments, the polypeptides for display are IgGI Fc fusion molecules. Thereafter, the display cells or particles can be screened for antibody reactivities with the displayed antigens.

Thus, in various embodiments, the present invention also includes a vector in which a nucleotide sequence encoding a polypeptide for display of the present invention is inserted. The art is replete with suitable vectors that are useful in the present invention.

In brief summary, the expression of a nucleotide construct is typically achieved by operably linking a nucleic acid sequence comprising a promoter to a nucleic acid sequence encoding an antigen or portions thereof, and incorporating the construct into an expression vector. In one embodiment, the vectors to be used are suitable for replication and, optionally, integration in eukaryotic cells. Typical vectors contain transcription and translation terminators, initiation sequences, and other regulatory sequences useful for regulation of the expression of the desired nucleic acid sequence.

The recombinant nucleotide sequences encoding an antigen for display of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method

In certain embodiments, the vector also includes conventional control elements which are operably linked to the encoded antigen sequence in a manner which permits its transcription, translation and/or expression in a cell transfected with the plasmid vector or infected with the virus produced by the invention. As used herein, “operably linked” sequences include both expression control sequences that are contiguous with the reporter molecule and expression control sequences that act in trans or at a distance to control the expression of the reporter molecule. Expression control sequences include appropriate transcription initiation, termination, and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. All of the above-described functional elements can be used in any combination to produce a suitable display vector.

In one embodiment, a display vector comprises an origin of replication capable of initiating DNA synthesis in a suitable host cell. In one embodiment, the origin of replication is selected based on the type of host cell. For instance, it can be eukaryotic (e.g., yeast) or prokaryotic (e.g., bacterial) or a suitable viral origin of replication may be used.

In one embodiment, a display vector comprises a selection marker gene to facilitate identification and selection of expressing cells from the population of cells sought to be transfected or infected through viral vectors. In other aspects, the selectable marker may be carried on a separate piece of DNA and used in a co-transfection procedure. Selectable marker genes may be flanked with appropriate regulatory sequences to enable expression in the host cells.

A selection marker sequence can be used to eliminate host cells in which the display vector has not been properly transfected. A selection marker sequence can be a positive selection marker or negative selection marker. Positive selection markers permit the selection for cells in which the gene product of the marker is expressed. This generally comprises contacting cells with an appropriate agent that, but for the expression of the positive selection marker, kills or otherwise selects against the cells.

Examples of selection markers also include, but are not limited to, proteins conferring resistance to compounds such as antibiotics, proteins conferring the ability to grow on selected substrates, proteins that produce detectable signals such as luminescence, catalytic RNAs and antisense RNAs. A wide variety of such markers are known and available, including, for example, a Zeocin™ resistance marker, a blasticidin resistance marker, a neomycin resistance (neo) marker (Southern & Berg, J. Mol. Appl. Genet. 1: 327-41 (1982)), a puromycin (puro) resistance marker; a hygromycin resistance (hyg) marker (Te Riele et al., Nature 348:649-651 (1990)), thymidine kinase (tk), hypoxanthine phosphoribosyltransferase (hprt), and the bacterial guanine/xanthine phosphoribosyltransferase (gpt), which permits growth on MAX (mycophenolic acid, adenine, and xanthine) medium. See Song et al., Proc. Nat'l Acad. Sci. U.S.A. 84:6820-6824 (1987). Other selection markers include histidinol-dehydrogenase, chloramphenicol-acetyl transferase (CAT), dihydrofolate reductase (DHFR), β-galactosyltransferase and fluorescent proteins such as GFP.

Expression of a fluorescent protein can be detected using a fluorescent activated cell sorter (FACS). Expression of 3-galactosyltransferase also can be sorted by FACS, coupled with staining of living cells with a suitable substrate for β-galactosidase. A selection marker also may be a cell-substrate adhesion molecule, such as integrins, which normally are not expressed by the host cell. In one embodiment, the cell selection marker is of mammalian origin, for example, thymidine kinase, aminoglycoside phosphotransferase, asparagine synthetase, adenosine deaminase or metallothionien. In one embodiment, the cell selection marker can be neomycin phosphotransferase, hygromycin phosphotransferase or puromycin phosphotransferase, which confer resistance to G418, hygromycin and puromycin, respectively.

Suitable prokaryotic and/or bacterial selection markers include proteins providing resistance to antibiotics, such as kanamycin, tetracycline, and ampicillin. In one embodiment, a bacterial selection marker includes a protein capable of conferring selectable traits to both a prokaryotic host cell and a mammalian target cell.

Negative selection markers permit the selection against cells in which the gene product of the marker is expressed. In some embodiments, the presence of appropriate agents causes cells that express “negative selection markers” to be killed or otherwise selected against. Alternatively, the expression of negative selection markers alone kills or selects against the cells.

Such negative selection markers include a polypeptide or a polynucleotide that, upon expression in a cell, allows for negative selection of the cell. Illustrative of suitable negative selection markers are (i) herpes simplex virusthymidine kinase (HSV-TK) marker, for negative selection in the presence of any of the nucleoside analogs acyclovir, gancyclovir, and 5-fluoroiodoamino-Uracil (FIAU), (ii) various toxin proteins such as the diphtheria toxin, the tetanus toxin, the cholera toxin and the pertussis toxin, (iii) hypoxanthine-guanine phosphoribosyl transferase (HPRT), for negative selection in the presence of 6-thioguanine, (iv) activators of apoptosis, or programmed cell death, such as the bc12-binding protein (BAX), (v) the cytidine deaminase (codA) gene of E. coli, and (vi) phosphotidyl choline phospholipase D. In one embodiment, the negative selection marker requires host genotype modification (e.g. ccdB, tolC, thyA, rpsl and thymidine kinases.)

In accordance with the present invention, the selection marker usually is selected based on the type of the cell undergoing selection. For instance, it can be eukaryotic (e.g., yeast), prokaryotic (e.g., bacterial) or viral. In such an embodiment, the selection marker sequence is operably linked to a promoter that is suited for that type of cell.

In one embodiment, the invention provides a plurality of at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000 or more than 10,000 recombinant nucleic acid molecules, wherein together the plurality of recombinant nucleic acid molecules encode at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000 or more than 10,000 different polypeptides for display in a cell display library. In one embodiment, the plurality of cells of the display library display proteins or polypeptides of the secretome, representing a plurality of secreted proteins, the exoproteome, representing a plurality of extracellular proteins, or a combination thereof. In one embodiment, together the plurality of recombinant nucleic acid molecules encodes at least 10, 20, 30, 40, 50, 60, 70, 80, 90, 100, 200, 300, 400, 500, 600, 700, 800, 900, 1000, 2000, 3000, 4000, 5000, 6000, or more than 6,000 extracellular and secreted polypeptides or proteins. In one embodiment, together the plurality of recombinant nucleic acid molecules encodes each of the polypeptide amino acid sequences set forth in SEQ ID NO:1-3092. In one embodiment, together the plurality of recombinant nucleic acid molecules comprises each of the nucleotide sequences set forth in SEQ ID NO:3093-6185.

In one embodiment, each of the recombinant nucleic acid molecules in the plurality of recombinant nucleic acid molecules encodes a polypeptide sequence for expression on a cell surface, and further comprises a unique nucleotide barcode sequence, which is then associated with the encoded polypeptide sequence. In various embodiments, the unique barcode sequence comprises a nucleotide sequence of at least 4, 5, 6, 7, 8, 9, 10, 11, 12, 13, 14, 15, 16, 17, 18, 19, 20 or more than 20 nucleotides which is non-redundant within the recombinant nucleotide sequences included in the library.

In some embodiments, the invention relates to methods of generating a display library for expression of a plurality of extracellular or secreted proteins on the surface of a plurality of cells. In some embodiments, the method comprises obtaining or generating a library of barcoded nucleic acid molecules, wherein each nucleic acid molecule comprises i) a nucleotide sequence encoding a polypeptide for display on the surface of a cell; and ii) a unique nucleotide barcode sequence; and introducing the plurality of recombinant nucleic acid molecules into a system for expression and/or display of the recombinant nucleic acid molecules. Display systems that can be used for expression and/or display of the recombinant nucleic acid library of the invention include, but are not limited to, phage display, mRNA display, ribosome display, yeast display, mammalian cell display, and the like.

Any method known in the art for introducing nucleic acid sequences into cells can be used to generate the display library of the invention. Exemplary methods of introducing nucleic acid molecules into cells include, but are not limited to, electroporation, cell squeezing, sonoporation, optical transfection, protoplast fusion, impalefection, hydrodynamic delivery, fusion, magnetofection, particle bombardment, nucleofection, heat shock, lipofection, viral transduction, nonviral transfection, lithium acetate/PEG chemical transformation, or any combination thereof.

In one embodiment, the method comprises generating a library of cells for displaying polypeptides which function as epitopes for antigen binding. Thus, in one embodiment, the method comprises generating a library of cells, wherein the library comprises cells comprising barcode-labeled nucleic acid sequences, wherein the barcode-labeled nucleic acid sequences encode polypeptides which function as epitopes for antigen binding.

### Screening Methods

In some embodiments, the invention provides methods for screening a display library comprising a plurality of proteins or polypeptides of the secretome, representing a plurality of secreted proteins, the exoproteome, representing a plurality of extracellular proteins, or a combination thereof, to identify those proteins or polypeptides which interact with at least one antibody. In one embodiment, the methods comprise contacting the plurality of displayed proteins or polypeptides with a sample comprising at least one antibody.

In one embodiment, the method comprises the step of contacting a library of display cells with a sample comprising at least one antibody, thus generating one or more antibody-bound cells. In various embodiments, the antibody is a purified antibody. In one embodiment, the antibody is purified from a biological sample. Biological samples may be of any biological tissue or fluid. Frequently the sample will be a “clinical sample” which is a sample derived from a subject. The biological sample may contain any biological material suitable for detecting the desired antibodies or targets thereof, and may comprise cellular and/or non-cellular material obtained from the subject. A biological sample can be obtained by appropriate methods, such as, by way of examples, blood draw, fluid draw, biopsy, or surgical resection. Examples of such samples include but are not limited to serum, blood, lymph, urine, gastrointestinal fluid, cerebrospinal fluid, semen, and samples from biopsies. Samples that are liquid in nature are referred to herein as “bodily fluids.” Body samples may be obtained from a subject by a variety of techniques including, for example, by scraping or swabbing an area or by using a needle to aspirate bodily fluids. Methods for collecting various body samples are well known in the art. Frequently, a sample will be a “clinical sample,” i.e., a sample derived from a subject. Such samples include, but are not limited to, bodily fluids which may or may not contain cells, e.g., blood (e.g., whole blood, serum or plasma), urine, saliva, cerebrospinal fluid, or fine needle biopsy samples, tissue sample obtained during surgical resection, and archival samples with known diagnosis, treatment and/or outcome history.

In one embodiment, the method comprises contacting the display cells with at least one antibody purified from a biological sample. In one embodiment, the antibody is purified from a biological sample by affinity purification. In some embodiment, the antibody is purified from a biological sample by affinity purification of the desired antibody isotype (e.g., IgG, IgA, IgE, etc.). In some embodiments, the antibody is purified from a biological sample using any method known in the art for the purification of specific antibodies from a biological sample. For example, in one embodiment, the antibody is purified from a serum by affinity purification. In some embodiments, the antibody is purified by a high-throughput and efficient method for antibody isolation from human serum or plasma. In one embodiment, the method comprises an affinity purification of the desired antibody isotype (IgG, IgA, IgE, etc.) in 96-well microtiter plates.

In one embodiment, the sample comprising at least one antibody is purified by removing at least one human serum component. In one embodiment, the sample comprising at least one antibody is purified by removing at least one antibody that may bind a display cell and interfere with a downstream selection procedure. For example, in one embodiment, the sample comprising at least one antibody of interest is purified by contacting the sample with at least one control cell or particle comprising an empty display vector, and removing any species that bind to the control cell or particle comprising the empty display vector from the sample.

In one embodiment, the sample goes through a two-step purification process which involves both a) purification or selection of the specific antibody isotype of interest using an affinity purification for the isotype of interest (e.g., IgG, IgA, IgE, etc.), and b) elimination of human serum components and display cell or particle-reactive antibodies that may bind the display cell or particle and interfere with downstream selection procedures by contacting the purified sample with at least one control cell or particle comprising an empty display vector, and removing any species that bind to the control cell or particle.

In one embodiment, the biological sample is a healthy, normal or control sample. In some embodiments, a healthy, normal or control sample is a sample from a subject who has not been diagnosed with a disease or disorder. In one embodiment, the biological sample is obtained from a subject having a disease or disorder. Thus, in some embodiments, the biological sample comprises at least one antibody associated with a disease or disorder. Exemplary diseases and disorders include, but are not limited to, an autoimmune disease or disorder, cancer, inflammatory disease or disorder, metabolic disease or disorder, neurodegenerative disease or disorder, organ tissue rejection, organ transplant rejection, or any combination thereof. In one embodiment, the antibody is an autoantibody.

In some embodiments, the sample is from a subject who shows good prognosis of a disease or disorder, has reduced symptoms associated with a disease or disorder, or has a mild form of a disease or disorder. In such an embodiment, the methods of the invention serve to identify therapeutic antibodies or antibody-antigen interactions for the treatment of the disease or disorder. In some embodiments, the disease or disorder is selected from antineutrophil cytoplasmic antibody (ANCA)-associated vasculitis, autoimmune polyendocrinopathy candidiasis ecto-dermal dystrophy, antiphospholipid antibody syndrome, chronic inflammatory demyelinating polyradiculoneuropathy, cutaneous lupus erythematosus, COVID-19, drug-induced lupus, dermatomyositis, glomerulonephritis, a disease or disorder associated with kidney transplant, malaria, mixed connective tissue disease, myasthenia gravis, malignant melanoma, neuromyelitis optica, non-small cell lung cancer, pediatric autoimmune neuropsychiatric disorders associated with streptococcal infections, systemic lupus erythematosus, sjogren's syndrome, scleroderma, susac syndrome, undifferentiated connective tissue disease, or any combination thereof, and therefore the antibody is a therapeutic antibody for the treatment of antineutrophil cytoplasmic antibody (ANCA)-associated vasculitis, autoimmune polyendocrinopathy candidiasis ecto-dermal dystrophy, antiphospholipid antibody syndrome, chronic inflammatory demyelinating polyradiculoneuropathy, cutaneous lupus erythematosus, COVID-19, drug-induced lupus, dermatomyositis, glomerulonephritis, a disease or disorder associated with kidney transplant, malaria, mixed connective tissue disease, myasthenia gravis, malignant melanoma, neuromyelitis optica, non-small cell lung cancer, pediatric autoimmune neuropsychiatric disorders associated with streptococcal infections, systemic lupus erythematosus, sjogren's syndrome, scleroderma, susac syndrome, undifferentiated connective tissue disease, or any combination thereof.

In some embodiments, the sample is from a subject who shows poor prognosis of a disease or disorder, has increased symptoms associated with a disease or disorder, or has a severe form of a disease or disorder. In such an embodiment, the methods of the invention serve to identify antibodies or antibody-antigen interactions that are therapeutic targets for the treatment or prevention of a disease or disorder. In some embodiments, the disease or disorder is selected from antineutrophil cytoplasmic antibody (ANCA)-associated vasculitis, autoimmune polyendocrinopathy candidiasis ecto-dermal dystrophy, antiphospholipid antibody syndrome, chronic inflammatory demyelinating polyradiculoneuropathy, cutaneous lupus erythematosus, COVID-19, drug-induced lupus, dermatomyositis, glomerulonephritis, a disease or disorder associated with kidney transplant, malaria, mixed connective tissue disease, myasthenia gravis, malignant melanoma, neuromyelitis optica, non-small cell lung cancer, pediatric autoimmune neuropsychiatric disorders associated with streptococcal infections, systemic lupus erythematosus, sjogren's syndrome, scleroderma, susac syndrome, undifferentiated connective tissue disease, or any combination thereof, and therefore the antibody is a therapeutic target for the treatment of antineutrophil cytoplasmic antibody (ANCA)-associated vasculitis, autoimmune polyendocrinopathy candidiasis ecto-dermal dystrophy, antiphospholipid antibody syndrome, chronic inflammatory demyelinating polyradiculoneuropathy, cutaneous lupus erythematosus, COVID-19, drug-induced lupus, dermatomyositis, glomerulonephritis, a disease or disorder associated with kidney transplant, malaria, mixed connective tissue disease, myasthenia gravis, malignant melanoma, neuromyelitis optica, non-small cell lung cancer, pediatric autoimmune neuropsychiatric disorders associated with streptococcal infections, systemic lupus erythematosus, sjogren's syndrome, scleroderma, susac syndrome, undifferentiated connective tissue disease, or any combination thereof.

In one embodiment, the screening method further comprises a step of isolating or purifying one or more antibody-bound display cell of the invention. Any method known in the art for separating or purifying an antibody-bound display cell can be used including, but not limited to, magnetic cell separation, fluorescent cell separation, affinity purification, bead based cell separation, column separation, or any combination thereof.

In some embodiments, the methods of the invention comprise a step of staining cells. Examples of stains include, but are not limited to: fluorescent dyes, propidium iodine, ethidium homodimer III, thiazole orange, acridine orange, Bismarck brown, carmine, coomassie blue, cresyl violet, crystal violet, DAPI, eosin, ethidium bromide, acid fuchsine, haematoxylin, Hoechst stains, iodine, malachite green, methyl green, methylene blue, neutral red, nile blue, nile red, osmium tetraoxide, rhodamine, safranine, biotin, or any combination thereof.

In some embodiments, the methods of the invention comprise a step of identifying cells bound to an antibody by contacting the library of cells with a secondary immunoglobulin binding molecule for recognition of a primary antibody isotype of interest. For example, in some embodiments, the secondary immunoglobulin binding molecule is an antibody, nanobody, VHH antibody, monobody, knottin, anticalin, peptide, cyclic peptide, aptamer, designed ankyrin repeat protein (DARPin), or any combination thereof.

In one embodiment, a cell bound by an antibody of interest is identified using any appropriate sorting or selection method. Exemplary sorting and selection methods include, but are not limited to, biotinylated labeled anti-immunoglobulin antibody, fluorescence activated cell sorting (FACS), fluorescently labeled anti-immunoglobulin antibody, magnetic bead-based selection, magnetic bead conjugated to an anti-immunoglobulin antibody, or any combination thereof.

In one embodiment, the method comprises isolating at least one antibody-bound cell or particle from a mixture. In one embodiment, the method comprises isolating at least one antibody-bound cell or particle from at least one non-antibody-bound cell or particle. In one embodiment, the isolating at least one antibody-bound cell or particle comprises washing to remove at least one non-specific binder, centrifuging, cell separation, or any combination thereof. In one embodiment, the isolating at least one antibody-bound cell or particle comprises washing to remove at least one non-specific binder, centrifuging, magnetic cell separation, fluorescent cell separation, high-throughput selection process based on 96-well magnetic columns, or any combination thereof. In one embodiment, the magnetic cell separation comprises magnetic columns for capturing cells. In one embodiment, the magnetic cell separation comprises magnetic columns for capturing antibody-bound cell or particles. In one embodiment, the fluorescent cell separation comprises fluorescence activated cell sorting (FACS). In some embodiments, the high-throughput selection process based on 96-well magnetic columns comprises cell or particle library selections, 96-well magnetic columns, large magnetic columns, FACS, washing, centrifuging, or any combination thereof.

In one embodiment, the method comprises enriching at least one antibody-bound cell or particle by magnetic column-based sorting. In one embodiment, the method comprises amplifying the barcoded recombinant nucleic acid molecule of the antibody-bound cell or particle. In one embodiment, the enrichment is quantified by sequencing. In one embodiment, the enrichment is quantified by next generation sequencing.

### High Throughput Identification of Autoantibody Reactivities

In one embodiment, the screening methods of the invention include methods of high throughput identification of antigen or autoantigen interactions with antibodies or autoantibodies (reactivities.) In some embodiments the screening methods of the invention include of high throughput identification of antibody or autoantibody reactivities include methods of contacting a sample comprising at least one antibody or autoantibody with a display library of the invention, isolating those cells or particles expressing polypeptides which interact with at least one antibody or autoantibody, and identifying the expressed antigen or autoantigen on at the isolated cells or particles.

In one embodiment, the screening methods of the invention include a step of isolating and sequencing the barcoded nucleic acid molecules from a plurality of antibody-bound cells or particles. In one embodiment, a polypeptide is identified to be an antigen or autoantigen of at least one antibody in the sample based on detection of an increased or enriched level of the associated encoding nucleotide sequence or associated barcode in sequencing data over an established threshold level. In some embodiments, the threshold level is a predetermined threshold level, a statistically determined threshold, a threshold level determined using z-scores, or an established cut-point.

In various embodiments of the methods of the invention, the level of the nucleic acid sequence barcode is determined to be increased when the number of associated sequencing reads from Next-gen sequencing data corresponding to the barcode is increased or enriched relative to a reference value or statistically determined cut-off value. In some embodiments, the level of the nucleic acid sequence barcode is determined to be increased when the number of associated sequencing reads Next-gen sequencing data corresponding to the barcode is increased or enriched by at least 0.01 fold, at least 0.05 fold, at least 0.07 fold, at least 0.076 fold, at least 0.1 fold, at least 0.18 fold, at least 0.19 fold, at least 0.3 fold, at least 0.36 fold, at least 0.37 fold, at least 0.38 fold, at least 0.4 fold, at least 0.43 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 16 fold, at least 16.3 fold, at least 16.31 fold, at least 20 fold, at least 25 fold, at least 26 fold, at least 26.7 fold, at least 26.72 fold, at least 30 fold, at least 40 fold, at least 50 fold, at least 75 fold, at least 100 fold, at least 192 fold, at least 192.4 fold, at least 192.44 fold, at least 200 fold, at least 250 fold, at least 500 fold, or at least 1000 fold, or at least 10000 fold, when compared with a comparator (e.g., a statistically determined threshold level or pre-determined cut-off).

In one embodiment, an increased level of a barcode nucleic acid sequence provides an indication that an associated encoded polypeptide serves as a target for antibody binding, or an antigen. In one embodiment, an increased level of a barcode nucleic acid sequence provides an indication that an associated encoded polypeptide serves as a target for autoantibody binding, or an autoantigen. In various embodiments, the associated encoded polypeptide is an extracellular protein, transmembrane protein, secreted protein, or any combination thereof. In one embodiment, the associated encoded polypeptide is selected from those provided in Table 1, or a fragment thereof For example, in some embodiments, the associated encoded polypeptide is BMPR2, BTN1A1, BTNL8, C1QTNF4, C6, CCL11, CCL15, CCL17, CCL2, CCL22, CCL24, CCL4L1, CD207, CD300E, CD3D, CD44, CD74, CD81, CDH19, CNTN5, COLEC12, CSPG5, CX3CL1, CXCL1, CXCL13, CXCL2, CXCL3, EDIL3, EPYC, EREG, FGF10, FGF21, FGF23, FGF7, FGFBP3, FGFRL1, IFNA13, IFNA14, IFNA17, IFNA2, IFNA5, IFNA6, IFNA8, IFNB1, IFNL2, IFNW1, IGF2, IGFBPL1, IGSF4B, IL15RA, IL16, IL17A, IL17F, IL17F, IL18RAP, IL19, IL1A, IL1F9, IL1RAP, IL20RB, IL22, IL22RA2, IL28B, IL29, IL33, IL34, IL4, IL4R, IL5, IL6, IL6R, ITGA5, JCHAIN, LAG3, LGR6, LIF, LRP11, LRRC3B, LRRC4, LRTM2, LY6G6D, LY6H, MADCAM1, MPZL3, MUC21, NGFR, NOTCH2NL, NTRK3, PDCD1LG2, PDGFB, PGLYRP1, REGlA, REG1B, REG4, RTN4RL1, SCARA3, SDC1, SDC4, STIM2, TGFA, TMEM149, TNF, TNFRSF10C, TNFRSF10D, TNFRSF19L, TNFRSF6, TRAILR4, TREM2, TREML1, TSLP, TSPAN2, TYRO3, VEGFB, VSIG4, VSTM2A, or any combination thereof.

In one embodiment, the method comprises identifying antibody reactivities based on quantitative next generation sequencing data. In one embodiment, the next generation sequencing can determine the total enrichment of antibody target proteins after selection, how many “antibody target protein clones” were enriched, or a combination thereof.

In one embodiment, the method comprises an incorporation of clonal enrichment into data analysis to eliminate false positive enrichments. In one embodiment, the method comprises an incorporation of clonal enrichment into data analysis to expedite identification of genuine autoantibody reactivities in samples. Thus, in one embodiment, the method comprises quantifying clonal enrichment for identification of antibody reactivities, elimination of non-specific enrichment of antibody target proteins (e.g., polyreactive cell or particle clones), elimination of stochastic variations in library distribution, or any combination thereof. In one embodiment, the clonal enrichment is a fraction of clones that were enriched above a set cutoff.

In one embodiment, the methods described herein can utilize next-generation sequencing technologies that allow multiple samples to be sequenced individually as genomic molecules (i.e., singleplex sequencing) or as pooled samples comprising indexed genomic molecules (e.g., multiplex sequencing) on a single sequencing run. These methods can generate up to several hundred million reads of DNA sequences. In various embodiments, the sequences of nucleic acid sequence barcodes can be determined using, for example, the next generation sequencing technologies described herein. In various embodiments, analysis of the massive amount of sequence data obtained using next-generation sequencing can be performed using one or more processors as described herein.

In some embodiments, the nucleic acid product can be sequenced by next generation sequencing methods. In some embodiments, the next generation sequencing method comprises a method selected from the group consisting of Ion Torrent, Illumina, SOLiD, 454; Massively Parallel Signature Sequencing, solid phase reversible dye terminator sequencing; and DNA nanoball sequencing may be included. In some embodiments, the first and second sequencing primers are compatible with the selected next generation sequencing method.

In some embodiments, sequencing can be performed by next generation sequencing methods. As used herein, “next generation sequencing” refers to the speeds that were possible with conventional sequencing methods (e.g., Sanger sequencing) by reading thousands of millions of sequencing reactions simultaneously. Means an oligonucleotide sequencing technique that has the ability to sequence oligonucleotides at a greater rate. Non-limiting examples of next generation sequencing methods/platforms include Massively Parallel Signature Sequencing (Lynx Therapeutics); pyrophosphate sequencing/454; 454 Life Sciences/Roche Diagnostics; Solid Phase Reversible Dye Terminator Sequencing (Solexa/illumina): SOLiD technology (Applied Biosystems); ion semiconductor sequencing (ION Torrent.); DNA nanoball sequencing (Complete Genomics); and technologies available from Pacific Biosciences, Intelligen Bio-systems, Oxford Nanopore Technologies, and Helicos Biosciences. In some embodiments, the sequencing primer may comprise a moiety that is compatible with the selected next generation sequencing method.

Next generation sequencing techniques and related sequencing primer constraints and design parameters are well known in the art (e.g., Shendure et al., 2008, Nature, 26:1135-1145; Mardis, 2007, Trends in Genetics, 24:133-141; Su et al., 2011, Expert. Rev. Mol. Diagn., 11:333-43; Zhang et al., 2011, J. Genet. Genomics, 38:95-109; Nyren P et al. 1993, Anal. Biochem., 208:17175; Bentley et al., 2006, Curr. Opin. Genet. Dev., 16:545-552; Strausberg et al., 2008, Drug Disc. Today, 13:569-577; U.S. Pat. Nos. 7,282,337; 7,279,563; 7,226,720; 7,220,549; 7,169,560; U. S. Patent Application Publication No. 20070070349; U.S. Pat. Nos. 6,818,395; 6,911,345; U.S. Patent Application Publication No. 2006/0252077; No. 2007/0070349).

Several targeted next generation sequencing methods are described in the literature (for review see e.g., Teer and Mullikin, 2010, Human Mol. Genet. 19:R145-151), all of which can be used in conjunction with the present invention. Many of these methods (described e.g. as genome capture, genome partitioning, genome enrichment etc.) use hybridization techniques and include array-based (e.g., Hodges et al., 2007, Nat. Genet., 39:1522-1527) and liquid based (e.g., Choi et al., 2009, Proc. Natl. Acad. Sci USA, 106:19096-19101) hybridization approaches. Commercial kits for DNA sample preparation are also available: for example, Illumina Inc. (San Diego, California) offers the TruSeq™ DNA Sample Preparation Kit and the Exome Enrichment Kit TruSeq™ Exome Enrichment Kit.

There are many methods known in the art for the detection, identification, and quantification of specific nucleic acid sequences (e.g., nucleic acid sequence barcodes) and new methods are continually reported. A great majority of the known specific nucleic acid detection, identification, and quantification methods utilize nucleic acid probes in specific hybridization reactions. Preferably, the detection of hybridization to the duplex form is a Southern blot technique. In the Southern blot technique, a nucleic acid sample is separated in an agarose gel based on size (molecular weight) and affixed to a membrane, denatured, and exposed to (admixed with) the labeled nucleic acid probe under hybridizing conditions. If the labeled nucleic acid probe forms a hybrid with the nucleic acid on the blot, the label is bound to the membrane.

In the Southern blot, the nucleic acid probe is preferably labeled with a tag. That tag can be a radioactive isotope, a fluorescent dye or the other well-known materials. Another type of process for the specific detection of nucleic acids in a biological sample known in the art are the hybridization methods as exemplified by U.S. Pat. Nos. 6,159,693 and 6,270,974, and related patents. To briefly summarize one of those methods, a nucleic acid probe of at least 10 nucleotides, preferably at least 15 nucleotides, more preferably at least 25 nucleotides, having a sequence complementary to a nucleic acid of interest is hybridized in a sample, subjected to depolymerizing conditions, and the sample is treated with an ATP/luciferase system, which will luminesce if the nucleic sequence is present. In quantitative Southern blotting, the level of the nucleic acid of interest can be compared with the level of a second nucleic acid of interest, and/or to one or more comparators nucleic acids (e.g., positive control, negative control, quantity control, etc.).

Many methods useful for the detection and quantification of nucleic acid takes advantage of the polymerase chain reaction (PCR). The PCR process is well known in the art (U.S. Pat. Nos. 4,683,195, 4,683,202, and 4,800,159). To briefly summarize PCR, nucleic acid primers, complementary to opposite strands of a nucleic acid amplification target sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed elsewhere herein. Most preferably the detection of the duplex is done using at least one primer directed to the nucleic acid of interest. In yet another embodiment of PCR, the detection of the hybridized duplex comprises electrophoretic gel separation followed by dye-based visualization.

Typical hybridization and washing stringency conditions depend in part on the size (i.e., number of nucleotides in length) of the oligonucleotide probe, the base composition and monovalent and divalent cation concentrations (Ausubel et al., 1994, eds Current Protocols in Molecular Biology).

In one embodiment, the process for determining the quantitative and qualitative profile of the nucleic acid of interest according to the present invention is characterized in that the amplifications are real-time amplifications performed using a labeled probe, preferably a labeled hydrolysis-probe, capable of specifically hybridizing in stringent conditions with a segment of the nucleic acid of interest. The labeled probe is capable of emitting a detectable signal every time each amplification cycle occurs, allowing the signal obtained for each cycle to be measured.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention is the Tm, which is in the range of about 65° C. to 75° C. Preferably, the Tm for any one of the hydrolysis-probes of the present invention is in the range of about 67° C. to about 70° C. Most preferably, the Tm applied for any one of the hydrolysis-probes of the present invention is about 67° C.

In one aspect, the invention includes a primer that is complementary to a nucleic acid of interest, and more particularly the primer includes 12 or more contiguous nucleotides substantially complementary to the nucleic acid of interest. Preferably, a primer featured in the invention includes a nucleotide sequence sufficiently complementary to hybridize to a nucleic acid sequence of about 12 to 25 nucleotides. More preferably, the primer differs by no more than 1, 2, or 3 nucleotides from the target flanking nucleotide sequence. In another aspect, the length of the primer can vary in length, preferably about 15 to 28 nucleotides in length (e.g., 16, 17, 18, 19, 20, 21, 22, 23, 24, 25, 26, or 27 nucleotides in length).

In one embodiment, the invention includes detecting one or more barcode-labeled nucleic acid sequences, one or more nucleic acid sequence barcodes, or a combination thereof in the DNA of the antibody-bound cell or particle. Such sequences generally can be measured and detected through a variety of assays, methods and detection systems known to one of skill in the art.

Various methods include but are not limited to immunoassays, microarray, PCR, RT-PCR, refractive index spectroscopy (RI), ultra-violet spectroscopy (UV), fluorescence analysis, electrochemical analysis, radiochemical analysis, near-infrared spectroscopy (near-IR), infrared (IR) spectroscopy, nuclear magnetic resonance spectroscopy (NMR), light scattering analysis (LS), mass spectrometry, pyrolysis mass spectrometry, nephelometry, dispersive Raman spectroscopy, gas chromatography, liquid chromatography, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, colorimetry and surface plasmon resonance (such as according to systems provided by Biacore Life Sciences). See also PCT Publications WO/2004/056456 and WO/2004/088309. In this regard, the nucleic acid sequence barcodes can be measured using the above-mentioned detection methods, or other methods known to the skilled artisan. Other nucleic acid sequence barcodes can be similarly detected using reagents that are specifically designed or tailored to detect them.

Different types of antibody targets and their measurements can be combined in the compositions and methods of the present invention. In various embodiments, the nucleic acid sequence encoding one or more antibody target is measured. In various embodiments, the nucleic acid sequence barcode is measured. In exemplary embodiments, the nucleic acid sequence barcode is DNA. In various embodiments, measurements of nucleic acid sequences encoding one or more antibody targets are used in conjunction with measurements of nucleic acid sequence barcodes.

In various embodiments of the invention, methods of measuring antibody target levels (e.g., the levels of barcode-labeled nucleic acid sequences, levels of nucleic acid sequences encoding one or more antibody targets, levels of the nucleic acid barcodes of the barcode-labeled nucleic acid sequences) include, but are not limited to, an immunochromatography assay, an immunodot assay, a Luminex assay, an ELISA assay, an ELISPOT assay, a protein microarray assay, a ligand-receptor binding assay, displacement of a ligand from a receptor assay, displacement of a ligand from a shared receptor assay, an immunostaining assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

Methods for detecting a nucleic acid sequence (e.g., nucleic acid sequence barcode, such as DNA, nucleic acid sequence encoding one or more antibody targets, and/or a barcode-labeled nucleic acid sequence encoding one or more antibody targets), such as RT-PCR, real time PCR, microarray, branch DNA, NASBA and others, are well known in the art. Using sequence information provided by the database entries for the nucleic acid sequences, expression of the nucleic acid sequences can be detected (if present) and measured using techniques well known to one of ordinary skill in the art. For example, sequences in sequence database entries or sequences disclosed herein can be used to construct probes for detecting nucleic acid sequence barcodes in, e.g., Northern blot hybridization analyses or methods which specifically, and, preferably, quantitatively amplify specific nucleic acid sequences. As another example, the sequences can be used to construct primers for specifically amplifying the nucleic acid sequence barcodes in, e.g., amplification-based detection methods such as reverse-transcription based polymerase chain reaction (RT-PCR). In addition to Northern blot and RT-PCR, the level of nucleic acid sequence barcodes can also be measured using, for example, other target amplification methods (e.g., TMA, SDA, NASBA), signal amplification methods (e.g., bDNA), nuclease protection assays, in situ hybridization and the like.

In various embodiments, quantitative hybridization methods, such as Southern analysis, Northern analysis, or in situ hybridizations, can be used (see Current Protocols in Molecular Biology, Ausubel, F. et al., eds., John Wiley & Sons, including all supplements). A “nucleic acid probe,” as used herein, can be a DNA probe or an RNA probe. The probe can be, for example, a gene, a gene fragment (e.g., one or more exons), a vector comprising the gene, a probe or primer, etc. For representative examples of use of nucleic acid probes, see, for example, U.S. Pat. Nos. 5,288,611 and 4,851,330. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate target mRNA or cDNA. The hybridization sample is maintained under conditions which are sufficient to allow specific hybridization of the nucleic acid probe to mRNA or cDNA. Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe having a mRNA or cDNA in the test sample, the level of the mRNA or cDNA in the sample can be assessed. More than one nucleic acid probe can also be used concurrently in this method. Specific hybridization of any one of the nucleic acid probes is indicative of the presence of the mRNA or cDNA of interest, as described herein.

Alternatively, a peptide nucleic acid (PNA) probe can be used instead of a nucleic acid probe in the quantitative hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a target nucleic acid sequence. Hybridization of the PNA probe to a nucleic acid sequence is used to determine the level of the target nucleic acid in the biological sample.

In another embodiment, arrays of oligonucleotide probes that are complementary to target nucleic acid sequence barcodes can be used to determine the level of one or more antibody targets. The array of oligonucleotide probes can be used to determine the level of one or more antibody targets alone or the level of the one or more antibody targets in relation to the level of one or more other nucleic acids in the biological sample. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a nucleic acid of interest is hybridized with the array and its level is quantified. Hybridization and quantification are generally carried out by methods described herein and also in, e.g., published PCT Application Nos. WO 92/10092 and WO 95/11995, and U.S. Pat. No. 5,424,186. In brief, a target nucleic acid sequence is amplified by well-known amplification techniques, e.g., PCR. Typically, this involves the use of primer sequences that are complementary to the target nucleic acid. Asymmetric PCR techniques may also be used. Amplified target, generally incorporating a label, is then hybridized with the array under appropriate conditions. Upon completion of hybridization and washing of the array, the array is scanned to determine the quantity of hybridized nucleic acid. The hybridization data obtained from the scan is typically in the form of fluorescence intensities as a function of quantity, or relative quantity, of the target nucleic acid in the biological sample. The target nucleic acid can be hybridized to the array in combination with one or more comparators (e.g., positive control, negative control, quantity control, etc.) to improve quantification of the target nucleic acid in the sample.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of ³²P, ³³P, ³⁵S or ³H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

Other suitable assays for determining the level of nucleic acid sequence barcode or level of barcode-labeled nucleic acid sequence may include one or more of the following methods, an enzyme assay, an immunoassay, mass spectrometry, chromatography, electrophoresis or an antibody microarray, or any combination thereof. Thus, as would be understood by one skilled in the art, the system and methods of the invention may include any method known in the art to detect a nucleic acid sequence and/or amino acid sequence in a sample.

In some embodiments, methods of identifying antibody targets, optionally, utilize methods that focus on cellular components (cellular examination), or methods that focus on examining extracellular components (fluid examination). In one embodiment, a cellular or fluid examination is used to detect or measure a variety of molecules including the nucleic acid barcode, RNA, protein, and a number of molecules that are modified as a result of the protein's function. Exemplary methods focusing on nucleic acids include but are not limited to amplification techniques, such as PCR and RT-PCR (including quantitative variants), and hybridization techniques, such as in situ hybridization, microarrays, and blots. Exemplary methods focusing on amino acid sequences (e.g., proteins) include but are not limited to binding techniques, such as ELISA, immunohistochemistry, microarray, and functional techniques, such as enzymatic assays. For example, in some embodiments, methods of identifying antibody targets, optionally, utilize ELISA, LIPS, or a combination thereof.

### Methods of Identifying Antibodies

In one aspect, the method comprises identifying at least one antibody that specifically binds to an extracellular or secreted protein. Thus, in one embodiment, the method comprises: isolating the antibodies that bound to the display library of the invention; and identifying the sequence of the antibodies that bound to the display library of the invention.

For example, in various embodiments, the antibody is an anti-BMPR2 antibody, anti-BTN1A1 antibody, anti-BTNL8 antibody, anti-C1QTNF4 antibody, anti-C6 antibody, anti-CCL11 antibody, anti-CCL15 antibody, anti-CCL17 antibody, anti-CCL2 antibody, anti-CCL22 antibody, anti-CCL24 antibody, anti-CCL4L1 antibody, anti-CD207 antibody, anti-CD300E antibody, anti-CD3D antibody, anti-CD44 antibody, anti-CD74 antibody, anti-CD81 antibody, anti-CDH19 antibody, anti-CNTN5 antibody, anti-COLEC12 antibody, anti-CSPG5 antibody, anti-CX3CL1 antibody, anti-CXCL1 antibody, anti-CXCL13 antibody, anti-CXCL2 antibody, anti-CXCL3 antibody, anti-EDIL3 antibody, anti-EPYC antibody, anti-EREG antibody, anti-FGF10 antibody, anti-FGF21 antibody, anti-FGF23 antibody, anti-FGF7 antibody, anti-FGFBP3 antibody, anti-FGFRL1 antibody, anti-IFNA13 antibody, anti-IFNA14 antibody, anti-IFNA17 antibody, anti-IFNA2 antibody, anti-IFNA5 antibody, anti-IFNA6 antibody, anti-IFNA8 antibody, anti-IFNB1 antibody, anti-IFNL2 antibody, anti-IFNW1 antibody, anti-IGF2 antibody, anti-IGFBPL1 antibody, anti-IGSF4B antibody, anti-IL15RA antibody, anti-IL16 antibody, anti-IL17A antibody, anti-IL17F antibody, anti-IL17F antibody, anti-IL18RAP antibody, anti-IL19 antibody, anti-IL1A antibody, anti-IL1F9 antibody, anti-ILIRAP antibody, anti-IL20RB antibody, anti-IL22 antibody, anti-IL22RA2 antibody, anti-IL28B antibody, anti-IL29 antibody, anti-IL33 antibody, anti-IL34 antibody, anti-IL4 antibody, anti-IL4R antibody, anti-IL5 antibody, anti-IL6 antibody, anti-IL6R antibody, anti-ITGA5 antibody, anti-JCHAIN antibody, anti-LAG3 antibody, anti-LGR6 antibody, anti-LIF antibody, anti-LRP11 antibody, anti-LRRC3B antibody, anti-LRRC4 antibody, anti-LRTM2 antibody, anti-LY6G6D antibody, anti-LY6H antibody, anti-MADCAM1 antibody, anti-MPZL3 antibody, anti-MUC21 antibody, anti-NGFR antibody, anti-NOTCH2NL antibody, anti-NTRK3 antibody, anti-PDCD1LG2 antibody, anti-PDGFB antibody, anti-PGLYRP1 antibody, anti-REG1A antibody, anti-REG1B antibody, anti-REG4 antibody, anti-RTN4RL1 antibody, anti-SCARA3 antibody, anti-SDC1 antibody, anti-SDC4 antibody, anti-STIM2 antibody, anti-TGFA antibody, anti-TMEM149 antibody, anti-TNF antibody, anti-TNFRSF10C antibody, anti-TNFRSF10D antibody, anti-TNFRSF19L antibody, anti-TNFRSF6 antibody, anti-TREM2 antibody, anti-TREML1 antibody, anti-TSLP antibody, anti-TSPAN2 antibody, anti-TYRO3 antibody, anti-VEGFB antibody, anti-VSIG4 antibody, anti-VSTM2A antibody, or any combination thereof.

Method of Identifying an Antibody or a Target Thereof Associated with a Disease or Disorder

The present invention provides, in part, a method of identifying disease associated antigen-antibody interactions. The present invention provides, in part, a method of identifying autoantigens that are targets of disease-associated autoantibodies. In one aspect, the method comprises contacting a display library of the invention with a biological sample from a subject who has been diagnosed as having a disease or disorder. In one embodiment, the disease or disorder is selected from an autoimmune disease or disorder, cancer, inflammatory disease or disorder, metabolic disease or disorder, neurodegenerative disease or disorder, organ tissue rejection, organ transplant rejection, an autoimmune or inflammatory disease or disorder associated with an infectious disease, or any combination thereof. In some embodiments, the disease or disorder is antineutrophil cytoplasmic antibody (ANCA)-associated vasculitis, autoimmune polyendocrinopathy candidiasis ecto-dermal dystrophy, antiphospholipid antibody syndrome, chronic inflammatory demyelinating polyradiculoneuropathy, cutaneous lupus erythematosus, COVID-19, drug-induced lupus, dermatomyositis, glomerulonephritis, a disease or disorder associated with kidney transplant, malaria, mixed connective tissue disease, myasthenia gravis, malignant melanoma, neuromyelitis optica, non-small cell lung cancer, pediatric autoimmune neuropsychiatric disorders associated with streptococcal infections, systemic lupus erythematosus, sjogren's syndrome, scleroderma, susac syndrome, undifferentiated connective tissue disease, or any combination thereof.

In one embodiment, the antibody is purified from a biological sample obtained from a subject having a disease or disorder.

In one embodiment, the antigen or autoantigen is identified to be reactive with an antibody or autoantibody associated with a disease or disorder when the level of nucleic acid sequence barcode is statistically different than an expected level based on comparison with a control or a threshold level (e.g., the predetermined threshold level). In one embodiment, the antibody target is identified to be the antibody target associated with the disease or disorder when the level of nucleic acid sequence barcode is higher than the threshold level (e.g., the predetermined threshold level). In some embodiments, the threshold level is obtained from control group samples.

In various embodiments of the methods of the invention, the level (e.g., activity, amount, concentration, expression, level, etc.) of nucleic acid sequence barcode is determined to be increased or to be higher when the level of nucleic acid sequence barcode is determined to be increased by at least 0.01 fold, at least 0.05 fold, at least 0.07 fold, at least 0.076 fold, at least 0.1 fold, at least 0.18 fold, at least 0.19 fold, at least 0.3 fold, at least 0.36 fold, at least 0.37 fold, at least 0.38 fold, at least 0.4 fold, at least 0.43 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 16 fold, at least 16.3 fold, at least 16.31 fold, at least 20 fold, at least 25 fold, at least 26 fold, at least 26.7 fold, at least 26.72 fold, at least 30 fold, at least 40 fold, at least 50 fold, at least 75 fold, at least 100 fold, at least 192 fold, at least 192.4 fold, at least 192.44 fold, at least 200 fold, at least 250 fold, at least 500 fold, or at least 1000 fold, or at least 10000 fold, when compared with a comparator.

In one embodiment, an antibody target is identified to be the antibody target associated with a disease or disorder when the expression level of nucleic acid sequence barcode is increased or higher as compared to a comparator (e.g., the predetermined threshold level). For example, in some embodiments, an antibody target is identified to be the antibody target associated with a disease or disorder when the level of nucleic acid sequence barcode is increased by at least 0.01 fold, or at least 0.18 fold. In some embodiments, an antibody target is identified to be the antibody target associated with a disease or disorder when the level of nucleic acid sequence barcode is increased in a range from 0.1 fold to 10,000 fold.

In one embodiment, the antibody target is identified to be the antibody target associated with the disease or disorder when the level of nucleic acid sequence barcode is lower than the threshold level (e.g., the predetermined threshold level).

In various embodiments of the methods of the invention, the level (e.g., activity, amount, concentration, expression, level, etc.) of nucleic acid sequence barcode is determined to be decreased or to be lower when the level of nucleic acid sequence barcode is determined to be decreased by at least 0.01 fold, at least 0.05 fold, at least 0.07 fold, at least 0.076 fold, at least 0.1 fold, at least 0.18 fold, at least 0.19 fold, at least 0.3 fold, at least 0.36 fold, at least 0.37 fold, at least 0.38 fold, at least 0.4 fold, at least 0.43 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 16 fold, at least 16.3 fold, at least 16.31 fold, at least 20 fold, at least 25 fold, at least 26 fold, at least 26.7 fold, at least 26.72 fold, at least 30 fold, at least 40 fold, at least 50 fold, at least 75 fold, at least 100 fold, at least 192 fold, at least 192.4 fold, at least 192.44 fold, at least 200 fold, at least 250 fold, at least 500 fold, or at least 1000 fold, or at least 10000 fold, when compared with a comparator.

In one embodiment, an antibody target is identified to be the antibody target associated with a disease or disorder when the expression level of nucleic acid sequence barcode is decreased or lower as compared to a comparator (e.g., the predetermined threshold level). For example, in some embodiments, an antibody target is identified to be the antibody target associated with a disease or disorder when the level of nucleic acid sequence barcode is decreased by at least 0.01 fold, or at least 0.18 fold. In some embodiments, an antibody target is identified to be the antibody target associated with a disease or disorder when the level of nucleic acid sequence barcode is decreased in a range from 0.1 fold to 10,000 fold.

In one aspect, the present invention provides, in part, a method of identifying an antibody associated with a disease or disorder. Thus, in one embodiment, the antibody is identified to be the antibody associated with the disease or disorder when the level of the target nucleic acid sequence barcode is different than the threshold level (e.g., the predetermined threshold level). In one embodiment, the antibody is identified to be the antibody associated with the disease or disorder when the level of the target nucleic acid sequence barcode is higher than the threshold level (e.g., the predetermined threshold level). In some embodiments, the threshold level is obtained from control group samples.

In one embodiment, an antibody is identified to be the antibody associated with a disease or disorder when the expression level of the target nucleic acid sequence barcode is increased or higher as compared to a comparator (e.g., the predetermined threshold level). For example, in some embodiments, an antibody is identified to be the antibody associated with a disease or disorder when the level of the target nucleic acid sequence barcode is increased by at least 0.01 fold, or at least 0.18 fold. In some embodiments, an antibody is identified to be the antibody associated with a disease or disorder when the level of nucleic acid sequence barcode is increased in a range from 0.1 fold to 10,000 fold.

In one embodiment, the antibody is identified to be the antibody associated with the disease or disorder when the level of the target nucleic acid sequence barcode is lower than the threshold level (e.g., the predetermined threshold level).

In one embodiment, an antibody is identified to be the antibody associated with a disease or disorder when the expression level of the target nucleic acid sequence barcode is decreased or lower as compared to a comparator (e.g., the predetermined threshold level). For example, in some embodiments, an antibody is identified to be the antibody associated with a disease or disorder when the level of nucleic acid sequence barcode is decreased by at least 0.01 fold, or at least 0.18 fold. In some embodiments, an antibody is identified to be the antibody associated with a disease or disorder when the level of nucleic acid sequence barcode is decreased in a range from 0.1 fold to 10,000 fold.

In some embodiments, the disease or disorder is an autoimmune disease or disorder, cancer, inflammatory disease or disorder, metabolic disease or disorder, neurodegenerative disease or disorder, organ tissue rejection, organ transplant rejection, or any combination thereof. In some embodiments, the disease or disorder is antineutrophil cytoplasmic antibody (ANCA)-associated vasculitis, autoimmune polyendocrinopathy candidiasis ecto-dermal dystrophy, antiphospholipid antibody syndrome, chronic inflammatory demyelinating polyradiculoneuropathy, cutaneous lupus erythematosus, COVID-19, drug-induced lupus, dermatomyositis, glomerulonephritis, a disease or disorder associated with kidney transplant, malaria, mixed connective tissue disease, myasthenia gravis, malignant melanoma, neuromyelitis optica, non-small cell lung cancer, pediatric autoimmune neuropsychiatric disorders associated with streptococcal infections, systemic lupus erythematosus, sjogren's syndrome, scleroderma, susac syndrome, undifferentiated connective tissue disease, or any combination thereof.

In one embodiment, the disease or disorder is a cancer. Examples of cancers include, but are not limited to: acute lymphoblastic; acute myeloid leukemia; adrenocortical carcinoma; adrenocortical carcinoma, childhood; appendix cancer; basal cell carcinoma; bile duct cancer, extrahepatic; bladder cancer; bone cancer; osteosarcoma and malignant fibrous histiocytoma; liposarcoma and anaplastic liposarcoma; brain stem glioma, childhood; brain tumor, adult; brain tumor, brain stem glioma, childhood; brain tumor, central nervous system atypical teratoid/rhabdoid tumor, childhood; central nervous system embryonal tumors; cerebellar astrocytoma; cerebral astrocytotna/malignant glioma; craniopharyngioma; ependymoblastoma; ependymoma; medulloblastoma; medulloepithelioma; pineal parenchymal tumors of intermediate differentiation; supratentorial primitive neuroectodermal tumors and pineoblastoma; visual pathway and hypothalamic glioma; brain and spinal cord tumors; breast cancer; bronchial tumors; Burkitt lymphoma; carcinoid tumor; carcinoid tumor, gastrointestinal; central nervous system atypical teratoid/rhabdoid tumor; central nervous system embryonal tumors; central nervous system lymphoma; cerebellar astrocytoma cerebral astrocytoma/malignant glioma, childhood; cervical cancer; chordoma, childhood; chronic lymphocytic leukemia; chronic myelogenous leukemia; chronic myeloproliferative disorders; colon cancer; colorectal cancer; craniopharyngioma; cutaneous T-cell lymphoma; esophageal cancer; Ewing family of tumors; extragonadal germ cell tumor; extrahepatic bile duct cancer; eye cancer, intraocular melanoma; eye cancer, retinoblastoma; biliary track cancer, cholangiocarcinoma, anal cancer, neuroendocrine tumors, small bowel cancer, gallbladder cancer; gastric (stomach) cancer; gastrointestinal carcinoid tumor; gastrointestinal stromal tumor (gist); germ cell tumor, extracranial; germ cell tumor, extragonadal; germ cell tumor, ovarian; gestational trophoblastic tumor; glioma; glioma, childhood brain stem; glioma, childhood cerebral astrocytoma; glioma, childhood visual pathway and hypothalamic; hairy cell leukemia; head and neck cancer; hepatocellular (liver) cancer; histiocytosis, langerhans cell; Hodgkin lymphoma; hypopharyngeal cancer; hypothalamic and visual pathway glioma; intraocular melanoma; islet cell tumors; kidney (renal cell) cancer; Langerhans cell histiocytosis; laryngeal cancer; leukemia, acute lymphoblastic; leukemia, acute myeloid; leukemia, chronic lymphocytic; leukemia, chronic myelogenous; leukemia, hairy cell; lip and oral cavity cancer; liver cancer; lung cancer, non-small cell; lung cancer, small cell; lymphoma, aids-related; lymphoma, burkitt; lymphoma, cutaneous T-cell; lymphoma, non-Hodgkin lymphoma; lymphoma, primary central nervous system; macroglobulinemia, Waldenstrom; malignant fibrous histiocvtoma of bone and osteosarcoma; medulloblastoma; melanoma; melanoma, intraocular (eye); Merkel cell carcinoma; mesothelioma; metastatic squamous neck cancer with occult primary; mouth cancer; multiple endocrine neoplasia syndrome, (childhood); multiple myeloma/plasma cell neoplasm; mycosis; fungoides; myelodysplastic syndromes; myelodysplastic/myeloproliferative diseases; myelogenous leukemia, chronic; myeloid leukemia, adult acute; myeloid leukemia, childhood acute; myeloma, multiple; myeloproliferative disorders, chronic; nasal cavity and paranasal sinus cancer; nasopharyngeal cancer; neuroblastoma; non-small cell lung cancer; oral cancer; oral cavity cancer; oropharyngeal cancer; osteosarcoma and malignant fibrous histiocytoma of bone; ovarian cancer; ovarian epithelial cancer; ovarian germ cell tumor; ovarian low malignant potential tumor; pancreatic cancer, islet cell tumors; papillomatosis; parathyroid cancer; penile cancer; pharyngeal cancer; pheochromocytoma; pineal parenchymal tumors of intermediate differentiation; pineoblastoma and supratentorial primitive neuroectodermal tumors; pituitary tumor; plasma celt neoplasm/multiple myeloma; pleuropulmonary blastoma; primary central nervous system lymphoma; prostate cancer; rectal cancer; renal cell (kidney) cancer; renal pelvis and ureter, transitional cell cancer; respiratory tract carcinoma involving the nut gene on chromosome 15; retinoblastoma; rhabdomyosarcoma; salivary gland cancer; sarcoma, ewing family of tumors; sarcoma, Kaposi; sarcoma, soft tissue; sarcoma, uterine; sezary syndrome; skin cancer (nonmelanoma); skin cancer (melanoma); skin carcinoma, Merkel cell; small cell lung cancer; small intestine cancer; soft tissue sarcoma; squamous cell carcinoma, squamous neck cancer with occult primary, metastatic; stomach (gastric) cancer; supratentorial primitive neuroectodermal tumors; T-cell lymphoma, cutaneous; testicular cancer; throat cancer; thymoma and thymic carcinoma; thyroid cancer; transitional cell cancer of the renal pelvis and ureter; trophoblastic tumor, gestational; urethral cancer; uterine cancer, endometrial; uterine sarcoma; vaginal cancer; vulvar cancer; Waldenstrom macroglobulinemia; Wilms tumor, and any combination thereof.

Control group samples may either be from a normal subject, samples from subjects with a known diagnosis of a disease or disorder associated with increased level of the antibody or the target thereof, samples from subjects with a known diagnosis of a disease or disorder associated with decreased level of the antibody or the target thereof, or any combination thereof. As described below, comparison of the expression patterns of the sample to be tested with those of the comparators can be used to assess the risk of developing a disease or disorder associated with decreased antibody level, increased level of the antibody or the target thereof, or any combination thereof in the subject. In some instances, the control groups are only for the purposes of establishing initial cutoffs or thresholds for the assays of the invention. Therefore, in some instances, the systems and methods of the invention can evaluate a treatment of a disease or disorder associated with decreased level of the antibody or target thereof, increased level of the antibody or target thereof, or any combination thereof without the need to compare with a control group.

### Method of Diagnosing a Disease or Disorder

The present invention further relates, in part, to a method of diagnosing a disease or disorder associated with at least one antibody or target thereof (e.g., an antibody level, antibody target level, antibody activity, or antibody target activity) in a subject in need thereof.

In one aspect, the present invention provides a method of diagnosing a disease or disorder in a subject, the method comprising assessing the presence of at least one antibody in the subject, wherein the at least one antibody is identified to be associated with the disease or disorder according to the method described above. In one aspect, the present invention provides a method of diagnosing a disease or disorder in a subject, the method comprising assessing the level or activity of at least one antibody in the subject, wherein the at least one antibody is identified to be associated with the disease or disorder according to the method described above.

In one embodiment, the subject is diagnosed with a disease or disorder when the level or activity of at least one antibody is different than the threshold level (e.g., the predetermined threshold level). In one embodiment, the subject is diagnosed with a disease or disorder when the level or activity of at least one antibody is higher than the threshold level (e.g., the predetermined threshold level). In some embodiments, the threshold level is obtained from control group samples. In one embodiment, the threshold is 0.

In one embodiment, the subject is diagnosed with a disease or disorder by detecting an altered or increased level of an antibody that binds to at least one antibody target associated with the disease or disorder, relative to a control level. In some embodiments, the control level is a level of a particular marker (i.e., an antibody that binds to at least one antibody target associated with the disease or disorder) in a subject or population known not to have the disease.

In various embodiments of the methods of the invention, the level (e.g., activity, amount, concentration, expression, level, etc.) of antibody is determined to be increased or to be higher when the level of antibody is determined to be more than 0.

In various embodiments of the methods of the invention, the level (e.g., activity, amount, concentration, expression, level, etc.) of antibody is determined to be increased or to be higher when the level of antibody is determined to be increased by at least 0.01 fold, at least 0.05 fold, at least 0.07 fold, at least 0.076 fold, at least 0.1 fold, at least 0.18 fold, at least 0.19 fold, at least 0.3 fold, at least 0.36 fold, at least 0.37 fold, at least 0.38 fold, at least 0.4 fold, at least 0.43 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 16 fold, at least 16.3 fold, at least 16.31 fold, at least 20 fold, at least 25 fold, at least 26 fold, at least 26.7 fold, at least 26.72 fold, at least 30 fold, at least 40 fold, at least 50 fold, at least 75 fold, at least 100 fold, at least 192 fold, at least 192.4 fold, at least 192.44 fold, at least 200 fold, at least 250 fold, at least 500 fold, or at least 1000 fold, or at least 10000 fold, when compared with a comparator (e.g., the level of antibody in control group samples).

In one embodiment, the subject is diagnosed with a disease or disorder when the level or activity of at least one antibody associated with the disease or disorder is increased or higher as compared to a comparator (e.g., the predetermined threshold level). For example, in some embodiments, the subject is diagnosed with a disease or disorder when at least one antibody associated with the disease or disorder is present in the subject (i.e., the level or activity of at least one antibody associated with the disease or disorder is more than 0). In some embodiments, the subject is diagnosed with a disease or disorder when the level or activity of at least one antibody associated with the disease or disorder is increased by at least 0.01 fold, or at least 0.18 fold. In some embodiments, the subject is diagnosed with a disease or disorder when the level or activity of at least one antibody associated with the disease or disorder is increased in a range from 0.1 fold to 10,000 fold.

For example, in some embodiments, the subject is diagnosed with ANCA-associated vasculitis by detecting an altered or increased level of an antibody that binds to EDIL3, LY6H, TREM2, or any combination thereof, relative to a control level.

In some embodiments, the subject is diagnosed with autoimmune polyendocrinopathy candidiasis ecto-dermal dystrophy by detecting an altered or increased level of an antibody that binds to FGF10, LRRC3B, VSTM2A, IL22, IL17F, IL17A, IL5, IL22RA2, IFNL2, IGSF4B, IL28B, IFNA13, IFNA14, IFNA17, IFNA2, IFNA5, IFNA6, IFNA8, or any combination thereof, relative to a control level.

In some embodiments, the subject is diagnosed with antiphospholipid antibody syndrome by detecting an altered or increased level of an antibody that binds to IFNA13, IFNA14, IFNA17, IFNA2, IFNA5, IFNA6, IFNA8, IL6R, or any combination thereof, relative to a control level.

In some embodiments, the subject is diagnosed with chronic inflammatory demyelinating polyradiculoneuropathy by detecting an altered or increased level of an antibody that binds to CXCL1, CXCL2, CXCL3, PDGFB, TMEM149, CD74, CXCL13, or any combination thereof, relative to a control level.

In some embodiments, the subject is diagnosed with cutaneous lupus erythematosus by detecting an altered or increased level of an antibody that binds to CCL11, CCL24, CD300E, IFNL2, TMEM149, TYRO3, VEGFB, or any combination thereof, relative to a control level.

In some embodiments, the subject is diagnosed with drug-induced lupus by detecting an altered or increased level of an antibody that binds to CXCL1, TNF, TSLP, or any combination thereof, relative to a control level.

In some embodiments, the subject is diagnosed with dermatomyositis by detecting an altered or increased level of an antibody that binds to CD81, relative to a control level.

In some embodiments, the subject is diagnosed with glomerulonephritis by detecting an altered or increased level of an antibody that binds to C1QTNF4, CCL17, CCL4L1, CXCL2, CXCL3, EDIL3, EPYC, IFNL2, IL34, PDGFB, RTN4RL1, TMEM149, TREM2, TSLP, or any combination thereof, relative to a control level.

In some embodiments, the subject is diagnosed with mixed connective tissue disease by detecting an altered or increased level of an antibody that binds to BTNL8, CXCL3, EPYC, JCHAIN, SDC4, TSPAN2, VEGFB, or any combination thereof, relative to a control level.

In some embodiments, the subject is diagnosed with myasthenia gravis by detecting an altered or increased level of an antibody that binds to CXCL2, PDGFB, REG4, CCL22, CCL2, or any combination thereof, relative to a control level.

In some embodiments, the subject is diagnosed with neuromyelitis optica by detecting an altered or increased level of an antibody that binds to CXCL2, CXCL3, IGFBPL1, CCL22, IL1F9, LY6G6D, or any combination thereof, relative to a control level.

In some embodiments, the subject is diagnosed with non-small cell lung cancer by detecting an altered or increased level of an antibody that binds to CCL17, CCL24, CXCL1, CXCL3, EDIL3, IFNA13, IFNA14, IFNA17, IFNA2, IFNA5, IFNA6, IFNA8, IFNL2, IFNW1, IL28B, IL34, MADCAM1, PDGFB, REG1A, SDC1, BTN1A1, C6, CD207, CD3D, CDH19, COLEC12, EREG, FGF23, FGF7, FGFBP3, IGFBPL1, IL15RA, IL17F, IL1RAP, IL22RA2, IL4, IL4R, ITGA5, LAG3, LRRC4, MPZL3, NOTCH2NL, NTRK3, REG4, SCARA3, STIM2, TNFRSF10C, TNFRSF19L, TREML1, or any combination thereof, relative to a control level.

In some embodiments, the subject is diagnosed with pediatric autoimmune neuropsychiatric disorders associated with streptococcal infections by detecting an altered or increased level of an antibody that binds to LRP 11, relative to a control level.

In some embodiments, the subject is diagnosed with sarcoidosis by detecting an altered or increased level of an antibody that binds to CX3CL1, EPYC, PGLYRP1, or any combination thereof, relative to a control level.

In some embodiments, the subject is diagnosed with systemic lupus erythematosus by detecting an altered or increased level of an antibody that binds to BMPR2, BTNL8, C1QTNF4, CCL11, CCL15, CCL17, CCL24, CCL4L1, CD300E, CD44, CSPG5, CX3CL1, CXCL1, CXCL2, CXCL3, EDIL3, EPYC, FGF21, FGFRL1, IFNA13, IFNA14, IFNA17, IFNA2, IFNA5, IFNA6, IFNA8, IFNB1, IFNL2, IFNW1, IGF2, IGSF4B, IL16, IL18RAP, IL19, IL1A, IL20RB, IL28B, IL29, L33, IL34, IL6, IL6R, JCHAIN, LGR6, LIF, LRTM2, LY6H, MADCAM1, MUC21, NGFR, PDCD1LG2, PDGFB, PGLYRP1, REG1A, REG1B, RTN4RL1, SDC1, SDC4, TGFA, TMEM149, TNF, TNFRSF10D, TNFRSF6, TREM2, TSLP, TSPAN2, TYRO3, VEGFB, or any combination thereof, relative to a control level.

In some embodiments, the subject is diagnosed with sjogren's syndrome by detecting an altered or increased level of an antibody that binds to CXCL1, CXCL3, PDCD1LG2, or any combination thereof, relative to a control level.

In some embodiments, the subject is diagnosed with susac syndrome by detecting an altered or increased level of an antibody that binds to CCL24, SDC4, TREML1, VSIG4, or any combination thereof, relative to a control level.

In some embodiments, the subject is diagnosed with undifferentiated connective tissue disease by detecting an altered or increased level of an antibody that binds to CNTN5, TNF, or any combination thereof, relative to a control level.

In one aspect, the present invention provides a method of diagnosing a disease or disorder in a subject, the method comprising assessing the presence of at least one antibody or autoantibody in a biological sample from the subject, wherein the at least one antibody or autoantibody is identified to be associated with the disease or disorder according to the methods described elsewhere herein. In one aspect, the present invention provides a method of diagnosing a disease or disorder in a subject, the method comprising detecting the binding of at least one autoantibody with at least one autoantigen as set forth in Table 3, and diagnosing the subject as having or at risk of having the associated disease or disorder as set forth in Table 3. In one aspect, the present invention provides a method of diagnosing a disease or disorder in a subject, the method comprising assessing detecting the binding of at least one autoantibody with at least one autoantigen as set forth in Table 4, and diagnosing the subject as having or at risk of having the associated disease or disorder as set forth in Table 4.

In one aspect, the present invention provides a method of evaluating the effectiveness of a treatment for a disease or disorder in a subject, the method comprising assessing the presence of at least one antibody or autoantibody in a biological sample from the subject, wherein the at least one antibody or autoantibody is identified to be associated with the disease or disorder according to the methods described elsewhere herein. In one aspect, the present invention provides a method of evaluating the effectiveness of a treatment for a disease or disorder in a subject, the method comprising detecting the binding of at least one autoantibody with at least one autoantigen as set forth in Table 3, in a subject pre administration of a treatment regimen, post administration of a treatment regimen, or both pre- and post-administration of a treatment regimen. For example, in one embodiment, the treatment regimen comprises administration of an antibody, and the method of the invention is used to evaluate the effectiveness of the treatment regimen by detecting the presence of or an increased level of antibody reactivity with a target antigen following treatment. In one embodiment, the treatment regimen comprises administering a therapeutic agent to reduce or eliminate one or more autoantibodies, and the method of the invention is used to evaluate the effectiveness of the treatment regimen by detecting the absence of or a reduced level of antibody reactivity with a target antigen following treatment.

In one embodiment, the subject is diagnosed with a disease or disorder when the level or activity of at least one antibody target associated with the disease or disorder is different than the threshold level. In one embodiment, the subject is diagnosed with a disease or disorder when the level or activity (e.g., activity, amount, concentration, expression, level, etc.) of at least one antibody target associated with the disease or disorder is higher than the threshold level. In some embodiments, the threshold level is obtained from control group samples.

In one embodiment, the subject is diagnosed with a disease or disorder by detecting an altered or increased level of an antibody target associated with the disease or disorder, relative to a control level. In some embodiments, the control level is a level of a particular marker (i.e., an antibody that binds to at least one antibody target associated with the disease or disorder) in a subject or population known not to have the disease. In various embodiments of the methods of the invention, the level (e.g., activity, amount, concentration, expression, level, etc.) of antibody target is determined to be increased or to be higher when the level of antibody target is determined to be increased by at least 0.01 fold, at least 0.05 fold, at least 0.07 fold, at least 0.076 fold, at least 0.1 fold, at least 0.18 fold, at least 0.19 fold, at least 0.3 fold, at least 0.36 fold, at least 0.37 fold, at least 0.38 fold, at least 0.4 fold, at least 0.43 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 16 fold, at least 16.3 fold, at least 16.31 fold, at least 20 fold, at least 25 fold, at least 26 fold, at least 26.7 fold, at least 26.72 fold, at least 30 fold, at least 40 fold, at least 50 fold, at least 75 fold, at least 100 fold, at least 192 fold, at least 192.4 fold, at least 192.44 fold, at least 200 fold, at least 250 fold, at least 500 fold, or at least 1000 fold, or at least 10000 fold, when compared with a comparator (e.g., the level of antibody target in control group samples).

In one embodiment, the subject is diagnosed with a disease or disorder when the level or activity of at least one antibody target associated with the disease or disorder is increased or higher as compared to a comparator (e.g., the predetermined threshold level). For example, in some embodiments, the subject is diagnosed with a disease or disorder when at least one antibody target associated with the disease or disorder is present in the subject (i.e., the level or activity of at least one antibody target associated with the disease or disorder is more than 0). In some embodiments, the subject is diagnosed with a disease or disorder when the level or activity of at least one antibody target associated with the disease or disorder is increased by at least 0.01 fold, or at least 0.18 fold. In some embodiments, the subject is diagnosed with a disease or disorder when the level or activity of at least one antibody target associated with the disease or disorder is increased in a range from 0.1 fold to 10,000 fold.

### Method of Preventing or Treating a Disease or Disorder

The present invention further relates, in part, to methods of preventing or treating a diseases or disorders associated with at least one antibody or target thereof (e.g., an antibody level, antibody target level, antibody activity, or antibody target activity) in a subject in need thereof. In one aspect, the method comprises administering a treatment to the subject comprising eliminating or modifying the level (e.g., activity, amount, concentration, expression, level, etc.) of at least one antibody target that is identified to be the antibody target associated with the disease or disorder according to the method of the present invention.

In one aspect, the present invention relates to a method of preventing or treating a disease or disorder associated with at least one antibody target in a subject in need thereof. In one embodiment, the method comprises administering a treatment to reduce the level (e.g., activity, amount, concentration, expression, level, etc.) of the antibody target identified to be associated with the disease or disorder according to the method of the present invention in the subject. In one embodiment, the treatment comprises inhibiting at least one antibody target associated with the disease or disorder. In one embodiment, the treatment comprises administering a therapeutically effective amount of an inhibitor of at least one antibody target associated with the disease or disorder. For example, in some embodiments, the inhibitor of the antibody target is an antibody, nucleic acid, peptide, small molecule, antagonist, aptamer, peptidomemetic, or a combination thereof.

In one aspect, the present invention relates to a method of preventing or treating a disease or disorder associated with an increased level of at least one antibody target in a subject in need thereof. In one embodiment, the method comprises administering a treatment to reduce the level (e.g., activity, amount, concentration, expression, level, etc.) of the antibody target identified to be associated with the disease or disorder according to the method of the present invention in the subject. In one embodiment, the treatment comprises inhibiting at least one antibody target associated with the disease or disorder. In one embodiment, the treatment comprises administering a therapeutically effective amount of an inhibitor of at least one antibody target associated with the disease or disorder. For example, in some embodiments, the inhibitor of the antibody target is an antibody For example, in some embodiments, the inhibitor of the antibody target is an antibody, nucleic acid, peptide, small molecule, antagonist, aptamer, peptidomemetic, or a combination thereof.

In one aspect, the present invention relates to a method of preventing or treating a disease or disorder associated with a decreased level of at least one antibody target in a subject in need thereof. In one embodiment, the method comprises administering a treatment to increase the level (e.g., activity, amount, concentration, expression, level, etc.) of the antibody target identified to be associated with the disease or disorder according to the method of the present invention in the subject. In one embodiment, the treatment comprises activating at least one antibody target associated with the disease or disorder. For example, in some embodiments, the treatment comprises increasing the level or activity of at least one antibody target associated with the disease or disorder by administering a therapeutically effective amount of at least one antibody target associated with the disease or disorder or a fragment thereof, nucleic acid sequences encoding the antibody target associated with the disease or disorder or a fragment thereof, inhibitor of the antibody that specifically binds to the antibody target, therapeutic agent, or a combination thereof. In some embodiments, the inhibitor of the antibody that specifically binds to the antibody target is an antibody, therapeutic agent, or a combination thereof.

The present invention also relates, in part, to methods of preventing or treating a disease or disorder associated with at least one antibody (e.g., antibody level or activity) in a subject in need thereof. In one aspect, the method comprises administering a treatment to the subject comprising modifying the level (e.g., activity, amount, concentration, expression, level, etc.) of at least one antibody that binds to an antigen associated with the disease or disorder according to the method of the present invention.

In one aspect, the present invention relates to a method of preventing or treating a disease or disorder associated with at least one antibody in a subject in need thereof. In one embodiment, the method comprises administering a treatment to reduce the level (e.g., activity, amount, concentration, expression, level, etc.) of the antibody identified to be associated with the disease or disorder according to the method of the present invention in the subject. In one embodiment, the treatment comprises inhibiting at least one antibody associated with the disease or disorder. In one embodiment, the treatment comprises administering a therapeutically effective amount of an inhibitor of at least one antibody associated with the disease or disorder. For example, in some embodiments, the inhibitor of the antibody is a composition comprising an antigen identified according to the methods of the invention, or a fragment thereof, that specifically binds to the antibody associated with the disease or disorder. In some embodiments, the composition comprising the antigen further comprises a therapeutic agent, a nucleic acid, a peptide, an antibody, a small molecule, or a combination thereof.

In one aspect, the present invention relates to a method of preventing or treating a disease or disorder associated with at least one antibody in a subject in need thereof. In one embodiment, the method comprises administering a therapeutic agent for decreasing the level (e.g., activity, amount, concentration, expression, level, etc.) of at least one antibody identified to be associated with the disease or disorder according to the method of the present invention in the subject. In one embodiment, the method comprises administering a therapeutic agent for inhibiting the reactivity of at least one antibody with at least one antigen identified to be associated with the disease or disorder according to the method of the present invention in the subject. In one embodiment, the method comprises inhibiting the reactivitiy of at least of antibody with at least one antigen for the treatment of the associated disease as set forth in Table 3. In one embodiment, the method comprises modulating the reactivitiy of at least of antibody with at least one antigen for the treatment of the associated disease as set forth in Table 3.

Exemplary therapeutic autoantigens whose reactivities with autoantibodies can be increased for the treatment of diseases and disorders include, but are not limited to, those autoantigens identified in Table 5, and associated diseases. Therefore, in one embodiment, the methods of the invention include methods of admininstering an autoantibody directed to autoantigen as set forth in Table 5, or a fragment thereof.

Exemplary autoantigens whose reactivities with autoantibodies can be inhibited or decreased for the treatment of diseases and disorders include, but are not limited to, those autoantigens identified in Table 6, and associated diseases. Therefore, in one embodiment, the methods of the invention include methods of admininstering an agent to decrease the level or activity of an autoantibody directed to autoantigen as set forth in Table 6, or a fragment thereof.

In one embodiment, the methods of the invention include methods of administering a fusion molecule comprising an antigen identified according to the methods of the invention fused to a domain to support degradation of an antibody. Exemplary domains to promote internalization and degradation of autoantibodies include, but are not limited to, an asialoglycoprotein receptor binding domain. In such an embodiment, binding of the autoantibody to the fusion antigen would result in targeted degradation of the bound autoantibody. Therefore, in some embodiments, the invention relates to fusion molecules comprising the antigens as set forth in Table 3 fused to a molecule for endocytosis and degradation, and their use for treating the associated disease or disorder as set forth in Table 3. In some embodiments, the invention relates to fusion molecules comprising the antigens as set forth in Table 6 fused to a molecule for endocytosis and degradation, and their use for treating the associated disease or disorder as set forth in Table 6.

In one embodiment, the methods of the invention include methods of directing T cells to B cells expressing autoantibodies. For example, in one embodiment, the invention provides compositions comprising engineered T cells expressing an autoantigen identified according to the methods of the invention, and their use to target auto-antigen expressing B cells for depletion or killing. Therefore, in various embodiments, the invention includes engineered T cells, including but not limited to, CAR-T cells and CAAR-T cells, expressing an antigen as set forth in Table 3, and the use thereof for the treatment of the associated disease or disorder as set forth in Table 3. Therefore, in various embodiments, the invention includes engineered T cells, including but not limited to, CAR-T cells and CAAR-T cells, expressing an antigen as set forth in Table 6, and the use thereof for the treatment of the associated disease or disorder as set forth in Table 6.

In some embodiments, the method of preventing or treating COVID-19 comprises administering a treatment to the subject for decreasing the level or activity of at least one autoantibody directed to IFITM10, IFNA13, IFNA14, IFNA17, IFNA2, IFNA5, IFNA6, IFNA8, IFNW1, KLRC1, KLRC2, KLRC3, CCR2, CD38, C5A, CCR4, CD3E, TNFRSF9, ADCYAP1, CGA, HCTR2, AZGP1, SCC41A2 or LAIR1 or any combination thereof. In some embodiments, the method of preventing or treating COVID-19 comprises administering a composition comprising at least one of IFITM10, IFNA13, IFNA14, IFNA17, IFNA2, IFNA5, IFNA6, IFNA8, IFNW1, KLRC1, KLRC2, KLRC3, CCR2, CD38, C5A, CCR4, CD3E, TNFRSF9, ADCYAP1, CGA, HCTR2, AZGP1, SCC41A2 and LAIR1, and further comprising a domain for degradation of an autoantibody directed to at least one of IFITM10, IFNA13, IFNA14, IFNA17, IFNA2, IFNA5, IFNA6, IFNA8, IFNW1, KLRC1, KLRC2, KLRC3, CCR2, CD38, C5A, CCR4, CD3E, TNFRSF9, ADCYAP1, CGA, HCTR2, AZGP1, SCC41A2 and LAIR1. In one embodiment, the method of preventing or treating COVID-19 comprises administering a composition comprising a CAR T cell expressing at least one of IFITM10, IFNA13, IFNA14, IFNA17, IFNA2, IFNA5, IFNA6, IFNA8, IFNW1, KLRC1, KLRC2, KLRC3, CCR2, CD38, C5A, CCR4, CD3E, TNFRSF9, ADCYAP1, CGA, HCTR2, AZGP1, SCC41A2 and LAIR1.

In some embodiments, the method of preventing or treating a disease or disorder associated with kidney transplant comprises administering a treatment to the subject for decreasing the level or activity of at least one autoantibody directed to IL4, EXOC3-AS1, IFNA13, CD99L2, OSTN, SYCN, LYG2, BTN1A1, or any combination thereof. In some embodiments, the method of preventing or treating a disease or disorder associated with kidney transplant comprises administering a composition comprising at least one of IL4, EXOC3-AS1, IFNA13, CD99L2, OSTN, SYCN, LYG2, and BTN1A1, and further comprising a domain for degradation of an autoantibody directed to at least one of IL4, EXOC3-AS1, IFNA13, CD99L2, OSTN, SYCN, LYG2, and BTN1A1. In one embodiment, the method of preventing or treating a disease or disorder associated with kidney transplant comprises administering a composition comprising a CAR T cell expressing at least one of IL4, EXOC3-AS1, IFNA13, CD99L2, OSTN, SYCN, LYG2, and BTN1A1.

In some embodiments, the method of preventing or treating malignant melanoma comprises administering a treatment to the subject for decreasing the level or activity of at least one autoantibody directed to IFNA13, OBP2B, TMEM108, CELAl, OTOL1, ATP4B, ICOSLG, REG1A, CCL24, TMEM91, LALBA, ITPRIPL1, LCN2, BTN1A1, OS9, FGF17 or any combination thereof. In some embodiments, the method of preventing or treating malignant melanoma comprises administering a composition comprising at least one of IFNA13, OBP2B, TMEM108, CELAl, OTOL1, ATP4B, ICOSLG, REG1A, CCL24, TMEM91, LALBA, ITPRIPL1, LCN2, BTN1A1, OS9, and FGF17, and further comprising a domain for degradation of an autoantibody directed to at least one of IFNA13, OBP2B, TMEM108, CELAl, OTOL1, ATP4B, ICOSLG, REG1A, CCL24, TMEM91, LALBA, ITPRIPL1, LCN2, BTN1A1, OS9, and FGF17. In one embodiment, the method of preventing or treating malignant melanoma comprises administering a composition comprising a CAR T cell expressing at least one of IFNA13, OBP2B, TMEM108, CELAl, OTOL1, ATP4B, ICOSLG, REG1A, CCL24, TMEM91, LALBA, ITPRIPL1, LCN2, BTN1A1, OS9, FGF17.

In some embodiments, the method of preventing or treating non-small cell lung cancer comprises administering a treatment to the subject for decreasing the level or activity of at least one autoantibody directed to IFNL2, VSTM2A, PDGFB or any combination thereof. In some embodiments, the method of preventing or treating non-small cell lung cancer comprises administering a composition comprising at least one of IFNL2, VSTM2A, and PDGFB, and further comprising a domain for degradation of an autoantibody directed to at least one of IFNL2, VSTM2A, and PDGFB. In one embodiment, the method of preventing or treating non-small cell lung cancer comprises administering a composition comprising a CAR T cell expressing at least one of IFNL2, VSTM2A, and PDGFB.

In some embodiments, the method of preventing or treating systemic lupus erythematosus comprises administering a treatment to the subject for decreasing the level or activity of at least one autoantibody directed to TMEM102, CCL8, CCL4L1, ACVR2B, FGF21, IGFBP2, RGMB, ACVR1B, ACRV1, SCGB1D1, TFF2, SFN, ANTXRL, SLC41A2, CD248 or any combination thereof. In some embodiments, the method of preventing or treating systemic lupus erythematosus comprises administering a composition comprising at least one of TMEM102, CCL8, CCL4L1, ACVR2B, FGF21, IGFBP2, RGMB, ACVR1B, ACRV1, SCGB1D1, TFF2, SFN, ANTXRL, SLC41A2, and CD248, and further comprising a domain for degradation of an autoantibody directed to at least one of TMEM102, CCL8, CCL4L1, ACVR2B, FGF21, IGFBP2, RGMB, ACVR1B, ACRV1, SCGB1D1, TFF2, SFN, ANTXRL, SLC41A2, and CD248. In one embodiment, the method of preventing or treating systemic lupus erythematosus comprises administering a composition comprising a CAR T cell expressing at least one of TMEM102, CCL8, CCL4L1, ACVR2B, FGF21, IGFBP2, RGMB, ACVR1B, ACRV1, SCGB1D1, TFF2, SFN, ANTXRL, SLC41A2, and CD248.

In one aspect, the present invention relates to a method of preventing or treating a disease or disorder associated with insufficient level of at least one antibody in a subject in need thereof. In one embodiment, the method comprises administering a treatment for decreasing the level (e.g., activity, amount, concentration, expression, level, etc.) of an antigen identified to be associated with the disease or disorder according to the method of the present invention in the subject. In one embodiment, the treatment comprises administering at least one antibody specific for binding to the antigen. For example, in some embodiments, the treatment comprises decreasing the level or activity of at least one autoantigen associated with a disease or disorder by administering a therapeutically effective amount of at least one antibody, or a fragment thereof, specific for binding to the antigen, a nucleic acid sequence encoding the antibody, or a fragment thereof, a therapeutic agent, nucleic acid, peptide, small molecule, antagonist, aptamer, peptidomemetic, or a combination thereof, or a combination thereof.

For example, in some embodiments, the method of preventing or treating autoimmune polyendocrinopathy candidiasis ecto-dermal dystrophy comprises administering a treatment to the subject for modulating the level or activity of IL22RA2, or administering an antibody that binds to IL22RA2.

In some embodiments, the method of preventing or treating cutaneous lupus erythematosus comprises administering a treatment to the subject for modulating the level or activity of CD300E, TYRO3, or any combination thereof, or administering an antibody that binds to CD300E, TYRO3, or any combination thereof.

In some embodiments, the method of preventing or treating COVID-19 comprises administering a treatment to the subject for modulating the level or activity of IL13, IL18RAP, TNFRSF8, CCR10, CD74, TNFRSF17, CCR9, CRTAM, C6, or any combination thereof, or administering an antibody that binds to IL13, IL18RAP, TNFRSF8, CCR10, CD74, TNFRSF17, CCR9, CRTAM, C6, or any combination thereof.

In some embodiments, the method of preventing or treating dermatomyositis comprises administering a treatment to the subject for modulating the level or activity of CD81, or administering an antibody that binds to CD81.

In some embodiments, the method of preventing or treating glomerulonephritis comprises administering a treatment to the subject for modulating the level or activity of IL34, or administering an antibody that binds to IL34.

In some embodiments, the method of preventing or treating a disease or disorder associated with kidney transplant comprises administering a treatment to the subject for modulating the level or activity of IGFBP1, IL15RA, NXPH1, CST5, C6, or any combination thereof, or administering an antibody that binds to IGFBP1, IL15RA, NXPH1, CST5, C6, or any combination thereof.

In some embodiments, the method of preventing or treating myasthenia gravis comprises administering a treatment to the subject for modulating the level or activity of CCL22, CCL2, or any combination thereof, or administering an antibody that binds to CCL22, CCL2, or any combination thereof.

In some embodiments, the method of preventing or treating malignant melanoma comprises administering a treatment to the subject for modulating the level or activity of PSORS1C2, LHFPL1, PTPRR, ZG16B, IGF1, IFLL1, LRIT3, VEGFB, or any combination thereof, or administering an antibody that binds to PSORS1C2, LHFPL1, PTPRR, ZG16B, IGF1, IFLL1, LRIT3, VEGFB, or any combination thereof.

In some embodiments, the method of preventing or treating neuromyelitis opticas comprises administering a treatment to the subject for modulating the level or activity of CCL22, IL1F9, or any combination thereof, or administering an antibody that binds to CCL22, IL1F9, or any combination thereof.

In some embodiments, the method of preventing or treating non-small cell lung cancer comprises administering a treatment to the subject for modulating the level or activity of CCL22, FGF23, FGF7, EREG, CXCL1, CXCL2, CXCL3, VEGFB, IL1A, LAG3, IFNA13, IFNA14, IFNA17, IFNA2, IFNA5, IFNA6, IFNA8, IFNL2, IFNW1, IL34, IL22RA2, IGFBPL1 or any combination thereof, or an administering antibody that binds to CCL22, FGF23, FGF7, EREG, CXCL1, CXCL2, CXCL3, VEGFB, IL1A, LAG3, IFNA13, IFNA14, IFNA17, IFNA2, IFNA5, IFNA6, IFNA8, IFNL2, IFNW1, IL34, IL22RA2, IGFBPL1 or any combination thereof.

In some embodiments, the method of preventing or treating systemic lupus erythematosus comprises administering a treatment to the subject for modulating the level or activity of PDCD1LG2, LIF, IFNA13, IFNA14, IFNA17, IFNA2, IFNA5, IFNA6, IFNA8, IFNB1, IFNL2, IFNW1, IL6, IL6R, IL33, IL34, IL16, IL19, IL20RB, IL18RAP, MADCAM1, TNF, TRAILR4, TYRO3, CD44, CD300E, FGF21, CXCL1, CXCL2, CXCL3, VEGFB, IL1A, LILRB2, LILRB4 or any combination thereof, or administering an antibody that binds to PDCD1LG2, LIF, IFNA13, IFNA14, IFNA17, IFNA2, IFNA5, IFNA6, IFNA8, IFNB1, IFNL2, IFNW1, IL6, TL6R, IL33, IL34, IL16, IL19, IL20RB, IL18RAP, MADCAM1, TNF, TRAILR4, TYRO3, CD44, CD300E, FGF21, CXCL1, CXCL2, CXCL3, VEGFB, IL1A, LILRB2, LILRB4 or any combination thereof.

In some embodiments, the method of preventing or treating sjogren's syndrome comprises administering a treatment to the subject for modulating the level or activity of PDCD1LG2, or administering an antibody that binds to PDCD1LG2.

In one embodiment, the invention relates to the use of therapeutic agent to modulate the reactivity of at least one autoantibody with at least one autoantigen of the invention. Examples of therapeutic agents include, but are not limited to, one or more drugs, metabolites, metabolic inhibitors, proteins, amino acids, peptides, antibodies, medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, anti-tumor agents, including cytotoxic/antineoplastic agents and anti-angiogenic agents, or any combination thereof.

Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

In one embodiment, the treatment comprises administering a therapeutically effective amount of at least one agent for modulating the reactivity of at least one antibody with at least one antigen.

In some embodiments, the treatment comprises decreasing or eliminating the level of at least one antibody associated with the disease or disorder by administering a therapeutically effective amount of an inhibitor of at least one antibody associated with the disease or disorder. For example, in one embodiment, the inhibitor of the antibody comprises an autoantigen identified using the methods of the invention.

Any drug or any combination of drugs disclosed herein may be administered to a subject to treat the disease or disorder. The drugs herein can be formulated in any number of ways, often according to various known formulations in the art or as disclosed or referenced herein.

In various embodiments, any drug or any combination of drugs disclosed herein is not administered to a subject to treat a disease. In these embodiments, the practitioner may refrain from administering the drug or any combination of drugs, may recommend that the subject not be administered the drug or any combination of drugs or may prevent the subject from being administered the drug or any combination of drugs.

In various embodiments, one or more additional drugs may be optionally administered in addition to those that are recommended or have been administered. An additional drug will typically not be any drug that is not recommended or that should be avoided.

In one aspect, the present invention also provides a method of alleviating toxicity of the treatment. In one embodiment, the method of alleviating toxicity of the treatment alleviates the toxicity of a cancer treatment. For example, in one embodiment, the method of alleviating toxicity of the treatment alleviates the toxicity of an immune-modifying checkpoint blockage therapies.

### Method of Assessing the Prognosis, Assessing the Effectiveness, or Alleviating the Toxicity of Treatment of a Disease or Disorder

The present invention further relates, in part, to a method of assessing the prognosis or assessing the effectiveness of treatment of a disease or disorder associated with at least one antibody or target thereof (e.g., an antibody level, antibody target level, antibody activity, or antibody target activity) in a subject in need thereof.

In one aspect, the present invention provides a method of assessing the prognosis or assessing the effectiveness of treatment of a disease or disorder in a subject, the method comprising assessing the presence of at least one antibody target in the subject, wherein the at least one antibody target is identified to be associated with the disease or disorder according to the method described above. In one aspect, the present invention provides a method of assessing the prognosis or assessing the effectiveness of treatment of a disease or disorder in a subject, the method comprising assessing the level or activity of at least one antibody target in the subject, wherein the at least one antibody target is identified to be associated with the disease or disorder according to the method described above.

In one embodiment, the method of assessing the prognosis or assessing the effectiveness of treatment of a disease or disorder comprises comparing the level of at least one antibody target, that is identified to be associated with the disease or disorder according to the method described above, to the threshold level. In some embodiments, the threshold level is obtained from control group samples.

The present invention further relates, in part, to a method of assessing the prognosis or assessing the effectiveness of treatment of a disease or disorder associated with at least one antibody in a subject in need thereof. In one aspect, the present invention provides a method of assessing the prognosis or assessing the effectiveness of treatment of a disease or disorder in a subject, the method comprising assessing the presence of at least one antibody in the subject, wherein the at least one antibody is identified to be associated with the disease or disorder according to the method described above. In one aspect, the present invention provides a method of assessing the prognosis or assessing the effectiveness of treatment of a disease or disorder in a subject, the method comprising assessing the level or activity of at least one antibody in the subject, wherein the at least one antibody is identified to be associated with the disease or disorder according to the method described above.

In one embodiment, the method of assessing the prognosis or assessing the effectiveness of treatment of a disease or disorder comprises comparing the level of at least one antibody, that is identified to be associated with the disease or disorder according to the method described above, to the threshold level. In some embodiments, the threshold level is obtained from control group samples. In one embodiment, the threshold is 0.

In another aspect, the present invention provides a method of predicting a response to the treatment.

Information obtained from the methods of the invention described herein can be used alone, or in combination with other information (e.g., age, family history, disease status, disease history, vital signs, blood chemistry, PSA level, Gleason score, primary tumor staging, lymph node staging, metastasis staging, expression of other gene signatures relevant to outcomes of a disease or disorder, such as autoimmune disease or disorder, cancer, inflammatory disease or disorder, metabolic disease or disorder, neurodegenerative disease or disorder, organ tissue rejection, organ transplant rejection, or any combination thereof, etc.) from the subject or from the biological sample obtained from the subject.

### Compositions

The present invention also provides various compositions comprising the antibodies or targets thereof identified by methods of the present invention. In one embodiment, the compositions modulate a reactivity between an autoantibody and at least one antigen. In one embodiment, the antigen is an antigen set forth in Table 1.

In some embodiments, the composition of the invention increases the reactivity of at least one antigen of the invention with an antibody. In some embodiments, the composition of the invention comprises at least one autoantibody directed to at least one antigen set forth in Table 1.

In some embodiments, the composition of the invention decreases the reactivity of at least one antigen of the invention with an antibody. In one embodiment, the invention provides compositions comprising at least one antigen of the invention linked to at least one domain for endocytosis, degradation, or a combination thereof. In one embodiment, the invention provides a composition comprising an antigen selected from the antigens set forth in Table 3, or a fragment thereof, linked to a domain for endocytosis, degradation, or a combination thereof. In one embodiment, the invention provides a composition comprising an antigen selected from the antigens set forth in Table 6, or a fragment thereof, linked to a domain for endocytosis, degradation, or a combination thereof.

In one embodiment, the invention provides a composition comprising a nucleic acid molecule encoding an antigen selected from the antigens set forth in Table 3, or a fragment thereof, linked to a domain for endocytosis, degradation, or a combination thereof. In one embodiment, the invention provides a composition comprising a nucleic acid molecule encoding an antigen selected from the antigens set forth in Table 6, or a fragment thereof, linked to a domain for endocytosis, degradation, or a combination thereof.

In one embodiment, the invention provides compositions comprising a cell or particle expressing at least one antigen of the invention, for example, a CAR T-cell expressing at least one antigen of the invention as described elsewhere herein.

In various aspects, the composition comprises: one or more antibodies or targets thereof of the present invention and one or more stabilizers. In various embodiments, the stabilizer to compound weight ratio is less than 50%. In one embodiment, the stabilizer comprises a biocompatible polymer. Examples of stabilizers include, but are not limited to, biocompatible polymer, a biodegradable polymer, a multifunctional linker, starch, modified starch, and starch derivatives, gums, including but not limited to polymers, polypeptides, albumin, amino acids, thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines, carboxylic acids, or combinations thereof.

The compositions may be formulated in a pharmaceutically acceptable excipient, such as wetting agents, buffers, disintegrants, binders, fillers, flavoring agents and liquid carrier media such as sterile water, water/ethanol etc. The compositions should be suitable for administration either by topical administration or injection or inhalation or catheterization or instillation or transdermal introduction into any of the various body cavities including the alimentary canal, the vagina, the rectum, the bladder, the ureter, the urethra, the mouth, etc. For oral administration, the pH of the composition is preferably in the acid range (e.g., 2 to 7) and buffers or pH adjusting agents may be used. The contrast media may be formulated in conventional pharmaceutical administration forms, such as tablets, capsules, powders, solutions, dispersion, syrups, suppositories etc.

The compositions of the invention can be formulated and administered to a subject, as now described. The invention encompasses the preparation and use of pharmaceutical compositions comprising the compositions of the invention useful for the delivery of a therapeutic agent to a cell. The invention also encompasses the preparation and use of pharmaceutical compositions comprising the compositions of the invention useful for the treatment of a disease or disorder. The invention also encompasses the preparation and use of pharmaceutical compositions comprising the compositions of the invention useful for improved cell penetration.

Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate therapeutic composition, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate modulator thereof, according to the methods of the invention.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal and other known routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient. In various embodiments, the composition comprises at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Parenteral administration of a pharmaceutical composition includes any route of administration characterized by physical breaching of a tissue of an individual and administration of the pharmaceutical composition through the breach in the tissue. Parental administration can be local, regional or systemic. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, intraocular, intravitreal, subcutaneous, intraperitoneal, intramuscular, intradermal, intrasternal injection, and intratumoral.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono-or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some embodiments having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 nanomaters to about 2000 micrometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa.

Administration of the compounds of the present invention or the compositions thereof may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art.

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into the muscle. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. In one embodiment, the composition described above is administered to the subject by subretinal injection. In other embodiments, the composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired. In another embodiments, route of administration is subretinal injection or intravitreal injection.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the composition in the particular host.

The pharmaceutical compositions useful for practicing the invention may be administered to deliver a dose of at least about 1 ng/kg, at least about 5 ng/kg, at least about 10 ng/kg, at least about 25 ng/kg, at least about 50 ng/kg, at least about 100 ng/kg, at least about 500 ng/kg, at least about 1 μg/kg, at least about 5 μg/kg, at least about 10 μg/kg, at least about 25 μg/kg, at least about 50 μg/kg, at least about 100 μg/kg, at least about 500 μg/kg, at least about 1 mg/kg, at least about 5 mg/kg, at least about 10 mg/kg, at least about 25 mg/kg, at least about 50 mg/kg, at least about 100 mg/kg, at least about 200 mg/kg, at least about 300 mg/kg, at least about 400 mg/kg, and at least about 500 mg/kg of body weight of the subject.

In some embodiments, the pharmaceutical compositions useful for practicing the invention may be administered to deliver a dose of no more than about 1 ng/kg, no more than about 5 ng/kg, no more than about 10 ng/kg, no more than about 25 ng/kg, no more than about 50 ng/kg, no more than about 100 ng/kg, no more than about 500 ng/kg, no more than about 1 μg/kg, no more than about 5 μg/kg, no more than about 10 μg/kg, no more than about 25 μg/kg, no more than about 50 μg/kg, no more than about 100 μg/kg, no more than about 500 μg/kg, no more than about 1 mg/kg, no more than about 5 mg/kg, no more than about 10 mg/kg, no more than about 25 mg/kg, no more than about 50 mg/kg, no more than about 100 mg/kg, no more than about 200 mg/kg, no more than about 300 mg/kg, no more than about 400 mg/kg, and no more than about 500 mg/kg of body weight of the subject. Also contemplated are dosage ranges between any of the doses disclosed herein.

Typically, dosages which may be administered in a method of the invention to a subject, in some embodiments a human, range in amount from 0.5 μg to about 100 g per kilogram of body weight of the subject. While the precise dosage administered will vary depending upon any number of factors, including but not limited to, the type of subject and type of disease state being treated, the age of the subject and the route of administration. In some embodiments, the dosage of the compound will vary from about 1 μg to about 10 mg per kilogram of body weight of the subject. In other embodiments, the dosage will vary from about 3 μg to about 1 mg per kilogram of body weight of the subject.

The compositions may be administered to a subject as frequently as several times daily, or it may be administered less frequently, such as once a day, twice a day, thrice a day, once a week, twice a week, thrice a week, once every two weeks, twice every two weeks, thrice every two weeks, once a month, twice a month, thrice a month, or even less frequently, such as once every several months or even once or a few times a year or less. The frequency of the dose will be readily apparent to the skilled artisan and will depend upon any number of factors, such as, but not limited to, the type and severity of the disease being treated, the type and age of the subject, etc. The formulations of the pharmaceutical compositions may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Individuals to which administration of the pharmaceutical compositions of the invention is contemplated include, but are not limited to, humans and other primates, mammals including commercially relevant mammals such as non-human primates, cattle, pigs, horses, sheep, cats, and dogs.

These compositions described herein are by no means all-inclusive, and further modifications to suit the specific application will be apparent to the ordinary skilled artisan. Moreover, the effective amount of the compositions can be further approximated through analogy to compounds known to exert the desired effect.

### Kits

The present invention also pertains to kits useful in the methods of the invention. Such kits comprise various combinations of components useful in any of the methods described elsewhere herein, including for example, materials for identifying at least one antibody target, quantitatively analyzing at least one antibody or a target thereof (e.g., quantitatively analyzing a nucleic acid sequence barcode), materials for diagnosing or assessing the prognosis of a disease or disorder associated with the antibody or target thereof, materials for preventing or treating a disease or disorder associated with the antibody or target thereof, materials for alleviating toxicity of the treatment, and instructional material. For example, in one embodiment, the kit comprises components useful for the identification of a desired antibody target in a biological sample. In another embodiment, the kit comprises components useful for the quantification of a desired antibody or a desired antibody target (e.g., quantification of a desired nucleic acid sequence barcode). In a further embodiment, the kit comprises components useful for diagnosing or assessing the prognosis of a disease or disorder associated with the antibody or target thereof In a further embodiment, the kit comprises components useful for preventing or treating a disease or disorder associated with the antibody or target thereof. In a further embodiment, the kit comprises components useful for alleviating toxicity of the treatment.

In a further embodiment, the kit comprises the components of an assay for monitoring the effectiveness of a treatment administered to a subject in need thereof, containing instructional material and the components for determining whether the level of an antibody or a target thereof of the invention in a biological sample obtained from the subject is modulated during or after administration of the treatment. In various embodiments, to determine whether the level of an antibody or a target thereof of the invention is modulated in a biological sample obtained from the subject, the level of the antibody or the target thereof is compared with the level of at least one comparator contained in the kit, such as a positive control, a negative control, a historical control, a historical norm, or the level of another reference molecule in the biological sample. In certain embodiments, the ratio of the antibody or the target thereof and a reference molecule is determined to aid in the monitoring of the treatment.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Rapid Extracellular Antibody Profiling (REAP)

Current high-throughput autoantibody discovery techniques have limited sensitivity towards extracellular and secreted proteins largely due to the biochemical challenges associated with producing these proteins in a high-throughput manner. In this regard, yeast cell surface display offers several important advantages over other common systems. Unlike in vitro translation or peptide-array-based approaches, yeast cell surface display can express full-length proteins in folded three-dimensional conformations, allowing for the identification of non-linear binding epitopes. Compared to phage or bacterial expression systems, yeast cell produced extracellular proteins in a eukaryotic cell system that included ER chaperones, glycosylation machinery, and disulfide “proofreading.” While mammalian systems may offer even superior quality control owing to more native glycosylation machinery and chaperones, a yeast cell surface display library is far more economical to maintain and expand. These advantages combine to make a yeast-displayed exoproteome library a robust solution that can maximize the sensitivity and throughput of extracellular autoantibody discovery.

The present study generated, characterized, and applied a high-quality yeast-display based platform to identify extracellular proteins that are targets of autoantibodies. The system was benchmarked using a well-characterized autoimmune syndrome with pathognomonic autoantibody targets and showed that it has high sensitivity and specificity. The method was additionally applied to a cohort of immunotherapy-treated NSCLC patients and another cohort of patients with SLE, UCTD, and sarcoidosis. In both cohorts several novel autoantibody reactivities were identified and validated.

REAP as a Novel Autoantibody Discovery Platform

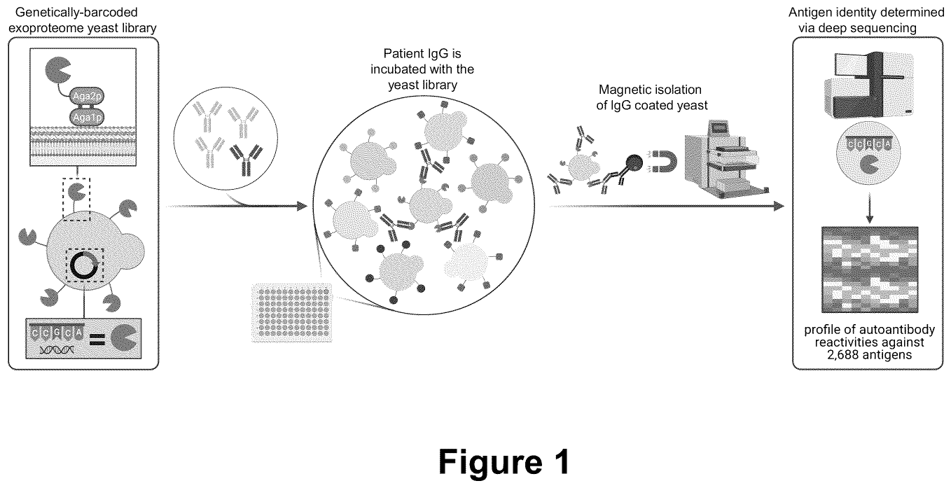

In order to leverage the power of yeast cell surface display systems for autoantibody discovery, a yeast-displayed “exoproteome” library of approximately 1400 human extracellular or secreted proteins, where each protein in the library was paired with unique DNA barcodes, was used. Using this library, REAP, a platform that allowed for sensitive high throughput identification of autoantibody reactivities against extracellular proteins, was developed. In it, purified patient antibodies were incubated with the library. Autoantibodies, if present, bound to yeast cell clones displaying their target antigen. These autoantibody-coated yeast cells were enriched by magnetic bead-based selection and enrichment was quantified through next generation sequencing of the unique DNA barcodes (FIG. 1).

In developing REAP, a number of novel methodologies had to be established. These include advances in antigen library preparation as well as advances in methodology for preparation of patient biological samples, high-throughput selection, and downstream data analysis. First, a necessary component of REAP was the defined linkage between a genetically encoded barcode that may be read out by next-generation sequencing and an associated gene. While multiple barcodes may be associated with the same gene, no barcode may be associated with multiple genes for the REAP assay to function. Additionally, REAP required a library composed of native, properly-folded proteins comprising individual extracellular domains (“ectodomains”). Therefore, approaches, such as peptide tiling, shotgun DNA cloning, or whole-cDNA cloning approaches, which have previously been used to generate libraries for autoantibody screening, did not offer the same specificity or coverage as the curated library since they did not present the full, properly folded tertiary structure of the secreted or ectodomain antigen. As such, these technologies cannot readily detect antibodies recognizing discontinuous, three-dimensional epitopes. These difficulties were overcome and generated a curated library of full-length ectodomains that were individually cloned, normalized during a pooling step, and confidently associated with multiple unique genetic barcodes.

Second, a high-throughput and efficient method for antibody isolation from human serum or plasma were developed. This method involved affinity purification of the desired antibody isotype (IgG, IgA, IgE, etc.) in 96-well microtiter plates. This allowed for the isolation of antibodies from hundreds of patient samples in a day. Importantly, after the antibodies were isolated, they were incubated with empty vector yeast. Since yeast cell contained conserved epitopes that may be targeted by endogenous anti-saccharomyces antibodies and proteins, such as complement/MBL, this step removed human serum components and yeast-reactive antibodies that may bind yeast cell and interfere with downstream selection procedures. Ultimately, the antibody isolation method allowed to rapidly process patient samples while generating antibody inputs that lead to minimal background in the REAP selection process.

Third, a novel high-throughput selection process based on 96-well magnetic columns were developed. Traditionally, yeast cell library selections for directed evolution purposes have been conducted with either large magnetic columns designed for capturing cells or fluorescence activated cell sorting (FACS). While this process was effective, it was entirely low-throughput. Using these large magnetic columns, only a few dozen selections can be performed at a time. Use of FACS was similarly limiting, as one FACS machine can only sort one sample at a time at a maximum speed of ˜17 minutes per 100 million cells. In order to achieve the desired level of throughput, 96-well magnetic columns designed for analytical scale isolations of proteins and nucleic acids were repurposed. Through optimization, a standard protocol for use of these columns that involved washing to remove non-specific binders as well as centrifugation for maximum elution efficiency was developed. Using this novel selection method, the entire selection process for 96 samples consisting of 100 million cells per sample can be completed in ˜40 minutes, while comparable sorting using FACS would take ˜27 hours.

Finally, a custom scoring algorithm was developed to identify genuine autoantibody reactivities based on quantitative next generation sequencing data. The data analysis method relied on the fact that each protein in the library was displayed on multiple yeast cell clones and each clone carried a unique DNA barcode. In other words, each protein in the library consisted of multiple “protein clones”. Through next generation sequencing, not only can the total enrichment of a protein after selection be determined, but also how many “protein clones” were enriched. This allows for quantifying “clonal enrichment”, which was defined as the fraction of clones that were enriched above a set cutoff. Incorporation of clonal enrichment in REAP data analysis was essential for identification of true reactivities because it allowed for the elimination of non-specific enrichment of proteins due to polyreactive “sticky” yeast cell clones or stochastic variations in library distribution. These factors may result in enrichment of a single protein clone, but it was extremely unlikely that they would result in enrichment of all of the “protein clones” for a protein. On the other hand, genuine enrichment of a protein due to the presence of autoantibodies targeting it would result in enrichment of many if not all protein clones. Thus, incorporation of clonal enrichment into data analysis allowed for elimination of false positive enrichments, expediting identification of genuine autoantibody reactivities in samples.

REAP Allows for Specific and Sensitive High-Throughput Autoantibody Discovery

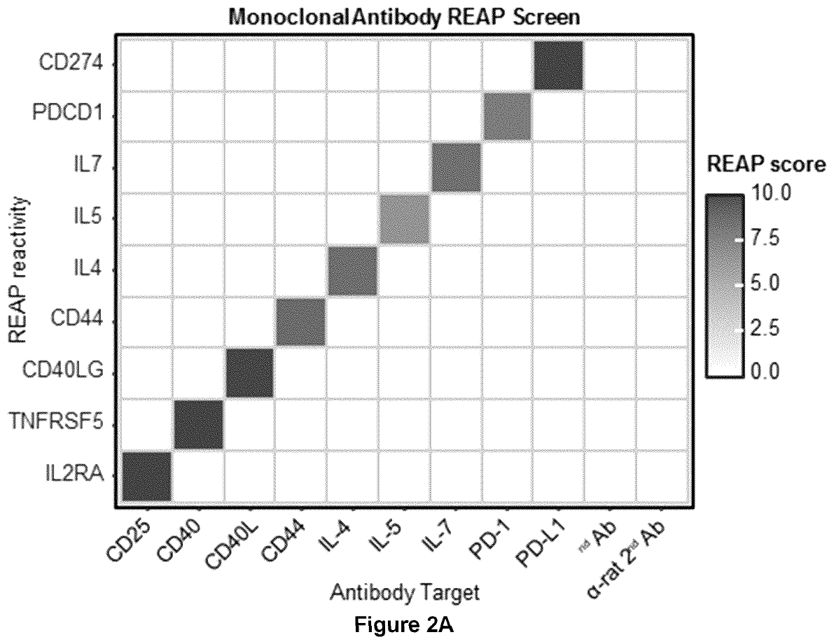

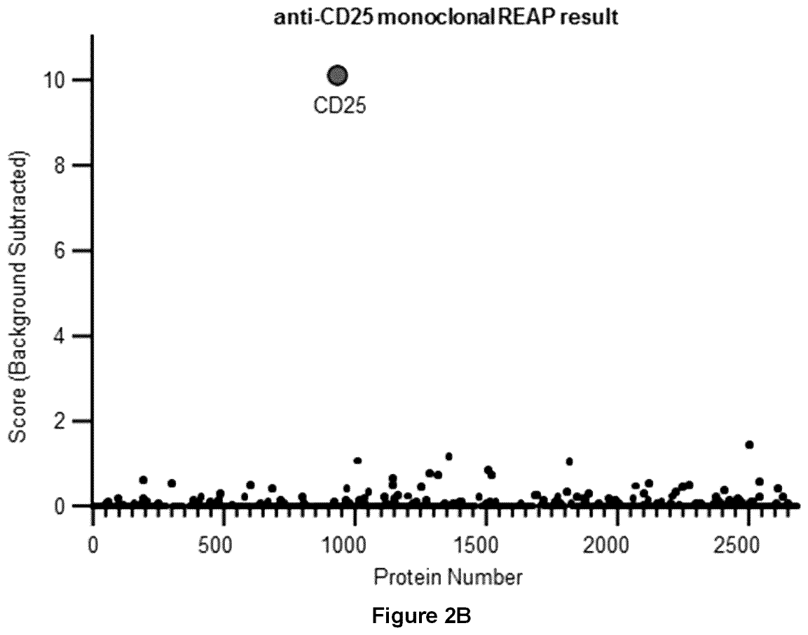

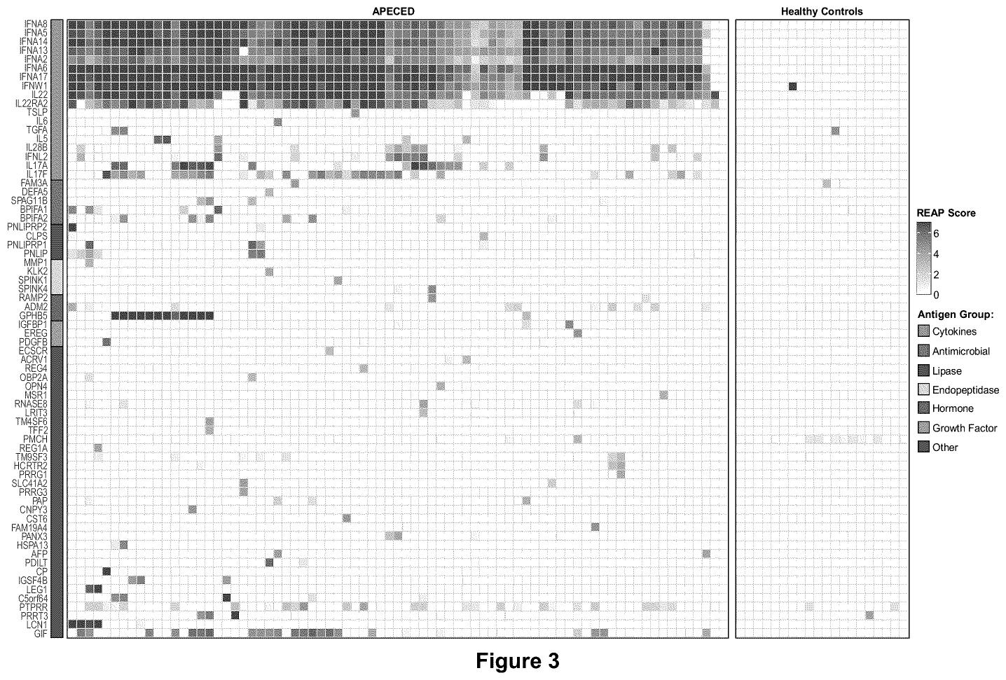

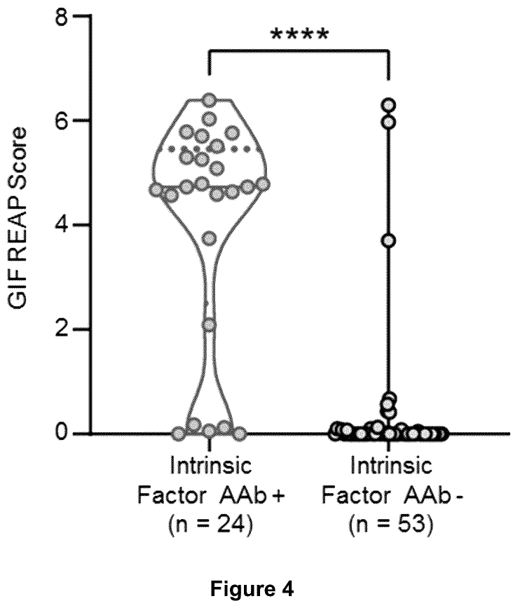

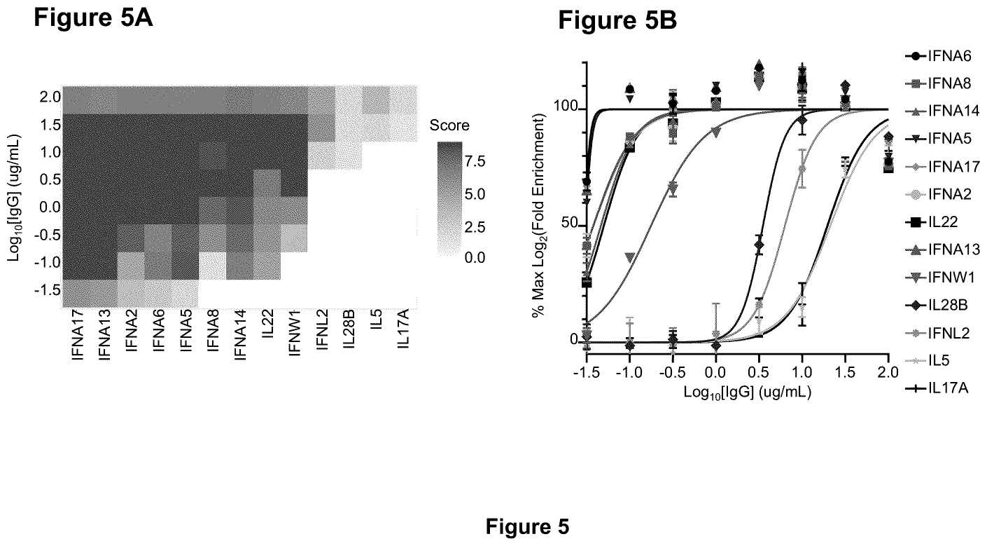

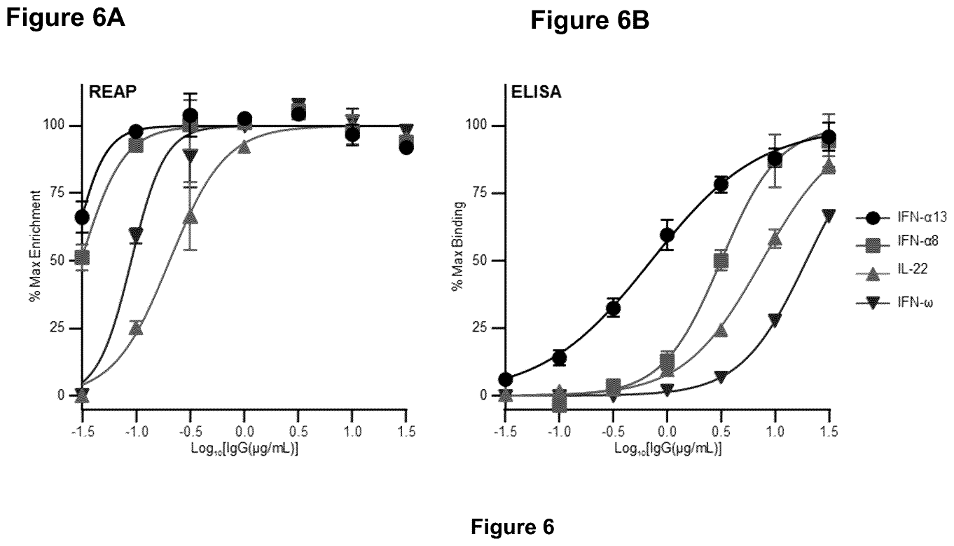

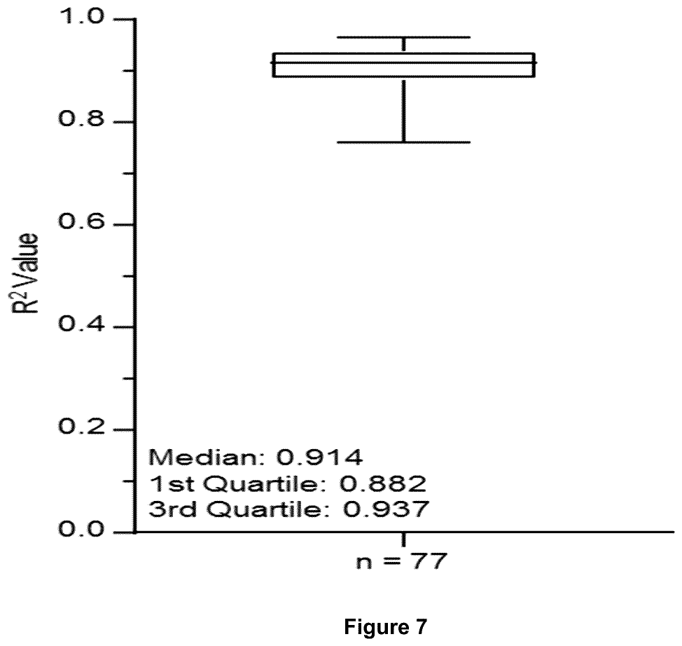

To validate that this method can accurately detect antibody targets, REAP was performed on a panel of 9 commercial monoclonal antibodies with known targets (FIG. 2). All antibody targets in this panel were detect accurately and specifically. Next, the assay was benchmarked using samples from patients with autoimmune-polyendocrinopathy-candidiasis-ectodermal dystrophy (APECED), an autoimmune disease characterized by near universal presence of high titer autoantibodies against type 1 interferons and IL22 and rarer autoantibodies against other cytokines. IgG was purified from the serum of twelve APECED patients along with 16 healthy donor samples and conducted REAP on them. This REAP screen revealed that all APECED samples exhibited robust enrichment of type 1 interferons (IFNA & IFNW1) and 1L22 and several exhibited enrichment of other known autoantibody targets in APECED such as IL17, IL5, and IL28 at frequencies comparable to previously described autoantibody distributions in the APECED patient population (FIG. 3). Little to no enrichment of these proteins was seen in the 20 healthy donor samples. Autoantibodies were identified against gastric intrinsic factor (GIF), lipocalin-1 (LCN1), IL-5, IL-6, protein disulfide-isomerase-like protein of the testis (PDILT), and BPI fold containing family member 1 and 2 (BPIFA1/2), which have been previously described in APECED. With respect to GIF reactivities, the results seen with REAP demonstrated strong concordance with clinical anti-GIF ELISA results from the same patients (FIG. 4). To quantify the sensitivity of the assay, REAP screens were conducted using serial dilutions of antibody from an APECED patient (FIG. 5) and compared the results to that of enzyme-linked immunosorbent assays (ELISAs), the “gold-standard” assay for autoantibody detection (FIG. 6). For the four protein targets tested, REAP exhibited higher sensitivity than ELISA, as seen by the left-shifted dose response curves in the REAP assay. To investigate the reproducibility of REAP, log 2[fold enrichment] was compared between technical (intra-assay) replicates across all APECED patient samples and strong positive correlations were found between replicates (median R2=0.914; FIG. 7). Together, these data show that REAP is a sensitive and specific assay for high-throughput autoantibody identification from patient serum.

REAP Identifies Novel Autoantibodies in a Wide Variety of Disease Contexts

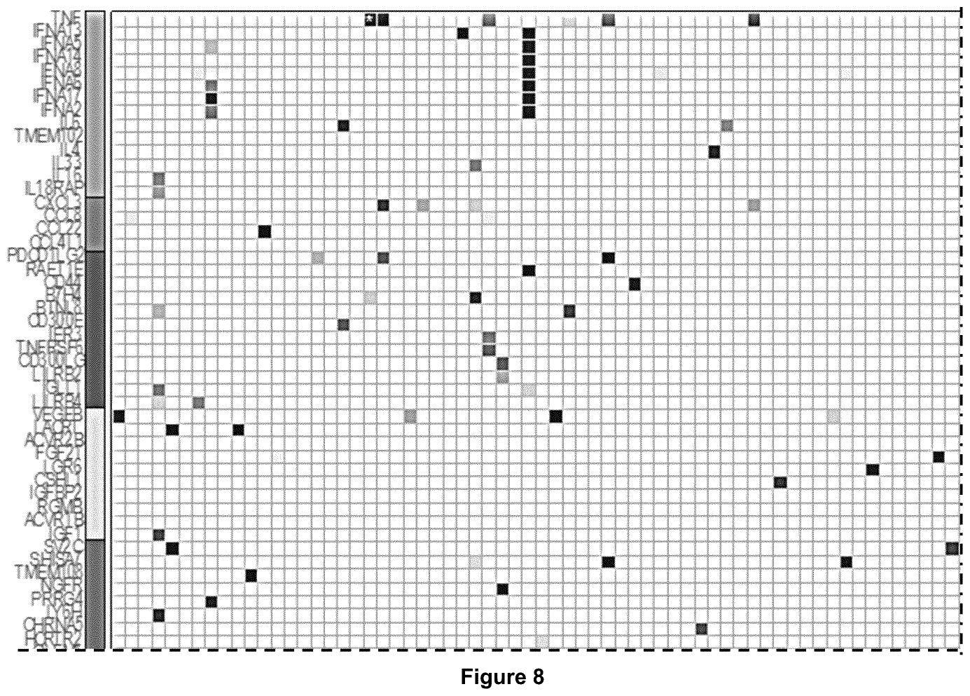

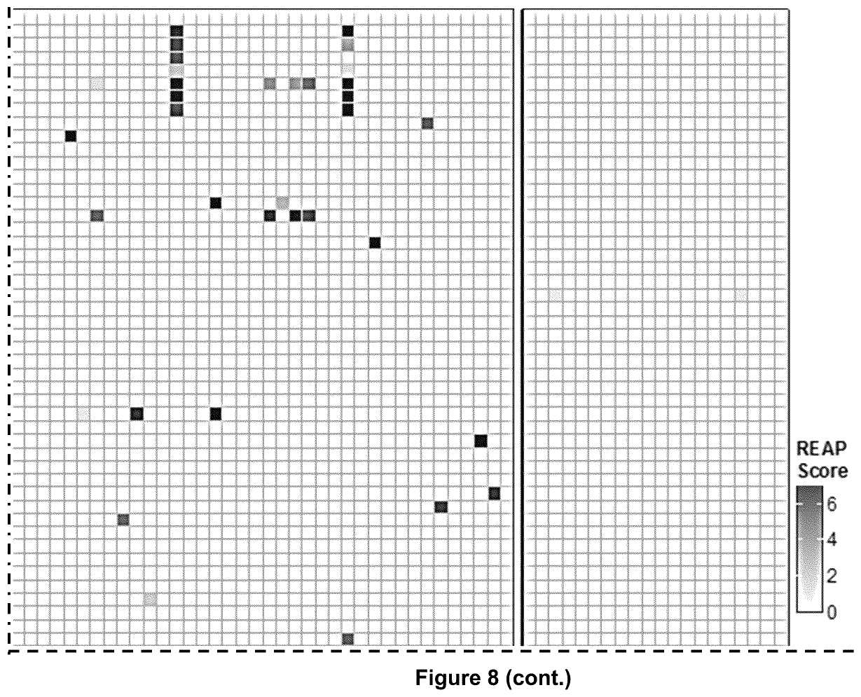

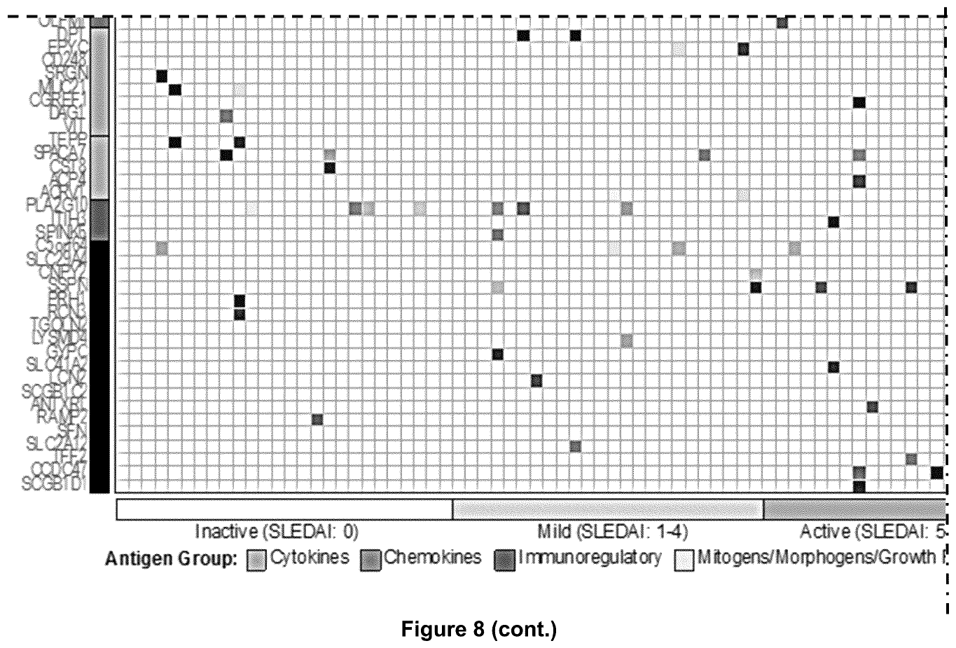

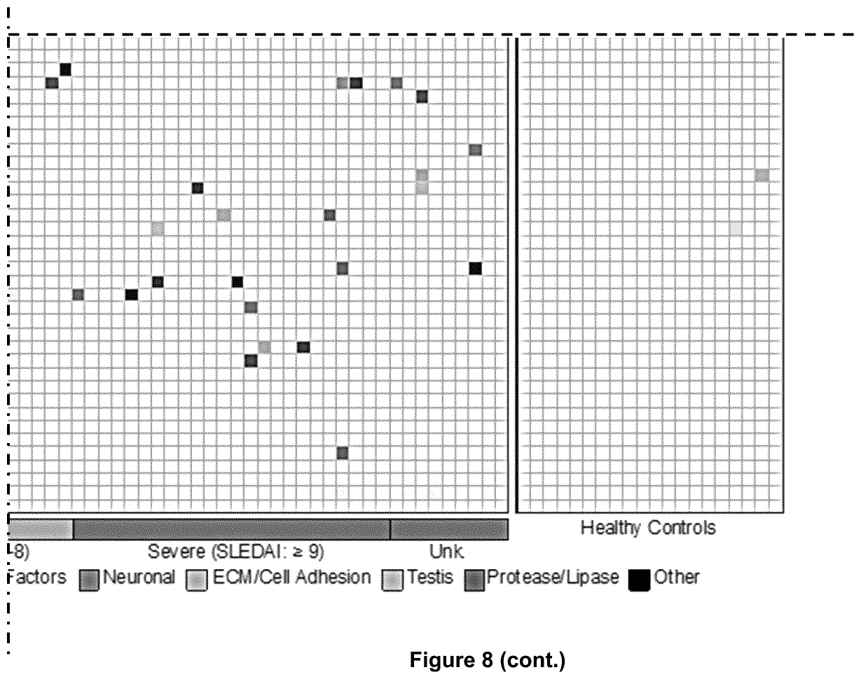

Using REAP, a cohort of patients with systemic lupus erythematosus (SLE) was screened (FIG. 8). THe screen identified autoantibody reactivities that are known to be present in SLE patients, such as those against TNF, IL6, and type I interferons. Importantly, many previously undescribed autoantibody reactivities were identified against proteins with a wide range of biological functions. For example, autoantibody reactivities were identified targeting cytokines (e.g., IL4, IL33), chemokines (e.g., CXCL3, CCL8), growth factors (e.g., VEGFB, FGF21), immunoregulatory proteins (e.g., PD-L2, B7H4), and extracellular matrix proteins (e.g., EPYC, CD248).

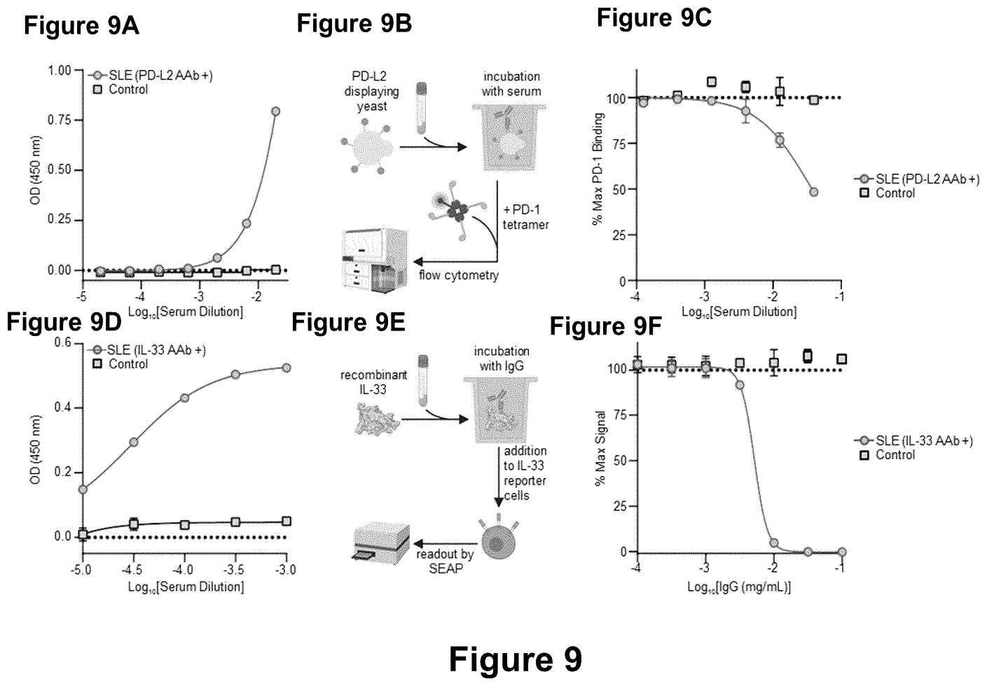

Two notable autoantibody reactivities uncovered in SLE patients were those against PD-L2 and IL-33. These were biochemically validated using ELISAs and the function of these autoantibodies was characterized. As the primary biological function of PD-L2 is mediated by its binding to its receptor PD-1, it was tested whether autoantibodies against PD-L2 could block this interaction. Serum samples from an SLE patient with anti-PD-L2 autoantibodies were present at titers >1:100 and inhibited the interaction between PD-L2 and PD-1 in a dose-dependent manner, while serum from a control patient without anti-PD-L2 autoantibodies did not (FIG. 9A-9C). To test the functional effects of anti-IL-33 autoantibodies, a HEK-Blue IL-33 reporter cell line was used, which produces secreted alkaline phosphatase downstream of an NFκB promoter that is activated by the IL-33 pathway. Bulk IgG (isolated via protein G) from the SLE patient harboring anti-IL-33 autoantibodies potently neutralized IL-33 signaling with an IC50 less than 0.01 mg/mL, while IgG from a control patient without anti-IL-33 autoantibodies had no neutralizing effect (FIG. 9D-9F). These findings underscore the ability of REAP to discover novel autoantibodies with functional biological effects.

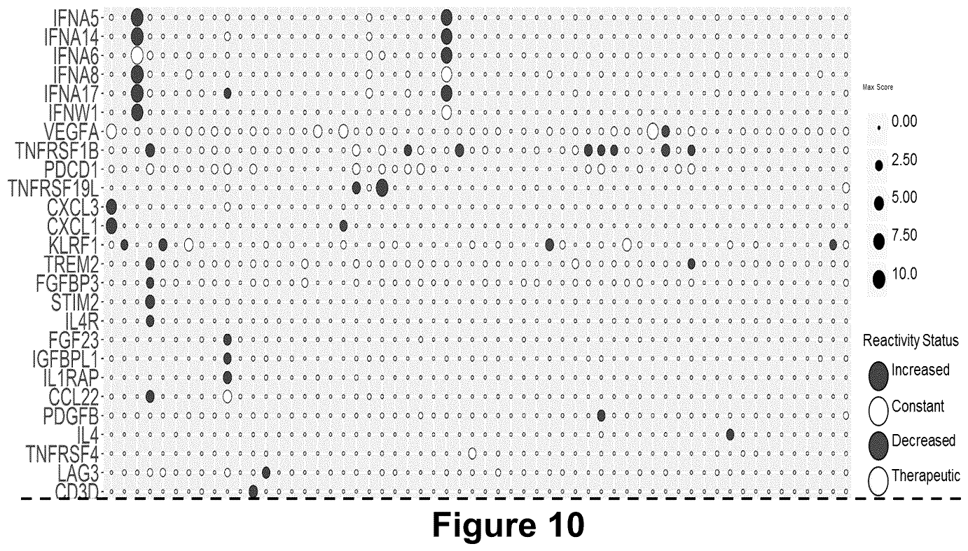

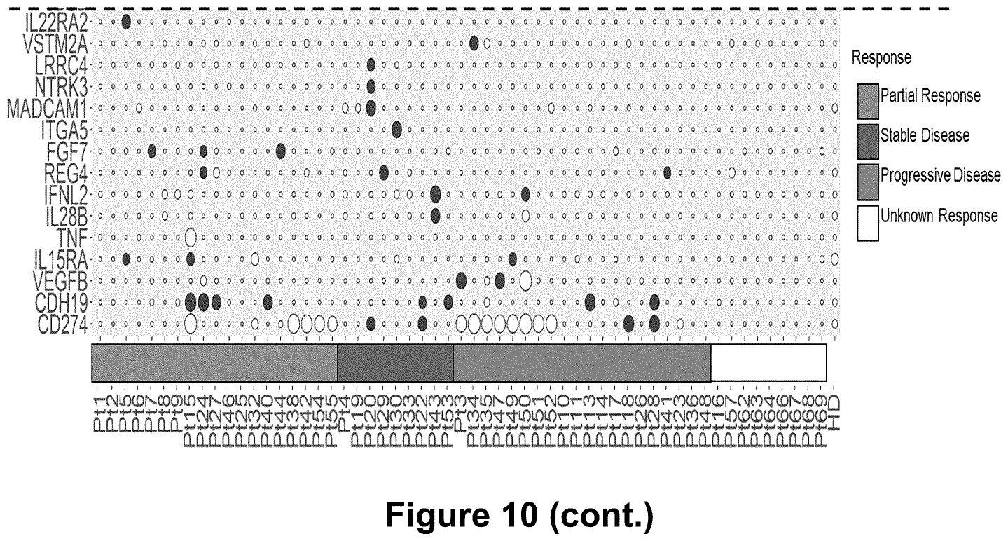

In addition, a longitudinal cohort of 63 non-small cell lung cancer (NSCLC) patients treated primarily with anti-PD-L1 and anti-PD-1 checkpoint inhibition along with a variety of other antibody immunotherapies (FIG. 10) was screened. From this screen, novel autoantibody reactivities against proteins that have not yet been described in the context of cancer and that could potentially have disease-modifying effects were identified. These include autoantibodies targeting chemokines (e.g., CXCL1/2/3), type 1 interferons, growth factors (e.g., VEGFB), and adhesion receptors (e.g., MADCAM1).

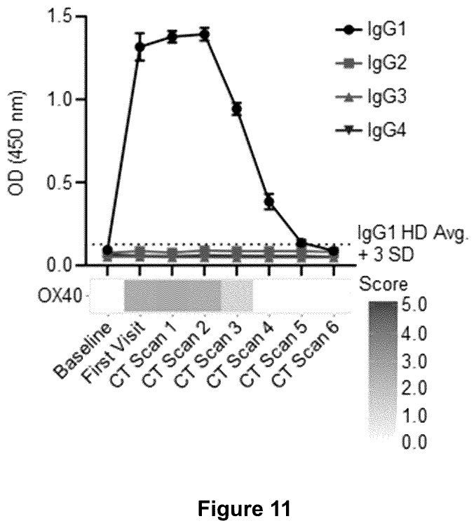

Using REAP, many of the therapeutic antibodies administered to these patients were accurately detected, which served as internal positive controls. The assay was able to detect therapeutic antibody presence with high sensitivity. In one patient, patient 9, bevacizumab (anti-VEGFA therapeutic antibody) was detected 6 months after their last dose. The assay was also able to accurately detect longitudinal changes in therapeutic antibody titer. For example, REAP score accurately reflected changes in therapeutic anti-OX40 antibody titers in one patient, as measured by ELISA (FIG. 11).

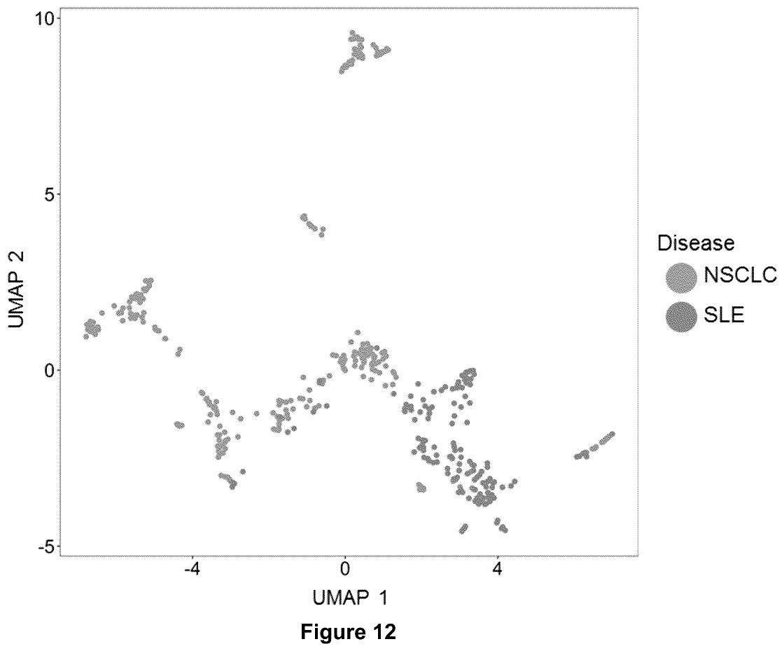

Combining these data with the SLE REAP data, the heterogeneity in REAP data was analyzed between different diseases by performing UMAP analysis on the NSCLC, SLE, and UCTD patient data (FIG. 12). While some NSCLC and SLE patients clustered together, some subsets of patients formed distinct disease-specific clusters.

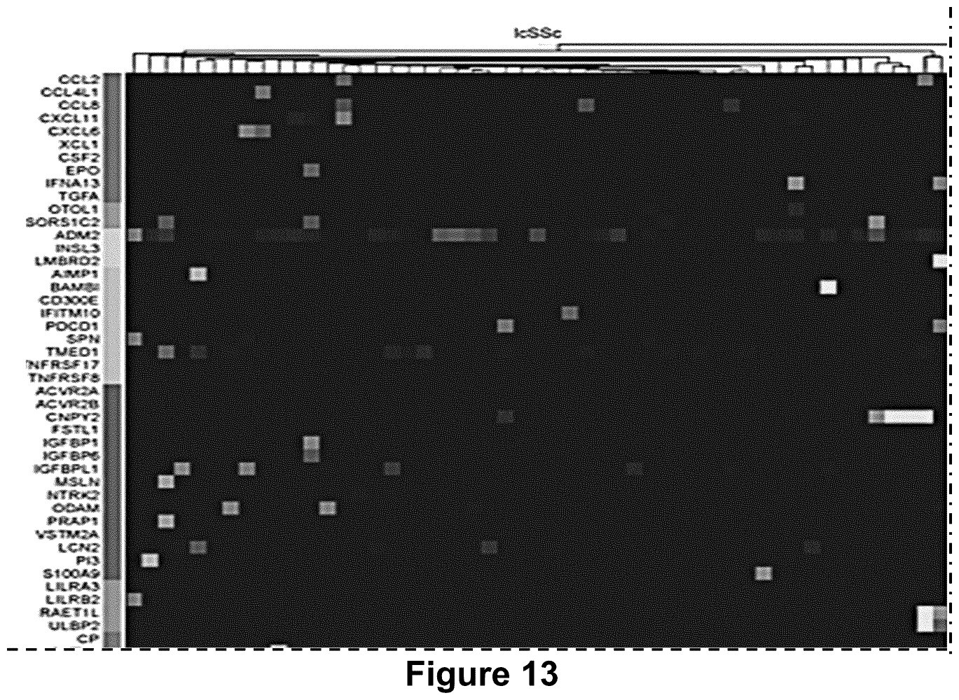

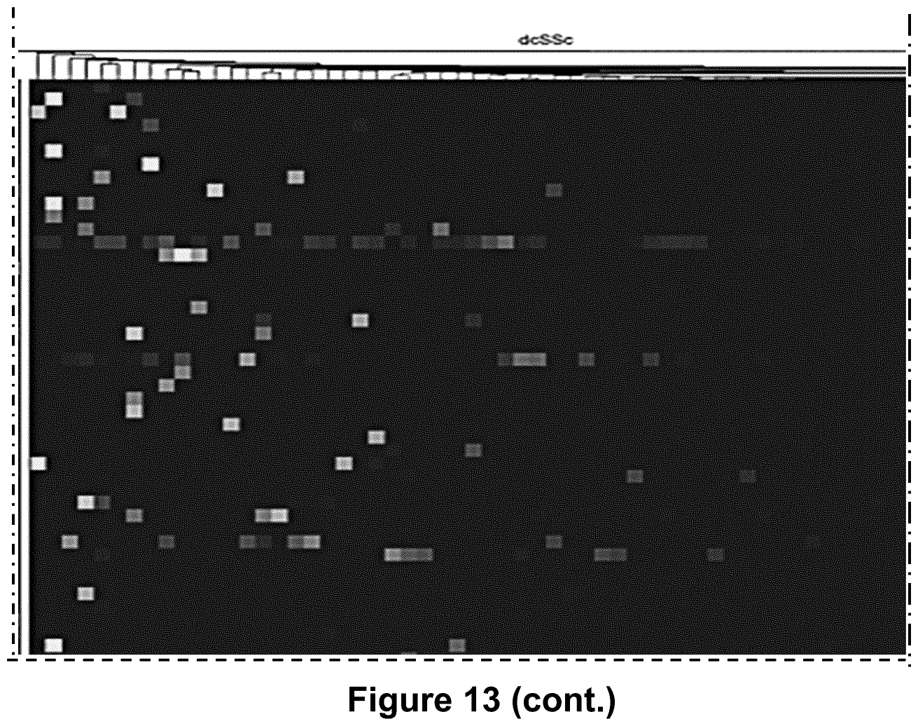

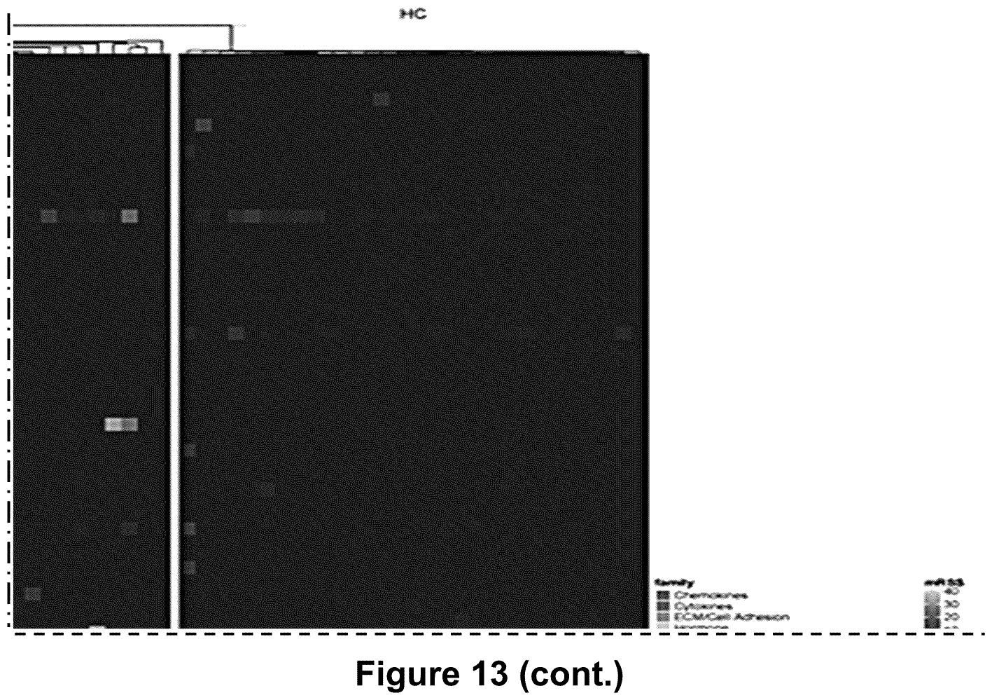

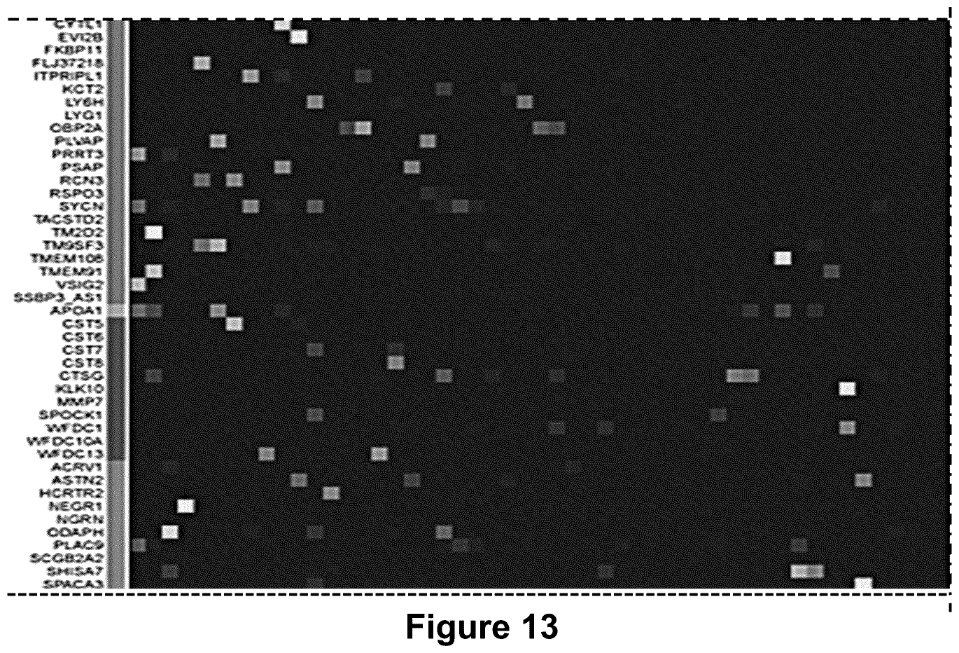

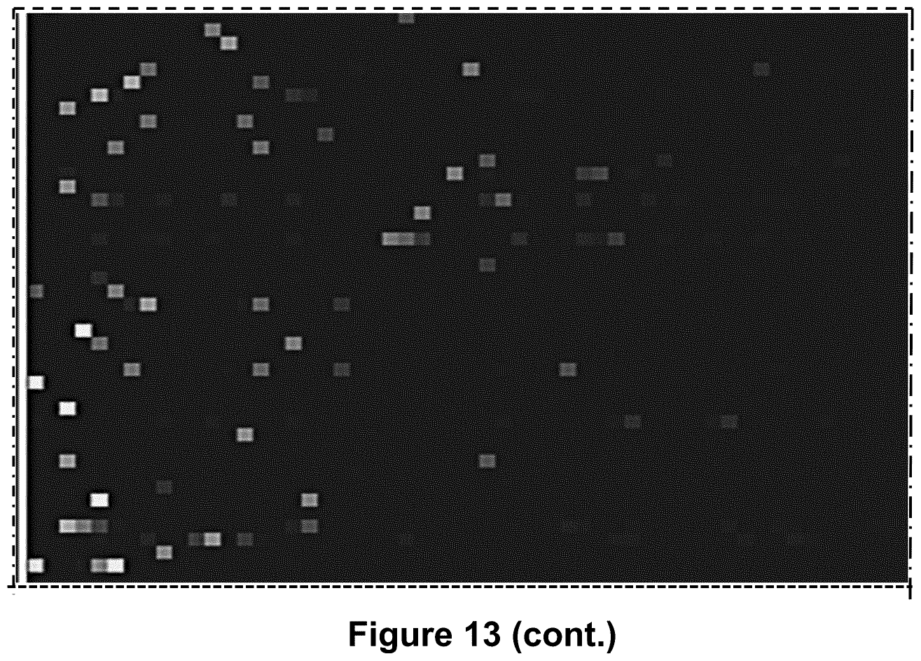

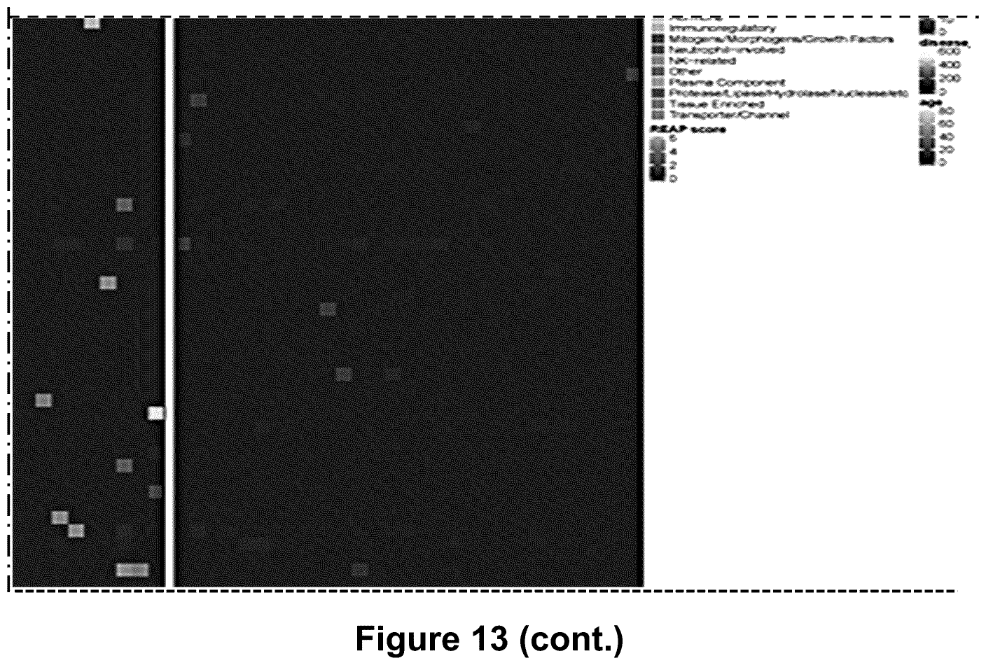

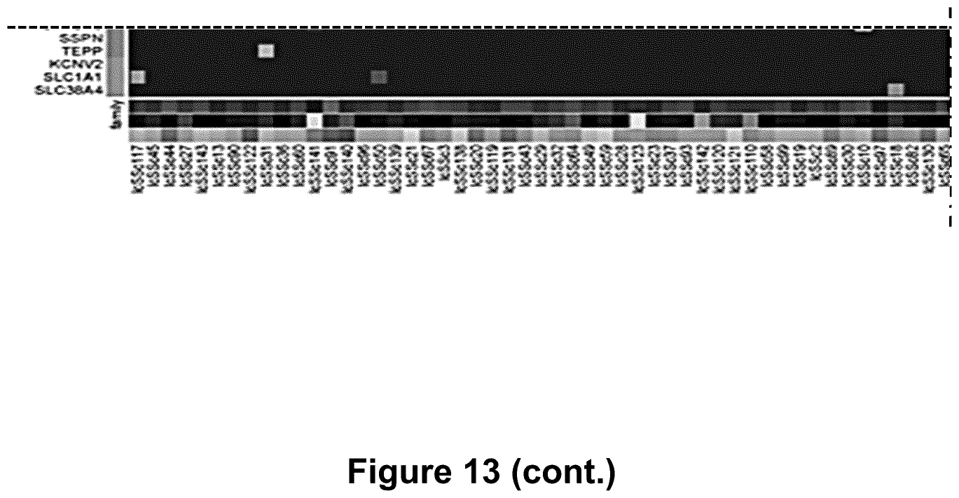

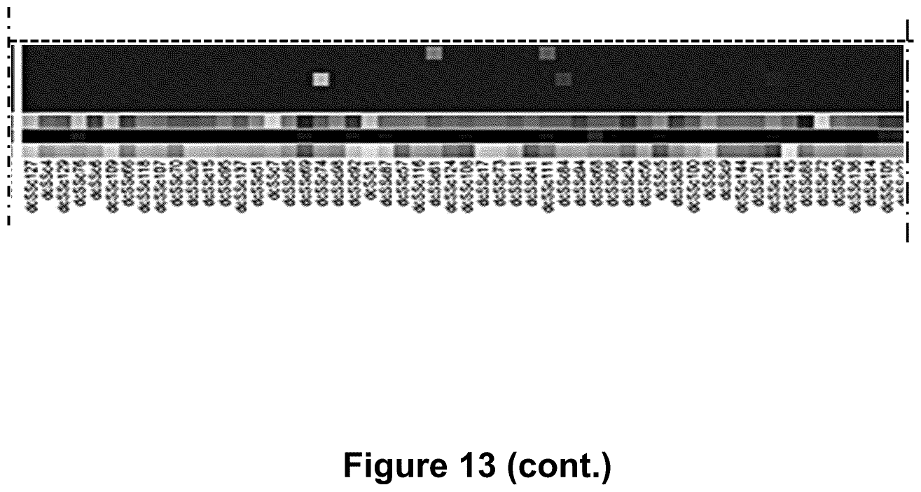

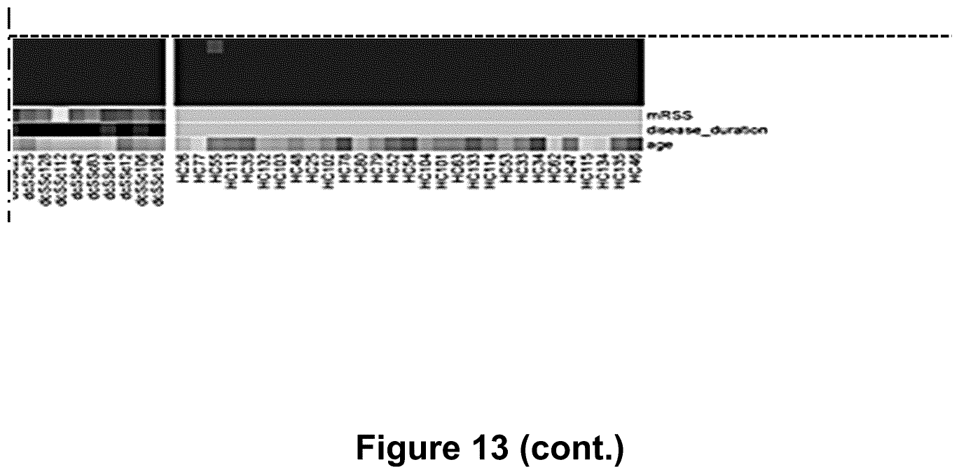

A cohort of patients was screened with systemic sclerosis, a chronic autoimmune rheumatic disorder (FIG. 13). Similar to the screen of SLE patients, numerous novel autoantibody reactivities targeting proteins involved in a wide variety of biological functions were found. Of note, many reactivities against NK cell related proteins (LILRA3, LILRB2, RAETIL, ULBP2) were identified and multiple patients had autoantibody reactivities against PD-1, an immune checkpoint receptor that plays an important role in inhibiting immune responses.

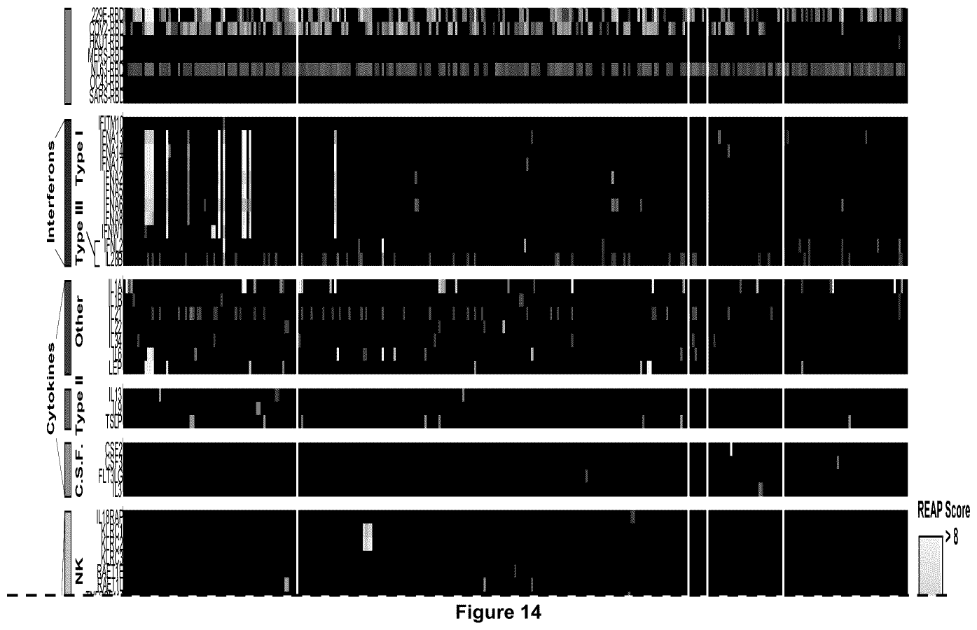

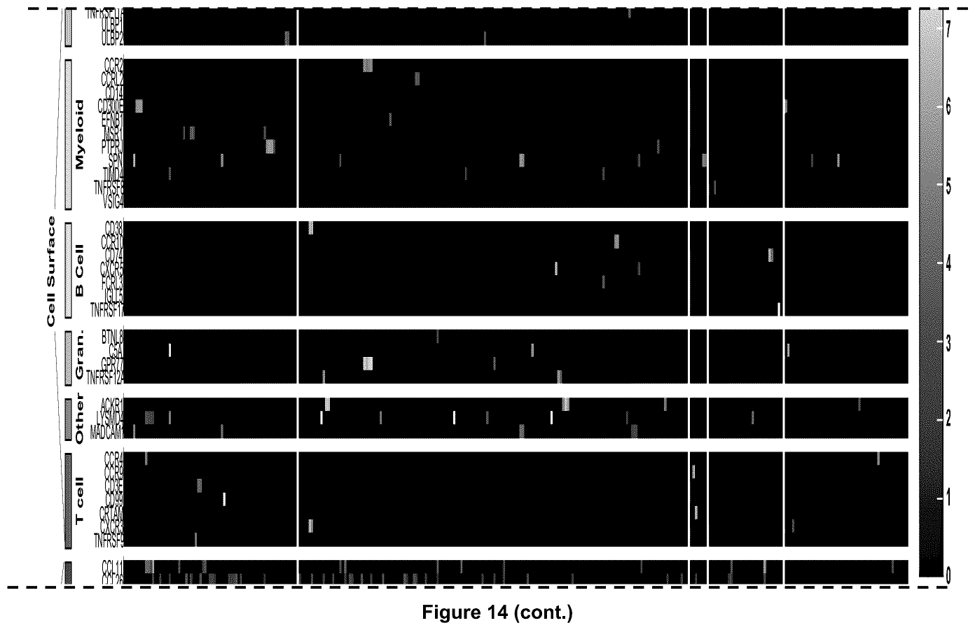

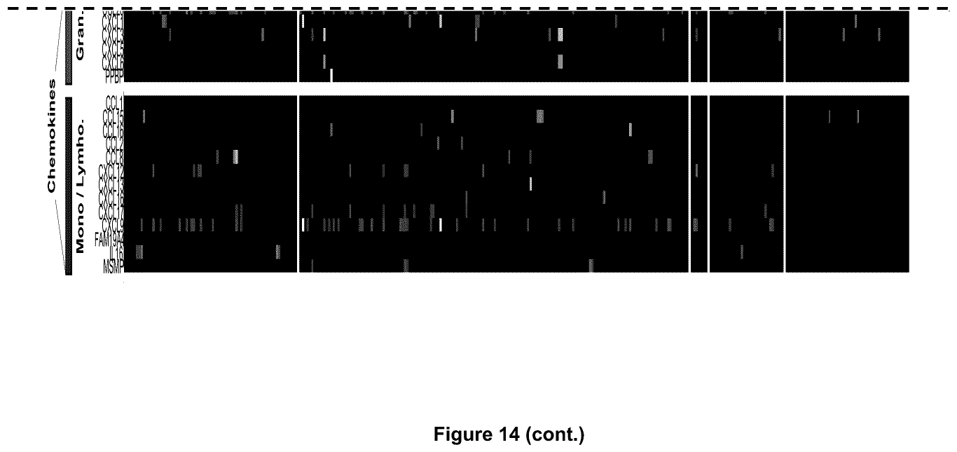

Finally, a longitudinal cohort of 194 COVID-19 patients were screened. It was found that autoantibodies in COVID-19 patients targeted proteins involved in diverse immunological functions such as acute phase response, type II immunity, leukocyte trafficking, interferon responses, and lymphocyte function/activation (FIG. 14). Cytokine autoantibody targets included type 1 and type 3 interferons, IL-1α/β, IL-6, IL-21, IL-22, GM-CSF (CSF2), IL-18Rβ (IL18RAP), and Leptin (LEP). Chemokine autoantibody targets included CXCL1, CXCL7 (PPBP), CCL2, CCL15, CCL16, and the chemokine decoy receptor ACKR1 (Duffy blood group antigen). Immunomodulatory cell surface autoantibody targets included NKG2D ligands (e.g., RAET1E/L, ULBP1/2), NK cell receptors NKG2A/C/E (e.g., KLRC1/2/3), B cell expressed proteins (e.g., CD38, FCMR, FCRL3, CXCR5), T cell expressed proteins (e.g., CD3E, CXCR3, CCR4), and myeloid expressed proteins (e.g., CCR2, CD300E).

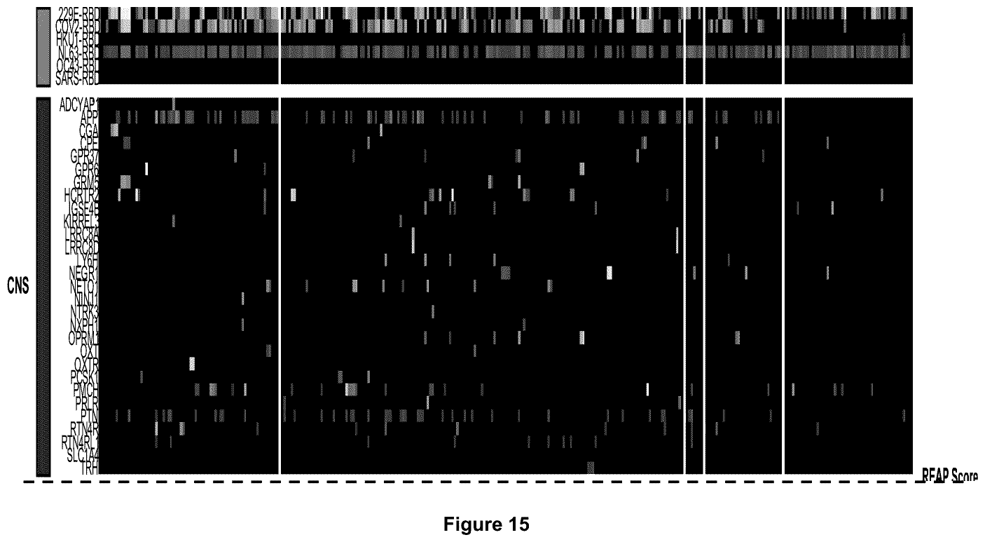

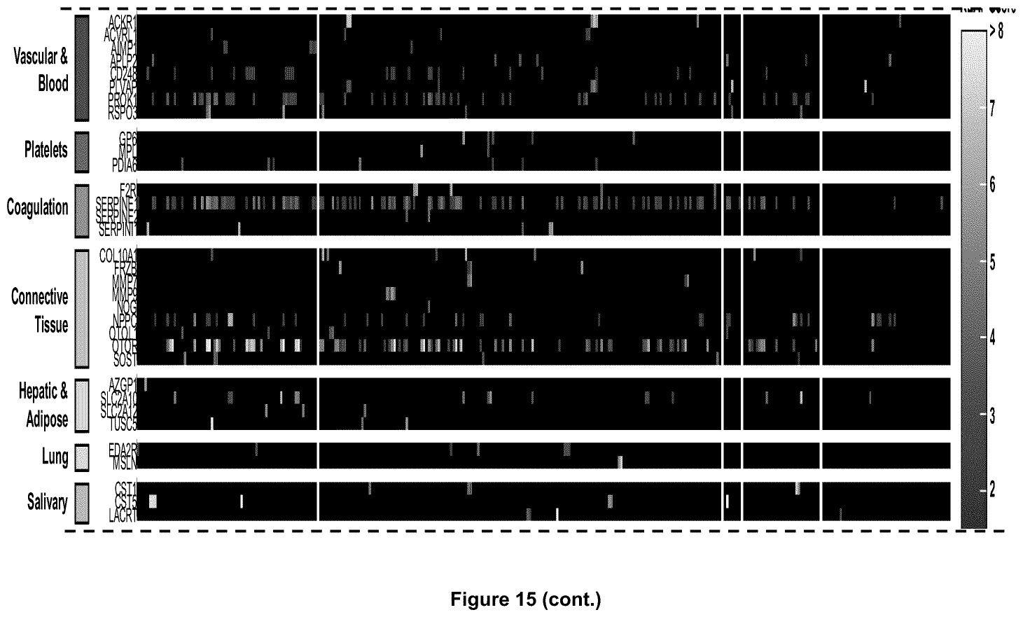

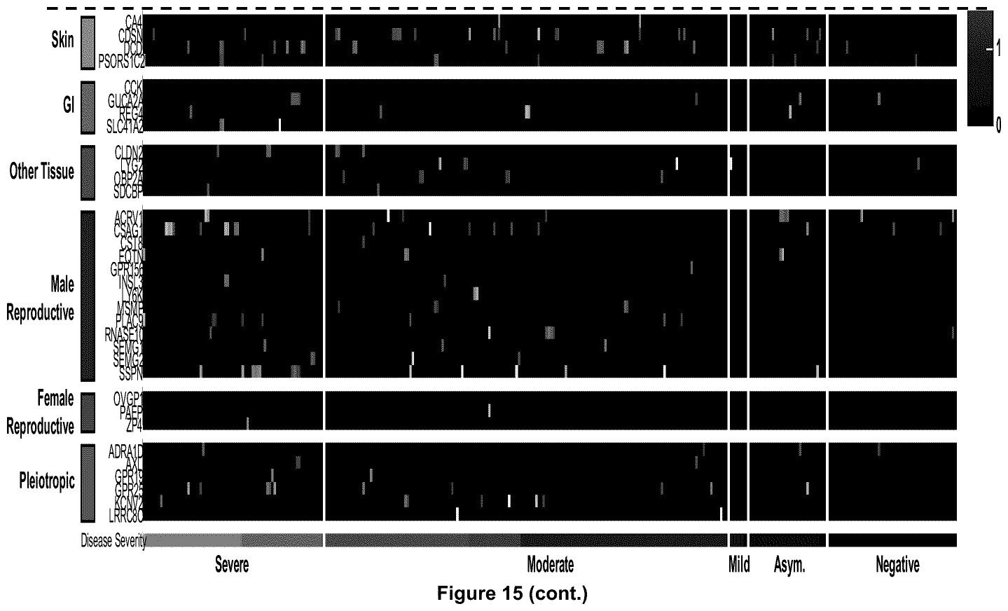

In addition to immune-targeting autoantibodies, a high prevalence of tissue-associated autoantibodies in COVID-19 patients (FIG. 15) was observed. A list of tissue associated antigens with significant differences in REAP signals was manually curated between uninfected controls and symptomatic patients, and a heatmap organized by COVID-19 disease severity was generated. Broadly, a high frequency of autoantibodies were found directed against vascular cell types (e.g., endothelial adhesion molecule PLVAP, regulator of angiogenesis RSPO3); against coagulation factors (e.g., coagulation factor II receptor F2R, SERPINEl and 2) and platelets (e.g., glycoprotein VI GP6); and against connective tissue and extracellular matrix targets (e.g., suspected regulator of cartilage maintenance OTOR, matrix metalloproteinases MMP7 and MMP9). In addition, REAP hits were observed against various organ systems including lung (e.g., ectodysplasin A2 Receptor EDA2R and mesothelin MSLN), the CNS compartment (e.g., orexin receptor HCRTR2, metabotropic glutamate receptor GRM5, neuronal injury marker NINJ1), skin (e.g., dermcidin DCD), gastrointestinal tract (e.g., regenerating family member 4 REG4, guanylate cyclase activator 2A GUCA2A), and other tissues.

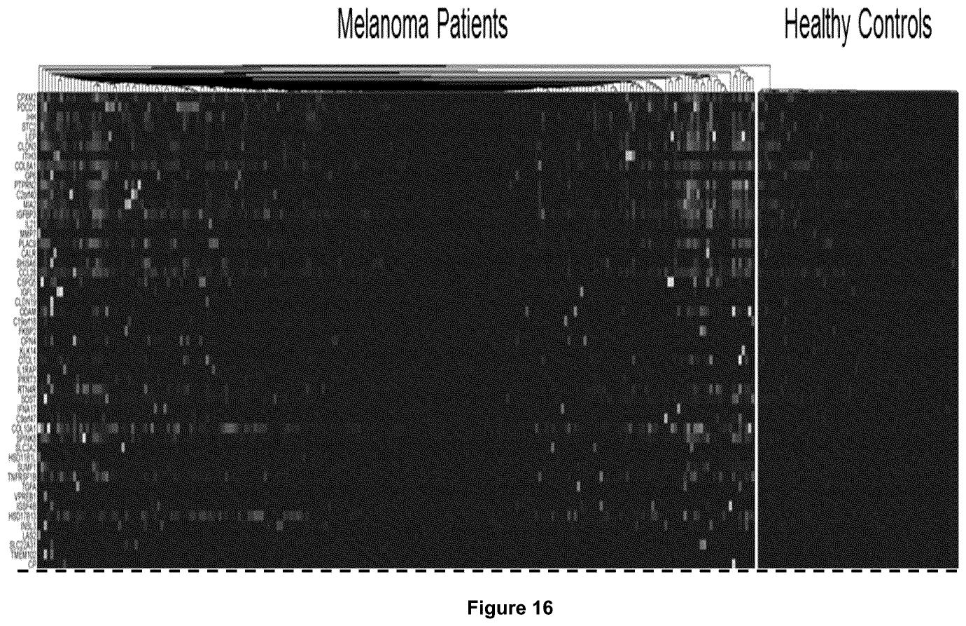

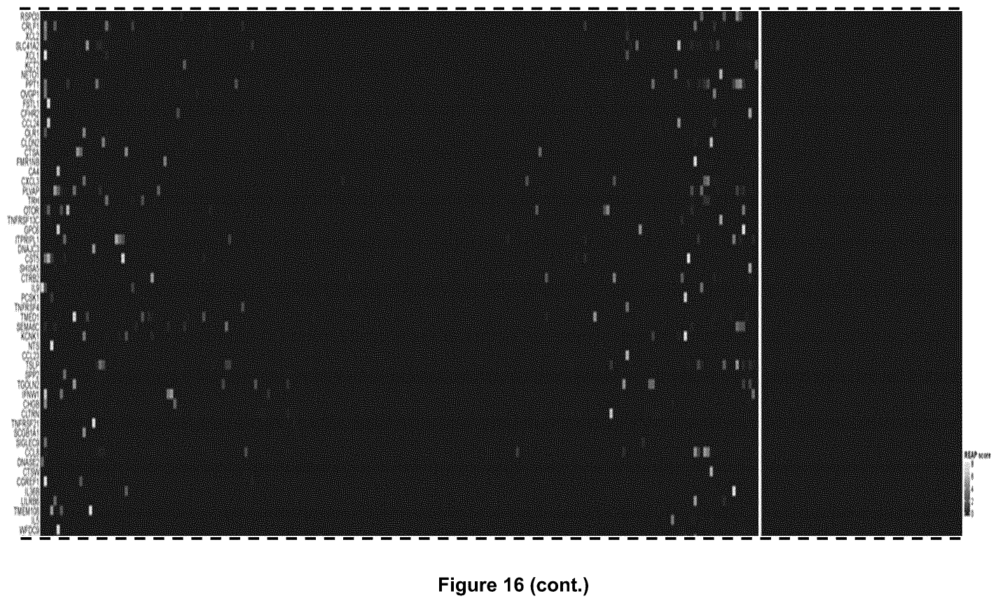

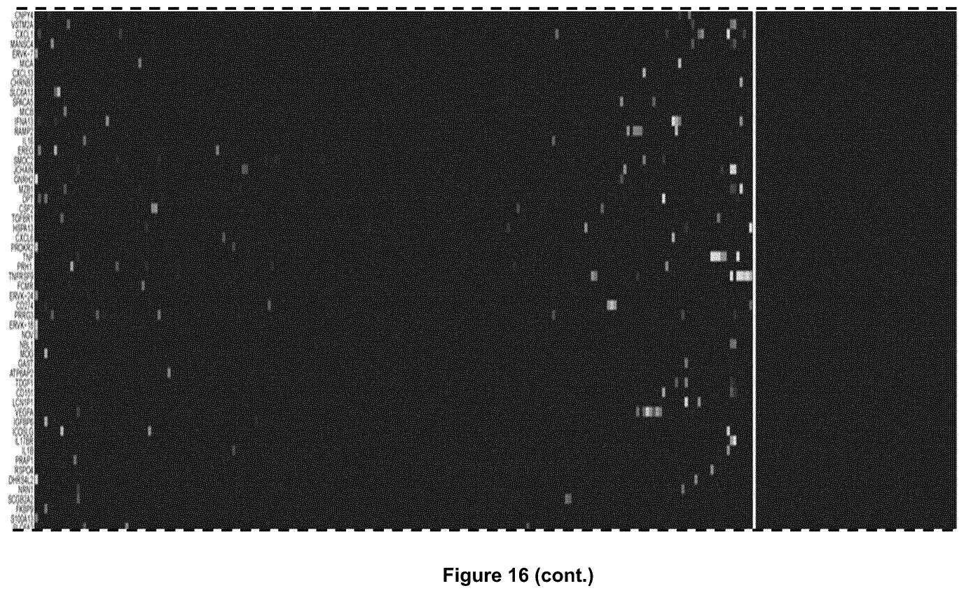

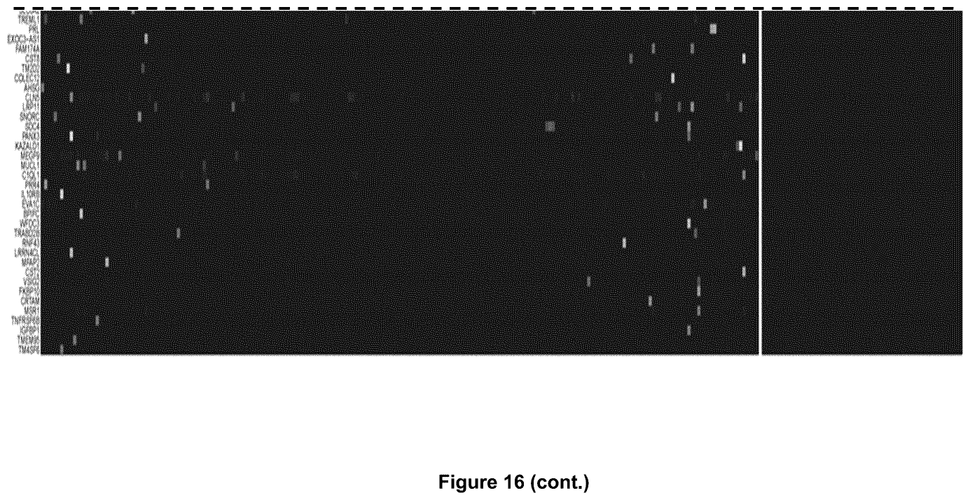

To explore the correlation of autoantibodies with disease progression/adverse events in cancer patients treated with immunotherapy, 1,454 longitudinal samples were screened from 222 CPI-treated melanoma patients (FIG. 16). Anti-CTLA4/PD1/PDL1 drugs were detected in most treated patients. Beyond these “controls”, more than 400 hits with significant REAP scores were observed across the samples. Many hits like ICOSLG, IL6, TNFa, and ILlA are present in multiple patients and these antibodies could have a modulation role in drug response and immune-related adverse events.

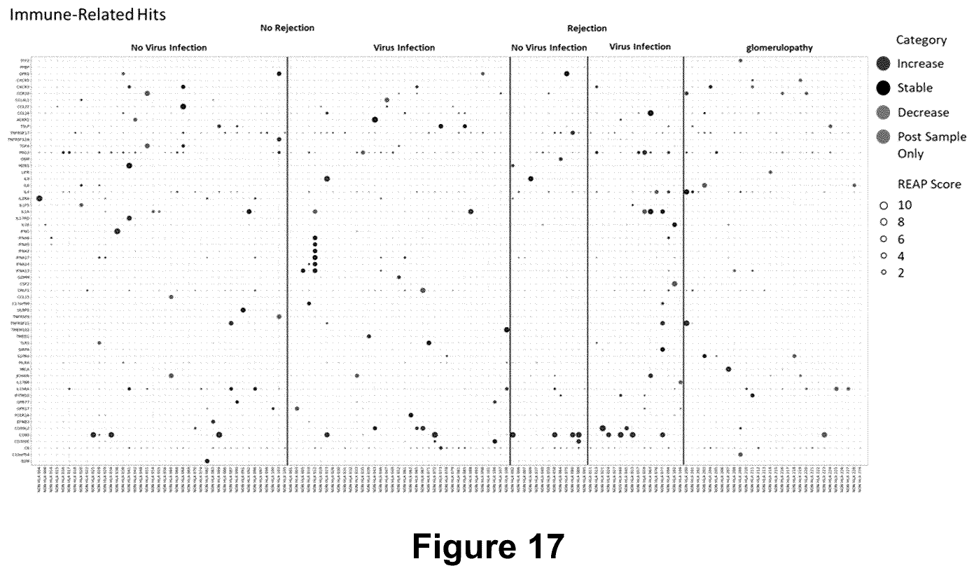

The broad autoantibody reactivity is also observed in kidney transplant patients (FIG. 17). 108 patients with pre and post transplantation serum samples were screened. Around 320 autoantibodies and 70/320 are immune-related hits were detected. Patients treated with Belatacept (CTLA-4 Fc) were accurately captured, with high CD80 scores. Patients are grouped by rejection and infection status after transplantation. Some hits like IFITM10, IL4, EXOC3-AS1 are highly associated with post-transplantation rejection while anti-IGFBP1 shows a potential protective role. Anti-IFNa family/CD99L2/OSTN/SYCN/LYG2/BTN1A1 autoantibodies are enriched in the infection group, suggesting a protective role of these proteins in virus infection. Anti-NXPH1/CST5 autoantibodies are observed in the non-infection group, indicates the potential immune-inhibitory role of these proteins. The existence of these autoantibodies is an opportunity to modulate patients' responses with kidney transplantation.

Custom Scoring Algorithm has High Sensitivity and Specificity

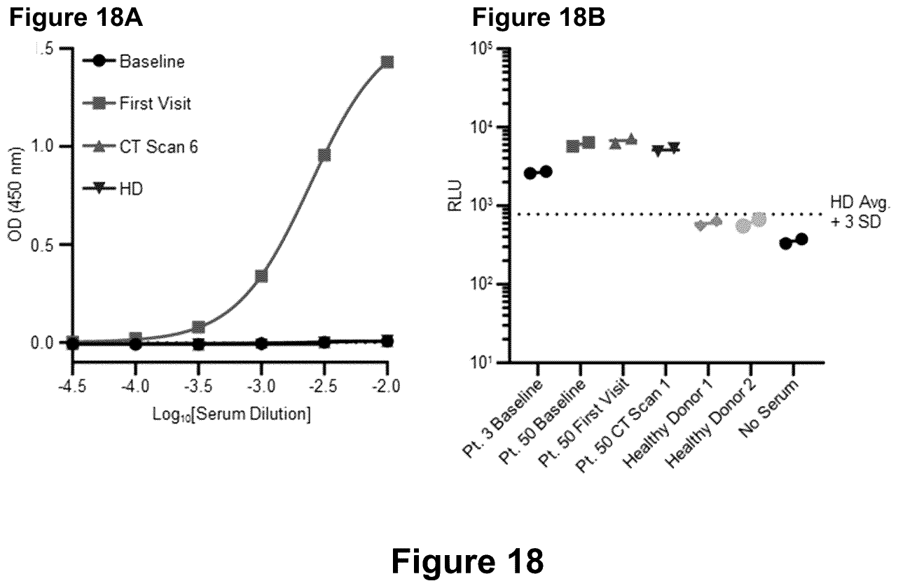

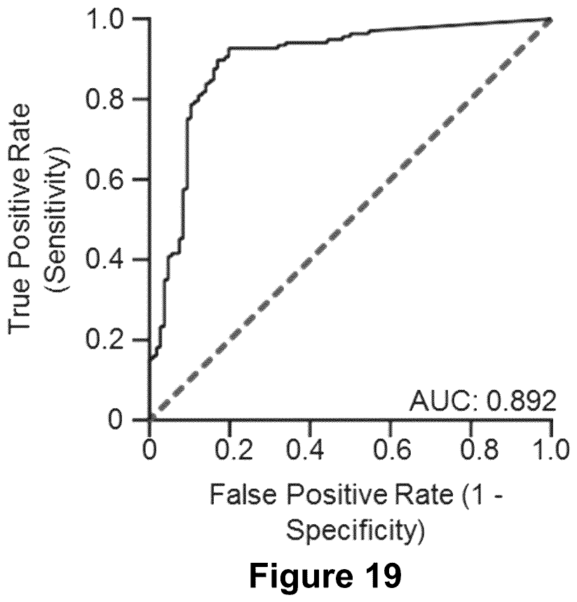

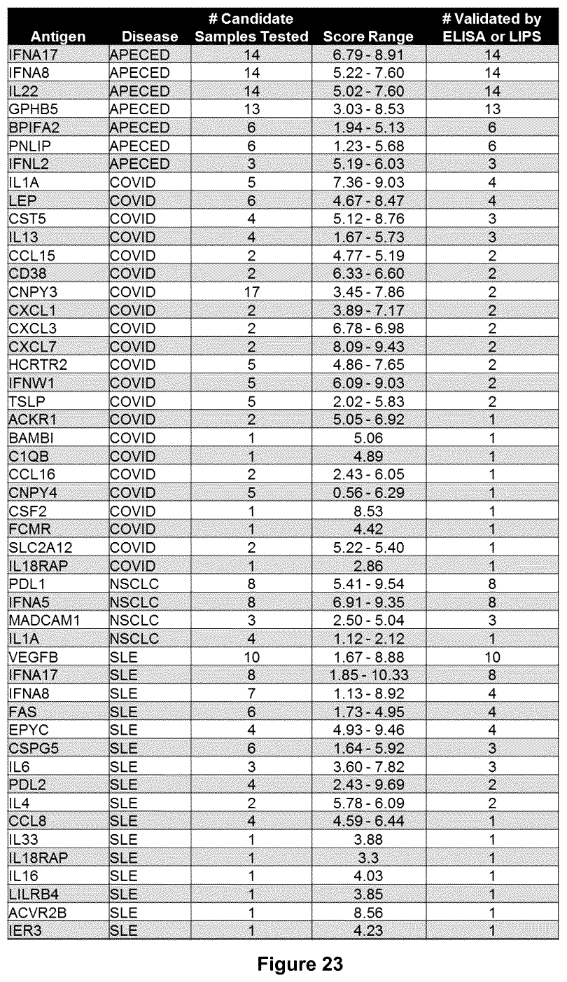

To validate the autoantibody reactivities that were discovered, two parallel and orthogonal assays were used. Luciferase Immunoprecipitation Systems (LIPS) offers a highly sensitive, higher-throughput validation process, but relies on luciferase fusions that may interfere with protein folding or lead to higher noise and variability between proteins. ELISA requires larger amounts of purified recombinant protein but is a “gold-standard” assay that is widely used. In both assays, valid autoantibody reactivities were defined as those with signals 3 standard deviations above the average healthy donor signal. Representative ELISA and LIPS validation plots can be seen in FIG. 18A and FIG. 18B. Using orthogonal validation data from APECED and SLE patients (247 test pairs across 25 different proteins), a receiver operating characteristic analysis was conducted and it was found that using the current scoring algorithm, REAP could distinguish autoantibody reactivities with an area under the curve of 0.892 (FIG. 19). A list of all REAP reactivities that have been orthogonally validated is provided in FIG. 23.

Pathogenic Autoantibodies Identified by REAP could be Specifically Targeted for Degradation in Clinical Settings

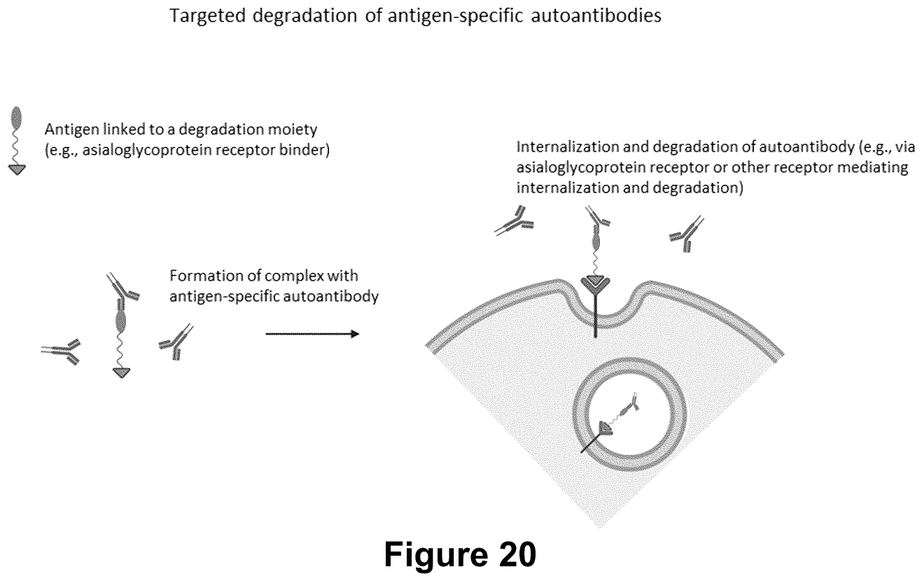

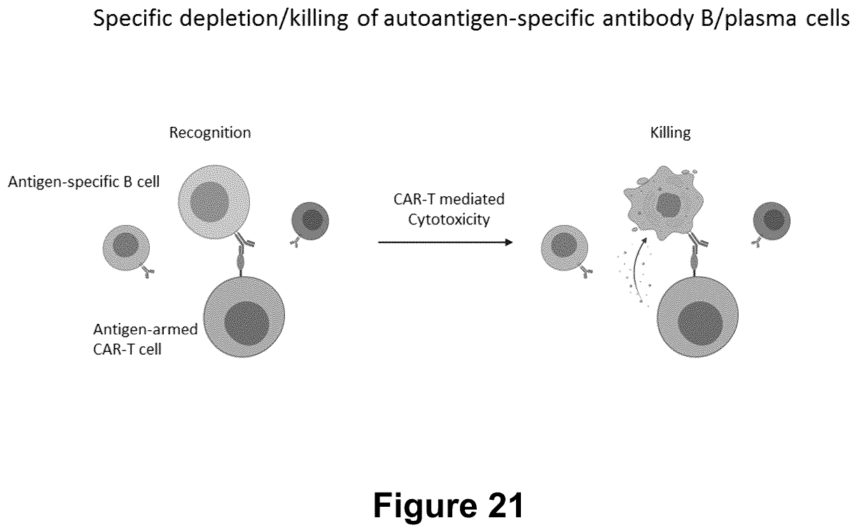

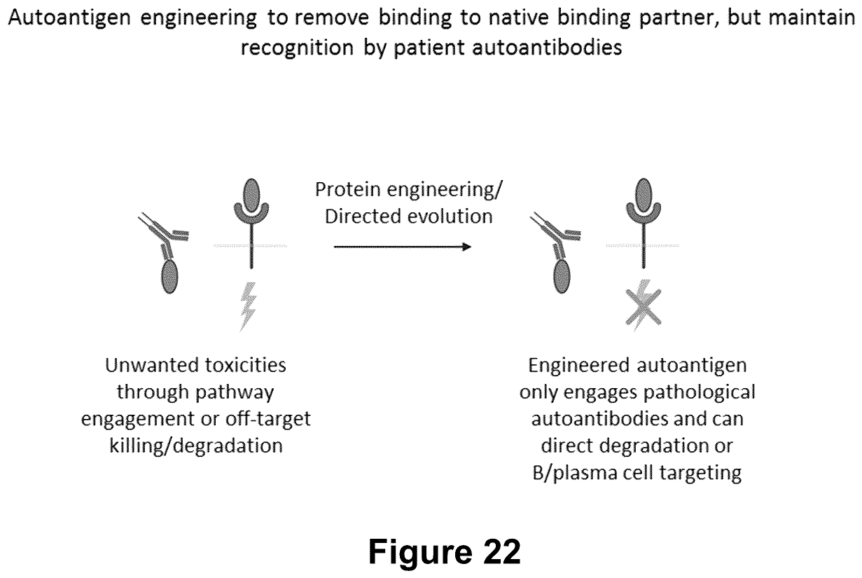

Autoantibodies that are identified in REAP screens and are further demonstrated to have pathogenic effects could be targeted for degradation in clinical settings using existing therapeutic modalities. For example, pathogenic autoantibodies could be removed from circulation in patients through the use of recombinant biologics in the form of autoantigens conjugated to endocytosis-promoting protein tags. Upon injection of these autoantigen conjugates into circulation, pathogenic autoantibodies will bind to their respective autoantigen, be trafficked to endosomal pathways, and ultimately be degraded intracellularly (FIG. 20). Chimeric autoantigen receptor (CAAR) T cells, a recently developed drug modality, could also be used to eliminate the B cells responsible for pathogenic autoantibody production. CAAR T cells display autoantigens on their cell surfaces that are connected to intracellular T cell activation domains. Inside a patient, CAAR T cells can bind to the B cell receptors of autoreactive B cells and initiate cytotoxic pathways that lead to lysis of the target autoreactive B cell (FIG. 21). In some cases, when autoantigens are proteins that have potentially harmful physiological effects when administered systemically and in large quantities (e.g., cytokines, chemokines, growth factors) or have native binding partners that are widely expressed, autoantigens could be engineered so that they do not interact with their native partner (FIG. 22). For example, if depletion of anti-IFNα autoantibodies was clinically indicated, IFNα could be engineered so that it does not bind to IFNAR1/2 and this engineered protein could be used as the autoantigen in the previously described therapeutic modalities.

The materials and methods employed in this experiment are now described.

Library Design:

An initial library of 3093 human extracellular proteins was assembled based on protein domains, immunological functions, and yeast-display compatibility. The extracellular portion of each protein was identified by manual inspection of topological domains annotated in the SwissProt database (January 2018). For proteins with uncertain topology, full sequences were run through SignalP 4, Topcons, and GPTPred to identify most likely topologies. For proteins with multiple extracellular portions, in general the longest individual region was chosen for initial amplification. cDNAs for chosen proteins were purchased from GE Dharmacon or DNASU. The protein sequences were further modified to match isoforms available in purchased cDNAs. An inventory of antigens included in the library are compiled in Table 1.

TABLE 1
Representative list of DNA and protein sequences
amplified for the initial and expanded libraries.

| Seq. Id. | Seq. Id. | Uniprot | Gene |
|---|---|---|---|
| No. (protein) | No. (DNA) | ID | Symbol |
| 1 | 3093 | P04217 | A1BG |
| 2 | 3094 | P01023 | A2M |
| 3 | 3095 | Q7Z7G0 | ABI3BP |
| 4 | 3096 | P16112 | ACAN |
| 5 | 3097 | Q9BYF1 | ACE2 |
| 6 | 3098 | O75078 | ADAM11 |
| 7 | 3099 | O43184 | ADAM12 |
| 8 | 3100 | Q13444 | ADAM15 |
| 9 | 3101 | Q9Y3Q7 | ADAM18 |
| 10 | 3102 | Q9H013 | ADAM19 |
| 11 | 3103 | Q99965 | ADAM2 |
| 12 | 3104 | O43506 | ADAM20 |
| 13 | 3105 | Q9UKJ8 | ADAM21 |
| 14 | 3106 | Q9P0K1 | ADAM22 |
| 15 | 3107 | O75077 | ADAM23 |
| 16 | 3108 | QOUKQ2 | ADAM28 |
| 17 | 3109 | Q9UKF5 | ADAM29 |
| 18 | 3110 | Q9UKF2 | ADAM30 |
| 19 | 3111 | Q8TC27 | ADAM32 |
| 20 | 3112 | Q9BZ11 | ADAM33 |
| 21 | 3113 | P78325 | ADAM8 |
| 22 | 3114 | Q13443 | ADAM9 |
| 23 | 3115 | P82987 | ADAMTSL3 |
| 24 | 3116 | Q9UHX3 | ADGRE2 |
| 25 | 3117 | Q9BY15 | ADGRE3 |
| 26 | 3118 | Q86SQ3 | ADGRE4P |
| 27 | 3119 | P48960 | ADGRE5 |
| 28 | 3120 | P35318 | ADM |
| 29 | 3121 | Q7Z4H4 | ADM2 |
| 30 | 3122 | Q15109 | AGER |
| 31 | 3123 | O00468 | AGRN |
| 32 | 3124 | Q13740 | ALCAM |
| 33 | 3125 | Q86YT9 | AMICA1 |
| 34 | 3126 | Q86WK6 | AMIGO1 |
| 35 | 3127 | Q86SJ2 | AMIGO2 |
| 36 | 3128 | Q86WK7 | AMIGO3 |
| 37 | 3129 | Q15389 | ANGPT1 |
| 38 | 3130 | O15123 | ANGPT2 |
| 39 | 3131 | Q9Y264 | ANGPT4 |
| 40 | 3132 | Q9UKU9 | ANGPTL2 |
| 41 | 3133 | Q9Y5C1 | ANGPTL3 |
| 42 | 3134 | Q9BY76 | ANGPTL4 |
| 43 | 3135 | Q9H6X2 | ANTXR1 |
| 44 | 3136 | P58335 | ANTXR2 |
| 45 | 3137 | A6NF34 | ANTXRL |
| 46 | 3138 | P15514 | AREG |
| 47 | 3139 | Q9H6B4 | ASAM |
| 48 | 3140 | P07306 | ASGR1 |
| 49 | 3141 | P07307 | ASGR2 |
| 50 | 3142 | Q9BXN1 | ASPN |
| 51 | 3143 | O14525 | ASTN1 |
| 52 | 3144 | O75129 | ASTN2 |
| 53 | 3145 | Q6UW56 | ATRAID |
| 54 | 3146 | O75882 | ATRN |
| 55 | 3147 | Q5VV63 | ATRNL1 |
| 56 | 3148 | P30530 | AXL |
| 57 | 3149 | P25311 | AZGP1 |
| 58 | 3150 | P61769 | B2M |
| 59 | 3151 | P50895 | BCAM |
| 60 | 3152 | Q96GW7 | BCAN |
| 61 | 3153 | P21810 | BGN |
| 62 | 3154 | P13497 | BMP1 |
| 63 | 3155 | O95393 | BMP10 |
| 64 | 3156 | O95972 | BMP15 |
| 65 | 3157 | P12643 | BMP2 |
| 66 | 3158 | P12645 | BMP3 |
| 67 | 3159 | P12644 | BMP4 |
| 68 | 3160 | P22003 | BMP5 |
| 69 | 3161 | P18075 | BMP7 |
| 70 | 3162 | Q7Z5Y6 | BMP8A |
| 71 | 3163 | P34820 | BMP8B |
| 72 | 3164 | P36894 | BMPR1A |
| 73 | 3165 | O00238 | BMPR1B |
| 74 | 3166 | Q13873 | BMPR2 |
| 75 | 3167 | Q9BWV1 | BOC |
| 76 | 3168 | P35613 | BSG |
| 77 | 3169 | Q075Z2 | BSPH1 |
| 78 | 3170 | P35070 | BTC |
| 79 | 3171 | Q7Z6A9 | BTLA |
| 80 | 3172 | Q13410 | BTN1A1 |
| 81 | 3173 | Q7KYR7 | BTN2A1 |
| 82 | 3174 | Q8WVV5 | BTN2A2 |
| 83 | 3175 | Q96KV6 | BTN2A3P |
| 84 | 3176 | O00481 | BTN3A1 |
| 85 | 3177 | P78410 | BTN3A2 |
| 86 | 3178 | O00478 | BTN3A3 |
| 87 | 3179 | A8MVZ5 | BTNL10 |
| 88 | 3180 | Q9UIR0 | BTNL2 |
| 89 | 3181 | Q6UXE8 | BTNL3 |
| 90 | 3182 | Q6UX41 | BTNL8 |
| 91 | 3183 | Q6UXG8 | BTNL9 |
| 92 | 3184 | O95971 | BY55 |
| 93 | 3185 | Q9H7M9 | C10orf54 |
| 94 | 3186 | Q5VYX0 | C10orf59 |
| 95 | 3187 | Q6UX52 | C17orf99 |
| 96 | 3188 | Q969H8 | C19orf10 |
| 97 | 3189 | F2Z333 | C1orf233 |
| 98 | 3190 | Q71H61 | C1orf32 |
| 99 | 3191 | O75973 | C1QL1 |
| 100 | 3192 | Q7Z5L3 | C1QL2 |
| 101 | 3193 | Q9NPY3 | C1QR1 |
| 102 | 3194 | Q9BXJ5 | C1QTNF2 |
| 103 | 3195 | Q9BXJ3 | C1QTNF4 |
| 104 | 3196 | Q9BXJ0 | C1QTNF5 |
| 105 | 3197 | P00736 | C1R |
| 106 | 3198 | P09871 | C1S |
| 107 | 3199 | P01024 | C3 |
| 108 | 3200 | P0C0L4 | C4A |
| 109 | 3201 | P01031 | C5 |
| 110 | 3202 | P13671 | C6 |
| 111 | 3203 | O95866 | C6orf25 |
| 112 | 3204 | P10643 | C7 |
| 113 | 3205 | P07357 | C8A |
| 114 | 3206 | P07358 | C8B |
| 115 | 3207 | P02748 | C9 |
| 116 | 3208 | Q9BY67 | CADM1 |
| 117 | 3209 | Q8N3J6 | CADM2 |
| 118 | 3210 | Q6UXH8 | CCBE1 |
| 119 | 3211 | P22362 | CCL1 |
| 120 | 3212 | P51671 | CCL11 |
| 121 | 3213 | Q99616 | CCL13 |
| 122 | 3214 | Q16627 | CCL14 |
| 123 | 3215 | Q16663 | CCL15 |
| 124 | 3216 | O15467 | CCL16 |
| 125 | 3217 | Q92583 | CCL17 |
| 126 | 3218 | P55774 | CCL18 |
| 127 | 3219 | Q99731 | CCL19 |
| 128 | 3220 | P13500 | CCL2 |
| 129 | 3221 | P78556 | CCL20 |
| 130 | 3222 | O00585 | CCL21 |
| 131 | 3223 | O00626 | CCL22 |
| 132 | 3224 | P55773 | CCL23 |
| 133 | 3225 | O00175 | CCL24 |
| 134 | 3226 | O15444 | CCL25 |
| 135 | 3227 | Q9Y258 | CCL26 |
| 136 | 3228 | Q9Y4X3 | CCL27 |
| 137 | 3229 | Q9NRJ3 | CCL28 |
| 138 | 3230 | P10147 | CCL3 |
| 139 | 3231 | P16619 | CCL3L3 |
| 140 | 3232 | P13236 | CCL4 |
| 141 | 3233 | Q8NHW4 | CCL4L1 |
| 142 | 3234 | P13501 | CCL5 |
| 143 | 3235 | P80098 | CCL7 |
| 144 | 3236 | P80075 | CCL8 |
| 145 | 3237 | P08571 | CD14 |
| 146 | 3238 | P48509 | CD151 |
| 147 | 3239 | Q86VB7 | CD163 |
| 148 | 3240 | Q9NR16 | CD163L1 |
| 149 | 3241 | Q99467 | CD180 |
| 150 | 3242 | P15391 | CD19 |
| 151 | 3243 | P06126 | CD1A |
| 152 | 3244 | P29016 | CD1B |
| 153 | 3245 | P29017 | CD1C |
| 154 | 3246 | P15813 | CD1D |
| 155 | 3247 | P15812 | CD1E |
| 156 | 3248 | P06729 | CD2 |
| 157 | 3249 | P41217 | CD200 |
| 158 | 3250 | Q8TD46 | CD200R1 |
| 159 | 3251 | Q6Q8B3 | CD200R1L |
| 160 | 3252 | Q9UJ71 | CD207 |
| 161 | 3253 | Q9NNX6 | CD209 |
| 162 | 3254 | P20273 | CD22 |
| 163 | 3255 | Q15762 | CD226 |
| 164 | 3256 | Q9BZW8 | CD244 |
| 165 | 3257 | Q9HCU0 | CD248 |
| 166 | 3258 | Q9NZQ7 | CD274 |
| 167 | 3259 | Q5ZPR3 | CD276 |
| 168 | 3260 | P10747 | CD28 |
| 169 | 3261 | Q9UGN4 | CD300A |
| 170 | 3262 | Q08708 | CD300C |
| 171 | 3263 | Q496F6 | CD300E |
| 172 | 3264 | A8K4G0 | CD300LB |
| 173 | 3265 | Q6UXZ3 | CD300LD |
| 174 | 3266 | Q8TDQ1 | CD300LF |
| 175 | 3267 | Q6UXG3 | CD300LG |
| 176 | 3268 | Q8IX05 | CD302 |
| 177 | 3269 | Q9NPF0 | CD320 |
| 178 | 3270 | P20138 | CD33 |
| 179 | 3271 | P28906 | CD34 |
| 180 | 3272 | P16671 | CD36 |
| 181 | 3273 | P11049 | CD37 |
| 182 | 3274 | P28907 | CD38 |
| 183 | 3275 | P04234 | CD3D |
| 184 | 3276 | P07766 | CD3E |
| 185 | 3277 | P09693 | CD3G |
| 186 | 3278 | P01730 | CD4 |
| 187 | 3279 | P29965 | CD40LG |
| 188 | 3280 | P16070 | CD44 |
| 189 | 3281 | Q08722 | CD47 |
| 190 | 3282 | P09326 | CD48 |
| 191 | 3283 | P06127 | CD5 |
| 192 | 3284 | P19397 | CD53 |
| 193 | 3285 | P08174 | CD55 |
| 194 | 3286 | P19256 | CD58 |
| 195 | 3287 | P13987 | CD59 |
| 196 | 3288 | P30203 | CD6 |
| 197 | 3289 | P08962 | CD63 |
| 198 | 3290 | Q07108 | CD69 |
| 199 | 3291 | P09564 | CD7 |
| 200 | 3292 | P32970 | CD70 |
| 201 | 3293 | P21854 | CD72 |
| 202 | 3294 | P04233 | CD74 |
| 203 | 3295 | P11912 | CD79A |
| 204 | 3296 | P40259 | CD79B |
| 205 | 3297 | P33681 | CD80 |
| 206 | 3298 | P60033 | CD81 |
| 207 | 3299 | P27701 | CD82 |
| 208 | 3300 | Q01151 | CD83 |
| 209 | 3301 | Q9UIB8 | CD84 |
| 210 | 3302 | P42081 | CD86 |
| 211 | 3303 | P01732 | CD8A |
| 212 | 3304 | P10966 | CD8B |
| 213 | 3305 | A6NJW9 | CD8B2 |
| 214 | 3306 | P21926 | CD9 |
| 215 | 3307 | P40200 | CD96 |
| 216 | 3308 | P14209 | CD99 |
| 217 | 3309 | P12830 | CDH1 |
| 218 | 3310 | Q9Y6N8 | CDH10 |
| 219 | 3311 | P55287 | CDH11 |
| 220 | 3312 | P55289 | CDH12 |
| 221 | 3313 | P55290 | CDH13 |
| 222 | 3314 | P55291 | CDH15 |
| 223 | 3315 | O75309 | CDH16 |
| 224 | 3316 | Q12864 | CDH17 |
| 225 | 3317 | Q13634 | CDH18 |
| 226 | 3318 | Q9H159 | CDH19 |
| 227 | 3319 | P19022 | CDH2 |
| 228 | 3320 | Q9HBT6 | CDH20 |
| 229 | 3321 | Q9UJ99 | CDH22 |
| 230 | 3322 | Q9H251 | CDH23 |
| 231 | 3323 | Q86UP0 | CDH24 |
| 232 | 3324 | Q8IXH8 | CDH26 |
| 233 | 3325 | P22223 | CDH3 |
| 234 | 3326 | P55283 | CDH4 |
| 235 | 3327 | P33151 | CDH5 |
| 236 | 3328 | P55285 | CDH6 |
| 237 | 3329 | Q9ULB5 | CDH7 |
| 238 | 3330 | P55286 | CDH8 |
| 239 | 3331 | Q9ULB4 | CDH9 |
| 240 | 3332 | Q4KMG0 | CDON |
| 241 | 3333 | O43827 | CDT6 |
| 242 | 3334 | P13688 | CEACAM1 |
| 243 | 3335 | Q2WEN9 | CEACAM16 |
| 244 | 3336 | A8MTB9 | CEACAM18 |
| 245 | 3337 | Q7Z692 | CEACAM19 |
| 246 | 3338 | Q6UY09 | CEACAM20 |
| 247 | 3339 | Q3KPI0 | CEACAM21 |
| 248 | 3340 | P40198 | CEACAM3 |
| 249 | 3341 | O75871 | CEACAM4 |
| 250 | 3342 | P06731 | CEACAM5 |
| 251 | 3343 | P40199 | CEACAM6 |
| 252 | 3344 | Q14002 | CEACAM7 |
| 253 | 3345 | P31997 | CEACAM8 |
| 254 | 3346 | P0CG37 | CFC1 |
| 255 | 3347 | P0CG36 | CFC1B |
| 256 | 3348 | P00746 | CFD |
| 257 | 3349 | P08603 | CFH |
| 258 | 3350 | Q92496 | CFHR4 |
| 259 | 3351 | P05156 | CFI |
| 260 | 3352 | O15335 | CHAD |
| 261 | 3353 | Q6NUI6 | CHADL |
| 262 | 3354 | O00533 | CHL1 |
| 263 | 3355 | Q9H9P2 | CHODL |
| 264 | 3356 | O75339 | CILP |
| 265 | 3357 | Q8IUL8 | CILP2 |
| 266 | 3358 | QOUQC9 | CLCA2 |
| 267 | 3359 | Q14CN2 | CLCA4 |
| 268 | 3360 | Q8WXI8 | CLEC-6 |
| 269 | 3361 | Q8IUN9 | CLEC10A |
| 270 | 3362 | Q9Y240 | CLEC11A |
| 271 | 3363 | Q5QGZ9 | CLEC12A |
| 272 | 3364 | Q2HXU8 | CLEC12B |
| 273 | 3365 | Q86T13 | CLEC14A |
| 274 | 3366 | Q6ZS10 | CLEC17A |
| 275 | 3367 | Q6UXF7 | CLEC18A |
| 276 | 3368 | A5D8T8 | CLEC18A |
| 277 | 3369 | Q6UXS0 | CLEC19A |
| 278 | 3370 | Q8NC01 | CLEC1A |
| 279 | 3371 | Q9P126 | CLEC1B |
| 280 | 3372 | Q92478 | CLEC2B |
| 281 | 3373 | Q9UHP7 | CLEC2D |
| 282 | 3374 | O75596 | CLEC3A |
| 283 | 3375 | Q9UMR7 | CLEC4A |
| 284 | 3376 | Q8WTT0 | CLEC4C |
| 285 | 3377 | Q9ULY5 | CLEC4E |
| 286 | 3378 | Q8N1N0 | CLEC4F |
| 287 | 3379 | Q6UXB4 | CLEC4G |
| 288 | 3380 | Q9H2X3 | CLEC4M |
| 289 | 3381 | Q9NY25 | CLEC5A |
| 290 | 3382 | Q6EIG7 | CLEC6A |
| 291 | 3383 | Q9BXN2 | CLEC7A |
| 292 | 3384 | Q6UXN8 | CLEC9A |
| 293 | 3385 | Q8IZS7 | CLECL1 |
| 294 | 3386 | P26992 | CNTFR |
| 295 | 3387 | Q12860 | CNTN1 |
| 296 | 3388 | Q02246 | CNTN2 |
| 297 | 3389 | Q9P232 | CNTN3 |
| 298 | 3390 | Q8IWV2 | CNTN4 |
| 299 | 3391 | O94779 | CNTN5 |
| 300 | 3392 | Q9UQ52 | CNTN6 |
| 301 | 3393 | P78357 | CNTNAP1 |
| 302 | 3394 | Q9UHC6 | CNTNAP2 |
| 303 | 3395 | Q9BZ76 | CNTNAP3 |
| 304 | 3396 | Q9C0A0 | CNTNAP4 |
| 305 | 3397 | Q8WYK1 | CNTNAP5 |
| 306 | 3398 | Q05707 | COL14A1 |
| 307 | 3399 | Q9P218 | COL20A1 |
| 308 | 3400 | Q9Y6Z7 | COLEC10 |
| 309 | 3401 | Q9BWP8 | COLEC11 |
| 310 | 3402 | Q5KU26 | COLEC12 |
| 311 | 3403 | P49747 | COMP |
| 312 | 3404 | Q8IZJ3 | CPAMD8 |
| 313 | 3405 | P22792 | CPN2 |
| 314 | 3406 | P82279 | CRB1 |
| 315 | 3407 | Q96HD1 | CRELD1 |
| 316 | 3408 | Q6UXH1 | CRELD2 |
| 317 | 3409 | O75462 | CRLF1 |
| 318 | 3410 | Q9HC73 | CRLF2 |
| 319 | 3411 | Q9NQ79 | CRTAC1 |
| 320 | 3412 | O95727 | CRTAM |
| 321 | 3413 | P07333 | CSF1R |
| 322 | 3414 | P04141 | CSF2 |
| 323 | 3415 | P15509 | CSF2RA |
| 324 | 3416 | P32927 | CSF2RB |
| 325 | 3417 | P09919 | CSF3 |
| 326 | 3418 | Q99062 | CSF3R |
| 327 | 3419 | O95196 | CSPG5 |
| 328 | 3420 | P16410 | CTLA4 |
| 329 | 3421 | P78423 | CX3CL1 |
| 330 | 3422 | P78310 | CXADR |
| 331 | 3423 | P09341 | CXCL1 |
| 332 | 3424 | P02778 | CXCL10 |
| 333 | 3425 | O14625 | CXCL11 |
| 334 | 3426 | P48061 | CXCL12 |
| 335 | 3427 | O43927 | CXCL13 |
| 336 | 3428 | O95715 | CXCL14 |
| 337 | 3429 | Q9H2A7 | CXCL16 |
| 338 | 3430 | Q6UXB2 | CXCL17 |
| 339 | 3431 | P19875 | CXCL2 |
| 340 | 3432 | P19876 | CXCL3 |
| 341 | 3433 | P42830 | CXCL5 |
| 342 | 3434 | P80162 | CXCL6 |
| 343 | 3435 | Q07325 | CXCL9 |
| 344 | 3436 | Q14118 | DAG1 |
| 345 | 3437 | Q8N907 | DAND5 |
| 346 | 3438 | P07585 | DCN |
| 347 | 3439 | Q5T197 | DCST1 |
| 348 | 3440 | Q9H295 | DCSTAMP |
| 349 | 3441 | P59665 | DEFA1 |
| 350 | 3442 | B2R9L8 | Delta |
| 351 | 3443 | P98153 | DGCR2 |
| 352 | 3444 | Q68D85 | DKFZp686O24166 |
| 353 | 3445 | P80370 | DLK1 |
| 354 | 3446 | Q6UY11 | DLK2 |
| 355 | 3447 | O00548 | DLL1 |
| 356 | 3448 | Q9NYJ7 | DLL3 |
| 357 | 3449 | Q9NR61 | DLL4 |
| 358 | 3450 | Q8NFT8 | DNER |
| 359 | 3451 | Q02487 | DSC2 |
| 360 | 3452 | Q14574 | DSC3 |
| 361 | 3453 | Q14126 | DSG2 |
| 362 | 3454 | P32926 | DSG3 |
| 363 | 3455 | Q86SJ6 | DSG4 |
| 364 | 3456 | Q14213 | EBI3 |
| 365 | 3457 | O94769 | ECM2 |
| 366 | 3458 | Q92838 | EDA |
| 367 | 3459 | Q9HAV5 | EDA2R |
| 368 | 3460 | Q9UNE0 | EDAR |
| 369 | 3461 | O43854 | EDIL3 |
| 370 | 3462 | Q12805 | EFEMP1 |
| 371 | 3463 | O95967 | EFEMP2 |
| 372 | 3464 | P20827 | EFNA1 |
| 373 | 3465 | O43921 | EFNA2 |
| 374 | 3466 | P52797 | EFNA3 |
| 375 | 3467 | P52803 | EFNA5 |
| 376 | 3468 | P98172 | EFNB1 |
| 377 | 3469 | P52799 | EFNB2 |
| 378 | 3470 | Q15768 | EFNB3 |
| 379 | 3471 | P01133 | EGF |
| 380 | 3472 | O75095 | EGFL3 |
| 381 | 3473 | Q8IUX8 | EGFL6 |
| 382 | 3474 | Q9UHF1 | EGFL7 |
| 383 | 3475 | Q63HQ2 | EGFLAM |
| 384 | 3476 | P00533 | EGFR |
| 385 | 3477 | P0C7U0 | ELFN1 |
| 386 | 3478 | Q5R3F8 | ELFN2 |
| 387 | 3479 | Q96BH3 | ELSPBP1 |
| 388 | 3480 | Q9HBW9 | ELTD1 |
| 389 | 3481 | Q6PCB8 | EMB |
| 390 | 3482 | Q9ULC0 | EMCN |
| 391 | 3483 | Q14246 | EMR1 |
| 392 | 3484 | P22413 | ENPP1 |
| 393 | 3485 | P49961 | ENTPD1 |
| 394 | 3486 | O75355 | ENTPD3 |
| 395 | 3487 | Q6UW88 | EPGN |
| 396 | 3488 | P21709 | EPHA1 |
| 397 | 3489 | Q5JZY3 | EPHA10 |
| 398 | 3490 | P29317 | EPHA2 |
| 399 | 3491 | P29320 | EPHA3 |
| 400 | 3492 | P54764 | EPHA4 |
| 401 | 3493 | P54756 | EPHA5 |
| 402 | 3494 | Q9UF33 | EPHA6 |
| 403 | 3495 | Q15375 | EPHA7 |
| 404 | 3496 | P29322 | EPHA8 |
| 405 | 3497 | P54762 | EPHB1 |
| 406 | 3498 | P29323 | EPHB2 |
| 407 | 3499 | P54753 | EPHB3 |
| 408 | 3500 | P54760 | EPHB4 |
| 409 | 3501 | O15197 | EPHB6 |
| 410 | 3502 | P01588 | EPO |
| 411 | 3503 | P19235 | EPOR |
| 412 | 3504 | Q99645 | EPYC |
| 413 | 3505 | P04626 | ERBB2 |
| 414 | 3506 | P21860 | ERBB3 |
| 415 | 3507 | Q15303 | ERBB4 |
| 416 | 3508 | O14944 | EREG |
| 417 | 3509 | Q96PL5 | ERMAP |
| 418 | 3510 | Q96AP7 | ESAM |
| 419 | 3511 | Q5T1H1 | EYS |
| 420 | 3512 | P00742 | F10 |
| 421 | 3513 | Q9Y624 | F11R |
| 422 | 3514 | P00748 | F12 |
| 423 | 3515 | P00488 | F13A1 |
| 424 | 3516 | P13726 | F3 |
| 425 | 3517 | P08709 | F7 |
| 426 | 3518 | P00740 | F9 |
| 427 | 3519 | Q4G0M1 | FAM132B |
| 428 | 3520 | Q5VUB5 | FAM171A1 |
| 429 | 3521 | A6NFU0 | FAM187A |
| 430 | 3522 | Q17R55 | FAM187B |
| 431 | 3523 | Q8IXL6 | FAM20C |
| 432 | 3524 | Q9NYQ8 | FAT2 |
| 433 | 3525 | P23142 | FBLN1 |
| 434 | 3526 | P98095 | FBLN2 |
| 435 | 3527 | Q9UBX5 | FBLN5 |
| 436 | 3528 | Q53RD9 | FBLN7 |
| 437 | 3529 | P35556 | FBN2 |
| 438 | 3530 | Q75N90 | FBN3 |
| 439 | 3531 | Q8WWV6 | FCAMR |
| 440 | 3532 | P24071 | FCAR |
| 441 | 3533 | P12319 | FCER1A |
| 442 | 3534 | P06734 | FCER2 |
| 443 | 3535 | P12314 | FCGR1A |
| 444 | 3536 | Q92637 | FCGR1B |
| 445 | 3537 | P12318 | FCGR2A |
| 446 | 3538 | P31994 | FCGR2B |
| 447 | 3539 | P31994 | FCGR2C |
| 448 | 3540 | P31995 | FCGR2C |
| 449 | 3541 | P08637 | FCGR3A |
| 450 | 3542 | P08637 | FCGR3A |
| 451 | 3543 | P55899 | FCGRT |
| 452 | 3544 | O60667 | FCMR |
| 453 | 3545 | Q96LA6 | FCRL1 |
| 454 | 3546 | Q96LA5 | FCRL2 |
| 455 | 3547 | Q96P31 | FCRL3 |
| 456 | 3548 | Q96PJ5 | FCRL4 |
| 457 | 3549 | Q96RD9 | FCRL5 |
| 458 | 3550 | Q6DN72 | FCRL6 |
| 459 | 3551 | Q6BAA4 | FCRLB |
| 460 | 3552 | Q7L513 | FCRLM1 |
| 461 | 3553 | P05230 | FGF1 |
| 462 | 3554 | O15520 | FGF10 |
| 463 | 3555 | O43320 | FGF16 |
| 464 | 3556 | O60258 | FGF17 |
| 465 | 3557 | O76093 | FGF18 |
| 466 | 3558 | O95750 | FGF19 |
| 467 | 3559 | Q9NP95 | FGF20 |
| 468 | 3560 | Q9NSA1 | FGF21 |
| 469 | 3561 | Q9GZV9 | FGF23 |
| 470 | 3562 | P11487 | FGF3 |
| 471 | 3563 | P12034 | FGF5 |
| 472 | 3564 | P10767 | FGF6 |
| 473 | 3565 | P21781 | FGF7 |
| 474 | 3566 | P31371 | FGF9 |
| 475 | 3567 | Q14512 | FGFBP1 |
| 476 | 3568 | Q8TAT2 | FGFBP3 |
| 477 | 3569 | P11362 | FGFR1 |
| 478 | 3570 | P21802 | FGFR2 |
| 479 | 3571 | P22607 | FGFR3 |
| 480 | 3572 | P22455 | FGFR4 |
| 481 | 3573 | Q8N441 | FGFRL1 |
| 482 | 3574 | O43915 | FIGF |
| 483 | 3575 | Q6NSJ5 | FLJ23420 |
| 484 | 3576 | Q9NZU1 | FLRT1 |
| 485 | 3577 | O43155 | FLRT2 |
| 486 | 3578 | Q9NZU0 | FLRT3 |
| 487 | 3579 | P17948 | FLT1 |
| 488 | 3580 | P36888 | FLT3 |
| 489 | 3581 | Q06828 | FMOD |
| 490 | 3582 | P02751 | FN1 |
| 491 | 3583 | Q9H6D8 | FNDC4 |
| 492 | 3584 | Q8NAU1 | FNDC5 |
| 493 | 3585 | Q5VTL7 | FNDC7 |
| 494 | 3586 | Q5H8C1 | FREM1 |
| 495 | 3587 | P23945 | FSHR |
| 496 | 3588 | Q6MZW2 | FSTL4 |
| 497 | 3589 | Q8N475 | FSTL5 |
| 498 | 3590 | P05161 | G1P2 |
| 499 | 3591 | Q14393 | GAS6 |
| 500 | 3592 | P55107 | GDF10 |
| 501 | 3593 | O95390 | GDF11 |
| 502 | 3594 | Q99988 | GDF15 |
| 503 | 3595 | Q9UK05 | GDF2 |
| 504 | 3596 | Q9NR23 | GDF3 |
| 505 | 3597 | P43026 | GDF5 |
| 506 | 3598 | Q6KF10 | GDF6 |
| 507 | 3599 | O14793 | GDF8 |
| 508 | 3600 | O60383 | GDF9 |
| 509 | 3601 | P39905 | GDNF |
| 510 | 3602 | P56159 | GFRA1 |
| 511 | 3603 | O00451 | GFRA2 |
| 512 | 3604 | O60609 | GFRA3 |
| 513 | 3605 | Q9GZZ7 | GFRA4 |
| 514 | 3606 | P10912 | GHR |
| 515 | 3607 | Q9Y5U5 | GITR |
| 516 | 3608 | Q99445 | GML |
| 517 | 3609 | P22749 | GNLY |
| 518 | 3610 | P07359 | GP1BA |
| 519 | 3611 | P13224 | GP1BB |
| 520 | 3612 | P55259 | GP2 |
| 521 | 3613 | P40197 | GP5 |
| 522 | 3614 | Q9HCN6 | GP6 |
| 523 | 3615 | P14770 | GP9 |
| 524 | 3616 | Q99795 | GPA33 |
| 525 | 3617 | P06744 | GPI |
| 526 | 3618 | Q8IV16 | GPIHBP1 |
| 527 | 3619 | Q14956 | GPNMB |
| 528 | 3620 | P08236 | GUSB |
| 529 | 3621 | Q14520 | HABP2 |
| 530 | 3622 | P81172 | HAMP |
| 531 | 3623 | P10915 | HAPLN1 |
| 532 | 3624 | Q9GZV7 | HAPLN2 |
| 533 | 3625 | Q96S86 | HAPLN3 |
| 534 | 3626 | Q86UW8 | HAPLN4 |
| 535 | 3627 | Q96D42 | HAVCR1 |
| 536 | 3628 | Q8TDQ0 | HAVCR2 |
| 537 | 3629 | Q99075 | HBEGF |
| 538 | 3630 | Q14CZ8 | HEPACAM |
| 539 | 3631 | A8MVW5 | HEPACAM2 |
| 540 | 3632 | Q30201 | HFE |
| 541 | 3633 | P14210 | HGF |
| 542 | 3634 | Q04756 | HGFAC |
| 543 | 3635 | Q96QV1 | HHIP |
| 544 | 3636 | Q9UM44 | HHLA2 |
| 545 | 3637 | P01893 | HLA |
| 546 | 3638 | P01889 | HLA |
| 547 | 3639 | P01891 | HLA |
| 548 | 3640 | P01892 | HLA |
| 549 | 3641 | P30685 | HLA |
| 550 | 3642 | P04439 | HLA-A |
| 551 | 3643 | P01889 | HLA-B |
| 552 | 3644 | P10321 | HLA-C |
| 553 | 3645 | P28067 | HLA-DMA |
| 554 | 3646 | P28068 | HLA-DMB |
| 555 | 3647 | P06340 | HLA-DOA |
| 556 | 3648 | P13765 | HLA-DOB |
| 557 | 3649 | P20036 | HLA-DPA1 |
| 558 | 3650 | P04440 | HLA-DPB1 |
| 559 | 3651 | P01909 | HLA-DQA1 |
| 560 | 3652 | P01920 | HLA-DQB1 |
| 561 | 3653 | P01903 | HLA-DRA |
| 562 | 3654 | P01911 | HLA-DRB1 |
| 563 | 3655 | P13747 | HLA-E |
| 564 | 3656 | P30511 | HLA-F |
| 565 | 3657 | P17693 | HLA-G |
| 566 | 3658 | P09429 | HMGB1 |
| 567 | 3659 | P26583 | HMGB2 |
| 568 | 3660 | Q12794 | HYAL1 |
| 569 | 3661 | Q12891 | HYAL2 |
| 570 | 3662 | O43820 | HYAL3 |
| 571 | 3663 | P05362 | ICAM1 |
| 572 | 3664 | P13598 | ICAM2 |
| 573 | 3665 | P32942 | ICAM3 |
| 574 | 3666 | Q14773 | ICAM4 |
| 575 | 3667 | Q9UMF0 | ICAM5 |
| 576 | 3668 | Q9Y6W8 | ICOS |
| 577 | 3669 | O75144 | ICOSLG |
| 578 | 3670 | A6NMD0 | IFITM10 |
| 579 | 3671 | P01566 | IFNA10 |
| 580 | 3672 | P01562 | IFNA13 |
| 581 | 3673 | P01570 | IFNA14 |
| 582 | 3674 | P05015 | IFNA16 |
| 583 | 3675 | P01571 | IFNA17 |
| 584 | 3676 | P01571 | IFNA17 |
| 585 | 3677 | P01563 | IFNA2 |
| 586 | 3678 | P01568 | IFNA21 |
| 587 | 3679 | P01567 | IFNA4 |
| 588 | 3680 | P01569 | IFNA5 |
| 589 | 3681 | P05013 | IFNA6 |
| 590 | 3682 | P32881 | IFNA8 |
| 591 | 3683 | P17181 | IFNAR1 |
| 592 | 3684 | P48551 | IFNAR2 |
| 593 | 3685 | P01574 | IFNB1 |
| 594 | 3686 | Q86WN2 | IFNE |
| 595 | 3687 | P01579 | IFNG |
| 596 | 3688 | P15260 | IFNGR1 |
| 597 | 3689 | P38484 | IFNGR2 |
| 598 | 3690 | Q9P0W0 | IFNK |
| 599 | 3691 | Q8IZJ0 | IFNL2 |
| 600 | 3692 | P05000 | IFNW1 |
| 601 | 3693 | Q8IVU1 | IGDCC3 |
| | 3694 | P08069 | IGF1R |
| 602 | 3695 | P01344 | IGF2 |
| 603 | 3696 | P11717 | IGF2R |
| 604 | 3697 | P35858 | IGFALS |
| 605 | 3698 | Q16270 | IGFBP7 |
| 606 | 3699 | Q8WX77 | IGFBPL1 |
| 607 | 3700 | Q6UW32 | IGFL1 |
| 608 | 3701 | Q6UWQ7 | IGFL2 |
| 609 | 3702 | Q6UXB1 | IGFL3 |
| 610 | 3703 | A6NJ69 | IGIP |
| 611 | 3704 | P15814 | IGLL1 |
| 612 | 3705 | B9A064 | IGLL5 |
| 613 | 3706 | A6NGN9 | IGLON5 |
| 614 | 3707 | Q8N6C5 | IGSF1 |
| 615 | 3708 | Q6WRI0 | IGSF10 |
| 616 | 3709 | Q5DX21 | IGSF11 |
| 617 | 3710 | Q96ID5 | IGSF21 |
| 618 | 3711 | O75054 | IGSF3 |
| 619 | 3712 | Q8N126 | IGSF4B |
| 620 | 3713 | Q8NFZ8 | IGSF4C |
| 621 | 3714 | Q9NSI5 | IGSF5 |
| 622 | 3715 | O95976 | IGSF6 |
| 623 | 3716 | Q969P0 | IGSF8 |
| 624 | 3717 | Q9P212 | IGSF9 |
| 625 | 3718 | P22301 | IL10 |
| 626 | 3719 | Q13651 | IL10RA |
| 627 | 3720 | Q08334 | IL10RB |
| 628 | 3721 | P20809 | IL11 |
| 629 | 3722 | Q14626 | IL11RA |
| 630 | 3723 | P29459 | IL12A |
| 631 | 3724 | P29460 | IL12B |
| 632 | 3725 | P42701 | IL12RB1 |
| 633 | 3726 | Q99665 | IL12RB2 |
| 634 | 3727 | P35225 | IL13 |
| 635 | 3728 | P78552 | IL13RA1 |
| 636 | 3729 | Q14627 | IL13RA2 |
| 637 | 3730 | P40933 | IL15 |
| 638 | 3731 | Q13261 | IL15RA |
| 639 | 3732 | Q14005 | IL16 |
| 640 | 3733 | Q16552 | IL17A |
| 641 | 3734 | Q9UHF5 | IL17B |
| 642 | 3735 | Q9NRM6 | IL17BR |
| 643 | 3736 | Q9P0M4 | IL17C |
| 644 | 3737 | Q8TAD2 | IL17D |
| 645 | 3738 | Q96PD4 | IL17F |
| 646 | 3739 | Q96F46 | IL17RA |
| 647 | 3740 | Q8NAC3 | IL17RC |
| 648 | 3741 | Q8NFM7 | IL17RD |
| 649 | 3742 | Q8NFR9 | IL17RE |
| 650 | 3743 | Q14116 | IL18 |
| 651 | 3744 | O95998 | IL18BP |
| 652 | 3745 | Q13478 | IL18R1 |
| 653 | 3746 | O95256 | IL18RAP |
| 654 | 3747 | Q9UHD0 | IL19 |
| 655 | 3748 | P01583 | IL1A |
| 656 | 3749 | P01584 | IL1B |
| 657 | 3750 | Q8WWZ1 | IL1F10 |
| 658 | 3751 | Q9UBH0 | IL1F5 |
| 659 | 3752 | Q9UHA7 | ILIF6 |
| 660 | 3753 | Q9NZH6 | IL1F7 |
| 661 | 3754 | Q9NZH8 | IL1F9 |
| 662 | 3755 | P14778 | ILIR1 |
| 663 | 3756 | P27930 | ILIR2 |
| 664 | 3757 | Q9NPH3 | IL1RAP |
| 665 | 3758 | Q9NZN1 | IL1RAPL1 |
| 666 | 3759 | Q9NP60 | IL1RAPL2 |
| 667 | 3760 | Q01638 | IL1RL1 |
| 668 | 3761 | Q9HB29 | IL1RL2 |
| 669 | 3762 | P18510 | IL1RN |
| 670 | 3763 | P60568 | IL2 |
| 671 | 3764 | Q9NYY1 | IL20 |
| 672 | 3765 | Q9UHF4 | IL20RA |
| 673 | 3766 | Q6UXL0 | IL20RB |
| 674 | 3767 | Q9HBE4 | IL21 |
| 675 | 3768 | Q9HBE5 | IL21R |
| 676 | 3769 | Q9GZX6 | IL22 |
| 677 | 3770 | Q8N6P7 | IL22RA1 |
| 678 | 3771 | Q969J5 | IL22RA2 |
| 679 | 3772 | Q9NPF7 | IL23A |
| 680 | 3773 | Q5VWK5 | IL23R |
| 681 | 3774 | Q13007 | IL24 |
| 682 | 3775 | Q9H293 | IL25 |
| 683 | 3776 | Q9NPH9 | IL26 |
| 684 | 3777 | Q8NEV9 | IL27 |
| 685 | 3778 | Q6UWB1 | IL27RA |
| 686 | 3779 | Q8IZI9 | IL28B |
| 687 | 3780 | Q8IU57 | IL28RA |
| 688 | 3781 | Q8IU54 | IL29 |
| 689 | 3782 | P01589 | IL2RA |
| 690 | 3783 | P14784 | IL2RB |
| 691 | 3784 | P31785 | IL2RG |
| 692 | 3785 | P08700 | IL3 |
| 693 | 3786 | Q6EBC2 | IL31 |
| 694 | 3787 | Q8NI17 | IL31RA |
| 695 | 3788 | P24001 | IL32 |
| 696 | 3789 | O95760 | IL33 |
| 697 | 3790 | Q6ZMJ4 | IL34 |
| 698 | 3791 | Q9NZH7 | IL36B |
| 699 | 3792 | P26951 | IL3RA |
| 700 | 3793 | P05112 | IL4 |
| 701 | 3794 | P24394 | IL4R |
| 702 | 3795 | P05113 | IL5 |
| 703 | 3796 | Q01344 | IL5RA |
| 704 | 3797 | P05231 | IL6 |
| 705 | 3798 | P08887 | IL6R |
| 706 | 3799 | P40189 | IL6ST |
| 707 | 3800 | P13232 | IL7 |
| 708 | 3801 | P16871 | IL7R |
| 709 | 3802 | P10145 | IL8 |
| 710 | 3803 | P25025 | IL8RB |
| 711 | 3804 | P15248 | IL9 |
| 712 | 3805 | Q01113 | IL9R |
| 713 | 3806 | Q86SU0 | ILDR1 |
| 714 | 3807 | Q9BZV3 | IMPG2 |
| 715 | 3808 | K9M1U5 | INFL4 |
| 716 | 3809 | P01308 | INS |
| 717 | 3810 | P51460 | INSL3 |
| 718 | 3811 | Q9Y5Q6 | INSL5 |
| 719 | 3812 | Q9Y581 | INSL6 |
| 720 | 3813 | P06213 | INSR |
| 721 | 3814 | O14498 | ISLR |
| 722 | 3815 | Q6UXK2 | ISLR2 |
| 723 | 3816 | P56199 | ITGA1 |
| 724 | 3817 | P17301 | ITGA2 |
| 725 | 3818 | P08514 | ITGA2B |
| 726 | 3819 | P26006 | ITGA3 |
| 727 | 3820 | P13612 | ITGA4 |
| 728 | 3821 | P08648 | ITGA5 |
| 729 | 3822 | P23229 | ITGA6 |
| 730 | 3823 | Q13683 | ITGA7 |
| 731 | 3824 | P53708 | ITGA8 |
| 732 | 3825 | Q13797 | ITGA9 |
| 733 | 3826 | P38570 | ITGAE |
| 734 | 3827 | P20701 | ITGAL |
| 735 | 3828 | P11215 | ITGAM |
| 736 | 3829 | P06756 | ITGAV |
| 737 | 3830 | P20702 | ITGAX |
| 738 | 3831 | P05556 | ITGB1 |
| 739 | 3832 | P05107 | ITGB2 |
| 740 | 3833 | P05106 | ITGB3 |
| 741 | 3834 | P18084 | ITGB5 |
| 742 | 3835 | P18564 | ITGB6 |
| 743 | 3836 | P26010 | ITGB7 |
| 744 | 3837 | P26012 | ITGB8 |
| 745 | 3838 | O95965 | ITGBL1 |
| 746 | 3839 | Q8IYV9 | IZUMO |
| 747 | 3840 | P78504 | JAG1 |
| 748 | 3841 | Q9Y219 | JAG2 |
| 749 | 3842 | P57087 | JAM2 |
| 750 | 3843 | Q9BX67 | JAM3 |
| 751 | 3844 | P01591 | JCHAIN |
| 752 | 3845 | P23352 | KAL1 |
| 753 | 3846 | Q96I82 | KAZALD1 |
| 754 | 3847 | Q6UW63 | KDELC1 |
| 755 | 3848 | Q7ZAH8 | KDELC2 |
| 756 | 3849 | P35968 | KDR |
| 757 | 3850 | O60938 | KERA |
| 758 | 3851 | Q5VV43 | KIAA0319 |
| 759 | 3852 | Q8IZA0 | KIAA0319L |
| 760 | 3853 | P43626 | KIR2DL2 |
| 761 | 3854 | P43627 | KIR2DL3 |
| 762 | 3855 | P43628 | KIR2DL3 |
| 763 | 3856 | Q99706 | KIR2DL4 |
| 764 | 3857 | Q8NHK3 | KIR2DL5B |
| 765 | 3858 | Q8N109 | KIR2DL5B |
| 766 | 3859 | P43631 | KIR2DS2 |
| 767 | 3860 | Q14952 | KIR2DS3 |
| 768 | 3861 | Q14954 | KIR2DS4 |
| 769 | 3862 | P43632 | KIR2DS4 |
| 770 | 3863 | Q14953 | KIR2DS5 |
| 771 | 3864 | P43629 | KIR3DL1 |
| 772 | 3865 | P43630 | KIR3DL2 |
| 773 | 3866 | Q8N743 | KIR3DL3 |
| 774 | 3867 | A8MWS1 | KIR3DP1 |
| 775 | 3868 | Q14943 | KIR3DS1 |
| 776 | 3869 | Q9H7L2 | KIR3DX1 |
| 777 | 3870 | Q96J84 | KIRREL |
| 778 | 3871 | Q6UWL6 | KIRREL2 |
| 779 | 3872 | Q8IZU9 | KIRREL3 |
| 780 | 3873 | P10721 | KIT |
| 781 | 3874 | P21583 | KITLG |
| 782 | 3875 | Q12918 | KLRB1 |
| 783 | 3876 | P26715 | KLRC1 |
| 784 | 3877 | P26717 | KLRC2 |
| 785 | 3878 | Q07444 | KLRC3 |
| 786 | 3879 | Q13241 | KLRD1 |
| 787 | 3880 | Q9NZS2 | KLRF1 |
| 788 | 3881 | D3W0D1 | KLRF2 |
| 789 | 3882 | Q96E93 | KLRG1 |
| 790 | 3883 | P26718 | KLRK1 |
| 791 | 3884 | Q9BYJ0 | KSP37 |
| 792 | 3885 | P32004 | LICAM |
| 793 | 3886 | P18627 | LAG3 |
| 794 | 3887 | Q6GTX8 | LAIR1 |
| 795 | 3888 | Q6ISS4 | LAIR2 |
| 796 | 3889 | P25391 | LAMA1 |
| 797 | 3890 | Q16787 | LAMA3 |
| 798 | 3891 | P07942 | LAMB1 |
| 799 | 3892 | Q13751 | LAMB3 |
| 800 | 3893 | A4D0S4 | LAMB4 |
| 801 | 3894 | P11047 | LAMC1 |
| 802 | 3895 | Q13753 | LAMC2 |
| 803 | 3896 | Q6UX15 | LAYN |
| 804 | 3897 | P01130 | LDLR |
| 805 | 3898 | P48357 | LEPR |
| 806 | 3899 | O95970 | LGI1 |
| 807 | 3900 | Q8N0V4 | LGI2 |
| 808 | 3901 | Q8N145 | LGI3 |
| 809 | 3902 | Q8N135 | LGI4 |
| 810 | 3903 | Q9BXB1 | LGR4 |
| 811 | 3904 | O75473 | LGR5 |
| 812 | 3905 | Q9HBX8 | LGR6 |
| 813 | 3906 | Q8WXD0 | LGR8 |
| 814 | 3907 | P22888 | LHCGR |
| 815 | 3908 | P15018 | LIF |
| 816 | 3909 | P42702 | LIFR |
| 817 | 3910 | O75019 | LILRA1 |
| 818 | 3911 | Q8N149 | LILRA2 |
| 819 | 3912 | Q8N6C8 | LILRA3 |
| 820 | 3913 | P59901 | LILRA4 |
| 821 | 3914 | A6NI73 | LILRA5 |
| 822 | 3915 | Q8NHL6 | LILRB1 |
| 823 | 3916 | Q8N423 | LILRB2 |
| 824 | 3917 | O75022 | LILRB3 |
| 825 | 3918 | Q8NHJ6 | LILRB4 |
| 826 | 3919 | O75023 | LILRB5 |
| 827 | 3920 | Q6PI73 | LILRB6 |
| 828 | 3921 | Q96FE5 | LINGO1 |
| 829 | 3922 | Q7L985 | LINGO2 |
| 830 | 3923 | P0C6S8 | LINGO3 |
| 831 | 3924 | Q6UY18 | LINGO4 |
| 832 | 3925 | Q8NCF0 | LOC348174 |
| 833 | 3926 | P28300 | LOX |
| 834 | 3927 | Q08397 | LOXL1 |
| 835 | 3928 | Q96II8 | LRCH3 |
| 836 | 3929 | Q9P244 | LRFN1 |
| 837 | 3930 | Q9ULH4 | LRFN2 |
| 838 | 3931 | Q9BTN0 | LRFN3 |
| 839 | 3932 | Q6PJG9 | LRFN4 |
| 840 | 3933 | Q96NI6 | LRFN5 |
| 841 | 3934 | P02750 | LRG1 |
| 842 | 3935 | Q96JA1 | LRIG1 |
| 843 | 3936 | O94898 | LRIG2 |
| 844 | 3937 | Q6UXM1 | LRIG3 |
| 845 | 3938 | A6NDA9 | LRIT2 |
| 846 | 3939 | Q3SXY7 | LRIT3 |
| 847 | 3940 | Q86VZ4 | LRP11 |
| 848 | 3941 | O75096 | LRP4 |
| 849 | 3942 | O75197 | LRP5 |
| 850 | 3943 | O75581 | LRP6 |
| 851 | 3944 | Q14114 | LRP8 |
| 852 | 3945 | Q8TF66 | LRRC15 |
| 853 | 3946 | Q8N6Y2 | LRRC17 |
| 854 | 3947 | Q9H756 | LRRC19 |
| 855 | 3948 | Q9P2V4 | LRRC21 |
| 856 | 3949 | Q50LG9 | LRRC24 |
| 857 | 3950 | Q8N386 | LRRC25 |
| 858 | 3951 | Q2I0M4 | LRRC26 |
| 859 | 3952 | Q9BY71 | LRRC3 |
| 860 | 3953 | Q14392 | LRRC32 |
| 861 | 3954 | A6NMS7 | LRRC37A |
| 862 | 3955 | O60309 | LRRC37A3 |
| 863 | 3956 | Q96QE4 | LRRC37B |
| 864 | 3957 | Q5VT99 | LRRC38 |
| 865 | 3958 | Q96PB8 | LRRC3B |
| 866 | 3959 | A6NJW4 | LRRC3C |
| 867 | 3960 | Q9HBW1 | LRRC4 |
| 868 | 3961 | Q9NT99 | LRRC4B |
| 869 | 3962 | Q9HCJ2 | LRRC4C |
| 870 | 3963 | Q8N7C0 | LRRC52 |
| 871 | 3964 | Q6ZSA7 | LRRC55 |
| 872 | 3965 | Q7Z2Q7 | LRRC70 |
| 873 | 3966 | Q8IWT6 | LRRC8A |
| 874 | 3967 | Q6P9F7 | LRRC8B |
| 875 | 3968 | Q8TDW0 | LRRC8C |
| 876 | 3969 | Q7L1W4 | LRRC8D |
| 877 | 3970 | Q6UXK5 | LRRN1 |
| 878 | 3971 | Q9H3W5 | LRRN3 |
| 879 | 3972 | Q8WUT4 | LRRN4 |
| 880 | 3973 | Q8ND94 | LRRN4CL |
| 881 | 3974 | O75325 | LRRN5 |
| 882 | 3975 | Q86UE6 | LRRTM1 |
| 883 | 3976 | O43300 | LRRTM2 |
| 884 | 3977 | Q86VH5 | LRRTM3 |
| 885 | 3978 | Q86VH4 | LRRTM4 |
| 886 | 3979 | Q9HBL6 | LRTM1 |
| 887 | 3980 | Q8N967 | LRTM2 |
| 888 | 3981 | Q13449 | LSAMP |
| 889 | 3982 | Q86X29 | LSR |
| 890 | 3983 | P01374 | LTA |
| 891 | 3984 | Q06643 | LTB |
| 892 | 3985 | Q14766 | LTBP1 |
| 893 | 3986 | P36941 | LTBR |
| 894 | 3987 | P02788 | LTF |
| 895 | 3988 | P29376 | LTK |
| 896 | 3989 | P51884 | LUM |
| 897 | 3990 | Q14210 | LY6D |
| 898 | 3991 | Q16553 | LY6E |
| 899 | 3992 | Q8NDX9 | LY6G5B |
| 900 | 3993 | Q5SRR4 | LY6G5C |
| 901 | 3994 | O95867 | LY6G6C |
| 902 | 3995 | O95868 | LY6G6D |
| 903 | 3996 | Q5SQ64 | LY6G6F |
| 904 | 3997 | O94772 | LY6H |
| 905 | 3998 | Q17RY6 | LY6K |
| 906 | 3999 | H3BQJ8 | Ly6L |
| 907 | 4000 | O60449 | LY75 |
| 908 | 4001 | Q9HBG7 | LY9 |
| 909 | 4002 | Q9BZG9 | LYNX1 |
| 910 | 4003 | Q8N2G4 | LYPD1 |
| 911 | 4004 | Q6UXB3 | LYPD2 |
| 912 | 4005 | O95274 | LYPD3 |
| 913 | 4006 | Q6UWN0 | LYPD4 |
| 914 | 4007 | Q6UWN5 | LYPD5 |
| 915 | 4008 | Q86Y78 | LYPD6 |
| 916 | 4009 | Q8NI32 | LYPD6B |
| 917 | 4010 | Q6UX82 | LYPD8 |
| 918 | 4011 | Q13477 | MADCAM1 |
| 919 | 4012 | P20916 | MAG |
| 920 | 4013 | O00462 | MANBA |
| 921 | 4014 | P48740 | MASP1 |
| 922 | 4015 | P21941 | MATN1 |
| 923 | 4016 | O00339 | MATN2 |
| 924 | 4017 | O15232 | MATN3 |
| 925 | 4018 | O95460 | MATN4 |
| 926 | 4019 | P11226 | MBL2 |
| 927 | 4020 | P43121 | MCAM |
| 928 | 4021 | P15529 | MCP |
| 929 | 4022 | Q8NFP4 | MDGA1 |
| 930 | 4023 | Q7Z553 | MDGA2 |
| 931 | 4024 | Q96KG7 | MEGF10 |
| 932 | 4025 | A6BM72 | MEGF11 |
| 933 | 4026 | Q9H1U4 | MEGF9 |
| 934 | 4027 | Q16819 | MEP1A |
| 935 | 4028 | Q16820 | MEP1B |
| 936 | 4029 | Q12866 | MERTK |
| 937 | 4030 | P08581 | MET |
| 938 | 4031 | P55082 | MFAP3 |
| 939 | 4032 | O75121 | MFAP3L |
| 940 | 4033 | Q08431 | MFGE8 |
| 941 | 4034 | P08582 | MFI2 |
| 942 | 4035 | Q29983 | MICA |
| 943 | 4036 | Q29980 | MICB |
| 944 | 4037 | P14174 | MIF |
| 945 | 4038 | Q7Z6M3 | MILR1 |
| 946 | 4039 | P51511 | MMP15 |
| 947 | 4040 | P51512 | MMP16 |
| 948 | 4041 | Q9ULZ9 | MMP17 |
| 949 | 4042 | P08253 | MMP2 |
| 950 | 4043 | Q9Y5R2 | MMP24 |
| 951 | 4044 | Q9H239 | MMP28 |
| 952 | 4045 | P14780 | MMP9 |
| 953 | 4046 | Q13201 | MMRN1 |
| 954 | 4047 | Q16653 | MOG |
| 955 | 4048 | P40238 | MPL |
| 956 | 4049 | P25189 | MPZ |
| 957 | 4050 | O95297 | MPZL1 |
| 958 | 4051 | O60487 | MPZL2 |
| 959 | 4052 | Q6UWV2 | MPZL3 |
| 960 | 4053 | Q95460 | MR1 |
| 961 | 4054 | P22897 | MRC1 |
| 962 | 4055 | Q9UBG0 | MRC2 |
| 963 | 4056 | P21757 | MSR1 |
| 964 | 4057 | P26927 | MST1 |
| 965 | 4058 | P15941 | MUC1 |
| 966 | 4059 | Q9H3R2 | MUC13 |
| 967 | 4060 | Q685J3 | MUC17 |
| 968 | 4061 | Q8N307 | MUC20 |
| 969 | 4062 | Q5SSG8 | MUC21 |
| 970 | 4063 | P98088 | MUC5AC |
| 971 | 4064 | O15146 | MUSK |
| 972 | 4065 | Q9BRK3 | MXRA8 |
| 973 | 4066 | Q9UK23 | NAGPA |
| 974 | 4067 | P13591 | NCAM1 |
| 975 | 4068 | O15394 | NCAM2 |
| 976 | 4069 | O14594 | NCAN |
| 977 | 4070 | O76036 | NCR1 |
| 978 | 4071 | O95944 | NCR2 |
| 979 | 4072 | O14931 | NCR3 |
| 980 | 4073 | Q8TB73 | NDNF |
| 981 | 4074 | Q7Z3B1 | NEGR1 |
| 982 | 4075 | Q92832 | NELL1 |
| 983 | 4076 | Q99435 | NELL2 |
| 984 | 4077 | Q92859 | NEO1 |
| 985 | 4078 | Q8NET5 | NFAM1 |
| 986 | 4079 | O94856 | NFASC |
| 987 | 4080 | P01138 | NGFB |
| 988 | 4081 | P08138 | NGFR |
| 989 | 4082 | P14543 | NID1 |
| 990 | 4083 | Q14112 | NID2 |
| 991 | 4084 | Q8NFZ3 | NLGN4Y |
| 992 | 4085 | Q8NFZ3 | NLGN4Y |
| 993 | 4086 | Q96P20 | NLRP3 |
| 994 | 4087 | Q8TDY8 | NOPE |
| 995 | 4088 | Q04721 | NOTCH2 |
| 996 | 4089 | Q7Z3S9 | NOTCH2NL |
| 997 | 4090 | Q99466 | NOTCH4 |
| 998 | 4091 | O60500 | NPHS1 |
| 999 | 4092 | Q6UXI9 | NPNT |
| 1000 | 4093 | Q9Y639 | NPTN |
| 1001 | 4094 | Q92823 | NRCAM |
| 1002 | 4095 | Q02297 | NRG1 |
| 1003 | 4096 | Q02297 | NRGI |
| 1004 | 4097 | O14511 | NRG2 |
| 1005 | 4098 | P56975 | NRG3 |
| 1006 | 4099 | Q8WWG1 | NRG4 |
| 1007 | 4100 | O14786 | NRP1 |
| 1008 | 4101 | O60462 | NRP2 |
| 1009 | 4102 | Q86YC3 | NRROS |
| 1010 | 4103 | P58400 | NRXN1 |
| 1011 | 4104 | Q9HDB5 | NRXN3 |
| 1012 | 4105 | P21589 | NT5E |
| 1013 | 4106 | P20783 | NTF3 |
| 1014 | 4107 | P34130 | NTF5 |
| 1015 | 4108 | Q9P121 | NTM |
| 1016 | 4109 | O95631 | NTN1 |
| 1017 | 4110 | O00634 | NTN3 |
| 1018 | 4111 | Q9HB63 | NTN4 |
| 1019 | 4112 | Q8WTR8 | NTN5 |
| 1020 | 4113 | Q9Y212 | NTNG1 |
| 1021 | 4114 | Q96CW9 | NTNG2 |
| 1022 | 4115 | P04629 | NTRK1 |
| 1023 | 4116 | Q16620 | NTRK2 |
| 1024 | 4117 | Q16288 | NTRK3 |
| 1025 | 4118 | Q8N323 | NXPE1 |
| 1026 | 4119 | Q969Y0 | NXPE3 |
| 1027 | 4120 | Q6UWF7 | NXPE4 |
| 1028 | 4121 | Q9GZU5 | NYX |
| 1029 | 4122 | P20774 | OGN |
| 1030 | 4123 | Q8WWZ8 | OIT3 |
| 1031 | 4124 | P78380 | OLR1 |
| 1032 | 4125 | Q99983 | OMD |
| 1033 | 4126 | P23515 | OMG |
| 1034 | 4127 | Q14982 | OPCML |
| 1035 | 4128 | Q9UBM4 | OPTC |
| 1036 | 4129 | Q8IYS5 | OSCAR |
| 1037 | 4130 | Q99650 | OSMR |
| 1038 | 4131 | Q6UXH9 | PAMR1 |
| 1039 | 4132 | Q06141 | PAP |
| 1040 | 4133 | O95428 | PAPLN |
| 1041 | 4134 | Q13219 | PAPPA |
| 1042 | 4135 | Q8WXA2 | PATE1 |
| 1043 | 4136 | Q6UY27 | PATE2 |
| 1044 | 4137 | B3GLJ2 | PATE3 |
| 1045 | 4138 | P0C8F1 | PATE4 |
| 1046 | 4139 | Q9P2E7 | PCDH10 |
| 1047 | 4140 | Q9NPG4 | PCDH12 |
| 1048 | 4141 | Q8N6Y1 | PCDH20 |
| 1049 | 4142 | Q9HC56 | PCDH9 |
| 1050 | 4143 | Q9Y5H5 | PCDHA9 |
| 1051 | 4144 | Q9Y5F3 | PCDHB1 |
| 1052 | 4145 | Q9Y5F2 | PCDHB11 |
| 1053 | 4146 | Q9UN66 | PCDHB13 |
| 1054 | 4147 | Q9Y5E8 | PCDHB15 |
| 1055 | 4148 | Q9NRJ7 | PCDHB16 |
| 1056 | 4149 | Q9Y5E6 | PCDHB3 |
| 1057 | 4150 | Q9Y5E4 | PCDHB5 |
| 1058 | 4151 | Q9Y5E3 | PCDHB6 |
| 1059 | 4152 | Q9Y5E2 | PCDHB7 |
| 1060 | 4153 | Q9Y5E1 | PCDHB9 |
| 1061 | 4154 | Q9Y5G9 | PCDHGA4 |
| 1062 | 4155 | Q9Y5G1 | PCDHGB3 |
| 1063 | 4156 | Q9Y5F9 | PCDHGB6 |
| 1064 | 4157 | Q9UN70 | PCDHGC3 |
| 1065 | 4158 | Q9UHG2 | PCSK1N |
| 1066 | 4159 | Q8NBP7 | PCSK9 |
| 1067 | 4160 | Q15116 | PDCD1 |
| 1068 | 4161 | Q9BQ51 | PDCD1LG2 |
| 1069 | 4162 | P04085 | PDGFA |
| 1070 | 4163 | P01127 | PDGFB |
| 1071 | 4164 | Q9NRA1 | PDGFC |
| 1072 | 4165 | Q9GZP0 | PDGFD |
| 1073 | 4166 | P16234 | PDGFRA |
| 1074 | 4167 | P09619 | PDGFRB |
| 1075 | 4168 | Q15198 | PDGFRL |
| 1076 | 4169 | P16284 | PECAM1 |
| 1077 | 4170 | P02776 | PF4 |
| 1078 | 4171 | P49763 | PGF |
| 1079 | 4172 | O75594 | PGLYRP1 |
| 1080 | 4173 | P01833 | PIGR |
| 1081 | 4174 | Q96FE7 | PIK3IP1 |
| 1082 | 4175 | Q9UKJ1 | PILRA |
| 1083 | 4176 | Q9UKJ0 | PILRB |
| 1084 | 4177 | A6NC86 | PINLYP |
| 1085 | 4178 | P12273 | PIP |
| 1086 | 4179 | Q504Y2 | PKDCC |
| 1087 | 4180 | P00750 | PLAT |
| 1088 | 4181 | P00749 | PLAU |
| 1089 | 4182 | Q03405 | PLAUR |
| 1090 | 4183 | Q9HCM2 | PLXNA4 |
| 1091 | 4184 | Q7Z5L7 | PODN |
| 1092 | 4185 | Q6PEZ8 | PODNL1 |
| 1093 | 4186 | P02775 | PPBP |
| 1094 | 4187 | Q99944 | PPT2 |
| 1095 | 4188 | P51888 | PRELP |
| 1096 | 4189 | P14222 | PRF1 |
| 1097 | 4190 | P13727 | PRG2 |
| 1098 | 4191 | Q9Y2Y8 | PRG3 |
| 1099 | 4192 | P16471 | PRLR |
| 1100 | 4193 | P04070 | PROC |
| 1101 | 4194 | Q9UNN8 | PROCR |
| 1102 | 4195 | P07225 | PROS1 |
| 1103 | 4196 | P22891 | PROZ |
| 1104 | 4197 | Q2VWP7 | PRTG |
| 1105 | 4198 | Q8N6Q3 | PRV1 |
| 1106 | 4199 | O43653 | PSCA |
| 1107 | 4200 | Q9UQ74 | PSG1 |
| 1108 | 4201 | P11464 | PSG1 |
| 1109 | 4202 | Q9UQ72 | PSG2 |
| 1110 | 4203 | P11465 | PSG2 |
| 1111 | 4204 | Q16557 | PSG4 |
| 1112 | 4205 | Q00888 | PSG4 |
| 1113 | 4206 | Q15238 | PSG5 |
| 1114 | 4207 | Q00889 | PSG6 |
| 1115 | 4208 | Q13046 | PSG8 |
| 1116 | 4209 | Q00887 | PSG9 |
| 1117 | 4210 | O60542 | PSPN |
| 1118 | 4211 | P23219 | PTGS1 |
| 1119 | 4212 | P35354 | PTGS2 |
| 1120 | 4213 | Q13308 | PTK7 |
| 1121 | 4214 | Q9H106 | PTPNS1L2 |
| 1122 | 4215 | P23467 | PTPRB |
| 1123 | 4216 | P08575 | PTPRC |
| 1124 | 4217 | P23468 | PTPRD |
| 1125 | 4218 | P10586 | PTPRF |
| 1126 | 4219 | P23470 | PTPRG |
| 1127 | 4220 | Q9HD43 | PTPRH |
| 1128 | 4221 | Q12913 | PTPRJ |
| 1129 | 4222 | Q15262 | PTPRK |
| 1130 | 4223 | Q16849 | PTPRN |
| 1131 | 4224 | Q16827 | PTPRO |
| 1132 | 4225 | Q15256 | PTPRR |
| 1133 | 4226 | Q13332 | PTPRS |
| 1134 | 4227 | P26022 | PTX3 |
| 1135 | 4228 | P15151 | PVR |
| 1136 | 4229 | Q15223 | PVRL1 |
| 1137 | 4230 | Q92692 | PVRL2 |
| 1138 | 4231 | Q9NQS3 | PVRL3 |
| 1139 | 4232 | Q96NY8 | PVRL4 |
| 1140 | 4233 | P20742 | PZP |
| 1141 | 4234 | P05451 | REG1A |
| 1142 | 4235 | P48304 | REG1B |
| 1143 | 4236 | Q6UW15 | REG3G |
| 1144 | 4237 | Q9BYZ8 | REG4 |
| 1145 | 4238 | Q9HCK4 | ROBO2 |
| 1146 | 4239 | Q8WZ75 | ROBO4 |
| 1147 | 4240 | Q01973 | ROR1 |
| 1148 | 4241 | Q01974 | ROR2 |
| 1149 | 4242 | P08922 | ROS1 |
| 1150 | 4243 | Q9BZR6 | RTN4R |
| 1151 | 4244 | Q86UN2 | RTN4RL1 |
| 1152 | 4245 | Q86UN3 | RTN4RL2 |
| 1153 | 4246 | Q9HBX9 | RXFP1 |
| 1154 | 4247 | Q6AZY7 | SCARA3 |
| 1155 | 4248 | Q14162 | SCARF1 |
| 1156 | 4249 | Q96GP6 | SCARF2 |
| 1157 | 4250 | Q07699 | SCN1B |
| 1158 | 4251 | O60939 | SCN2B |
| 1159 | 4252 | Q9NY72 | SCN3B |
| 1160 | 4253 | Q8IWT1 | SCN4B |
| 1161 | 4254 | Q8IWY4 | SCUBE1 |
| 1162 | 4255 | Q9NQ36 | SCUBE2 |
| 1163 | 4256 | Q8IX30 | SCUBE3 |
| 1164 | 4257 | P18827 | SDC1 |
| 1165 | 4258 | P34741 | SDC2 |
| 1166 | 4259 | P31431 | SDC4 |
| 1167 | 4260 | Q58EX2 | SDK2 |
| 1168 | 4261 | Q8WVN6 | SECTM1 |
| 1169 | 4262 | P16581 | SELE |
| 1170 | 4263 | P14151 | SELL |
| 1171 | 4264 | P16109 | SELP |
| 1172 | 4265 | Q14563 | SEMA3A |
| 1173 | 4266 | Q13214 | SEMA3B |
| 1174 | 4267 | Q99985 | SEMA3C |
| 1175 | 4268 | O95025 | SEMA3D |
| 1176 | 4269 | O15041 | SEMA3E |
| 1177 | 4270 | Q13275 | SEMA3F |
| 1178 | 4271 | Q9NS98 | SEMA3G |
| 1179 | 4272 | Q9H3S1 | SEMA4A |
| 1180 | 4273 | Q9NPR2 | SEMA4B |
| 1181 | 4274 | Q9C0C4 | SEMA4C |
| 1182 | 4275 | Q92854 | SEMA4D |
| 1183 | 4276 | O95754 | SEMA4F |
| 1184 | 4277 | Q9NTN9 | SEMA4G |
| 1185 | 4278 | Q9P283 | SEMA5B |
| 1186 | 4279 | Q9H2E6 | SEMA6A |
| 1187 | 4280 | Q9H3T3 | SEMA6B |
| 1188 | 4281 | Q9H3T2 | SEMA6C |
| 1189 | 4282 | O75326 | SEMA7A |
| 1190 | 4283 | Q8IWL2 | SFTPA1 |
| 1191 | 4284 | Q8IWL1 | SFTPA2 |
| 1192 | 4285 | P35247 | SFTPD |
| 1193 | 4286 | Q6IA17 | SIGIRR |
| 1194 | 4287 | Q96LC7 | SIGLEC10 |
| 1195 | 4288 | Q96RL6 | SIGLEC11 |
| 1196 | 4289 | Q96PQ1 | SIGLEC12 |
| 1197 | 4290 | Q08ET2 | SIGLEC14 |
| 1198 | 4291 | Q6ZMC9 | SIGLEC15 |
| 1199 | 4292 | A6NMB1 | SIGLEC16 |
| 1200 | 4293 | O15389 | SIGLEC5 |
| 1201 | 4294 | O43699 | SIGLEC6 |
| 1202 | 4295 | Q9Y286 | SIGLEC7 |
| 1203 | 4296 | Q9NYZ4 | SIGLEC8 |
| 1204 | 4297 | Q9Y336 | SIGLEC9 |
| 1205 | 4298 | P78324 | SIRPA |
| 1206 | 4299 | O00241 | SIRPB1 |
| 1207 | 4300 | Q5JXA9 | SIRPB2 |
| 1208 | 4301 | Q9P1W8 | SIRPG |
| 1209 | 4302 | Q13291 | SLAMF1 |
| 1210 | 4303 | Q96DU3 | SLAMF6 |
| 1211 | 4304 | Q9NQ25 | SLAMF7 |
| 1212 | 4305 | Q9P0V8 | SLAMF8 |
| 1213 | 4306 | Q96A28 | SLAMF9 |
| 1214 | 4307 | O94813 | SLIT2 |
| 1215 | 4308 | O75094 | SLIT3 |
| 1216 | 4309 | Q96PX8 | SLITRK1 |
| 1217 | 4310 | Q9H156 | SLITRK2 |
| 1218 | 4311 | O94933 | SLITRK3 |
| 1219 | 4312 | Q8IW52 | SLITRK4 |
| 1220 | 4313 | O94991 | SLITRK5 |
| 1221 | 4314 | Q9H5Y7 | SLITRK6 |
| 1222 | 4315 | P55000 | SLURP1 |
| 1223 | 4316 | Q8TER0 | SNED1 |
| 1224 | 4317 | Q8TDM5 | SPACA4 |
| 1225 | 4318 | W5XKT8 | SPACA6P |
| 1226 | 4319 | O43278 | SPINT1 |
| 1227 | 4320 | P78539 | SRPX |
| 1228 | 4321 | O60687 | SRPX2 |
| 1229 | 4322 | Q8WTU2 | SSC4D |
| 1230 | 4323 | Q13586 | STIM1 |
| 1231 | 4324 | Q9P246 | STIM2 |
| 1232 | 4325 | Q6UWL2 | SUSD1 |
| 1233 | 4326 | Q9UGT4 | SUSD2 |
| 1234 | 4327 | Q5VX71 | SUSD4 |
| 1235 | 4328 | Q86UU9 | TAC4 |
| 1236 | 4329 | B6A8C7 | TARM1 |
| 1237 | 4330 | P13385 | TDGF1 |
| 1238 | 4331 | Q02763 | TEK |
| 1239 | 4332 | Q9UKZ4 | TENM1 |
| 1240 | 4333 | Q9BY14 | TEX101 |
| 1241 | 4334 | P02787 | TF |
| 1242 | 4335 | Q9UP52 | TFR2 |
| 1243 | 4336 | P02786 | TFRC |
| 1244 | 4337 | P01135 | TGFA |
| 1245 | 4338 | P01137 | TGFB1 |
| 1246 | 4339 | P61812 | TGFB2 |
| 1247 | 4340 | P10600 | TGFB3 |
| 1248 | 4341 | Q15582 | TGFBI |
| 1249 | 4342 | P36897 | TGFBR1 |
| 1250 | 4343 | P37173 | TGFBR2 |
| 1251 | 4344 | Q03167 | TGFBR3 |
| 1252 | 4345 | P07204 | THBD |
| 1253 | 4346 | P07996 | THBS1 |
| 1254 | 4347 | P35442 | THBS2 |
| 1255 | 4348 | P49746 | THBS3 |
| 1256 | 4349 | P35443 | THBS4 |
| 1257 | 4350 | P04216 | THY1 |
| 1258 | 4351 | P35590 | TIE1 |
| 1259 | 4352 | Q495A1 | TIGIT |
| 1260 | 4353 | Q96H15 | TIMD4 |
| 1261 | 4354 | O43897 | TLL1 |
| 1262 | 4355 | Q9Y6L7 | TLL2 |
| 1263 | 4356 | Q15399 | TLR1 |
| 1264 | 4357 | Q9BXR5 | TLR10 |
| 1265 | 4358 | O60603 | TLR2 |
| 1266 | 4359 | O15455 | TLR3 |
| 1267 | 4360 | O00206 | TLR4 |
| 1268 | 4361 | O60602 | TLR5 |
| 1269 | 4362 | Q9Y2C9 | TLR6 |
| 1270 | 4363 | Q9NYK1 | TLR7 |
| 1271 | 4364 | Q9NR97 | TLR8 |
| 1272 | 4365 | Q9NR96 | TLR9 |
| 1273 | 4366 | O43657 | TM4SF6 |
| 1274 | 4367 | Q8IYR6 | TMEFF1 |
| 1275 | 4368 | Q9UIK5 | TMEFF2 |
| 1276 | 4369 | Q8N3G9 | TMEM130 |
| 1277 | 4370 | Q9H665 | TMEM149 |
| 1278 | 4371 | Q86YD3 | TMEM25 |
| 1279 | 4372 | Q9HCN3 | TMEM8 |
| 1280 | 4373 | Q6P7N7 | TMEM81 |
| 1281 | 4374 | A6NDV4 | TMEM8B |
| 1282 | 4375 | Q6UXZ0 | TMIGD1 |
| 1283 | 4376 | Q96BF3 | TMIGD2 |
| 1284 | 4377 | P05452 | TNA |
| 1285 | 4378 | P01375 | TNF |
| 1286 | 4379 | O00220 | TNFRSF10A |
| 1287 | 4380 | O14763 | TNFRSF10B |
| 1288 | 4381 | O14798 | TNFRSF10C |
| 1289 | 4382 | Q9UBN6 | TNFRSF10D |
| 1290 | 4383 | Q9Y6Q6 | TNFRSF11A |
| 1291 | 4384 | O00300 | TNFRSF11B |
| 1292 | 4385 | Q9NP84 | TNFRSF12A |
| 1293 | 4386 | O14836 | TNFRSF13B |
| 1294 | 4387 | Q96RJ3 | TNFRSF13C |
| 1295 | 4388 | Q92956 | TNFRSF14 |
| 1296 | 4389 | Q02223 | TNFRSF17 |
| 1297 | 4390 | Q9NS68 | TNFRSF19 |
| 1298 | 4391 | Q969Z4 | TNFRSF19L |
| 1299 | 4392 | P19438 | TNFRSF1A |
| 1300 | 4393 | P20333 | TNFRSF1B |
| 1301 | 4394 | O75509 | TNFRSF21 |
| 1302 | 4395 | Q93038 | TNFRSF25 |
| 1303 | 4396 | P43489 | TNFRSF4 |
| 1304 | 4397 | P25942 | TNFRSF5 |
| 1305 | 4398 | P25445 | TNFRSF6 |
| 1306 | 4399 | O95407 | TNFRSF6B |
| 1307 | 4400 | P26842 | TNFRSF7 |
| 1308 | 4401 | P28908 | TNFRSF8 |
| 1309 | 4402 | Q07011 | TNFRSF9 |
| 1310 | 4403 | P50591 | TNFSF10 |
| 1311 | 4404 | O14788 | TNFSF11 |
| 1312 | 4405 | O43508 | TNFSF12 |
| 1313 | 4406 | O75888 | TNFSF13 |
| 1314 | 4407 | Q9Y275 | TNFSF13B |
| 1315 | 4408 | O43557 | TNFSF14 |
| 1316 | 4409 | O95150 | TNFSF15 |
| 1317 | 4410 | Q9UNG2 | TNFSF18 |
| 1318 | 4411 | P23510 | TNFSF4 |
| 1319 | 4412 | P48023 | TNFSF6 |
| 1320 | 4413 | P32971 | TNFSF8 |
| 1321 | 4414 | P41273 | TNFSF9 |
| 1322 | 4415 | Q9UQP3 | TNN |
| 1323 | 4416 | Q92752 | TNR |
| 1324 | 4417 | P22105 | TNXB |
| 1325 | 4418 | Q13641 | TPBG |
| 1326 | 4419 | P0DKB5 | TPBGL |
| 1327 | 4420 | P07202 | TPO |
| 1328 | 4421 | Q86V40 | TRABD2A |
| 1329 | 4422 | Q9NP99 | TREM1 |
| 1330 | 4423 | Q9NZC2 | TREM2 |
| 1331 | 4424 | Q86YW5 | TREML1 |
| 1332 | 4425 | Q5T2D2 | TREML2 |
| 1333 | 4426 | Q6UXN2 | TREML4 |
| 1334 | 4427 | Q7L0X0 | TRIL |
| 1335 | 4428 | P16473 | TSHR |
| 1336 | 4429 | Q8WUA8 | TSKU |
| 1337 | 4430 | Q969D9 | TSLP |
| 1338 | 4431 | O60635 | TSPAN1 |
| 1339 | 4432 | O95859 | TSPAN12 |
| 1340 | 4433 | O95857 | TSPAN13 |
| 1341 | 4434 | O95858 | TSPAN15 |
| 1342 | 4435 | Q96FV3 | TSPAN17 |
| 1343 | 4436 | Q96SJ8 | TSPAN18 |
| 1344 | 4437 | O60636 | TSPAN2 |
| 1345 | 4438 | O60637 | TSPAN3 |
| 1346 | 4439 | Q12999 | TSPAN31 |
| 1347 | 4440 | Q86UF1 | TSPAN33 |
| 1348 | 4441 | O14817 | TSPAN4 |
| 1349 | 4442 | P62079 | TSPAN5 |
| 1350 | 4443 | P41732 | TSPAN7 |
| 1351 | 4444 | P19075 | TSPAN8 |
| 1352 | 4445 | O75954 | TSPAN9 |
| 1353 | 4446 | Q06418 | TYRO3 |
| 1354 | 4447 | O43914 | TYROBP |
| 1355 | 4448 | P07911 | UMOD |
| 1356 | 4449 | Q6ZN44 | UNC5A |
| 1357 | 4450 | Q8IZJ1 | UNC5B |
| 1358 | 4451 | O95185 | UNC5C |
| 1359 | 4452 | Q6UXZ4 | UNC5D |
| 1360 | 4453 | O00322 | UPK1A |
| 1361 | 4454 | O75841 | UPK1B |
| 1362 | 4455 | Q6EMK4 | VASN |
| 1363 | 4456 | P19320 | VCAM1 |
| 1364 | 4457 | P15692 | VEGFA |
| 1365 | 4458 | P49765 | VEGFB |
| 1366 | 4459 | P49767 | VEGFC |
| 1367 | 4460 | P98155 | VLDLR |
| 1368 | 4461 | Q86XK7 | VSIG1 |
| 1369 | 4462 | Q8N0Z9 | VSIG10 |
| 1370 | 4463 | Q96IQ7 | VSIG2 |
| 1371 | 4464 | Q9Y279 | VSIG4 |
| 1372 | 4465 | Q5VU13 | VSIG8 |
| 1373 | 4466 | Q6UX27 | VSTM1 |
| 1374 | 4467 | Q8TAG5 | VSTM2A |
| 1375 | 4468 | A6NLU5 | VSTM2B |
| 1376 | 4469 | Q96N03 | VSTM2L |
| 1377 | 4470 | Q8IW00 | VSTM4 |
| 1378 | 4471 | A8MXK1 | VSTM5 |
| 1379 | 4472 | Q7Z7D3 | VTCN1 |
| 1380 | 4473 | Q6PCB0 | VWA1 |
| 1381 | 4474 | Q5GFL6 | VWA2 |
| 1382 | 4475 | Q96DN2 | VWCE |
| 1383 | 4476 | Q96NZ8 | WFIKKN1 |
| 1384 | 4477 | Q8TEU8 | WFIKKN2 |
| 1385 | 4478 | Q9Y5W5 | WIF1 |
| 1386 | 4479 | P47992 | XCL1 |
| 1387 | 4480 | Q9UBD3 | XCL2 |
| 1388 | 4481 | Q9BS86 | ZPBP |
| 1389 | 4482 | Q6X784 | ZPBP2 |
| 1390 | 4483 | Q96GS6 | ABHD17A |
| 1391 | 4484 | Q5VST6 | ABHD17B |
| 1392 | 4485 | Q0P651 | ABHD18 |
| 1393 | 4486 | Q9C0K3 | ACTR3C |
| 1394 | 4487 | O15204 | ADAMDEC1 |
| 1395 | 4488 | Q6ZMM2 | ADAMTSL5 |
| 1396 | 4489 | Q9UKB5 | AJAP1 |
| 1397 | 4490 | Q6UX46 | ALKAL2 |
| 1398 | 4491 | P03971 | AMH |
| 1399 | 4492 | Q9BXJ7 | AMN |
| 1400 | 4493 | P04746 | AMY2A |
| 1401 | 4494 | P19961 | AMY2B |
| 1402 | 4495 | O95841 | ANGPTL1 |
| 1403 | 4496 | Q86XS5 | ANGPTL5 |
| 1404 | 4497 | Q8NI99 | ANGPTL6 |
| 1405 | 4498 | Q6UXH0 | ANGPTL8 |
| 1406 | 4499 | A6NMY6 | ANXA2P2 |
| 1407 | 4500 | P28039 | AOAH |
| 1408 | 4501 | Q8NCL9 | APCDD1L |
| 1409 | 4502 | P06727 | APOA4 |
| 1410 | 4503 | P15848 | ARSB |
| 1411 | 4504 | Q5T4W7 | ARTN |
| 1412 | 4505 | Q16515 | ASIC2 |
| 1413 | 4506 | Q86Y30 | BAGE2 |
| 1414 | 4507 | Q86Y29 | BAGE3 |
| 1415 | 4508 | P23560 | BDNF |
| 1416 | 4509 | P22004 | BMP6 |
| 1417 | 4510 | Q9BQP9 | BPIFA3 |
| 1418 | 4511 | Q86YQ2 | BPIFA4P |
| 1419 | 4512 | Q8NFQ6 | BPIFC |
| 1420 | 4513 | A6NE02 | BTBD17 |
| 1421 | 4514 | Q8N8P7 | C11orf44 |
| 1422 | 4515 | C9JXX5 | C11orf94 |
| 1423 | 4516 | Q9H972 | C14orf93 |
| 1424 | 4517 | A6NNL5 | C15orf61 |
| 1425 | 4518 | Q96HA4 | C1orf159 |
| 1426 | 4519 | P02745 | C1QA |
| 1427 | 4520 | P02746 | C1QB |
| 1428 | 4521 | P02747 | C1QC |
| 1429 | 4522 | Q5VWW1 | C1QL3 |
| 1430 | 4523 | Q5T7M4 | C1QTNF12 |
| 1431 | 4524 | Q9NYP8 | C21orf62 |
| 1432 | 4525 | C9J442 | C22orf46 |
| 1433 | 4526 | Q8N8R5 | C2orf69 |
| 1434 | 4527 | Q7Z4R8 | C6orf120 |
| 1435 | 4528 | Q5VTT2 | C9orf135 |
| 1436 | 4529 | Q6ZRZ4 | C9orf47 |
| 1437 | 4530 | P23280 | CA6 |
| 1438 | 4531 | Q9NYX4 | CALY |
| 1439 | 4532 | Q8IUK8 | CBLN2 |
| 1440 | 4533 | Q6UW01 | CBLN3 |
| 1441 | 4534 | P0C854 | CECR9 |
| 1442 | 4535 | Q8N7Q2 | CELF2-AS1 |
| 1443 | 4536 | Q9UKY3 | CES1P1 |
| 1444 | 4537 | Q5XG92 | CES4A |
| 1445 | 4538 | Q6NT32 | CES5A |
| 1446 | 4539 | P01215 | CGA |
| 1447 | 4540 | A6NKQ9 | CGB1 |
| 1448 | 4541 | Q6NT52 | CGB2 |
| 1449 | 4542 | P0DN86 | CGB3 |
| 1450 | 4543 | P0DN87 | CGB7 |
| 1451 | 4544 | Q9BZP6 | CHIA |
| 1452 | 4545 | P02708 | CHRNA1 |
| 1453 | 4546 | Q15822 | CHRNA2 |
| 1454 | 4547 | Q04844 | CHRNE |
| 1455 | 4548 | P07510 | CHRNG |
| 1456 | 4549 | Q9Y6N3 | CLCA3P |
| 1457 | 4550 | Q6UVW9 | CLEC2A |
| 1458 | 4551 | Q6UWE3 | CLPSL2 |
| 1459 | 4552 | Q9HBJ8 | CLTRN |
| 1460 | 4553 | Q15846 | CLUL1 |
| 1461 | 4554 | O43405 | COCH |
| 1462 | 4555 | Q96A83 | COL26A1 |
| 1463 | 4556 | Q2VPA4 | CR1L |
| 1464 | 4557 | P54107 | CRISP1 |
| 1465 | 4558 | O76096 | CST7 |
| 1466 | 4559 | Q5W188 | CST9LP1 |
| 1467 | 4560 | Q5H943 | CT83 |
| 1468 | 4561 | Q16619 | CTF1 |
| 1469 | 4562 | Q9UBX1 | CTSF |
| 1470 | 4563 | P25774 | CTSS |
| 1471 | 4564 | P56202 | CTSW |
| 1472 | 4565 | O60888 | CUTA |
| 1473 | 4566 | A0A087X1C5 | CYP2D7 |
| 1474 | 4567 | P81605 | DCD |
| 1475 | 4568 | Q9BYW3 | DEFB126 |
| 1476 | 4569 | Q7Z7B8 | DEFB128 |
| 1477 | 4570 | Q6IED9 | DGAT2L7P |
| 1478 | 4571 | Q6UWP2 | DHRS11 |
| 1479 | 4572 | Q6UX07 | DHRS13 |
| 1480 | 4573 | Q6PKH6 | DHRS4L2 |
| 1481 | 4574 | Q9BPW9 | DHRS9 |
| 1482 | 4575 | Q9H7Y0 | DIPK2B |
| 1483 | 4576 | Q9H4A9 | DPEP2 |
| 1484 | 4577 | Q8NBI3 | DRAXIN |
| 1485 | 4578 | Q8N1N2 | DYNAP |
| 1486 | 4579 | P52798 | EFNA4 |
| 1487 | 4580 | O94919 | ENDOD1 |
| 1488 | 4581 | P21128 | ENDOU |
| 1489 | 4582 | Q5NDL2 | EOGT |
| 1490 | 4583 | P60507 | ERVFC1 |
| 1491 | 4584 | M5A8F1 | ERVH48-1 |
| 1492 | 4585 | O42043 | ERVK-18 |
| 1493 | 4586 | P61566 | ERVK-24 |
| 1494 | 4587 | P61567 | ERVK-7 |
| 1495 | 4588 | Q9NX77 | ERVK13-1 |
| 1496 | 4589 | B6SEH8 | ERVV-1 |
| 1497 | 4590 | B6SEH9 | ERVV-2 |
| 1498 | 4591 | P22794 | EVI2A |
| 1499 | 4592 | Q8N2X6 | EXOC3-AS1 |
| 1500 | 4593 | A1KXE4 | FAM168B |
| 1501 | 4594 | Q7Z5A7 | FAM19A5 |
| 1502 | 4595 | A6NFZ4 | FAM24A |
| 1503 | 4596 | P98173 | FAM3A |
| 1504 | 4597 | Q15485 | FCN2 |
| 1505 | 4598 | Q9UGM5 | FETUB |
| 1506 | 4599 | Q9HCT0 | FGF22 |
| 1507 | 4600 | P08620 | FGF4 |
| 1508 | 4601 | P55075 | FGF8 |
| 1509 | 4602 | A5D6W6 | FITM1 |
| 1510 | 4603 | Q86VR8 | FJX1 |
| 1511 | 4604 | Q71RG6 | FP248 |
| 1512 | 4605 | O95633 | FSTL3 |
| 1513 | 4606 | Q14332 | FZD2 |
| 1514 | 4607 | P14867 | GABRA1 |
| 1515 | 4608 | P47869 | GABRA2 |
| 1516 | 4609 | P78334 | GABRE |
| 1517 | 4610 | Q99928 | GABRG3 |
| 1518 | 4611 | A8MPY1 | GABRR3 |
| 1519 | 4612 | P54826 | GAS1 |
| 1520 | 4613 | Q9UFP1 | GASKIA |
| 1521 | 4614 | P27539 | GDF1 |
| 1522 | 4615 | Q7Z4P5 | GDF7 |
| 1523 | 4616 | Q8N9F7 | GDPD1 |
| 1524 | 4617 | Q7L5L3 | GDPD3 |
| 1525 | 4618 | Q3B7J2 | GFOD2 |
| 1526 | 4619 | Q6UXV0 | GFRAL |
| 1527 | 4620 | A6NGU5 | GGT3P |
| 1528 | 4621 | Q8N2G8 | GHDC |
| 1529 | 4622 | P0CG01 | GKN3P |
| 1530 | 4623 | Q6ZMI3 | GLDN |
| 1531 | 4624 | Q5JXX5 | GLRA4 |
| 1532 | 4625 | Q96MS3 | GLT1D1 |
| 1533 | 4626 | Q86YW7 | GPHB5 |
| 1534 | 4627 | Q9NPR9 | GPR108 |
| 1535 | 4628 | Q6UXU4 | GSG1L |
| 1536 | 4629 | A8MUP6 | GSG1L2 |
| 1537 | 4630 | Q8N7I0 | GVQW1 |
| 1538 | 4631 | Q9BXW7 | HDHD5 |
| 1539 | 4632 | C9JL84 | HHLA1 |
| 1540 | 4633 | A8MTL9 | HMSD |
| 1541 | 4634 | P22626 | HNRNPA2B1 |
| 1542 | 4635 | P00738 | HP |
| 1543 | 4636 | P00739 | HPR |
| 1544 | 4637 | P02790 | HPX |
| 1545 | 4638 | Q7Z5J1 | HSD11B1L |
| 1546 | 4639 | Q70Z44 | HTR3D |
| 1547 | 4640 | Q92743 | HTRA1 |
| 1548 | 4641 | P22304 | IDS |
| 1549 | 4642 | P05019 | IGF1 |
| 1550 | 4643 | Q6B9Z1 | IGFL4 |
| 1551 | 4644 | Q14623 | IHH |
| 1552 | 4645 | P09529 | INHBB |
| 1553 | 4646 | B1AKI9 | ISM1 |
| 1554 | 4647 | Q8IWB1 | ITPRIP |
| 1555 | 4648 | Q6GPH6 | ITPRIPL1 |
| 1556 | 4649 | Q6PHW0 | IYD |
| 1557 | 4650 | A6ND01 | IZUMO1R |
| 1558 | 4651 | Q6UXV1 | IZUMO2 |
| 1559 | 4652 | Q5VZ72 | IZUMO3 |
| 1560 | 4653 | P17658 | KCNA6 |
| 1561 | 4654 | Q8WWG9 | KCNE4 |
| 1562 | 4655 | Q16558 | KCNMB1 |
| 1563 | 4656 | Q9UBX7 | KLK11 |
| 1564 | 4657 | Q9UKR0 | KLK12 |
| 1565 | 4658 | O60259 | KLK8 |
| 1566 | 4659 | Q8NCW0 | KREMEN2 |
| 1567 | 4660 | Q8IYD9 | LAS2 |
| 1568 | 4661 | P04180 | LCAT |
| 1569 | 4662 | P31025 | LCN1 |
| 1570 | 4663 | Q6JVE6 | LCN10 |
| 1571 | 4664 | Q6JVE5 | LCN12 |
| 1572 | 4665 | Q5VSP4 | LCN1P1 |
| 1573 | 4666 | Q5SZI1 | LDLRAD2 |
| 1574 | 4667 | Q86YD5 | LDLRAD3 |
| 1575 | 4668 | Q6P5S2 | LEG1 |
| 1576 | 4669 | P01229 | LHB |
| 1577 | 4670 | Q7Z4B0 | LINC00305 |
| 1578 | 4671 | Q9UJ94 | LINC00527 |
| 1579 | 4672 | Q5VYY2 | LIPM |
| 1580 | 4673 | Q5VXI9 | LIPN |
| 1581 | 4674 | Q96L11 | LLCFC1 |
| 1582 | 4675 | Q16609 | LPAL2 |
| 1583 | 4676 | A6NCL2 | LRCOL1 |
| 1584 | 4677 | Q5XG99 | LYSMD4 |
| 1585 | 4678 | A6NHS7 | MANSC4 |
| 1586 | 4679 | Q9BUN1 | MENT |
| 1587 | 4680 | Q9UJH8 | METRN |
| 1588 | 4681 | Q641Q3 | METRNL |
| 1589 | 4682 | Q5JXM2 | METTL24 |
| 1590 | 4683 | Q6UX53 | METTL7B |
| 1591 | 4684 | Q9BY79 | MFRP |
| 1592 | 4685 | P08493 | MGP |
| 1593 | 4686 | P24347 | MMP11 |
| 1594 | 4687 | Q8N119 | MMP21 |
| 1595 | 4688 | Q9NPA2 | MMP25 |
| 1596 | 4689 | A6NHM9 | MOXD2P |
| 1597 | 4690 | Q1L6U9 | MSMP |
| 1598 | 4691 | Q3MIW9 | MUCL3 |
| 1599 | 4692 | Q02083 | NAAA |
| 1600 | 4693 | P41271 | NBL1 |
| 1601 | 4694 | Q8TDF5 | NETO1 |
| 1602 | 4695 | Q9NPE2 | NGRN |
| 1603 | 4696 | Q0D2K0 | NIPAL4 |
| 1604 | 4697 | Q6P988 | NOTUM |
| 1605 | 4698 | Q9HBY0 | NOX3 |
| 1606 | 4699 | A6NHN6 | NPIPB15 |
| 1607 | 4700 | O75200 | NPIPB7 |
| 1608 | 4701 | P16860 | NPPB |
| 1609 | 4702 | P17342 | NPR3 |
| 1610 | 4703 | Q9NPD7 | NRN1 |
| 1611 | 4704 | Q99748 | NRTN |
| 1612 | 4705 | Q02818 | NUCB1 |
| 1613 | 4706 | P80303 | NUCB2 |
| 1614 | 4707 | P00973 | OAS1 |
| 1615 | 4708 | Q9NY56 | OBP2A |
| 1616 | 4709 | Q02509 | OC90 |
| 1617 | 4710 | A1E959 | ODAM |
| 1618 | 4711 | Q17RF5 | ODAPH |
| 1619 | 4712 | A8MZH6 | OOSP1 |
| 1620 | 4713 | Q86WS3 | OOSP2 |
| 1621 | 4714 | A6NHN0 | OTOL1 |
| 1622 | 4715 | Q8NHW6 | OTOS |
| 1623 | 4716 | Q7RTZ1 | OVCH2 |
| 1624 | 4717 | Q9UBL9 | P2RX2 |
| 1625 | 4718 | Q8NBM8 | PCYOX1L |
| 1626 | 4719 | Q15084 | PDIA6 |
| 1627 | 4720 | Q96S96 | PEBP4 |
| 1628 | 4721 | P0DJD8 | PGA3 |
| 1629 | 4722 | P20142 | PGC |
| 1630 | 4723 | Q96PD5 | PGLYRP2 |
| 1631 | 4724 | Q96LB8 | PGLYRP4 |
| 1632 | 4725 | Q6UXB8 | PI16 |
| 1633 | 4726 | Q8NCC3 | PLA2G15 |
| 1634 | 4727 | Q5R387 | PLA2G2C |
| 1635 | 4728 | Q6P4A8 | PLBD1 |
| 1636 | 4729 | Q8NHP8 | PLBD2 |
| 1637 | 4730 | Q6UQ28 | PLET1 |
| 1638 | 4731 | Q15195 | PLGLA |
| 1639 | 4732 | Q02325 | PLGLB1 |
| 1640 | 4733 | Q6GTS8 | PM20D1 |
| 1641 | 4734 | P54315 | PNLIPRP1 |
| 1642 | 4735 | Q86SH4 | PRNT |
| 1643 | 4736 | Q99946 | PRRT1 |
| 1644 | 4737 | O95084 | PRSS23 |
| 1645 | 4738 | Q9BQR3 | PRSS27 |
| 1646 | 4739 | P35030 | PRSS3 |
| 1647 | 4740 | Q8NHM4 | PRSS3P2 |
| 1648 | 4741 | Q7RTY9 | PRSS41 |
| 1649 | 4742 | E7EML9 | PRSS44 |
| 1650 | 4743 | A8MTI9 | PRSS47 |
| 1651 | 4744 | Q6UWB4 | PRSS55 |
| 1652 | 4745 | Q8IYP2 | PRSS58 |
| 1653 | 4746 | Q6NUJ1 | PSAPL1 |
| 1654 | 4747 | Q9UIG4 | PSORS1C2 |
| 1655 | 4748 | P01270 | PTH |
| 1656 | 4749 | Q96A99 | PTX4 |
| 1657 | 4750 | Q6H3X3 | RAET1G |
| 1658 | 4751 | Q5VY80 | RAET1L |
| 1659 | 4752 | Q5W5W9 | RESP18 |
| 1660 | 4753 | Q86XS8 | RNF130 |
| 1661 | 4754 | Q8N7C7 | RNF148 |
| 1662 | 4755 | Q9H6Y7 | RNF167 |
| 1663 | 4756 | Q96EX2 | RNFT2 |
| 1664 | 4757 | Q6UXX9 | RSPO2 |
| 1665 | 4758 | P80511 | S100A12 |
| 1666 | 4759 | Q6ZMJ2 | SCARA5 |
| 1667 | 4760 | Q8TD33 | SCGB1C1 |
| 1668 | 4761 | O75056 | SDC3 |
| 1669 | 4762 | P0C7V7 | SEC11B |
| 1670 | 4763 | P04279 | SEMG1 |
| 1671 | 4764 | Q6UXR4 | SERPINA13P |
| 1672 | 4765 | P20848 | SERPINA2 |
| 1673 | 4766 | P36952 | SERPINB5 |
| 1674 | 4767 | P01008 | SERPINC1 |
| 1675 | 4768 | A8MV23 | SERPINE3 |
| 1676 | 4769 | Q99574 | SERPINI1 |
| 1677 | 4770 | P0C7M3 | SFTA3 |
| 1678 | 4771 | Q13326 | SGCG |
| 1679 | 4772 | Q96LD1 | SGCZ |
| 1680 | 4773 | Q8N114 | SHISA5 |
| 1681 | 4774 | Q6ZSJ9 | SHISA6 |
| 1682 | 4775 | A6NL88 | SHISA7 |
| 1683 | 4776 | B8ZZ34 | SHISA8 |
| 1684 | 4777 | B4DS77 | SHISA9 |
| 1685 | 4778 | Q5TFQ8 | SIRPB1 |
| 1686 | 4779 | Q63ZE4 | SLC22A10 |
| 1687 | 4780 | Q9Y226 | SLC22A13 |
| 1688 | 4781 | O15244 | SLC22A2 |
| 1689 | 4782 | A6NK97 | SLC22A20P |
| 1690 | 4783 | Q6T423 | SLC22A25 |
| 1691 | 4784 | A6NKX4 | SLC22A31 |
| 1692 | 4785 | P11168 | SLC2A2 |
| 1693 | 4786 | Q8N130 | SLC34A3 |
| 1694 | 4787 | Q969I6 | SLC38A4 |
| 1695 | 4788 | A6NLE4 | SMIM23 |
| 1696 | 4789 | Q92485 | SMPDL3B |
| 1697 | 4790 | Q2M3V2 | SOWAHA |
| 1698 | 4791 | Q96QH8 | SPACA5 |
| 1699 | 4792 | Q96KW9 | SPACA7 |
| 1700 | 4793 | Q6PDA7 | SPAG11A |
| 1701 | 4794 | Q08648 | SPAG11B |
| 1702 | 4795 | P09486 | SPARC |
| 1703 | 4796 | P0C7L1 | SPINK8 |
| 1704 | 4797 | Q6UDR6 | SPINT4 |
| 1705 | 4798 | Q9BUD6 | SPON2 |
| 1706 | 4799 | Q13103 | SPP2 |
| 1707 | 4800 | Q7Z2R9 | SSBP3-AS1 |
| 1708 | 4801 | A6NDD5 | SYNDIG1L |
| 1709 | 4802 | H3BTG2 | TEX46 |
| 1710 | 4803 | P10646 | TFPI |
| 1711 | 4804 | H3BV60 | TGFBR3L |
| 1712 | 4805 | Q8WUY1 | THEM6 |
| 1713 | 4806 | Q86YJ6 | THNSL2 |
| 1714 | 4807 | P40225 | THPO |
| 1715 | 4808 | Q9NS93 | TM7SF3 |
| 1716 | 4809 | Q9HD45 | TM9SF3 |
| 1717 | 4810 | Q4V9L6 | TMEM119 |
| 1718 | 4811 | Q9BXJ8 | TMEM120A |
| 1719 | 4812 | Q8N614 | TMEM156 |
| 1720 | 4813 | Q8WZ71 | TMEM158 |
| 1721 | 4814 | Q8NBL3 | TMEM178A |
| 1722 | 4815 | H3BS89 | TMEM178B |
| 1723 | 4816 | Q9H813 | TMEM206 |
| 1724 | 4817 | Q86XT9 | TMEM219 |
| 1725 | 4818 | A6NFC5 | TMEM235 |
| 1726 | 4819 | Q9P0T7 | TMEM9 |
| 1727 | 4820 | Q6ZNR0 | TMEM91 |
| 1728 | 4821 | Q8N816 | TMEM99 |
| 1729 | 4822 | Q6ZWK6 | TMPRSS11F |
| 1730 | 4823 | Q9H1E5 | TMX4 |
| 1731 | 4824 | Q9H2S6 | TNMD |
| 1732 | 4825 | Q8N2E6 | TOR2A |
| 1733 | 4826 | Q8NBR0 | TP53I13 |
| 1734 | 4827 | Q15661 | TPSAB1 |
| 1735 | 4828 | Q9BZJ3 | TPSD1 |
| 1736 | 4829 | A6NFA1 | TRABD2B |
| 1737 | 4830 | O00294 | TULP1 |
| 1738 | 4831 | O75386 | TULP3 |
| 1739 | 4832 | P10599 | TXN |
| 1740 | 4833 | Q8WVF2 | UCMA |
| 1741 | 4834 | Q9Y4X1 | UGT2A1 |
| 1742 | 4835 | P36537 | UGT2B10 |
| 1743 | 4836 | Q9BY64 | UGT2B28 |
| 1744 | 4837 | Q16880 | UGT8 |
| 1745 | 4838 | Q9BZM4 | ULBP3 |
| 1746 | 4839 | Q6UY13 | UNQ5830/ |
| | | | PRO19650/ |
| | | | PRO19816 |
| 1747 | 4840 | Q6UXV3 | UNQ6126/ |
| | | | PRO20091 |
| 1748 | 4841 | Q6UXQ8 | UNQ6190/ |
| | | | PRO20217 |
| 1749 | 4842 | Q6UXR6 | UNQ6494/ |
| | | | PRO21346 |
| 1750 | 4843 | Q6UXU0 | UNQ9165/ |
| | | | PRO28630 |
| 1751 | 4844 | Q9N2K0 | ENH3 |
| 1752 | 4845 | Q9N2J8 | ENH1 |
| 1753 | 4846 | Q8N1Y9 | FLJ37218 |
| 1754 | 4847 | Q6ZRU5 | FLJ46089 |
| 1755 | 4848 | Q8N9W7 | FLJ36131 |
| 1756 | 4849 | A6NDX4 | ENSP00000320207 |
| 1757 | 4850 | A8MUN3 | ENSP00000381830 |
| 1758 | 4851 | Q8TAT8 | LOC644613 |
| 1759 | 4852 | B0FP48 | UPK3BL1 |
| 1760 | 4853 | Q86V25 | VASH2 |
| 1761 | 4854 | Q9NY84 | VNN3 |
| 1762 | 4855 | Q8IUB5 | WFDC13 |
| 1763 | 4856 | Q8IUA0 | WFDC8 |
| 1764 | 4857 | O95388 | WISP1 |
| 1765 | 4858 | P56703 | WNT3 |
| 1766 | 4859 | Q9Y6F9 | WNT6 |
| 1767 | 4860 | Q9H1J5 | WNT8A |
| 1768 | 4861 | O14905 | WNT9B |
| 1769 | 4862 | P21754 | ZP3 |
| 1770 | 4863 | Q12836 | ZP4 |
| 1771 | 4864 | A1L453 | PRSS38 |
| 1772 | 4865 | A2RUU4 | CLPSL1 |
| 1773 | 4866 | A4D0V7 | CPED1 |
| 1774 | 4867 | A4D1T9 | PRSS37 |
| 1775 | 4868 | A5X5Y0 | HTR3E |
| 1776 | 4869 | A6NNS2 | DHRS7C |
| 1777 | 4870 | A8K7I4 | CLCA1 |
| 1778 | 4871 | A8MVS5 | HIDE1 |
| 1779 | 4872 | B2RNN3 | C1QTNF9B |
| 1780 | 4873 | B2RUY7 | VWC2L |
| 1781 | 4874 | C9JUS6 | ADM5 |
| 1782 | 4875 | O00115 | DNASE2 |
| 1783 | 4876 | O00144 | FZD9 |
| 1784 | 4877 | O00180 | KCNK1 |
| 1785 | 4878 | O00182 | LGALS9 |
| 1786 | 4879 | O00253 | AGRP |
| 1787 | 4880 | O00292 | LEFTY2 |
| 1788 | 4881 | O00295 | TULP2 |
| 1789 | 4882 | O00515 | LAD1 |
| 1790 | 4883 | O00560 | SDCBP |
| 1791 | 4884 | O00584 | RNASET2 |
| 1792 | 4885 | O00590 | ACKR2 |
| 1793 | 4886 | O00591 | GABRP |
| 1794 | 4887 | O00592 | PODXL |
| 1795 | 4888 | O00602 | FCN1 |
| 1796 | 4889 | O00622 | CYR61 |
| 1797 | 4890 | O00744 | WNT10B |
| 1798 | 4891 | O00748 | CES2 |
| 1799 | 4892 | O00754 | MAN2B1 |
| 1800 | 4893 | O00755 | WNT7A |
| 1801 | 4894 | O14493 | CLDN4 |
| 1802 | 4895 | O14638 | ENPP3 |
| 1803 | 4896 | O14656 | TOR1A |
| 1804 | 4897 | O14657 | TOR1B |
| 1805 | 4898 | O14668 | PRRG1 |
| 1806 | 4899 | O14756 | HSD17B6 |
| 1807 | 4900 | O14764 | GABRD |
| 1808 | 4901 | O14773 | TPP1 |
| 1809 | 4902 | O14791 | APOL1 |
| 1810 | 4903 | O14792 | HS3ST1 |
| 1811 | 4904 | O14904 | WNT9A |
| 1812 | 4905 | O14958 | CASQ2 |
| 1813 | 4906 | O14960 | LECT2 |
| 1814 | 4907 | O15120 | AGPAT2 |
| 1815 | 4908 | O15245 | SLC22A1 |
| 1816 | 4909 | O15321 | TM9SF1 |
| 1817 | 4910 | O15393 | TMPRSS2 |
| 1818 | 4911 | O15431 | SLC31A1 |
| 1819 | 4912 | O15460 | P4HA2 |
| 1820 | 4913 | O15496 | PLA2G10 |
| 1821 | 4914 | O15537 | RS1 |
| 1822 | 4915 | O15547 | P2RX6 |
| 1823 | 4916 | O15551 | CLDN3 |
| 1824 | 4917 | O43240 | KLK10 |
| 1825 | 4918 | O43280 | TREH |
| 1826 | 4919 | O43291 | SPINT2 |
| 1827 | 4920 | O43323 | DHH |
| 1828 | 4921 | O43493 | TGOLN2 |
| 1829 | 4922 | O43555 | GNRH2 |
| 1830 | 4923 | O43556 | SGCE |
| 1831 | 4924 | O43570 | CA12 |
| 1832 | 4925 | O43614 | HCRTR2 |
| 1833 | 4926 | O43692 | PI15 |
| 1834 | 4927 | O43852 | CALU |
| 1835 | 4928 | O43866 | CD5L |
| 1836 | 4929 | O43908 | KLRC4 |
| 1837 | 4930 | O60218 | AKR1B10 |
| 1838 | 4931 | O60235 | TMPRSS11D |
| 1839 | 4932 | O60565 | GREM1 |
| 1840 | 4933 | O60568 | PLOD3 |
| 1841 | 4934 | O60575 | SPINK4 |
| 1842 | 4935 | O60656 | UGT1A9 |
| 1843 | 4936 | O60676 | CST8 |
| 1844 | 4937 | O60844 | ZG16 |
| 1845 | 4938 | O60882 | MMP20 |
| 1846 | 4939 | O60894 | RAMP1 |
| 1847 | 4940 | O60895 | RAMP2 |
| 1848 | 4941 | O60896 | RAMP3 |
| 1849 | 4942 | O60911 | CTSV |
| 1850 | 4943 | O75084 | FZD7 |
| 1851 | 4944 | O75106 | AOC2 |
| 1852 | 4945 | O75185 | ATP2C2 |
| 1853 | 4946 | O75310 | UGT2B11 |
| 1854 | 4947 | O75311 | GLRA3 |
| 1855 | 4948 | O75356 | ENTPD5 |
| 1856 | 4949 | O75398 | DEAF1 |
| 1857 | 4950 | O75487 | GPC4 |
| 1858 | 4951 | O75493 | CA11 |
| 1859 | 4952 | O75503 | CLN5 |
| 1860 | 4953 | O75508 | CLDN11 |
| 1861 | 4954 | O75556 | SCGB2A1 |
| 1862 | 4955 | O75610 | LEFTY1 |
| 1863 | 4956 | O75629 | CREG1 |
| 1864 | 4957 | O75636 | FCN3 |
| 1865 | 4958 | O75711 | SCRG1 |
| 1866 | 4959 | O75715 | GPX5 |
| 1867 | 4960 | O75718 | CRTAP |
| 1868 | 4961 | O75787 | ATP6AP2 |
| 1869 | 4962 | O75795 | UGT2B17 |
| 1870 | 4963 | O75830 | SERPINI2 |
| 1871 | 4964 | O75951 | LYZL6 |
| 1872 | 4965 | O76038 | SCGN |
| 1873 | 4966 | O76061 | STC2 |
| 1874 | 4967 | O76076 | WISP2 |
| 1875 | 4968 | O76082 | SLC22A5 |
| 1876 | 4969 | O76095 | JTB |
| 1877 | 4970 | O94907 | DKK1 |
| 1878 | 4971 | O94956 | SLCO2B1 |
| 1879 | 4972 | O94985 | CLSTN1 |
| 1880 | 4973 | O95156 | NXPH2 |
| 1881 | 4974 | O95157 | NXPH3 |
| 1882 | 4975 | O95158 | NXPH4 |
| 1883 | 4976 | O95264 | HTR3B |
| 1884 | 4977 | O95302 | FKBP9 |
| 1885 | 4978 | O95389 | WISP3 |
| 1886 | 4979 | O95436 | SLC34A2 |
| 1887 | 4980 | O95445 | APOM |
| 1888 | 4981 | O95471 | CLDN7 |
| 1889 | 4982 | O95484 | CLDN9 |
| 1890 | 4983 | O95497 | VNN1 |
| 1891 | 4984 | O95498 | VNN2 |
| 1892 | 4985 | O95500 | CLDN14 |
| 1893 | 4986 | O95502 | NPTXR |
| 1894 | 4987 | O95528 | SLC2A10 |
| 1895 | 4988 | O95622 | ADCY5 |
| 1896 | 4989 | O95711 | LY86 |
| 1897 | 4990 | O95813 | CER1 |
| 1898 | 4991 | O95832 | CLDN1 |
| 1899 | 4992 | O95881 | TXNDC12 |
| 1900 | 4993 | O95897 | OLFM2 |
| 1901 | 4994 | O95925 | EPPIN |
| 1902 | 4995 | O95968 | SCGB1D1 |
| 1903 | 4996 | O95969 | SCGB1D2 |
| 1904 | 4997 | O95994 | AGR2 |
| 1905 | 4998 | O96005 | CLPTM1 |
| 1906 | 4999 | O96009 | NAPSA |
| 1907 | 5000 | O96014 | WNT11 |
| 1908 | 5001 | P00450 | CP |
| 1909 | 5002 | P00709 | LALBA |
| 1910 | 5003 | P00734 | F2 |
| 1911 | 5004 | P00751 | CFB |
| 1912 | 5005 | P00797 | REN |
| 1913 | 5006 | P00995 | SPINK1 |
| 1914 | 5007 | P01009 | SERPINA1 |
| 1915 | 5008 | P01011 | SERPINA3 |
| 1916 | 5009 | P01019 | AGT |
| 1917 | 5010 | P01033 | TIMP1 |
| 1918 | 5011 | P01034 | CST3 |
| 1919 | 5012 | P01036 | CST4 |
| 1920 | 5013 | P01037 | CST1 |
| 1921 | 5014 | P01148 | GNRH1 |
| 1922 | 5015 | P01178 | OXT |
| 1923 | 5016 | P01185 | AVP |
| 1924 | 5017 | P01189 | POMC |
| 1925 | 5018 | P01222 | TSHB |
| 1926 | 5019 | P01225 | FSHB |
| 1927 | 5020 | P01236 | PRL |
| 1928 | 5021 | P01241 | GH1 |
| 1929 | 5022 | P01275 | GCG |
| 1930 | 5023 | P01350 | GAST |
| 1931 | 5024 | P02647 | APOA1 |
| 1932 | 5025 | P02649 | APOE |
| 1933 | 5026 | P02652 | APOA2 |
| 1934 | 5027 | P02654 | APOC1 |
| 1935 | 5028 | P02655 | APOC2 |
| 1936 | 5029 | P02656 | APOC3 |
| 1937 | 5030 | P02675 | FGB |
| 1938 | 5031 | P02679 | FGG |
| 1939 | 5032 | P02724 | GYPA |
| 1940 | 5033 | P02741 | CRP |
| 1941 | 5034 | P02743 | APCS |
| 1942 | 5035 | P02749 | APOH |
| 1943 | 5036 | P02753 | RBP4 |
| 1944 | 5037 | P02760 | AMBP |
| 1945 | 5038 | P02763 | ORM1 |
| 1946 | 5039 | P02765 | AHSG |
| 1947 | 5040 | P02766 | TTR |
| 1948 | 5041 | P02768 | ALB |
| 1949 | 5042 | P02771 | AFP |
| 1950 | 5043 | P02774 | GC |
| 1951 | 5044 | P02810 | PRH1; |
| 1952 | 5045 | P02814 | SMR3B |
| 1953 | 5046 | P02818 | BGLAP |
| 1954 | 5047 | P03950 | ANG |
| 1955 | 5048 | P03951 | F11 |
| 1956 | 5049 | P03952 | KLKB1 |
| 1957 | 5050 | P03956 | MMP1 |
| 1958 | 5051 | P03973 | SLP1 |
| 1959 | 5052 | P04001 | OPN1MW |
| 1960 | 5053 | P04003 | C4BPA |
| 1961 | 5054 | P04004 | VTN |
| 1962 | 5055 | P04054 | PLA2G1B |
| 1963 | 5056 | P04062 | GBA |
| 1964 | 5057 | P04066 | FUCA1 |
| 1965 | 5058 | P04083 | ANXA1 |
| 1966 | 5059 | P04090 | RLN2 |
| 1967 | 5060 | P04118 | CLPS |
| 1968 | 5061 | P04155 | TFF1 |
| 1969 | 5062 | P04156 | PRNP |
| 1970 | 5063 | P04196 | HRG |
| 1971 | 5064 | P04278 | SHBG |
| 1972 | 5065 | P04628 | WNT1 |
| 1973 | 5066 | P04745 | AMY1A |
| 1974 | 5067 | P04808 | RLN1 |
| 1975 | 5068 | P04920 | SLC4A2 |
| 1976 | 5069 | P04921 | GYPC |
| 1977 | 5070 | P05023 | ATP1A1 |
| 1978 | 5071 | P05026 | ATP1B1 |
| 1979 | 5072 | P05060 | CHGB |
| 1980 | 5073 | P05067 | APP |
| 1981 | 5074 | P05090 | APOD |
| 1982 | 5075 | P05109 | S100A8 |
| 1983 | 5076 | P05111 | INHA |
| 1984 | 5077 | P05120 | SERPINB2 |
| 1985 | 5078 | P05121 | SERPINE1 |
| 1986 | 5079 | P05154 | SERPINA5 |
| 1987 | 5080 | P05155 | SERPING1 |
| 1988 | 5081 | P05160 | F13B |
| 1989 | 5082 | P05164 | MPO |
| 1990 | 5083 | P05186 | ALPL |
| 1991 | 5084 | P05187 | ALPP |
| 1992 | 5085 | P05408 | SCG5 |
| 1993 | 5086 | P05543 | SERPINA7 |
| 1994 | 5087 | P05546 | SERPIND1 |
| 1995 | 5088 | P05814 | CSN2 |
| 1996 | 5089 | P05981 | HPN |
| 1997 | 5090 | P06133 | UGT2B4 |
| 1998 | 5091 | P06276 | BCHE |
| 1999 | 5092 | P06280 | GLA |
| 2000 | 5093 | P06307 | CCK |
| 2001 | 5094 | P06396 | GSN |
| 2002 | 5095 | P06681 | C2 |
| 2003 | 5096 | P06702 | S100A9 |
| 2004 | 5097 | P06858 | LPL |
| 2005 | 5098 | P06865 | HEXA |
| 2006 | 5099 | P06870 | KLK1 |
| 2007 | 5100 | P07093 | SERPINE2 |
| 2008 | 5101 | P07098 | LIPF |
| 2009 | 5102 | P07237 | P4HB |
| 2010 | 5103 | P07288 | KLK3 |
| 2011 | 5104 | P07339 | CTSD |
| 2012 | 5105 | P07355 | ANXA2 |
| 2013 | 5106 | P07360 | C8G |
| 2014 | 5107 | P07477 | PRSS1 |
| 2015 | 5108 | P07478 | PRSS2 |
| 2016 | 5109 | P07498 | CSN3 |
| 2017 | 5110 | P07602 | PSAP |
| 2018 | 5111 | P07686 | HEXB |
| 2019 | 5112 | P07711 | CTSL |
| 2020 | 5113 | P07949 | RET |
| 2021 | 5114 | P07988 | SFTPB |
| 2022 | 5115 | P07998 | RNASE1 |
| 2023 | 5116 | P08118 | MSMB |
| 2024 | 5117 | P08185 | SERPINA6 |
| 2025 | 5118 | P08217 | CELA2A |
| 2026 | 5119 | P08218 | CELA2B |
| 2027 | 5120 | P08246 | ELANE |
| 2028 | 5121 | P08254 | MMP3 |
| 2029 | 5122 | P08294 | SOD3 |
| 2030 | 5123 | P08311 | CTSG |
| 2031 | 5124 | P08473 | MME |
| 2032 | 5125 | P08476 | INHBA |
| 2033 | 5126 | P08572 | COL4A2 |
| 2034 | 5127 | P08697 | SERPINF2 |
| 2035 | 5128 | P08833 | IGFBP1 |
| 2036 | 5129 | P08861 | CELA3B |
| 2037 | 5130 | P08910 | ABHD2 |
| 2038 | 5131 | P09093 | CELA3A |
| 2039 | 5132 | P09228 | CST2 |
| 2040 | 5133 | P09237 | MMP7 |
| 2041 | 5134 | P09238 | MMP10 |
| 2042 | 5135 | P09382 | LGALS1 |
| 2043 | 5136 | P09466 | PAEP |
| 2044 | 5137 | P09544 | WNT2 |
| 2045 | 5138 | P09668 | CTSH |
| 2046 | 5139 | P09758 | TACSTD2 |
| 2047 | 5140 | P09923 | ALPI |
| 2048 | 5141 | P09958 | FURIN |
| 2049 | 5142 | P0C862 | C1QTNF9 |
| 2050 | 5143 | P0DJD7 | PGA4 |
| 2051 | 5144 | P0DJD9 | PGA5 |
| 2052 | 5145 | P0DJI8 | SAA1 |
| 2053 | 5146 | P0DJI9 | SAA2 |
| 2054 | 5147 | P0DML2 | CSH |
| 2055 | 5148 | P0DML3 | CSH2 |
| 2056 | 5149 | P0DMR2 | SCGB1C2 |
| 2057 | 5150 | P10124 | SRGN |
| 2058 | 5151 | P10144 | GZMB |
| 2059 | 5152 | P10153 | RNASE2 |
| 2060 | 5153 | P10253 | GAA |
| 2061 | 5154 | P10323 | ACR |
| 2062 | 5155 | P10451 | SPP1 |
| 2063 | 5156 | P10619 | CTSA |
| 2064 | 5157 | P10645 | CHGA |
| 2065 | 5158 | P10696 | ALPPL2 |
| 2066 | 5159 | P10720 | PF4V1 |
| 2067 | 5160 | P10909 | CLU |
| 2068 | 5161 | P11021 | HSPA5 |
| 2069 | 5162 | P11150 | LIPC |
| 2070 | 5163 | P11230 | CHRNB1 |
| 2071 | 5164 | P11597 | CETP |
| 2072 | 5165 | P11684 | SCGB1A1 |
| 2073 | 5166 | P12018 | VPREB1 |
| 2074 | 5167 | P12110 | COL6A2 |
| 2075 | 5168 | P12259 | F5 |
| 2076 | 5169 | P12272 | PTHLH |
| 2077 | 5170 | P12544 | GZMA |
| 2078 | 5171 | P12724 | RNASE3 |
| 2079 | 5172 | P12872 | MLN |
| 2080 | 5173 | P13284 | IFI30 |
| 2081 | 5174 | P13521 | SCG2 |
| 2082 | 5175 | P13637 | ATP1A3 |
| 2083 | 5176 | P13667 | PDIA4 |
| 2084 | 5177 | P13674 | P4HA1 |
| 2085 | 5178 | P13686 | ACP5 |
| 2086 | 5179 | P13725 | OSM |
| 2087 | 5180 | P13762 | HLA-DRB4 |
| 2088 | 5181 | P13866 | SLC5A1 |
| 2089 | 5182 | P14091 | CTSE |
| 2090 | 5183 | P14207 | FOLR2 |
| 2091 | 5184 | P14384 | CPM |
| 2092 | 5185 | P14415 | ATP1B2 |
| 2093 | 5186 | P14555 | PLA2G2A |
| 2094 | 5187 | P14625 | HSP90B1 |
| 2095 | 5188 | P14735 | IDE |
| 2096 | 5189 | P15085 | CPA1 |
| 2097 | 5190 | P15086 | CPB1 |
| 2098 | 5191 | P15088 | CPA3 |
| 2099 | 5192 | P15169 | CPN1 |
| 2100 | 5193 | P15289 | ARSA |
| 2101 | 5194 | P15309 | ACPP |
| 2102 | 5195 | P15328 | FOLR1 |
| 2103 | 5196 | P15586 | GNS |
| 2104 | 5197 | P16035 | TIMP2 |
| 2105 | 5198 | P16150 | SPN |
| 2106 | 5199 | P16233 | PNLIP |
| 2107 | 5200 | P16278 | GLB1 |
| 2108 | 5201 | P16422 | EPCAM |
| 2109 | 5202 | P16444 | DPEP1 |
| 2110 | 5203 | P16519 | PCSK2 |
| 2111 | 5204 | P16562 | CRISP2 |
| 2112 | 5205 | P16662 | UGT2B7 |
| 2113 | 5206 | P16870 | CPE |
| 2114 | 5207 | P17050 | NAGA |
| 2115 | 5208 | P17213 | BPI |
| 2116 | 5209 | P17787 | CHRNB2 |
| 2117 | 5210 | P17813 | ENG |
| 2118 | 5211 | P17900 | GM2A |
| 2119 | 5212 | P17931 | LGALS3 |
| 2120 | 5213 | P17936 | IGFBP3 |
| 2121 | 5214 | P18065 | IGFBP2 |
| 2122 | 5215 | P18433 | PTPRA |
| 2123 | 5216 | P18505 | GABRB1 |
| 2124 | 5217 | P18507 | GABRG2 |
| 2125 | 5218 | P18509 | ADCYAP1 |
| 2126 | 5219 | P19224 | UGT1A6 |
| 2127 | 5220 | P19440 | GGT1 |
| 2128 | 5221 | P19652 | ORM2 |
| 2129 | 5222 | P19883 | FST |
| 2130 | 5223 | P19957 | PI3 |
| 2131 | 5224 | P20023 | CR2 |
| 2132 | 5225 | P20061 | TCN1 |
| 2133 | 5226 | P20062 | TCN2 |
| 2134 | 5227 | P20151 | KLK2 |
| 2135 | 5228 | P20160 | AZU1 |
| 2136 | 5229 | P20231 | TPSB2 |
| 2137 | 5230 | P20382 | PMCH |
| 2138 | 5231 | P20396 | TRH |
| 2139 | 5232 | P20718 | GZMH |
| 2140 | 5233 | P20851 | C4BPB |
| 2141 | 5234 | P20933 | AGA |
| 2142 | 5235 | P21246 | PTN |
| 2143 | 5236 | P21741 | MDK |
| 2144 | 5237 | P21815 | IBSP |
| 2145 | 5238 | P21964 | COMT |
| 2146 | 5239 | P22309 | UGT1A1 |
| 2147 | 5240 | P22310 | UGT1A4 |
| 2148 | 5241 | P22692 | IGFBP4 |
| 2149 | 5242 | P22748 | CA4 |
| 2150 | 5243 | P22894 | MMP8 |
| 2151 | 5244 | P23141 | CES1 |
| 2152 | 5245 | P23276 | KEL |
| 2153 | 5246 | P23284 | PPIB |
| 2154 | 5247 | P23327 | HRC |
| 2155 | 5248 | P23415 | GLRA1 |
| 2156 | 5249 | P23416 | GLRA2 |
| 2157 | 5250 | P23435 | CBLN1 |
| 2158 | 5251 | P23582 | NPPC |
| 2159 | 5252 | P23946 | CMA1 |
| 2160 | 5253 | P23975 | SLC6A2 |
| 2161 | 5254 | P24046 | GABRR1 |
| 2162 | 5255 | P24158 | PRTN3 |
| 2163 | 5256 | P24387 | CRHBP |
| 2164 | 5257 | P24592 | IGFBP6 |
| 2165 | 5258 | P24593 | IGFBP5 |
| 2166 | 5259 | P24855 | DNASE1 |
| 2167 | 5260 | P25092 | GUCY2C |
| 2168 | 5261 | P26436 | ACRV1 |
| 2169 | 5262 | P26885 | FKBP2 |
| 2170 | 5263 | P27037 | ACVR2A |
| 2171 | 5264 | P27169 | PON1 |
| 2172 | 5265 | P27352 | GIF |
| 2173 | 5266 | P27658 | COL8A1 |
| 2174 | 5267 | P27797 | CALR |
| 2175 | 5268 | P27918 | CFP |
| 2176 | 5269 | P28325 | CST5 |
| 2177 | 5270 | P28472 | GABRB3 |
| 2178 | 5271 | P28476 | GABRR2 |
| 2179 | 5272 | P28799 | GRN |
| 2180 | 5273 | P29120 | PCSK1 |
| 2181 | 5274 | P29279 | CTGF |
| 2182 | 5275 | P29622 | SERPINA4 |
| 2183 | 5276 | P29973 | CNGA1 |
| 2184 | 5277 | P30040 | ERP29 |
| 2185 | 5278 | P30101 | PDIA3 |
| 2186 | 5279 | P30531 | SLC6A1 |
| 2187 | 5280 | P30532 | CHRNA5 |
| 2188 | 5281 | P30533 | LRPAP1 |
| 2189 | 5282 | P30926 | CHRNB4 |
| 2190 | 5283 | P30990 | NTS |
| 2191 | 5284 | P31151 | S100A7 |
| 2192 | 5285 | P31415 | CASQ1 |
| 2193 | 5286 | P31644 | GABRA5 |
| 2194 | 5287 | P31947 | SFN |
| 2195 | 5288 | P32297 | CHRNA3 |
| 2196 | 5289 | P32455 | GBP1 |
| 2197 | 5290 | P34059 | GALNS |
| 2198 | 5291 | P34096 | RNASE4 |
| 2199 | 5292 | P34810 | CD68 |
| 2200 | 5293 | P34903 | GABRA3 |
| 2201 | 5294 | P34910 | EVI2B |
| 2202 | 5295 | P34925 | RYK |
| 2203 | 5296 | P35052 | GPC1 |
| 2204 | 5297 | P35503 | UGT1A3 |
| 2205 | 5298 | P35542 | SAA4 |
| 2206 | 5299 | P35625 | TIMP3 |
| 2207 | 5300 | P36222 | CHI3L1 |
| 2208 | 5301 | P36269 | GGT5 |
| 2209 | 5302 | P36896 | ACVR1B |
| 2210 | 5303 | P36955 | SERPINF1 |
| 2211 | 5304 | P36980 | CFHR2 |
| 2212 | 5305 | P37023 | ACVRL1 |
| 2213 | 5306 | P37840 | SNCA |
| 2214 | 5307 | P38567 | SPAM1 |
| 2215 | 5308 | P38571 | LIPA |
| 2216 | 5309 | P39086 | GRIK1 |
| 2217 | 5310 | P39877 | PLA2G5 |
| 2218 | 5311 | P39900 | MMP12 |
| 2219 | 5312 | P40313 | CTRL |
| 2220 | 5313 | P41159 | LEP |
| 2221 | 5314 | P41221 | WNT5A |
| 2222 | 5315 | P41222 | PTGDS |
| 2223 | 5316 | P41439 | FOLR3 |
| 2224 | 5317 | P42127 | ASIP |
| 2225 | 5318 | P42261 | GRIA1 |
| 2226 | 5319 | P42263 | GRIA3 |
| 2227 | 5320 | P42658 | DPP6 |
| 2228 | 5321 | P42785 | PRCP |
| 2229 | 5322 | P42892 | ECE1 |
| 2230 | 5323 | P43005 | SLC1A1 |
| 2231 | 5324 | P43007 | SLC1A4 |
| 2232 | 5325 | P43234 | CTSO |
| 2233 | 5326 | P43235 | CTSK |
| 2234 | 5327 | P43251 | BTD |
| 2235 | 5328 | P43490 | NAMPT |
| 2236 | 5329 | P43652 | AFM |
| 2237 | 5330 | P43681 | CHRNA4 |
| 2238 | 5331 | P45452 | MMP13 |
| 2239 | 5332 | P45844 | ABCG1 |
| 2240 | 5333 | P46059 | SLC15A1 |
| 2241 | 5334 | P46098 | HTR3A |
| 2242 | 5335 | P46695 | IER3 |
| 2243 | 5336 | P47710 | CSN1S1 |
| 2244 | 5337 | P47870 | GABRB2 |
| 2245 | 5338 | P47929 | LGALS7 |
| 2246 | 5339 | P47972 | NPTX2 |
| 2247 | 5340 | P48029 | SLC6A8 |
| 2248 | 5341 | P48052 | CPA2 |
| 2249 | 5342 | P48060 | GLIPR1 |
| 2250 | 5343 | P48065 | SLC6A12 |
| 2251 | 5344 | P48066 | SLC6A11 |
| 2252 | 5345 | P48067 | SLC6A9 |
| 2253 | 5346 | P48167 | GLRB |
| 2254 | 5347 | P48169 | GABRA4 |
| 2255 | 5348 | P48307 | TFPI2 |
| 2256 | 5349 | P48723 | HSPA13 |
| 2257 | 5350 | P48745 | NOV |
| 2258 | 5351 | P48995 | TRPC1 |
| 2259 | 5352 | P49184 | DNASE1L1 |
| 2260 | 5353 | P49662 | CASP4 |
| 2261 | 5354 | P49771 | FLT3LG |
| 2262 | 5355 | P49862 | KLK7 |
| 2263 | 5356 | P49863 | GZMK |
| 2264 | 5357 | P50281 | MMP14 |
| 2265 | 5358 | P50443 | SLC26A2 |
| 2266 | 5359 | P50454 | SERPINH1 |
| 2267 | 5360 | P50897 | PPT1 |
| 2268 | 5361 | P51124 | GZMM |
| 2269 | 5362 | P51164 | ATP4B |
| 2270 | 5363 | P51168 | SCNN1B |
| 2271 | 5364 | P51170 | SCNN1G |
| 2272 | 5365 | P51575 | P2RX1 |
| 2273 | 5366 | P51654 | GPC3 |
| 2274 | 5367 | P51674 | GPM6A |
| 2275 | 5368 | P51686 | CCR9 |
| 2276 | 5369 | P51688 | SGSH |
| 2277 | 5370 | P51689 | ARSD |
| 2278 | 5371 | P51690 | ARSE |
| 2279 | 5372 | P51693 | APLP1 |
| 2280 | 5373 | P51811 | XK |
| 2281 | 5374 | P51841 | GUCY2F |
| 2282 | 5375 | P52823 | STC1 |
| 2283 | 5376 | P52961 | ART1 |
| 2284 | 5377 | P53634 | CTSC |
| 2285 | 5378 | P53801 | PTTG1IP |
| 2286 | 5379 | P54108 | CRISP3 |
| 2287 | 5380 | P54317 | PNLIPRP2 |
| 2288 | 5381 | P54709 | ATP1B3 |
| 2289 | 5382 | P54793 | ARSF |
| 2290 | 5383 | P54803 | GALC |
| 2291 | 5384 | P54855 | UGT2B15 |
| 2292 | 5385 | P55001 | MFAP2 |
| 2293 | 5386 | P55056 | APOC4 |
| 2294 | 5387 | P55058 | PLTP |
| 2295 | 5388 | P55083 | MFAP4 |
| 2296 | 5389 | P55103 | INHBC |
| 2297 | 5390 | P55145 | MANF |
| 2298 | 5391 | P55808 | XG |
| 2299 | 5392 | P56373 | P2RX3 |
| 2300 | 5393 | P56704 | WNT3A |
| 2301 | 5394 | P56705 | WNT4 |
| 2302 | 5395 | P56706 | WNT7B |
| 2303 | 5396 | P56748 | CLDN8 |
| 2304 | 5397 | P56749 | CLDN12 |
| 2305 | 5398 | P56750 | CLDN17 |
| 2306 | 5399 | P56817 | BACE1 |
| 2307 | 5400 | P56851 | EDDM3B |
| 2308 | 5401 | P56856 | CLDN18 |
| 2309 | 5402 | P56880 | CLDN20 |
| 2310 | 5403 | P56937 | HSD17B7 |
| 2311 | 5404 | P57727 | TMPRSS3 |
| 2312 | 5405 | P57739 | CLDN2 |
| 2313 | 5406 | P58062 | SPINK7 |
| 2314 | 5407 | P58166 | INHBE |
| 2315 | 5408 | P58294 | PROK1 |
| 2316 | 5409 | P58417 | NXPH1 |
| 2317 | 5410 | P58499 | FAM3B |
| 2318 | 5411 | P58658 | EVA1C |
| 2319 | 5412 | P59666 | DEFA3 |
| 2320 | 5413 | P59826 | BPIFB3 |
| 2321 | 5414 | P60153 | RNASE9 |
| 2322 | 5415 | P60508 | ERVFRD-1 |
| 2323 | 5416 | P60827 | C1QTNF8 |
| 2324 | 5417 | P60985 | KRTDAP |
| 2325 | 5418 | P61366 | OSTN |
| 2326 | 5419 | P61626 | LYZ |
| 2327 | 5420 | P61916 | NPC2 |
| 2328 | 5421 | P62502 | LCN6 |
| 2329 | 5422 | P62937 | PPIA |
| 2330 | 5423 | P67809 | YBX1 |
| 2331 | 5424 | P78333 | GPC5 |
| 2332 | 5425 | P78348 | ASIC1 |
| 2333 | 5426 | P78369 | CLDN10 |
| 2334 | 5427 | P78562 | PHEX |
| 2335 | 5428 | P79483 | HLA-DRB3 |
| 2336 | 5429 | P80108 | GPLD1 |
| 2337 | 5430 | P80188 | LCN2 |
| 2338 | 5431 | P83105 | HTRA4 |
| 2339 | 5432 | P83110 | HTRA3 |
| 2340 | 5433 | P98066 | TNFAIP6 |
| 2341 | 5434 | Q00604 | NDP |
| 2342 | 5435 | Q01459 | CTBS |
| 2343 | 5436 | Q01523 | DEFA5 |
| 2344 | 5437 | Q02383 | SEMG2 |
| 2345 | 5438 | Q02413 | DSG1 |
| 2346 | 5439 | Q02747 | GUCA2A |
| 2347 | 5440 | Q02809 | PLOD1 |
| 2348 | 5441 | Q02846 | GUCY2D |
| 2349 | 5442 | Q02985 | CFHR3 |
| 2350 | 5443 | Q03403 | TFF2 |
| 2351 | 5444 | Q03591 | CFHR1 |
| 2352 | 5445 | Q03692 | COL10A1 |
| 2353 | 5446 | Q04771 | ACVR1 |
| 2354 | 5447 | Q04900 | CD164 |
| 2355 | 5448 | Q05901 | CHRNB3 |
| 2356 | 5449 | Q05996 | ZP2 |
| 2357 | 5450 | Q06033 | ITIH3 |
| 2358 | 5451 | Q06481 | APLP2 |
| 2359 | 5452 | Q06495 | SLC34A1 |
| 2360 | 5453 | Q07001 | CHRND |
| 2361 | 5454 | Q07021 | C1QBP |
| 2362 | 5455 | Q07075 | ENPEP |
| 2363 | 5456 | Q07507 | DPT |
| 2364 | 5457 | Q07837 | SLC3A1 |
| 2365 | 5458 | Q08345 | DDR1 |
| 2366 | 5459 | Q08380 | LGALS3BP |
| 2367 | 5460 | Q08554 | DSC1 |
| 2368 | 5461 | Q08629 | SPOCK1 |
| 2369 | 5462 | Q08830 | FGL1 |
| 2370 | 5463 | Q0P5P2 | C17orf67 |
| 2371 | 5464 | Q0VAF6 | SYCN |
| 2372 | 5465 | Q10588 | BST1 |
| 2373 | 5466 | Q10589 | BST2 |
| 2374 | 5467 | Q12841 | FSTL1 |
| 2375 | 5468 | Q12884 | FAP |
| 2376 | 5469 | Q12889 | OVGP1 |
| 2377 | 5470 | Q12904 | AIMP1 |
| 2378 | 5471 | Q13003 | GRIK3 |
| 2379 | 5472 | Q13087 | PDIA2 |
| 2380 | 5473 | Q13093 | PLA2G7 |
| 2381 | 5474 | Q13145 | BAMBI |
| 2382 | 5475 | Q13162 | PRDX4 |
| 2383 | 5476 | Q13217 | DNAJC3 |
| 2384 | 5477 | Q13231 | CHIT1 |
| 2385 | 5478 | Q13253 | NOG |
| 2386 | 5479 | Q13296 | SCGB2A2 |
| 2387 | 5480 | Q13316 | DMP1 |
| 2388 | 5481 | Q13361 | MFAP5 |
| 2389 | 5482 | Q13421 | MSLN |
| 2390 | 5483 | Q13438 | OS9 |
| 2391 | 5484 | Q13445 | TMED1 |
| 2392 | 5485 | Q13467 | FZD5 |
| 2393 | 5486 | Q13507 | TRPC3 |
| 2394 | 5487 | Q13508 | ART3 |
| 2395 | 5488 | Q13530 | SERINC3 |
| 2396 | 5489 | Q13563 | PKD2 |
| 2397 | 5490 | Q13609 | DNASE1L3 |
| 2398 | 5491 | Q13705 | ACVR2B |
| 2399 | 5492 | Q13790 | APOF |
| 2400 | 5493 | Q13822 | ENPP2 |
| 2401 | 5494 | Q14050 | COL9A3 |
| 2402 | 5495 | Q14242 | SELPLG |
| 2403 | 5496 | Q14257 | RCN2 |
| 2404 | 5497 | Q14314 | FGL2 |
| 2405 | 5498 | Q14406 | CSHL1 |
| 2406 | 5499 | Q14507 | EDDM3A |
| 2407 | 5500 | Q14508 | WFDC2 |
| 2408 | 5501 | Q14515 | SPARCL1 |
| 2409 | 5502 | Q14696 | MESD |
| 2410 | 5503 | Q14714 | SSPN |
| 2411 | 5504 | Q14832 | GRM3 |
| 2412 | 5505 | Q14993 | COL19A1 |
| 2413 | 5506 | Q14C87 | TMEM132D |
| 2414 | 5507 | Q15043 | SLC39A14 |
| 2415 | 5508 | Q15046 | KARS |
| 2416 | 5509 | Q15063 | POSTN |
| 2417 | 5510 | Q15113 | PCOLCE |
| 2418 | 5511 | Q15165 | PON2 |
| 2419 | 5512 | Q15166 | PON3 |
| 2420 | 5513 | Q15293 | RCN1 |
| 2421 | 5514 | Q15465 | SHH |
| 2422 | 5515 | Q15517 | CDSN |
| 2423 | 5516 | Q15726 | KISS1 |
| 2424 | 5517 | Q15758 | SLC1A5 |
| 2425 | 5518 | Q15782 | CHI3L2 |
| 2426 | 5519 | Q15818 | NPTX1 |
| 2427 | 5520 | Q15825 | CHRNA6 |
| 2428 | 5521 | Q15828 | CST6 |
| 2429 | 5522 | Q15848 | ADIPOQ |
| 2430 | 5523 | Q15884 | FAM189A2 |
| 2431 | 5524 | Q15904 | ATP6AP1 |
| 2432 | 5525 | Q16281 | CNGA3 |
| 2433 | 5526 | Q16378 | PRR4 |
| 2434 | 5527 | Q16445 | GABRA6 |
| 2435 | 5528 | Q16549 | PCSK7 |
| 2436 | 5529 | Q16568 | CARTPT |
| 2437 | 5530 | Q16570 | ACKR1 |
| 2438 | 5531 | Q16585 | SGCB |
| 2439 | 5532 | Q16586 | SGCA |
| 2440 | 5533 | Q16610 | ECM1 |
| 2441 | 5534 | Q16651 | PRSS8 |
| 2442 | 5535 | Q16671 | AMHR2 |
| 2443 | 5536 | Q16674 | MIA |
| 2444 | 5537 | Q16769 | QPCT |
| 2445 | 5538 | Q16790 | CA9 |
| 2446 | 5539 | Q16832 | DDR2 |
| 2447 | 5540 | Q16853 | AOC3 |
| 2448 | 5541 | Q17R60 | IMPG1 |
| 2449 | 5542 | Q17RR3 | PNLIPRP3 |
| 2450 | 5543 | Q19T08 | ECSCR |
| 2451 | 5544 | Q1HG43 | DUOXA1 |
| 2452 | 5545 | Q1HG44 | DUOXA2 |
| 2453 | 5546 | Q1W4C9 | SPINK13 |
| 2454 | 5547 | Q1ZYL8 | IZUMO4 |
| 2455 | 5548 | Q24JP5 | TMEM132A |
| 2456 | 5549 | Q2I0M5 | RSPO4 |
| 2457 | 5550 | Q2M2E5 | C5orf64 |
| 2458 | 5551 | Q2M385 | MPEG1 |
| 2459 | 5552 | Q2M3T9 | HYAL4 |
| 2460 | 5553 | Q2MKA7 | RSPO1 |
| 2461 | 5554 | Q2MV58 | TCTN1 |
| 2462 | 5555 | Q2TAL6 | VWC2 |
| 2463 | 5556 | Q30154 | HLA-DRB5 |
| 2464 | 5557 | Q30KP8 | DEFB136 |
| 2465 | 5558 | Q30KP9 | DEFB135 |
| 2466 | 5559 | Q30KQ4 | DEFB116 |
| 2467 | 5560 | Q30KQ5 | DEFB115 |
| 2468 | 5561 | Q30KQ7 | DEFB113 |
| 2469 | 5562 | Q30KQ8 | DEFB112 |
| 2470 | 5563 | Q32M45 | ANO4 |
| 2471 | 5564 | Q3KNT9 | TMEM95 |
| 2472 | 5565 | Q3SXP7 | SHISAL1 |
| 2473 | 5566 | Q3SY77 | UGT3A2 |
| 2474 | 5567 | Q401N2 | ZACN |
| 2475 | 5568 | Q496H8 | NRN1L |
| 2476 | 5569 | Q496J9 | SV2C |
| 2477 | 5570 | Q49AH0 | CDNF |
| 2478 | 5571 | Q4G0G5 | SCGB2B2 |
| 2479 | 5572 | Q4KMQ2 | ANO6 |
| 2480 | 5573 | Q4U2R8 | SLC22A6 |
| 2481 | 5574 | Q4W5P6 | TMEM155 |
| 2482 | 5575 | Q504Y0 | SLC39A12 |
| 2483 | 5576 | Q53EL9 | SEZ6 |
| 2484 | 5577 | Q53H76 | PLA1A |
| 2485 | 5578 | Q53RT3 | ASPRV1 |
| 2486 | 5579 | Q5DT21 | SPINK9 |
| 2487 | 5580 | Q5EBL8 | PDZD11 |
| 2488 | 5581 | Q5FWE3 | PRRT3 |
| 2489 | 5582 | Q5FYB0 | ARSJ |
| 2490 | 5583 | Q5FYB1 | ARSI |
| 2491 | 5584 | Q5GAN3 | RNASE13 |
| 2492 | 5585 | Q5GAN4 | RNASE12 |
| 2493 | 5586 | Q5GAN6 | RNASE10 |
| 2494 | 5587 | Q5J5C9 | DEFB121 |
| 2495 | 5588 | Q5JS37 | NHLRC3 |
| 2496 | 5589 | Q5JTB6 | PLAC9 |
| 2497 | 5590 | Q5MY95 | ENTPD8 |
| 2498 | 5591 | Q5PT55 | SLC10A5 |
| 2499 | 5592 | Q5T742 | C10orf25 |
| 2500 | 5593 | Q5TF21 | SOGA3 |
| 2501 | 5594 | Q5UCC4 | EMC10 |
| 2502 | 5595 | Q5VXJ0 | LIPK |
| 2503 | 5596 | Q5VXM1 | CDCP2 |
| 2504 | 5597 | Q5W186 | CST9 |
| 2505 | 5598 | Q68BL8 | OLFML2B |
| 2506 | 5599 | Q68DH5 | LMBRD2 |
| 2507 | 5600 | Q68DV7 | RNF43 |
| 2508 | 5601 | Q695T7 | SLC6A19 |
| 2509 | 5602 | Q6E0U4 | DMKN |
| 2510 | 5603 | Q6FHJ7 | SFRP4 |
| 2511 | 5604 | Q6GPI1 | CTRB2 |
| 2512 | 5605 | Q6H9L7 | ISM2 |
| 2513 | 5606 | Q6HA08 | ASTL |
| 2514 | 5607 | Q6IE38 | SPINK14 |
| 2515 | 5608 | Q6ISU1 | PTCRA |
| 2516 | 5609 | Q6J4K2 | SLC8B1 |
| 2517 | 5610 | Q6MZM9 | PRR27 |
| 2518 | 5611 | Q6NSJ0 | MYORG |
| 2519 | 5612 | Q6NSX1 | CCDC70 |
| 2520 | 5613 | Q6NUM9 | RETSAT |
| 2521 | 5614 | Q6NUS6 | TCTN3 |
| 2522 | 5615 | Q6NUS8 | UGT3A1 |
| 2523 | 5616 | Q6NVV3 | NIPAL1 |
| 2524 | 5617 | Q6NW40 | RGMB |
| 2525 | 5618 | Q6P093 | AADACL2 |
| 2526 | 5619 | Q6P4Q7 | CNNM4 |
| 2527 | 5620 | Q6P5W5 | SLC39A4 |
| 2528 | 5621 | Q6P995 | FAM171B |
| 2529 | 5622 | Q6P9G4 | TMEM154 |
| 2530 | 5623 | Q6PB30 | CSAG1 |
| 2531 | 5624 | Q6PL45 | BRICD5 |
| 2532 | 5625 | Q6Q788 | APOA5 |
| 2533 | 5626 | Q6SPF0 | SAMD1 |
| 2534 | 5627 | Q6URK8 | TEPP |
| 2535 | 5628 | Q6UW10 | SFTA2 |
| 2536 | 5629 | Q6UW49 | SPESP1 |
| 2537 | 5630 | Q6UWF9 | FAM180A |
| 2538 | 5631 | Q6UWH4 | FAM198B |
| 2539 | 5632 | Q6UWI2 | PARM1 |
| 2540 | 5633 | Q6UWI4 | SHISA2 |
| 2541 | 5634 | Q6UWJ1 | TMCO3 |
| 2542 | 5635 | Q6UWJ8 | CD164L2 |
| 2543 | 5636 | Q6UWM5 | GLIPR1L1 |
| 2544 | 5637 | Q6UWM7 | LCTL |
| 2545 | 5638 | Q6UWM9 | UGT2A3 |
| 2546 | 5639 | Q6UWN8 | SPINK6 |
| 2547 | 5640 | Q6UWQ5 | LYZL1 |
| 2548 | 5641 | Q6UWR7 | ENPP6 |
| 2549 | 5642 | Q6UWU4 | C6orf89 |
| 2550 | 5643 | Q6UWV6 | ENPP7 |
| 2551 | 5644 | Q6UWW0 | LCN15 |
| 2552 | 5645 | Q6UWW8 | CES3 |
| 2553 | 5646 | Q6UWY0 | ARSK |
| 2554 | 5647 | Q6UWY2 | PRSS57 |
| 2555 | 5648 | Q6UWY5 | OLFML1 |
| 2556 | 5649 | Q6UX06 | OLFM4 |
| 2557 | 5650 | Q6UX34 | SNORC |
| 2558 | 5651 | Q6UX39 | AMTN |
| 2559 | 5652 | Q6UX71 | PLXDC2 |
| 2560 | 5653 | Q6UXA7 | C6orf15 |
| 2561 | 5654 | Q6UXF1 | TMEM108 |
| 2562 | 5655 | Q6UXI7 | VIT |
| 2563 | 5656 | Q6UXQ4 | C2orf66 |
| 2564 | 5657 | Q6UXT8 | ALKAL1 |
| 2565 | 5658 | Q6UXT9 | ABHD15 |
| 2566 | 5659 | Q6UXX5 | ITIH6 |
| 2567 | 5660 | Q6WN34 | CHRDL2 |
| 2568 | 5661 | Q6X4U4 | SOSTDC1 |
| 2569 | 5662 | Q6XE38 | SCGB1D4 |
| 2570 | 5663 | Q6XZB0 | LIPI |
| 2571 | 5664 | Q6ZMH5 | SLC39A5 |
| 2572 | 5665 | Q6ZMR5 | TMPRSS11A |
| 2573 | 5666 | Q6ZNF0 | ACP7 |
| 2574 | 5667 | Q6ZP80 | TMEM182 |
| 2575 | 5668 | Q6ZQN7 | SLCO4C1 |
| 2576 | 5669 | Q6ZTQ4 | CDHR3 |
| 2577 | 5670 | Q75V66 | ANO5 |
| 2578 | 5671 | Q76B58 | BRINP3 |
| 2579 | 5672 | Q7L0J3 | SV2A |
| 2580 | 5673 | Q7L1I2 | SV2B |
| 2581 | 5674 | Q7L8A9 | VASH1 |
| 2582 | 5675 | Q7RTT9 | SLC29A4 |
| 2583 | 5676 | Q7RTW8 | OTOA |
| 2584 | 5677 | Q7RTX0 | TAS1R3 |
| 2585 | 5678 | Q7RTY5 | PRSS48 |
| 2586 | 5679 | Q7RTY7 | OVCH1 |
| 2587 | 5680 | Q7Z304 | MAMDC2 |
| 2588 | 5681 | Q7Z3D4 | LYSMD3 |
| 2589 | 5682 | Q7Z3S7 | CACNA2D4 |
| 2590 | 5683 | Q7Z404 | TMC4 |
| 2591 | 5684 | Q7Z410 | TMPRSS9 |
| 2592 | 5685 | Q7Z4F1 | LRP10 |
| 2593 | 5686 | Q7Z4W2 | LYZL2 |
| 2594 | 5687 | Q7Z5A4 | PRSS42 |
| 2595 | 5688 | Q7Z5A8 | FAM19A3 |
| 2596 | 5689 | Q7Z5A9 | FAM19A1 |
| 2597 | 5690 | Q7Z5L0 | VMO1 |
| 2598 | 5691 | Q7Z5M5 | TMC3 |
| 2599 | 5692 | Q7Z5P4 | HSD17B13 |
| 2600 | 5693 | Q7Z7B7 | DEFB132 |
| 2601 | 5694 | Q86SG7 | LYG2 |
| 2602 | 5695 | Q86SI9 | C5orf38 |
| 2603 | 5696 | Q86T26 | TMPRSS11B |
| 2604 | 5697 | Q86TE4 | LUZP2 |
| 2605 | 5698 | Q86TW2 | ADCK1 |
| 2606 | 5699 | Q86TY3 | C14orf37 |
| 2607 | 5700 | Q86U17 | SERPINA11 |
| 2608 | 5701 | Q86UD1 | OAF |
| 2609 | 5702 | Q86UL3 | GPAT4 |
| 2610 | 5703 | Q86W47 | KCNMB4 |
| 2611 | 5704 | Q86WD7 | SERPINA9 |
| 2612 | 5705 | Q86WI0 | LHFPL1 |
| 2613 | 5706 | Q86WS5 | TMPRSS12 |
| 2614 | 5707 | Q86XP6 | GKN2 |
| 2615 | 5708 | Q86YB8 | ERO1B |
| 2616 | 5709 | Q86YL7 | PDPN |
| 2617 | 5710 | Q86Z14 | KLB |
| 2618 | 5711 | Q86Z23 | CIQL4 |
| 2619 | 5712 | Q8IU80 | TMPRSS6 |
| 2620 | 5713 | Q8IU99 | CALHM1 |
| 2621 | 5714 | Q8IUB2 | WFDC3 |
| 2622 | 5715 | Q8IUH2 | CREG2 |
| 2623 | 5716 | Q8IUK5 | PLXDC1 |
| 2624 | 5717 | Q8IVL6 | P3H3 |
| 2625 | 5718 | Q8IVL8 | CPO |
| 2626 | 5719 | Q8IVM8 | SLC22A9 |
| 2627 | 5720 | Q8IVN8 | SBSPON |
| 2628 | 5721 | Q8IW75 | SERPINA12 |
| 2629 | 5722 | Q8IW92 | GLB1L2 |
| 2630 | 5723 | Q8IWF2 | FOXRED2 |
| 2631 | 5724 | Q8IWU5 | SULF2 |
| 2632 | 5725 | Q8IWU6 | SULF1 |
| 2633 | 5726 | Q8IX19 | MCEMP1 |
| 2634 | 5727 | Q8IXA5 | SPACA3 |
| 2635 | 5728 | Q8IXB1 | DNAJC10 |
| 2636 | 5729 | Q8IXB3 | TUSC5 |
| 2637 | 5730 | Q8IYJ0 | PIANP |
| 2638 | 5731 | Q8IYK4 | COLGALT2 |
| 2639 | 5732 | Q8IYS2 | KIAA2013 |
| 2640 | 5733 | Q8IZS8 | CACNA2D3 |
| 2641 | 5734 | Q8J025 | APCDD1 |
| 2642 | 5735 | Q8N0W4 | NLGN4X |
| 2643 | 5736 | Q8N0W7 | FMR1NB |
| 2644 | 5737 | Q8N129 | CNPY4 |
| 2645 | 5738 | Q8N131 | TMEM123 |
| 2646 | 5739 | Q8N158 | GPC2 |
| 2647 | 5740 | Q8N1C3 | GABRG1 |
| 2648 | 5741 | Q8N1E2 | LYG1 |
| 2649 | 5742 | Q8N2K0 | ABHD12 |
| 2650 | 5743 | Q8N2Q7 | NLGN1 |
| 2651 | 5744 | Q8N302 | AGGF1 |
| 2652 | 5745 | Q8N387 | MUC15 |
| 2653 | 5746 | Q8N3H0 | FAM19A2 |
| 2654 | 5747 | Q8N3Z0 | PRSS35 |
| 2655 | 5748 | Q8N436 | CPXM2 |
| 2656 | 5749 | Q8N474 | SFRP1 |
| 2657 | 5750 | Q8N4F0 | BPIFB2 |
| 2658 | 5751 | Q8N4T0 | CPA6 |
| 2659 | 5752 | Q8N539 | FIBCD1 |
| 2660 | 5753 | Q8N5I4 | DHRSX |
| 2661 | 5754 | Q8N5W8 | FAM24B |
| 2662 | 5755 | Q8N608 | DPP10 |
| 2663 | 5756 | Q8N695 | SLC5A8 |
| 2664 | 5757 | Q8N6F1 | CLDN19 |
| 2665 | 5758 | Q8N766 | EMC1 |
| 2666 | 5759 | Q8N807 | PDILT |
| 2667 | 5760 | Q8N9M5 | TMEM102 |
| 2668 | 5761 | Q8NA29 | MFSD2A |
| 2669 | 5762 | Q8NA58 | PNLDC1 |
| 2670 | 5763 | Q8NB37 | GATD1 |
| 2671 | 5764 | Q8NBJ9 | SIDT2 |
| 2672 | 5765 | Q8NBK3 | SUMF1 |
| 2673 | 5766 | Q8NBL1 | POGLUT1 |
| 2674 | 5767 | Q8NBQ5 | HSD17B11 |
| 2675 | 5768 | Q8NC42 | RNF149 |
| 2676 | 5769 | Q8NC54 | KCT2 |
| 2677 | 5770 | Q8NC67 | NETO2 |
| 2678 | 5771 | Q8NCS7 | SLC44A5 |
| 2679 | 5772 | Q8NCW5 | NAXE |
| 2680 | 5773 | Q8NDZ4 | C3orf58 |
| 2681 | 5774 | Q8NE79 | BVES |
| 2682 | 5775 | Q8NEA5 | C19orf18 |
| 2683 | 5776 | Q8NEB7 | ACRBP |
| 2684 | 5777 | Q8NER1 | TRPV1 |
| 2685 | 5778 | Q8NER5 | ACVR1C |
| 2686 | 5779 | Q8NET1 | DEFB108B |
| 2687 | 5780 | Q8NEX5 | WFDC9 |
| 2688 | 5781 | Q8NEX6 | WFDC11 |
| 2689 | 5782 | Q8NF86 | PRSS33 |
| 2690 | 5783 | Q8NFJ6 | PROKR2 |
| 2691 | 5784 | Q8NFQ5 | BPIFB6 |
| 2692 | 5785 | Q8NFU4 | FDCSP |
| 2693 | 5786 | Q8NFZ6 | VN1R2 |
| 2694 | 5787 | Q8NI22 | MCFD2 |
| 2695 | 5788 | Q8TAA1 | RNASE11 |
| 2696 | 5789 | Q8TAF8 | LHFPL5 |
| 2697 | 5790 | Q8TAL6 | FIBIN |
| 2698 | 5791 | Q8TAV5 | C11orf45 |
| 2699 | 5792 | Q8TAX7 | MUC7 |
| 2700 | 5793 | Q8TB22 | SPATA20 |
| 2701 | 5794 | Q8TB96 | ITFG1 |
| 2702 | 5795 | Q8TBP5 | FAM174A |
| 2703 | 5796 | Q8TCC7 | SLC22A8 |
| 2704 | 5797 | Q8TCP9 | FAM200A |
| 2705 | 5798 | Q8TCW7 | ZPLD1 |
| 2706 | 5799 | Q8TCW9 | PROKR1 |
| 2707 | 5800 | Q8TCZ2 | CD99L2 |
| 2708 | 5801 | Q8TD06 | AGR3 |
| 2709 | 5802 | Q8TD07 | RAET1E |
| 2710 | 5803 | Q8TD20 | SLC2A12 |
| 2711 | 5804 | Q8TDE3 | RNASE8 |
| 2712 | 5805 | Q8TDL5 | BPIFB1 |
| 2713 | 5806 | Q8TDN2 | KCNV2 |
| 2714 | 5807 | Q8TE23 | TAS1R2 |
| 2715 | 5808 | Q8TE56 | ADAMTS17 |
| 2716 | 5809 | Q8TE57 | ADAMTS16 |
| 2717 | 5810 | Q8TE58 | ADAMTS15 |
| 2718 | 5811 | Q8TE60 | ADAMTS18 |
| 2719 | 5812 | Q8TEB7 | RNF128 |
| 2720 | 5813 | Q8TEB9 | RHBDD1 |
| 2721 | 5814 | Q8WTR4 | GDPD5 |
| 2722 | 5815 | Q8WTV0 | SCARB1 |
| 2723 | 5816 | Q8WU39 | MZB1 |
| 2724 | 5817 | Q8WUF8 | FAM172A |
| 2725 | 5818 | Q8WUJ1 | CYB5D2 |
| 2726 | 5819 | Q8WUM4 | PDCD6IP |
| 2727 | 5820 | Q8WUM9 | SLC20A1 |
| 2728 | 5821 | Q8WWA0 | ITLN1 |
| 2729 | 5822 | Q8WWF1 | C1orf54 |
| 2730 | 5823 | Q8WWQ2 | HPSE2 |
| 2731 | 5824 | Q8WWU7 | ITLN2 |
| 2732 | 5825 | Q8WWY7 | WFDC12 |
| 2733 | 5826 | Q8WWY8 | LIPH |
| 2734 | 5827 | Q8WX39 | LCN9 |
| 2735 | 5828 | Q8WXA8 | HTR3C |
| 2736 | 5829 | Q8WXD2 | SCG3 |
| 2737 | 5830 | Q8WXQ8 | CPA5 |
| 2738 | 5831 | Q8WXS8 | ADAMTS14 |
| 2739 | 5832 | Q8WXW3 | PIBF1 |
| 2740 | 5833 | Q8WZ59 | TMEM190 |
| 2741 | 5834 | Q8WZ79 | DNASE2B |
| 2742 | 5835 | Q92484 | SMPDL3A |
| 2743 | 5836 | Q92520 | FAM3C |
| 2744 | 5837 | Q92537 | SUSD6 |
| 2745 | 5838 | Q92542 | NCSTN |
| 2746 | 5839 | Q92563 | SPOCK2 |
| 2747 | 5840 | Q92629 | SGCD |
| 2748 | 5841 | Q92765 | FRZB |
| 2749 | 5842 | Q92781 | RDH5 |
| 2750 | 5843 | Q92820 | GGH |
| 2751 | 5844 | Q92874 | DNASE1L2 |
| 2752 | 5845 | Q92876 | KLK6 |
| 2753 | 5846 | Q92911 | SLC5A5 |
| 2754 | 5847 | Q92932 | PTPRN2 |
| 2755 | 5848 | Q92959 | SLCO2A1 |
| 2756 | 5849 | Q92982 | NINJ1 |
| 2757 | 5850 | Q93070 | ART4 |
| 2758 | 5851 | Q93086 | P2RX5 |
| 2759 | 5852 | Q93091 | RNASE6 |
| 2760 | 5853 | Q93098 | WNT8B |
| 2761 | 5854 | Q96A33 | CCDC47 |
| 2762 | 5855 | Q96A84 | EMID1 |
| 2763 | 5856 | Q96AY3 | FKBP10 |
| 2764 | 5857 | Q96B33 | CLDN23 |
| 2765 | 5858 | Q96B86 | RGMA |
| 2766 | 5859 | Q96BD0 | SLCO4A1 |
| 2767 | 5860 | Q96BQ1 | FAM3D |
| 2768 | 5861 | Q96CG8 | CTHRC1 |
| 2769 | 5862 | Q96D15 | RCN3 |
| 2770 | 5863 | Q96DA0 | ZG16B |
| 2771 | 5864 | Q96DB9 | FXYD5 |
| 2772 | 5865 | Q96DD7 | SHISA4 |
| 2773 | 5866 | Q96DN0 | ERP27 |
| 2774 | 5867 | Q96DR5 | BPIFA2 |
| 2775 | 5868 | Q96DR8 | MUCL1 |
| 2776 | 5869 | Q96DX4 | RSPRY1 |
| 2777 | 5870 | Q96DZ1 | ERLEC1 |
| 2778 | 5871 | Q96EE4 | CCDC126 |
| 2779 | 5872 | Q96EG1 | ARSG |
| 2780 | 5873 | Q96EP9 | SLC10A4 |
| 2781 | 5874 | Q96F05 | C11orf24 |
| 2782 | 5875 | Q96FT7 | ASIC4 |
| 2783 | 5876 | Q96GC9 | VMP1 |
| 2784 | 5877 | Q96GX1 | TCTN2 |
| 2785 | 5878 | Q96HE7 | ERO1A |
| 2786 | 5879 | Q96HF1 | SFRP2 |
| 2787 | 5880 | Q96HH4 | TMEM169 |
| 2788 | 5881 | Q96HP4 | OXNAD1 |
| 2789 | 5882 | Q96HV5 | TMEM41A |
| 2790 | 5883 | Q96HY6 | DDRGK1 |
| 2791 | 5884 | Q96IY4 | CPB2 |
| 2792 | 5885 | Q96J42 | TXNDC15 |
| 2793 | 5886 | Q96JB6 | LOXL4 |
| 2794 | 5887 | Q96JW4 | SLC41A2 |
| 2795 | 5888 | Q96K78 | ADGRG7 |
| 2796 | 5889 | Q96KA5 | CLPTM1L |
| 2797 | 5890 | Q96KN2 | CNDP1 |
| 2798 | 5891 | Q96KX0 | LYZL4 |
| 2799 | 5892 | Q96L08 | SUSD3 |
| 2800 | 5893 | Q96L12 | CALR3 |
| 2801 | 5894 | Q96L15 | ART5 |
| 2802 | 5895 | Q96LB9 | PGLYRP3 |
| 2803 | 5896 | Q96LR4 | FAM19A4 |
| 2804 | 5897 | Q96LT7 | C9orf72 |
| 2805 | 5898 | Q96MK3 | FAM20A |
| 2806 | 5899 | Q96MU5 | C17orf77 |
| 2807 | 5900 | Q96NZ9 | PRAP1 |
| 2808 | 5901 | Q96P44 | COL21A1 |
| 2809 | 5902 | Q96PB7 | OLFM3 |
| 2810 | 5903 | Q96PC5 | MIA2 |
| 2811 | 5904 | Q96PD2 | DCBLD2 |
| 2812 | 5905 | Q96PH1 | NOX5 |
| 2813 | 5906 | Q96PL1 | SCGB3A2 |
| 2814 | 5907 | Q96PL2 | TECTB |
| 2815 | 5908 | Q96PS8 | AQP10 |
| 2816 | 5909 | Q96PZ7 | CSMD1 |
| 2817 | 5910 | Q96QD8 | SLC38A2 |
| 2818 | 5911 | Q96QE2 | SLC2A13 |
| 2819 | 5912 | Q96QR1 | SCGB3A1 |
| 2820 | 5913 | Q96QZ0 | PANX3 |
| 2821 | 5914 | Q96RQ9 | IL4I1 |
| 2822 | 5915 | Q96S42 | NODAL |
| 2823 | 5916 | Q96S66 | CLCC1 |
| 2824 | 5917 | Q96SL4 | GPX7 |
| 2825 | 5918 | Q96T91 | GPHA2 |
| 2826 | 5919 | Q99217 | AMELX |
| 2827 | 5920 | Q99218 | AMELY |
| 2828 | 5921 | Q99470 | SDF2 |
| 2829 | 5922 | Q99519 | NEU1 |
| 2830 | 5923 | Q99523 | SORT1 |
| 2831 | 5924 | Q99538 | LGMN |
| 2832 | 5925 | Q99542 | MMP19 |
| 2833 | 5926 | Q99571 | P2RX4 |
| 2834 | 5927 | Q99572 | P2RX7 |
| 2835 | 5928 | Q99584 | S100A13 |
| 2836 | 5929 | Q99674 | CGREF1 |
| 2837 | 5930 | Q99727 | TIMP4 |
| 2838 | 5931 | Q99784 | OLFM1 |
| 2839 | 5932 | Q99835 | SMO |
| 2840 | 5933 | Q99884 | SLC6A7 |
| 2841 | 5934 | Q99895 | CTRC |
| 2842 | 5935 | Q99943 | AGPAT1 |
| 2843 | 5936 | Q99954 | SMR3A |
| 2844 | 5937 | Q99969 | RARRES2 |
| 2845 | 5938 | Q99972 | MYOC |
| 2846 | 5939 | Q9BPW4 | APOL4 |
| 2847 | 5940 | Q9BQ08 | RETNLB |
| 2848 | 5941 | Q9BQ16 | SPOCK3 |
| 2849 | 5942 | Q9BQB4 | SOST |
| 2850 | 5943 | Q9BQI4 | CCDC3 |
| 2851 | 5944 | Q9BQS7 | HEPH |
| 2852 | 5945 | Q9BQT9 | CLSTN3 |
| 2853 | 5946 | Q9BQY6 | WFDC6 |
| 2854 | 5947 | Q9BRK5 | SDF4 |
| 2855 | 5948 | Q9BRN9 | TM2D3 |
| 2856 | 5949 | Q9BRR6 | ADPGK |
| 2857 | 5950 | Q9BS26 | ERP44 |
| 2858 | 5951 | Q9BSA4 | TTYH2 |
| 2859 | 5952 | Q9BSG0 | PRADC1 |
| 2860 | 5953 | Q9BSG5 | RTBDN |
| 2861 | 5954 | Q9BSJ5 | C17orf80 |
| 2862 | 5955 | Q9BSN7 | TMEM204 |
| 2863 | 5956 | Q9BT09 | CNPY3 |
| 2864 | 5957 | Q9BT56 | SPX |
| 2865 | 5958 | Q9BTY2 | FUCA2 |
| 2866 | 5959 | Q9BU40 | CHRDL1 |
| 2867 | 5960 | Q9BUR5 | APOO |
| 2868 | 5961 | Q9BV94 | EDEM2 |
| 2869 | 5962 | Q9BWS9 | CHID1 |
| 2870 | 5963 | Q9BX73 | TM2D2 |
| 2871 | 5964 | Q9BX74 | TM2D1 |
| 2872 | 5965 | Q9BX93 | PLA2G12B |
| 2873 | 5966 | Q9BX97 | PLVAP |
| 2874 | 5967 | Q9BXI9 | C1QTNF6 |
| 2875 | 5968 | Q9BXJ1 | C1QTNF1 |
| 2876 | 5969 | Q9BXJ2 | C1QTNF7 |
| 2877 | 5970 | Q9BXJ4 | C1QTNF3 |
| 2878 | 5971 | Q9BXR6 | CFHR5 |
| 2879 | 5972 | Q9BXS4 | TMEM59 |
| 2880 | 5973 | Q9BXY4 | RSPO3 |
| 2881 | 5974 | Q9BYE2 | TMPRSS13 |
| 2882 | 5975 | Q9BYE9 | CDHR2 |
| 2883 | 5976 | Q9BZD6 | PRRG4 |
| 2884 | 5977 | Q9BZD7 | PRRG3 |
| 2885 | 5978 | Q9BZG2 | ACP4 |
| 2886 | 5979 | Q9BZM1 | PLA2G12A |
| 2887 | 5980 | Q9BZM2 | PLA2G2F |
| 2888 | 5981 | Q9BZM5 | ULBP2 |
| 2889 | 5982 | Q9BZM6 | ULBP1 |
| 2890 | 5983 | Q9C0B6 | BRINP2 |
| 2891 | 5984 | Q9C0H2 | TTYH3 |
| 2892 | 5985 | Q9C0K1 | SLC39A8 |
| 2893 | 5986 | Q9GZM7 | TINAGL1 |
| 2894 | 5987 | Q9GZN4 | PRSS22 |
| 2895 | 5988 | Q9GZT5 | WNT10A |
| 2896 | 5989 | Q9GZX9 | TWSG1 |
| 2897 | 5990 | Q9GZZ6 | CHRNA10 |
| 2898 | 5991 | Q9GZZ8 | LACRT |
| 2899 | 5992 | Q9H015 | SLC22A4 |
| 2900 | 5993 | Q9H0B8 | CRISPLD2 |
| 2901 | 5994 | Q9H0U3 | MAGT1 |
| 2902 | 5995 | Q9H0X4 | FAM234A |
| 2903 | 5996 | Q9H112 | CST11 |
| 2904 | 5997 | Q9H114 | CSTL1 |
| 2905 | 5998 | Q9H173 | SIL1 |
| 2906 | 5999 | Q9H1A3 | METTL9 |
| 2907 | 6000 | Q9H1E1 | RNASE7 |
| 2908 | 6001 | Q9H1F0 | WFDC10A |
| 2909 | 6002 | Q9H1J7 | WNT5B |
| 2910 | 6003 | Q9H1M3 | DEFB129 |
| 2911 | 6004 | Q9H1Z8 | C2orf40 |
| 2912 | 6005 | Q9H221 | ABCG8 |
| 2913 | 6006 | Q9H2J7 | SLC6A15 |
| 2914 | 6007 | Q9H2R5 | KLK15 |
| 2915 | 6008 | Q9H2U9 | ADAM7 |
| 2916 | 6009 | Q9H306 | MMP27 |
| 2917 | 6010 | Q9H336 | CRISPLD1 |
| 2918 | 6011 | Q9H3G5 | CPVL |
| 2919 | 6012 | Q9H3N1 | TMX1 |
| 2920 | 6013 | Q9H3S3 | TMPRSS5 |
| 2921 | 6014 | Q9H3U7 | SMOC2 |
| 2922 | 6015 | Q9H3Y0 | R3HDML |
| 2923 | 6016 | Q9H461 | FZD8 |
| 2924 | 6017 | Q9H497 | TOR3A |
| 2925 | 6018 | Q9H4A4 | RNPEP |
| 2926 | 6019 | Q9H4B8 | DPEP3 |
| 2927 | 6020 | Q9H4D0 | CLSTN2 |
| 2928 | 6021 | Q9H4F8 | SMOC1 |
| 2929 | 6022 | Q9H4G1 | CST9L |
| 2930 | 6023 | Q9H5V8 | CDCP1 |
| 2931 | 6024 | Q9H6B9 | EPHX3 |
| 2932 | 6025 | Q9H6E4 | CCDC134 |
| 2933 | 6026 | Q9H741 | C12orf49 |
| 2934 | 6027 | Q9H772 | GREM2 |
| 2935 | 6028 | Q9H7B7 | C7orf69 |
| 2936 | 6029 | Q9H8H3 | METTL7A |
| 2937 | 6030 | Q9H8J5 | MANSC1 |
| 2938 | 6031 | Q9H9K5 | ERVMER34-1 |
| 2939 | 6032 | Q9HAT2 | SIAE |
| 2940 | 6033 | Q9HAW8 | UGT1A10 |
| 2941 | 6034 | Q9HAW9 | UGT1A8 |
| 2942 | 6035 | Q9HB40 | SCPEP1 |
| 2943 | 6036 | Q9HBJ0 | PLAC1 |
| 2944 | 6037 | Q9HBL7 | PLGRKT |
| 2945 | 6038 | Q9HBV2 | SPACA1 |
| 2946 | 6039 | Q9HC23 | PROK2 |
| 2947 | 6040 | Q9HC57 | WFDC1 |
| 2948 | 6041 | Q9HC58 | SLC24A3 |
| 2949 | 6042 | Q9HCB6 | SPON1 |
| 2950 | 6043 | Q9HCC8 | GDPD2 |
| 2951 | 6044 | Q9HCN8 | SDF2L1 |
| 2952 | 6045 | Q9HCX4 | TRPC7 |
| 2953 | 6046 | Q9HD89 | RETN |
| 2954 | 6047 | Q9HDC9 | APMAP |
| 2955 | 6048 | Q9NNX1 | TUFT1 |
| 2956 | 6049 | Q9NP55 | BPIFA1 |
| 2957 | 6050 | Q9NP70 | AMBN |
| 2958 | 6051 | Q9NP91 | SLC6A20 |
| 2959 | 6052 | Q9NPA0 | EMC7 |
| 2960 | 6053 | Q9NPA1 | KCNMB3 |
| 2961 | 6054 | Q9NPD5 | SLCO1B3 |
| 2962 | 6055 | Q9NPH5 | NOX4 |
| 2963 | 6056 | Q9NPH6 | OBP2B |
| 2964 | 6057 | Q9NQ30 | ESM1 |
| 2965 | 6058 | Q9NQ34 | TMEM9B |
| 2966 | 6059 | Q9NQ38 | SPINK5 |
| 2967 | 6060 | Q9NQ40 | SLC52A3 |
| 2968 | 6061 | Q9NQ60 | EQTN |
| 2969 | 6062 | Q9NQ76 | MEPE |
| 2970 | 6063 | Q9NQ90 | ANO2 |
| 2971 | 6064 | Q9NQE7 | PRSS16 |
| 2972 | 6065 | Q9NQX5 | NPDC1 |
| 2973 | 6066 | Q9NRC9 | OTOR |
| 2974 | 6067 | Q9NRE1 | MMP26 |
| 2975 | 6068 | Q9NRM1 | ENAM |
| 2976 | 6069 | Q9NRN5 | OLFML3 |
| 2977 | 6070 | Q9NRR1 | CYTL1 |
| 2978 | 6071 | Q9NRS4 | TMPRSS4 |
| 2979 | 6072 | Q9NS71 | GKN1 |
| 2980 | 6073 | Q9NSA0 | SLC22A11 |
| 2981 | 6074 | Q9NSD5 | SLC6A13 |
| 2982 | 6075 | Q9NT22 | EMILIN3 |
| 2983 | 6076 | Q9NTU7 | CBLN4 |
| 2984 | 6077 | Q9NU53 | GINM1 |
| 2985 | 6078 | Q9NUN5 | LMBRD1 |
| 2986 | 6079 | Q9NW15 | ANO10 |
| 2987 | 6080 | Q9NWH7 | SPATA6 |
| 2988 | 6081 | Q9NWM8 | FKBP14 |
| 2989 | 6082 | Q9NX61 | TMEM161A |
| 2990 | 6083 | Q9NXC2 | GFOD1 |
| 2991 | 6084 | Q9NY37 | ASIC5 |
| 2992 | 6085 | Q9NY91 | SLC5A4 |
| 2993 | 6086 | Q9NYL4 | FKBP11 |
| 2994 | 6087 | Q9NZ20 | PLA2G3 |
| 2995 | 6088 | Q9NZ53 | PODXL2 |
| 2996 | 6089 | Q9NZ94 | NLGN3 |
| 2997 | 6090 | Q9NZG7 | NINJ2 |
| 2998 | 6091 | Q9NZK5 | ADA2 |
| 2999 | 6092 | Q9NZK7 | PLA2G2E |
| 3000 | 6093 | Q9NZP8 | C1RL |
| 3001 | 6094 | Q9NZQ8 | TRPM5 |
| 3002 | 6095 | Q9P0G3 | KLK14 |
| 3003 | 6096 | Q9P0L9 | PKD2L1 |
| 3004 | 6097 | Q9P2E8 | MARCHF4 |
| 3005 | 6098 | Q9P2K2 | TXNDC16 |
| 3006 | 6099 | Q9UBC7 | GALP |
| 3007 | 6100 | Q9UBD9 | CLCF1 |
| 3008 | 6101 | Q9UBN1 | CACNG4 |
| 3009 | 6102 | Q9UBN4 | TRPC4 |
| 3010 | 6103 | Q9UBP4 | DKK3 |
| 3011 | 6104 | Q9UBR2 | CTSZ |
| 3012 | 6105 | Q9UBS3 | DNAJB9 |
| 3013 | 6106 | Q9UBS4 | DNAJB11 |
| 3014 | 6107 | Q9UBT3 | DKK4 |
| 3015 | 6108 | Q9UBU2 | DKK2 |
| 3016 | 6109 | Q9UBV4 | WNT16 |
| 3017 | 6110 | Q9UEW3 | MARCO |
| 3018 | 6111 | Q9UGM1 | CHRNA9 |
| 3019 | 6112 | Q9UHC3 | ASIC3 |
| 3020 | 6113 | Q9UHG3 | PCYOX1 |
| 3021 | 6114 | Q9UHI8 | ADAMTS1 |
| 3022 | 6115 | Q9UHL4 | DPP7 |
| 3023 | 6116 | Q9UHM6 | OPN4 |
| 3024 | 6117 | Q9UI38 | PRSS50 |
| 3025 | 6118 | Q9UI42 | CPA4 |
| 3026 | 6119 | Q9UIG8 | SLCO3A1 |
| 3027 | 6120 | Q9UJ14 | GGT7 |
| 3028 | 6121 | Q9UJA9 | ENPP5 |
| 3029 | 6122 | Q9UJJ9 | GNPTG |
| 3030 | 6123 | Q9UJQ1 | LAMP5 |
| 3031 | 6124 | Q9UJW2 | TINAG |
| 3032 | 6125 | Q9UK28 | TMEM59L |
| 3033 | 6126 | Q9UK55 | SERPINA10 |
| 3034 | 6127 | Q9UK85 | DKKL1 |
| 3035 | 6128 | Q9UKI3 | VPREB3 |
| 3036 | 6129 | Q9UKQ9 | KLK9 |
| 3037 | 6130 | Q9UKR3 | KLK13 |
| 3038 | 6131 | Q9UKU6 | TRHDE |
| 3039 | 6132 | Q9UKY0 | PRND |
| 3040 | 6133 | Q9UKZ9 | PCOLCE2 |
| 3041 | 6134 | Q9UL01 | DSE |
| 3042 | 6135 | Q9UL52 | TMPRSS11E |
| 3043 | 6136 | Q9UL62 | TRPC5 |
| 3044 | 6137 | Q9ULV1 | FZD4 |
| 3045 | 6138 | Q9ULW2 | FZD10 |
| 3046 | 6139 | Q9ULX7 | CA14 |
| 3047 | 6140 | Q9UM22 | EPDR1 |
| 3048 | 6141 | Q9UMR5 | PPT2 |
| 3049 | 6142 | Q9UMX5 | NENF |
| 3050 | 6143 | Q9UN76 | SLC6A14 |
| 3051 | 6144 | Q9UN88 | GABRQ |
| 3052 | 6145 | Q9UNI1 | CELA1 |
| 3053 | 6146 | Q9UNK4 | PLA2G2D |
| 3054 | 6147 | Q9UNQ0 | ABCG2 |
| 3055 | 6148 | Q9UNW1 | MINPP1 |
| 3056 | 6149 | Q9UQF0 | ERVW-1 |
| 3057 | 6150 | Q9UQQ1 | NAALADL1 |
| 3058 | 6151 | Q9Y215 | COLQ |
| 3059 | 6152 | Q9Y251 | HPSE |
| 3060 | 6153 | Q9Y267 | SLC22A14 |
| 3061 | 6154 | Q9Y2B0 | CNPY2 |
| 3062 | 6155 | Q9Y2B1 | RXYLT1 |
| 3063 | 6156 | Q9Y2E5 | MAN2B2 |
| 3064 | 6157 | Q9Y2G5 | POFUT2 |
| 3065 | 6158 | Q9Y2G8 | DNAJC16 |
| 3066 | 6159 | Q9Y320 | TMX2 |
| 3067 | 6160 | Q9Y337 | KLK5 |
| 3068 | 6161 | Q9Y345 | SLC6A5 |
| 3069 | 6162 | Q9Y394 | DHRS7 |
| 3070 | 6163 | Q9Y4K0 | LOXL2 |
| 3071 | 6164 | Q9Y561 | LRP12 |
| 3072 | 6165 | Q9Y5I7 | CLDN16 |
| 3073 | 6166 | Q9Y5K2 | KLK4 |
| 3074 | 6167 | Q9Y5L3 | ENTPD2 |
| 3075 | 6168 | Q9Y5S8 | NOX1 |
| 3076 | 6169 | Q9Y5X9 | LIPG |
| 3077 | 6170 | Q9Y5Y6 | ST14 |
| 3078 | 6171 | Q9Y5Y7 | LYVE1 |
| 3079 | 6172 | Q9Y5Z0 | BACE2 |
| 3080 | 6173 | Q9Y625 | GPC6 |
| 3081 | 6174 | Q9Y646 | CPQ |
| 3082 | 6175 | Q9Y680 | FKBP7 |
| 3083 | 6176 | Q9Y691 | KCNMB2 |
| 3084 | 6177 | Q9Y693 | LHFPL6 |
| 3085 | 6178 | Q9Y6C5 | PTCH2 |
| 3086 | 6179 | Q9Y6I9 | TEX264 |
| 3087 | 6180 | Q9Y6L6 | SLCO1B1 |
| 3088 | 6181 | Q9Y6M0 | PRSS21 |
| 3089 | 6182 | Q9Y6M7 | SLC4A7 |
| 3090 | 6183 | Q9Y6U7 | RNF215 |
| 3091 | 6184 | Q9Y6X5 | ENPP4 |
| 3092 | 6185 | Q9Y6Y9 | LY96 |

Library Construction:

A two-step PCR process was used to amplify cDNAs for cloning into a barcoded yeast-display vector. cDNAs were amplified with gene-specific primers, with the forward primer containing a 5′ sequence (CTGTTATTGCTAGCGTTTTAGCA (SEQ ID NO: 6186)) and the reverse primer containing a 5′ sequence (GCCACCAGAAGCGGCCGC (SEQ ID NO: 6187)) for template addition in the second step of PCR. PCR reactions were conducted using 1 μL pooled cDNA, gene-specific primers, and the following PCR settings: 98° C. denaturation, 58° C. annealing, 72° C. extension, 35 rounds of amplification. 1 μL of PCR product was used for direct amplification by common primers Aga2FOR and 159REV, and the following PCR settings: 98° C. denaturation, 58° C. annealing, 72° C. extension, 35 rounds of amplification. PCR product was purified using magnetic PCR purification beads (AvanBio). 90 μL beads were added to the PCR product and supernatant was removed. Beads were washed twice with 200 μL 70% ethanol and resuspended in 50 μL water to elute PCR products from the beads. Beads were removed from purified PCR products. The 15 bp barcode fragment was constructed by overlap PCR. 4 primers (bc1, bc2, bc3, bc4) were mixed in equimolar ratios and used as template for a PCR reaction using the following PCR settings: 98° C. denaturation, 55° C. annealing, 72° C. extension, 35 rounds of amplification. Purified product was reamplified with the first and fourth primer using identical PCR conditions. PCR products were run on 2% agarose gels and purified by gel extraction (Qiagen). Purified barcode and gene products were combined with linearized yeast-display vector (pDD003 digested with EcoRI and BamHI) and electroporated into JAR300 yeast cell using a 96-well electroporater (BTX Harvard Apparatus) using the following electroporation conditions: Square wave, 500 V, 5 ms pulse, 2 mm gap. Yeast cell were immediately recovered into 1 mL liquid synthetic dextrose medium lacking uracil (SDO-Ura) in 96-well deepwell blocks and grown overnight at 30° C. Yeast cell were passaged once by 1:10 dilution in SDO-Ura, then frozen as glycerol stocks. To construct the final library, 2.5 μL of all wells except 32 containing genes previously identified as incompatible with high-quality yeast cell display were pooled and counted. A limited dilution of 56,000 clones was sub-sampled and expanded in SDO-Ura. Expression was induced by passaging into synthetic galactose medium lacking uracil (SGO-Ura) at a 1:10 dilution and growing at 30° C. overnight. 10⁸ yeast cell were pelleted and resuspend in 1 mL PBE (PBS with 0.5% BSA and 0.5 mM EDTA) containing 1:100 anti-FLAG PE antibody (BioLegend). Yeast cell were stained at 4° C. for 75 minutes, then washed twice with 1 mL PBE and sorted for FLAG display on a Sony SH800Z cell sorter. Sorted cells were expanded in SDO-Ura supplemented with 35 μg/mL chloramphenicol, expanded, and frozen as the final library.

(SEQ ID NO: 6188)
bc1-TTGTTAATATACCTCTATACTTTAACGTCAAGGAGAAAAAACCCCG
GATC
(SEQ ID NO: 6189)
bc2-CTGCATCCTTTAGTGAGGGTTGAANNNNNNNNNNNNNNNTTCGATC
CGGGGTTTTTTCTCCTTG
(SEQ ID NO: 6190)
bc3-TTCAACCCTCACTAAAGGATGCAGTTACTTCGCTGTTTTTCAATAT
TTTCTGTTATTGC
(SEQ ID NO: 6191)
bc4-TGCTAAAACGCTAGCAATAACAGAAAATATTGAAAAACAGCG

Barcode Identification:

Barcode-gene pairings were identified using a custom Tn5-based sequence approach. Tn5 transposase was purified as previously described, using the on-column assembly method for loading oligos. DNA was extracted from the yeast library using Zymoprep-96 Yeast Plasmid Miniprep kits or Zymoprep Yeast Plasmid Miniprep II kits (Zymo Research) according to standard manufacturer protocols. 5 μL of purified plasmid DNA was digested with Tn5 in a 20 μL total reaction as previously described. 2 μL of digested DNA was amplified using primers index1 and index2, using the following PCR settings: 98° C. denaturation, 56° C. annealing, 72° C. extension, 25 rounds of amplification. The product was run on a 2% gel and purified by gel extraction (Qiagen). Purified product was amplified using primers index3 and index4, using the following PCR settings: 98° C. denaturation, 60° C. annealing, 72° C. extension, 25 rounds of amplification. In parallel, the barcode region alone was amplified using primers index1 and index5, using the following PCR settings: 98° C. denaturation, 56° C. annealing, 72° C. extension, 25 rounds of amplification. The product was run on a 2% gel and purified by gel extraction (Qiagen). Purified product was amplified using primers index3 and index6, using the following PCR settings: 98° C. denaturation, 60° C. annealing, 72° C. extension, 20 rounds of amplification. Both barcode and digested fragment products were run on a 2% gel and purified by gel extraction (Qiagen). NGS library was sequenced using an Illumina MiSeq and Illumina v3 MiSeq Reagent Kits with 150 base pair single-end sequencing according to standard manufacturer protocols. Gene-barcode pairings were identified using custom code. Briefly, from each read, the barcode sequence was extracted based on the identification of the flanking constant vector backbone sequences, and the first 25 bp of sequence immediately following the constant vector backbone-derived signal peptide were extracted and mapped to a gene identity based on the first 25 bp of all amplified cDNA constructs. The number of times each barcode was paired with an identified gene was calculated. Barcode-gene pairings that were identified more than twice, with an overall observed barcode frequency of greater than 0.0002% were compiled. For barcodes with multiple gene pairings matching the above criteria, the best-fit gene was manually identified by inspection of all barcode-gene pairing frequencies and, in general, identification of the most abundant gene pairing. In the final library, 2,688 genes were confidently mapped to 35,835 barcodes.

Rapid Extracellular Antigen Profiling.

Antibody Purification and Yeast Cell Adsorption

20 μL protein G magnetic resin (Lytic Solutions) was washed twice with 100 μL sterile PBS, resuspended in 50 μL PBS, and added to 50 μL serum or plasma. Serum-resin mixture was incubated for three hours at 4° C. with shaking. Resin was washed five times with 200 μL PBS, resuspended in 90 μL 100 mM glycine pH 2.7, and incubated for five minutes at room temperature. Supernatant was extracted and added to 10 μL sterile 1M Tris pH 8.0 (purified IgG). Empty vector (pDD003) yeast cell were expanded in SDO-Ura at 30° C. One day later, yeast cell were induced by 1:10 dilution in SGO-Ura for 24 hours. 10⁸ induced yeast cell were washed twice with 200 μL PBE (PBS with 0.5% BSA and 0.5 mM EDTA), resuspended with 100 μL purified IgG, and incubated for three hours at 4° C. with shaking. Yeast-IgG mixtures were placed into 96 well 0.45 um filter plates (Thomas Scientific) and yeast-depleted IgG was eluted into sterile 96 well plates by centrifugation at 3000 g for 3 minutes.

Antibody Yeast Library Selections.

Transformed yeast were expanded in SDO-Ura at 30° C. One day later, at an optical density (OD) below 8, yeast were induced by resuspension at an OD of 1 in SGO-Ura supplemented with ten percent SDO-Ura and culturing at 30° C. for 20 hours. Prior to selection, 400 μL pre-selection library was set aside to allow for comparison to post-selection libraries. 10⁸ induced yeast were washed twice with 200 μL PBE and added to wells of a sterile 96-well v-bottom microtiter plate. Yeast were resuspended in 100 μL PBE containing appropriate antibody concentration and incubated with shaking for 1 hour at 4° C. Unless otherwise indicated, 10 μg antibody per well was used for human serum or plasma derived antibodies and 1 μg antibody was used for monoclonal antibodies. Yeast were washed twice with 200 μL PBE, resuspended in 100 μL PBE with a 1:100 dilution of biotin anti-human IgG Fc antibody (clone HP6017, BioLegend) for human serum or plasma derived antibodies or a 1:25 dilution of biotin goat anti-rat or anti-mouse IgG antibody (A16088, Thermo Fisher Scientific; A18869, Thermo Fisher Scientific) for monoclonal antibodies. Yeast-antibody mixtures were incubated with shaking for 30 minutes at 4° C. Yeast were washed twice with 200 μL PBE, resuspended in 100 μL PBE with a 1:20 dilution of Streptavidin MicroBeads (Miltenyi Biotec), and incubated with shaking for 30 minutes at 4° C. Yeast were then pelleted and kept on ice. Multi-96 Columns (Miltenyi Biotec) were placed into a MultiMACS M96 Separator (Miltenyi Biotec) and the separator was placed into positive selection mode. All following steps were carried out at room temperature. Columns were equilibrated with 400 μL 70% ethanol followed by 700 μL degassed PBE. Yeast were resuspended in 200 μL degassed PBE and placed into the columns. After the mixture had completely passed through, columns were washed three times with 700 μL degassed PBE. To elute the selected yeast, columns were removed from the separator and placed over 96-well deep well plates. 700 μL degassed PBE was added to each well of the column and the column and deep well plate were spun at 50 g for 30 seconds. This process was repeated 3 times. Selected yeast were pelleted, and recovered in 1 mL SDO-Ura at 30° C.

Recombinant Protein Yeast Library Selections.

All pre-selection and yeast induction steps were performed identically as those of the antibody yeast library selections. 10⁸ induced yeast were washed twice with 200 μL PBE and added to wells of a sterile 96-well v-bottom microtiter plate. Yeast were resuspended in 100 μL PBE containing 75 μL clarified protein expression supernatant and incubated with shaking for 1 hour at 4° C. Yeast were washed twice with 200 μL PBE, resuspended in 100 μL PBE with 5 μL MACS Protein G MicroBeads (Miltenyi Biotec), and incubated with shaking for 30 minutes at 4° C. Selection of yeast using the MultiMACS M96 Separator and subsequent steps were performed identically as those of the antibody yeast library selections.

Next Generation Sequencing Library Preparation and Sequencing.

DNA was extracted from yeast libraries using Zymoprep-96 Yeast Plasmid Miniprep kits or Zymoprep Yeast Plasmid Miniprep II kits (Zymo Research) according to standard manufacturer protocols. A first round of PCR was used to amplify a DNA sequence containing the protein display barcode on the yeast plasmid. PCR reactions were conducted using 1 μL plasmid DNA, 159_DIF2 and 159_DIR2 primers (sequences listed below), and the following PCR settings: 98° C. denaturation, 58° C. annealing, 72° C. extension, 25 rounds of amplification. PCR product was purified using magnetic PCR purification beads (AvanBio). 45 μL beads were added to the PCR product and supernatant was removed. Beads were washed twice with 100 μL 70% ethanol and resuspended in 25 μL water to elute PCR products from the beads. Beads were removed from purified PCR products. A second round of PCR was conducted using 1 μL purified PCR product, Nextera i5 and i7 dual-index library primers (Illumina), and the following PCR settings: 98° C. denaturation, 58° C. annealing, 72° C. extension, 25 rounds of amplification. PCR products were pooled and run on a 1% agarose gel. The band corresponding to 257 base pairs was cut out and DNA (NGS library) was extracted using a QlAquick Gel Extraction Kit (Qiagen) according to standard manufacturer protocols. NGS library was sequenced using an Illumina MiSeq and Illumina v3 MiSeq Reagent Kits with 75 base pair single-end sequencing or using an Illumina NovaSeq 6000 and Illumina NovaSeq S4 200 cycle kit with 101 base pair paired-end sequencing according to standard manufacturer protocols. A minimum of 50,000 reads per sample was collected and the pre-selection library was sampled at ten times greater depth than other samples.

(SEQ ID NO: 6192)
159_DIF2-TCGTCGGCAGCGTCAGATGTGTATAAGAGACAGNNNNNNNN
NNGAGAAAAAACCCCGGATCG
(SEQ ID NO: 6193)
159_DIR2-GTCTCGTGGGCTCGGAGATGTGTATAAGAGACAGNNNNNNN
NNNACGCTAGCAATAACAGAAAATATTG

Data Analysis.

REAP scores were calculated as follows. First, barcode counts were extracted from raw NGS data using custom codes and counts from technical replicates were summed. Next, aggregate and clonal enrichment was calculated using edgeR⁶² and custom codes. For aggregate enrichment, barcode counts across all unique barcodes associated with a given protein were summed, library sizes across samples were normalized using default edgeR parameters, common and tagwise dispersion were estimated using default edgeR parameters, and exact tests comparing each sample to the pre-selection library were performed using default edgeR parameters. Aggregate enrichment is thus the log 2 fold change values from these exact tests with zeroes in the place of negative fold changes. Log 2 fold change values for clonal enrichment were calculated in an identical manner, but barcode counts across all unique barcodes associated with a given protein were not summed. Clonal enrichment for a given reactivity was defined as the fraction of clones out of total clones that were enriched (log 2 fold change ≥2). Aggregate (Eₐ) and clonal enrichment (E_{c}) for a given protein, a scaling factor (βᵤ) based on the number of unique yeast clones (yeast that have a unique DNA barcode) displaying a given protein, and a scaling factor (β_{f}) based on the overall frequency of yeast in the library displaying a given protein were used as inputs to calculate the REAP score, which is defined as follows.

REAP score=Eₐ*(E_{c})²*βᵤ*β_{f}

βᵤ and β_{f} are logarithmic scaling factors that progressively penalize the REAP score of proteins with low numbers of unique barcodes or low frequencies in the library. βᵤ is applied to proteins with ≤5 unique yeast clones in the library and β_{f} is applied to proteins with a frequency ≤0.0001 in the library. β_{f} was implemented to mitigate spurious enrichment signals from low frequency proteins, which could occur due to sequencing errors or stochasticity in the selection process. βᵤ was implemented because the clonal enrichment metric is less valid for proteins with low numbers of unique yeast clones, decreasing confidence in the validity of the reactivity. βᵤ and β_{f} are defined as follows where xᵤ is the number of unique yeast clones for a given protein and x_{f} is the log 10 transformed frequency of a given protein in the library.

$${β}_{u} = \frac{ln ( {x}_{u} + 0.5 )}{1.705}$$

$${β}_{f} = \frac{ln ( {x}_{f} + 7.1 )}{1.16}$$

Recombinant Protein Production.

REAP Recombinant Protein Production.

Proteins were produced as human IgG1 Fc fusions to enable binding of secondary antibody and magnetic beads to the produced proteins during the REAP process. Sequences encoding the extracellular portions of proteins-of-interests that were present in the yeast display library were cloned by Gibson assembly into a modified pD2610-v12 plasmid (ATUM). Modifications include addition of an H7 signal sequence followed by a (GGGGS)₃ linker and a truncated human IgG1 Fc (N297A). Protein-of-interest sequences were inserted directly downstream of the H7 leader sequence. Protein was produced by transfection into Expi293 cells (Thermo Fisher Scientific) in 96-well plate format. One day prior to transfection, cells were seeded at a density of 2 million cells per mL in Expi293 Expression Medium (Thermo Fisher Scientific). In a 96-well plate, 0.5 μg plasmid DNA was diluted added to 25 μL Opti-MEM (Thermo Fisher Scientific) and mixed gently. In a separate 96-well plate, 1.35 μL ExpiFectamine was added to 25 μL Opti-MEM and mixed gently. The ExpiFectamine-Opti-MEM mixture was added to the diluted DNA, mixed gently, and incubated for 20 minutes at room temperature. Expi293 cells were diluted to a density of 2.8 million cells per mL and 500 L of cells were added to each well of a 96-well deep well plate. 50 μL of the DNA-ExpiFectamine-Opti-MEM mixture was added to each well. The plate was sealed with Breathe-Easier sealing film (Diversified Biotech) and incubated in a humidified tissue culture incubator (37° C., 8% CO₂) with shaking at 1,200 rpm so that cells were kept in suspension. 18-20 hours post-transfection, 25 μL enhancer 2 and 2.5 μL enhancer 1 (Thermo Fisher Scientific) were added to each well. 4 days post-transfection, media was clarified by centrifugation at 3000-4000 g for 5 minutes. Clarified media was used for recombinant protein REAP.

ELISA Protein Production.

Sequences encoding the extracellular portions of proteins-of-interests that were present in the yeast display library were cloned by Gibson assembly into pEZT_Dlux, a modified pEZT-BM vector. The pEZT-BM vector was a gift from Ryan Hibbs (Addgene plasmid #74099). Modifications included insertion of an H7 Leader Sequence followed by an AviTag (Avidity), HRV 3C site, protein C epitope, and an 8×his tag. Protein-of-interest sequences were inserted directly downstream of the H7 leader sequence. Protein was produced by transfection into Expi293 cells (Thermo Fisher Scientific) according to standard manufacturer protocols. Transfected cells were maintained according to manufacturer protocols. 4 days post-transfection, media was clarified by centrifugation at 300 g for 5 minutes. Protein was purified from clarified media by nickel-nitrilotriacetic acid (Ni-NTA) chromatography and desalted into HEPES buffered saline+100 mM sodium chloride, pH 7.5. Protein purity was verified by SDS-PAGE.

Biotinylated Protein Production.

Sequences encoding the extracellular portions of proteins-of-interests were cloned into pEZT_Dlux as described above. Protein was expressed and purified as described above minus desalting. Enzymatic biotinylation with BirA ligase was performed and protein was purified by size-exclusion fast protein liquid chromatography using a NGC Quest 10 Chromatography System (Bio-Rad).

LIPS Protein Production.

Sequences encoding Lucia luciferase (InvivoGen) fused by a GGSG linker to the N-terminus of the protein-of-interest extracellular portion (as defined above) were cloned by Gibson assembly into pEZT-BM. Protein was produced by transfection into Expi293 cells (Thermo Fisher Scientific) according to standard manufacturer protocols. Transfected cells were maintained according to manufacturer protocols. 3 days post-transfection, media was clarified by centrifugation at 300 g for 5 minutes. Clarified media was used in luciferase immunoprecipitation systems assays.

Enzyme-Linked Immunosorbent Assays (ELISAs).

200 or 400 ng of purchased or independently produced recombinant protein in 100 μL of PBS pH 7.0 was added to 96-well flat bottom Immulon 2HB plates (Thermo Fisher Scientific) and placed at 4° C. overnight. Plates were washed once with 225 μL ELISA wash buffer (PBS+0.05% Tween 20) and 150 μL ELISA blocking buffer (PBS+2% Human Serum Albumin) was added to the well. Plates were incubated with shaking for 2 hours at room temperature. ELISA blocking buffer was removed from the wells and appropriate dilutions of sample serum in 100 μL ELISA blocking buffer were added to each well. Plates were incubated with shaking for 2 hours at room temperature. Plates were washed 6 times with 225 μL ELISA wash buffer and 1:5000 goat anti-human IgG HRP (Millipore Sigma) or anti-human IgG isotype specific HRP (Southern Biotech; IgG1: clone HP6001, IgG2: clone 31-7-4, IgG3: clone HP6050, IgG4: clone HP6025) in 100 μL ELISA blocking buffer was added to the wells. Plates were incubated with shaking for 1 hour at room temperature. Plates were washed 6 times with 225 μL ELISA wash buffer. 50 μL TMB substrate (BD Biosciences) was added to the wells and plates were incubated for 15 minutes (pan-IgG ELISAs) or 20 minutes (isotype specific IgG ELISAs) in the dark at room temperature. 50 μL 1 M sulfuric acid was added to the wells and absorbance at 450 nm was measured in a Synergy HTX Multi-Mode Microplate Reader (BioTek).

Luciferase Immunoprecipitation Systems (LIPS) Assays.

Pierce Protein A/G Ultralink Resin (5 μL; Thermo Fisher Scientific) and 1 μL sample serum in 100 μL Buffer A (50 mM Tris, 150 mM NaCl, 0.1% Triton X-100, pH 7.5) was added to 96-well opaque Multiscreen HTS 96 HV 0.45 um filter plates (Millipore Sigma). Plates were incubated with shaking at 300 rpm for 1 hour at room temperature. Supernatant in wells was removed by centrifugation at 2000 g for 1 minute. Luciferase fusion protein (10⁶ RLU) was added to the wells in 100 μL Buffer A. Plates were incubated with shaking at 300 rpm for 1 hour at room temperature. Using a vacuum manifold, wells were washed 8 times with 100 μL Buffer A followed by 2 washes with 100 μL PBS. Remaining supernatant in wells was removed by centrifugation at 2000 g for 1 minute. Plates were dark adapted for 5 minutes. An autoinjector equipped Synergy HTX Multi-Mode Microplate Reader (BioTek) was primed with QUANTI-Luc Gold (InvivoGen). Plates were read using the following per well steps: 50 μL QUANTI-Luc Gold injection, 4 second delay with shaking, read luminescence with an integration time of 0.1 seconds and a read height of 1 mm.

PD-L2 Blocking Assay.

A single clone of PD-L2 displaying yeast was isolated from the library and expanded in SDO-Ura at 30° C. Yeast were induced by 1:10 dilution into SGO-Ura and culturing at 30° C. for 24 hours. 105 induced PD-L1 yeast were washed twice with 200 μL PBE and added to wells of a 96-well v-bottom microtiter plate. Yeast were resuspended in 25 μL PBE containing serial dilutions of sample serum and incubated with shaking for 1 hour at 4° C. PD-1 tetramers were prepared by incubating a 5:1 ratio of biotinylated PD-1 and PE streptavidin (BioLegend) for 10 minutes on ice in the dark. Yeast were washed twice with 200 μL PBE, resuspended in 25 μL PBE containing 10 nM previously prepared PD-1 tetramers, and incubated with shaking for 1 hour at 4° C. Yeast were washed twice with 200 μL PBE and resuspended in 75 μL PBE. PE fluorescent intensity was quantified by flow cytometry using a Sony SA3800 Spectral Cell Analyzer. Percent max binding was calculated based on fluorescent PD-1 tetramer binding in the absence of any serum.

IL-33 Neutralization Assay.

IL-33 Reporter Cell Line Construction.

The full-length coding sequence for ST2 was cloned by Gibson assembly into the lentiviral transfer plasmid pL-SFFV.Reporter.RFP657.PAC, a kind gift from Benjamin Ebert (Addgene plasmid #61395). REK-293FT cells were seeded into a 6-well plate in 2 mL growth media (DMEM with 10% (v/v) FBS, 100 units/mL penicillin, and 0.1 mg/mL streptomycin) and were incubated at 37° C., 5% CO2. Once cells achieved 70-80% confluence approximately one day later, cells were transfected using TransIT-LT1 (Mirus Bio) in Opti-MEM media (Life Technologies). TransIT-LTI Reagent was pre-warmed to room temperature and vortexed gently. For each well, 0.88 ug lentiviral transfer plasmid along with 0.66 ug pSPAX2 (Addgene plasmid #12260) and 0.44 ug pMD2.G (Addgene plasmid #12259), kind gifts from Didier Trono, were added to 250 μL Opti-MEM media and mixed gently. TransIT-LT1 reagent (6 μl) was added to the DNA mixture, mixed gently, and incubated at room temperature for 15-20 minutes. The mixture was added dropwise to different areas of the well Plates were incubated at 37° C., 5% CO2; 48 hrs later, the virus-containing media was collected and filtered with a 0.45 μm low protein-binding filter. H1EK-BIlue IL-18 cells (InvivoGen) were seeded into a 6-well plate in 1 mL growth media (DMEM with 10% (v/v) FBS, 100 units/mL penicillin, and 0.1 mg/mL streptomycin) and 1 mL virus-containing media. Cells were incubated at 37° C., 5% CO2 for two days before the media was changed.

Reporter Cell Stimulation and Reading.

Purified IgG titrations and 2 nM IL-33 were mixed in 50 μL assay media (DMEM with 10% (v/v) FBS, 100 units/mL penicillin, and 0.1 mg/mL streptomycin) and incubated with shaking for 1 hour at room temperature. Approximately 50,000 IL-33 reporter cells in 50 μl assay media were added to wells of a sterile tissue culture grade flat-bottom 96-well plate. IgG-IL-33 mixtures were added to respective wells (1 nM IL-33 final concentration). Plates were incubated at 37° C. 5% CO2 for 20 hours, then 20 μL media from each well was added to 180 μL room temperature QUANTI-Blue Solution (InvivoGen) in a separate flat-bottom 96-well plate and incubated at 37° C. for 3 hours. Absorbance at 655 nm was measured in a Synergy HTX Multi-Mode Microplate Reader (BioTek). Percent max signal was calculated based on signal generated by IL-33 in the absence of any serum.

ROC Analysis of REAP Score Performance.

Orthogonal validation data for the receiver operator curve (ROC) analysis was obtained by ELISA, LIPS, or clinical autoantibody tests. For ELISA and LIPS, valid reactivities were defined as those 3 standard deviations above the healthy donor average for a given protein in each assay. ROC analysis was performed using 247 test pairs across 25 different proteins.

Statistical Analysis.

Statistical details of experiments can be found in the figure legends. All error bars in figures indicate standard deviation. Data analysis was performed using R, Python, Excel, and GraphPad Prism.

In summary, autoantibodies targeting extracellular proteins are known to mediate autoimmune diseases and paraneoplastic syndromes in cancer. However, discovery of new autoantibodies against extracellular (transmembrane and secreted) proteins in high throughput remained difficult due to a lack of methods for screening the thousands of extracellular proteins in the human proteome. The autoantibodies can mediate new forms of autoimmune disease, predict response to therapy, or mediate toxicity or responses in cancer in response to immune-modifying checkpoint blockade therapies.

The essence of the invention is the discovery of extracellular antibody targets using a yeast-displayed library of proteins and next-generation sequencing, which enabled high-throughput interrogation of natively folded proteins by total human serum. Moreover, yeast cell display is a technique well-suited to display of human extracellular proteins, and amenable to high-throughput screening due to the ease of handling yeast. This allowed unbiased assessment of autoantibody repertoires in any human patient or healthy population at a previously unattainable scale and cost. Furthermore, it was accomplished by (Step I) using a yeast-displayed library of extracellular antigens as a substrate to interrogate whole sero-reactivities, (Step II) optimizing an antibody isolation protocol, (Step III) staining and selecting conditions for yeast cell selection with total serum antibodies, and (Step IV) next-generation sequencing pipelines to identify the antigen targets. Consequently, this technique enabled screening against thousands of candidate antigens simultaneously

More specifically, (Step I) standard methods were used to identify and amplify the ectodomains of human extracellular proteins, and individually transformed them into standard yeast-display strains for fusion to cell-wall associated proteins in yeast. A random nucleotide barcode was additionally incorporated into the display vector to enable tracking of proteins by next-generation sequencing. These individual strains were then pooled to create a single library encompassing all proteins of interest.

(Step II) Antibodies were isolated from human serum by affinity purification. For example, antibodies were purified with Protein A or Protein G, using either magnetic or agarose beads, and via standard methods. If other isotypes of antibody besides IgG were desired, appropriate affinity purification methods were used in place of Protein A or Protein G. After antibody purification, yeast-reactive antibodies present in human serum were removed by incubation with parental yeast cell strains and filtration. The final elution was suitable for yeast cell staining and selection.

(Step III) Yeast cell were stained with a normalized concentration of purified, non-yeast-reactive antibody from 1-10 μg per reaction. Stained yeast cell were identified with any appropriate secondary antibody recognizing immunoglobulins of the isotype used, such as a biotinylated or fluorescently labeled anti-immunoglobulin antibody. Stained yeast cell were then selected via magnetic separation using standard methods and appropriate magnetic reagents or by FACS. Stained yeast cell were also directly selected with appropriate anti-immunoglobulin magnetic particles. Selected yeast cell were expanded following selection and their DNA isolated via standard methods.

(Step IV) Yeast cell DNA was amplified and prepared for next-generation sequencing by standard methods appropriate from the next-generation sequencing method of interest (e.g. Illumina sequencing-by-synthesis). The frequencies of each protein were measured in the initial library and in all samples following selection, by tabulating the frequencies of all barcodes corresponding to an individual protein. An enrichment score was calculated based on the total enrichment of each protein in each sample and the fraction of associated barcodes that enrich. Different thresholds were applied to this enrichment score depending on the desired level of sensitivity or specificity. Proteins with scores above a particular threshold were predicted as candidate autoantigens.

Accordingly, the primary novel feature of the present invention is, in part, the design of the display library to improve display success and quality of results over previous methods, such as shotgun cDNA library preparations. A high-quality curation of the library greatly improved the specificity and sensitivity by removing out-of-frame or truncated protein products. Additional novelty comes, in part, from the next-generation sequencing approach and analytical methods, which increased confidence in the predicted candidate autoantigens. Finally, the optimized method for staining and selection was more amenable to high-throughput screening of hundreds of serum samples due to applicability to 96-well formats.

As described above, the herein described technique used a more advanced library with higher display success rates that can cover the full complement of well-folded ectodomains in the human proteome. It was additionally scalable, sensitive, and amenable to high-throughput screening and even automation. Compared to the gold-standard approaches, such as protein arrays, it was found that known and novel autoantibody responses can be detected that were previously undectable. As the technique was amenable to high-throughput screening approaches and requires small samples volumes, it can rapidly query large patient cohorts for a small fraction of the cost of previous methods, such as protein arrays.

### Diagnostic or Prognostic Antibodies

TABLE 2
List of Diseases or Disorders and the Corresponding Abbreviations

| Abbreviation | Full Name |
|---|---|
| AAV | ANCA-Associated vasculitis |
| APECED | Autoimmune Polyendocrinopathy Candidiasis |
| | Ecto-Dermal Dystrophy |
| APS | Antiphospholipid Antibody Syndome |
| CIDP | Chronic Inflammatory Demyelinating |
| | Polyradiculoneuropathy |
| COVID-19 | Coronavirus Disease 2019 |
| DIL | Drug-Induced Lupus |
| DM | Dermatomyositis |
| KT | Kidney Transplant |
| Malaria | Malaria |
| MG | Myasthenia Gravis |
| MM | Malignant Melanoma |
| NMO | Neuromyelitis Optica |
| NSCLC | Non-Small Cell Lung Cancer |
| PANDAS | Pediatric Autoimmune Neuropsychiatric |
| | Disorders Associated with Streptococcal |
| | Infections |
| SLE | Systemic Lupus Erythematosus |
| SS | Sjogren's Syndrome |
| SSC | Scleroderma |
| SUSAC | Susac Syndrome |

TABLE 3
List of Autoantigens and the Corresponding Diseases or Disorders

| Disease | Target |
|---|---|
| AAV | EDIL3 |
| AAV | LY6H |
| AAV | TREM2 |
| APECED | ACRV1 |
| APECED | ADM2 |
| APECED | AFP |
| APECED | APOA4 |
| APECED | APOO |
| APECED | BPIFA1 |
| APECED | BPIFA2 |
| APECED | BTN1A1 |
| APECED | C5orf64 |
| APECED | CASQ1 |
| APECED | CCDC47 |
| APECED | CCL11 |
| APECED | CCL15 |
| APECED | CCL17 |
| APECED | CCL18 |
| APECED | CCL7 |
| APECED | CCL8 |
| APECED | CDSN |
| APECED | CELA2B |
| APECED | CLCC1 |
| APECED | CLPS |
| APECED | CLSTN1 |
| APECED | CLU |
| APECED | CNPY2 |
| APECED | CNPY3 |
| APECED | CP |
| APECED | CSHL1 |
| APECED | CSN2 |
| APECED | CSPG5 |
| APECED | CST4 |
| APECED | CST5 |
| APECED | CST6 |
| APECED | CTSG |
| APECED | DEFA5 |
| APECED | DKK1 |
| APECED | DRAXIN |
| APECED | ECSCR |
| APECED | EPHA4 |
| APECED | EREG |
| APECED | FAM19A4 |
| APECED | FAM3A |
| APECED | FGF1 |
| APECED | FGFR2 |
| APECED | FKBP14 |
| APECED | GFRAL |
| APECED | GIF |
| APECED | GPHB5 |
| APECED | HCRTR2 |
| APECED | HSPA13 |
| APECED | IBSP |
| APECED | IFNA13 |
| APECED | IFNA14 |
| APECED | IFNA17 |
| APECED | IFNA2 |
| APECED | IFNA5 |
| APECED | IFNA6 |
| APECED | IFNA8 |
| APECED | IFNL2 |
| APECED | IFNW1 |
| APECED | IGF1 |
| APECED | IGFBP1 |
| APECED | IGSF4B |
| APECED | IL17A |
| APECED | IL17F |
| APECED | IL22 |
| APECED | IL22RA2 |
| APECED | IL28B |
| APECED | IL5 |
| APECED | IL6 |
| APECED | KAL1 |
| APECED | KLK2 |
| APECED | LAIR2 |
| APECED | LCN1 |
| APECED | LEG1 |
| APECED | LIPF |
| APECED | LRIT3 |
| APECED | LRRC3B |
| APECED | LY6H |
| APECED | MMP1 |
| APECED | MMP7 |
| APECED | MPZL3 |
| APECED | MSMP |
| APECED | MSR1 |
| APECED | OBP2A |
| APECED | ODAPH |
| APECED | OPN4 |
| APECED | OTOL1 |
| APECED | OTOR |
| APECED | PANX3 |
| APECED | PAP |
| APECED | PDGFB |
| APECED | PDILT |
| APECED | PGC |
| APECED | PLA2G10 |
| APECED | PLA2G2E |
| APECED | PLAC9 |
| APECED | PLVAP |
| APECED | PMCH |
| APECED | PNLIP |
| APECED | PNLIPRP1 |
| APECED | PNLIPRP2 |
| APECED | PPT1 |
| APECED | PRG3 |
| APECED | PRLR |
| APECED | PRRG1 |
| APECED | PRRG3 |
| APECED | PRRT1 |
| APECED | PRRT3 |
| APECED | PSAP |
| APECED | PTPRN2 |
| APECED | PTPRR |
| APECED | RAMP2 |
| APECED | REG1A |
| APECED | REG3G |
| APECED | REG4 |
| APECED | RNASE8 |
| APECED | RTBDN |
| APECED | SERPINE1 |
| APECED | SLC2A10 |
| APECED | SLC41A2 |
| APECED | SMR3A |
| APECED | SOSTDC1 |
| APECED | SPACA7 |
| APECED | SPAG11B |
| APECED | SPINK1 |
| APECED | SPINK4 |
| APECED | SPINK8 |
| APECED | SRGN |
| APECED | SYCN |
| APECED | TEPP |
| APECED | TEX264 |
| APECED | TFF2 |
| APECED | TGFA |
| APECED | TM4SF6 |
| APECED | TM9SF3 |
| APECED | TMEM119 |
| APECED | TMEM149 |
| APECED | TNFRSF12A |
| APECED | TSLP |
| APECED | TXNDC12 |
| APECED | VSTM2A |
| APS | IL6R |
| APS | IFNA13 |
| APS | IFNA14 |
| APS | IFNA17 |
| APS | IFNA2 |
| APS | IFNA5 |
| APS | IFNA6 |
| APS | IFNA8 |
| APS | IL6R |
| CIDP | CXCL1 |
| CIDP | CXCL2 |
| CIDP | CXCL3 |
| CIDP | PDGFB |
| CIDP | TMEM149 |
| CIDP | CD74 |
| CIDP | CXCL13 |
| COVID-19 | APOO |
| COVID-19 | OPRL1 |
| COVID-19 | IFNA14 |
| COVID-19 | MIA2 |
| COVID-19 | FKBP2 |
| COVID-19 | GPR1 |
| COVID-19 | IL29 |
| COVID-19 | PTPRR |
| COVID-19 | RCN2 |
| COVID-19 | IFNA13 |
| COVID-19 | IFNW1 |
| COVID-19 | IL1A |
| COVID-19 | TSPAN9 |
| COVID-19 | SHISA7 |
| COVID-19 | IFNA17 |
| COVID-19 | LEP |
| COVID-19 | CALU |
| COVID-19 | SSPN |
| COVID-19 | LPAL2 |
| COVID-19 | OBP2B |
| COVID-19 | CST5 |
| COVID-19 | IL6 |
| COVID-19 | CCDC47 |
| COVID-19 | ACRV1 |
| COVID-19 | PGA3 |
| COVID-19 | LRRC8C |
| COVID-19 | PMCH |
| COVID-19 | GPR6 |
| COVID-19 | CSF2 |
| COVID-19 | RCN3 |
| COVID-19 | LYSMD4 |
| COVID-19 | CD99 |
| COVID-19 | IFNA5 |
| COVID-19 | IFNL2 |
| COVID-19 | CXCL9 |
| COVID-19 | SLC41A2 |
| COVID-19 | EPYC |
| COVID-19 | DUOXA1 |
| COVID-19 | LACRT |
| COVID-19 | CNPY2 |
| COVID-19 | KLK8 |
| COVID-19 | MZB1 |
| COVID-19 | LYG2 |
| COVID-19 | MUCL3 |
| COVID-19 | LALBA |
| COVID-19 | ZG16B |
| COVID-19 | ODAM |
| COVID-19 | PILRA |
| COVID-19 | HRC |
| COVID-19 | PPBP |
| COVID-19 | CSPG5 |
| COVID-19 | PTPRN2 |
| COVID-19 | CST4 |
| COVID-19 | FAM168B |
| COVID-19 | TNFRSF17 |
| COVID-19 | OTOS |
| COVID-19 | SPINK9 |
| COVID-19 | KLRC2 |
| COVID-19 | IFNA8 |
| COVID-19 | TMEM119 |
| COVID-19 | CSAG1 |
| COVID-19 | OTOR |
| COVID-19 | KCT2 |
| COVID-19 | PGA4 |
| COVID-19 | SPINK4 |
| COVID-19 | FCGR2A |
| COVID-19 | CNPY3 |
| COVID-19 | NEGR1 |
| COVID-19 | ERP27 |
| COVID-19 | AGRP |
| COVID-19 | PRR27 |
| COVID-19 | MCFD2 |
| COVID-19 | IGFBP6 |
| COVID-19 | IFNA2 |
| COVID-19 | LGALS3 |
| COVID-19 | SPOCK1 |
| COVID-19 | KCNV2 |
| COVID-19 | HCRTR2 |
| COVID-19 | LECT2 |
| COVID-19 | PLA2G2E |
| COVID-19 | FAM19A3 |
| COVID-19 | SPACA7 |
| COVID-19 | NENF |
| COVID-19 | IL6R |
| COVID-19 | SPX |
| COVID-19 | IGFBP1 |
| COVID-19 | SRGN |
| COVID-19 | LAIR2 |
| COVID-19 | CPXM2 |
| COVID-19 | CCL17 |
| COVID-19 | TUSC5 |
| COVID-19 | LOC644613 |
| COVID-19 | TNFRSF21 |
| COVID-19 | GPR77 |
| COVID-19 | C2orf40 |
| COVID-19 | C5A |
| COVID-19 | IFNA6 |
| COVID-19 | SPP1 |
| COVID-19 | SERPINA3 |
| COVID-19 | OXTR |
| COVID-19 | KLRC1 |
| COVID-19 | SEMG2 |
| COVID-19 | APOH |
| COVID-19 | PRRG1 |
| COVID-19 | BTC |
| COVID-19 | MSLN |
| COVID-19 | FAM19A2 |
| COVID-19 | CXCL1 |
| COVID-19 | PRSS55 |
| COVID-19 | SLCO2B1 |
| COVID-19 | BTN1A1 |
| COVID-19 | COV2-RBD |
| COVID-19 | OS9 |
| COVID-19 | PGLYRP1 |
| COVID-19 | DKK3 |
| COVID-19 | TOR1B |
| COVID-19 | CST1 |
| COVID-19 | LRRC8D |
| COVID-19 | ACKR1 |
| COVID-19 | COL8A1 |
| COVID-19 | CXCL3 |
| COVID-19 | ODAPH |
| COVID-19 | PIANP |
| COVID-19 | PSORS1C2 |
| COVID-19 | RNASE10 |
| COVID-19 | CXCR7 |
| COVID-19 | PLVAP |
| COVID-19 | CDSN |
| COVID-19 | SDF2L1 |
| COVID-19 | TFF2 |
| COVID-19 | HSPA13 |
| COVID-19 | CXCR5 |
| COVID-19 | C5orf64 |
| COVID-19 | EPO |
| COVID-19 | GNLY |
| COVID-19 | OPRM1 |
| COVID-19 | TGFA |
| COVID-19 | SLC2A10 |
| COVID-19 | CXCL13 |
| COVID-19 | CD99L2 |
| COVID-19 | AGER |
| COVID-19 | CGA |
| COVID-19 | CRTAM |
| COVID-19 | SLC1A1 |
| COVID-19 | CDH19 |
| COVID-19 | GPR25 |
| COVID-19 | CCL8 |
| COVID-19 | SERPINI1 |
| COVID-19 | SPINK8 |
| COVID-19 | SLPI |
| COVID-19 | HRH3 |
| COVID-19 | TMEM149 |
| COVID-19 | CD38 |
| COVID-19 | REG4 |
| COVID-19 | IGFBP5 |
| COVID-19 | FKBP7 |
| COVID-19 | GRM5 |
| COVID-19 | CXCR3 |
| COVID-19 | PTHLH |
| COVID-19 | LY6K |
| COVID-19 | PLAC9 |
| COVID-19 | LPL |
| COVID-19 | CCKAR |
| COVID-19 | RTN4R |
| COVID-19 | GYPA |
| COVID-19 | TMED1 |
| COVID-19 | DRAXIN |
| COVID-19 | CCL13 |
| COVID-19 | LRRC8A |
| COVID-19 | ANGPTL4 |
| COVID-19 | NPPC |
| COVID-19 | IL22 |
| COVID-19 | CCL21 |
| COVID-19 | RCN1 |
| COVID-19 | CD74 |
| COVID-19 | FGF17 |
| COVID-19 | PAEP |
| COVID-19 | CNPY4 |
| COVID-19 | APOC3 |
| COVID-19 | SPINK1 |
| COVID-19 | AZGP1 |
| COVID-19 | STC2 |
| COVID-19 | S1PR4 |
| COVID-19 | IBSP |
| COVID-19 | CEACAM18 |
| COVID-19 | SLC38A4 |
| COVID-19 | CSN2 |
| COVID-19 | VSIG2 |
| COVID-19 | ENSP00000381830 |
| COVID-19 | CSHL1 |
| COVID-19 | CASQ1 |
| COVID-19 | XG |
| COVID-19 | ENDOU |
| COVID-19 | RAET1L |
| COVID-19 | COL10A1 |
| COVID-19 | PTH |
| COVID-19 | SLC15A1 |
| COVID-19 | SLC6A2 |
| COVID-19 | PRRT1 |
| COVID-19 | CLCC1 |
| COVID-19 | F2R |
| COVID-19 | JTB |
| COVID-19 | TGOLN2 |
| COVID-19 | CCL16 |
| COVID-19 | MIA |
| COVID-19 | TNF |
| COVID-19 | TMEM91 |
| COVID-19 | RTBDN |
| COVID-19 | MPL |
| COVID-19 | RSPO1 |
| COVID-19 | RSPO3 |
| COVID-19 | PRSS3 |
| COVID-19 | GPR17 |
| COVID-19 | CCR9 |
| COVID-19 | GP6 |
| COVID-19 | PRH1; |
| COVID-19 | EQTN |
| COVID-19 | RNF43 |
| COVID-19 | SPN |
| COVID-19 | IGSF4B |
| COVID-19 | CFD |
| COVID-19 | SPACA5 |
| COVID-19 | CHGA |
| COVID-19 | UNQ6190/PRO20217 |
| COVID-19 | APOA1 |
| COVID-19 | PRG3 |
| COVID-19 | SLC2A2 |
| COVID-19 | CCL11 |
| COVID-19 | TSLP |
| COVID-19 | SMOC2 |
| COVID-19 | HTR5 |
| COVID-19 | PRAP1 |
| COVID-19 | LY6H |
| COVID-19 | IMPG1 |
| COVID-19 | TNFRSF12A |
| COVID-19 | SSTR2 |
| COVID-19 | IGFBP3 |
| COVID-19 | PRLR |
| COVID-19 | PRR4 |
| COVID-19 | IL13 |
| COVID-19 | HCTR1 |
| COVID-19 | IGF1 |
| COVID-19 | CD300E |
| COVID-19 | LINC00305 |
| COVID-19 | SPESP1 |
| COVID-19 | FRZB |
| COVID-19 | IL28B |
| COVID-19 | MMP9 |
| COVID-19 | GAST |
| COVID-19 | FGF1 |
| COVID-19 | IL15RA |
| COVID-19 | CCR10 |
| COVID-19 | VEGFB |
| COVID-19 | SERPINE1 |
| COVID-19 | EXOC3-AS1 |
| COVID-19 | PRRT3 |
| COVID-19 | NETO1 |
| COVID-19 | VSTM2B |
| COVID-19 | CCR4 |
| COVID-19 | APP |
| COVID-19 | AMTN |
| COVID-19 | CXCL6 |
| COVID-19 | NINJ1 |
| COVID-19 | KLK9 |
| COVID-19 | SDF4 |
| COVID-19 | CPE |
| COVID-19 | AMELX |
| COVID-19 | DCD |
| COVID-19 | ANTXRL |
| COVID-19 | CCR2 |
| COVID-19 | PCSK1 |
| COVID-19 | QRFP |
| COVID-19 | RGMB |
| COVID-19 | NPY2R |
| COVID-19 | IGFBP7 |
| COVID-19 | SLC2A12 |
| COVID-19 | PPT1 |
| COVID-19 | CCL7 |
| COVID-19 | JCHAIN |
| COVID-19 | ADCYAP1 |
| COVID-19 | PDZD11 |
| COVID-19 | CP |
| COVID-19 | MANF |
| COVID-19 | GZMA |
| COVID-19 | TXNDC12 |
| COVID-19 | PGC |
| COVID-19 | ACVR1 |
| COVID-19 | WFDC13 |
| COVID-19 | SFRP4 |
| COVID-19 | REG1A |
| COVID-19 | GPR37 |
| COVID-19 | NOPE |
| COVID-19 | Cllorf94 |
| COVID-19 | SCARA5 |
| COVID-19 | GPR19 |
| COVID-19 | EMC7 |
| COVID-19 | CCL15 |
| COVID-19 | CA4 |
| COVID-19 | RNASE8 |
| COVID-19 | MLN |
| COVID-19 | UNQ9165/PRO28630 |
| COVID-19 | NTRK3 |
| COVID-19 | TREML1 |
| COVID-19 | CDH15 |
| COVID-19 | SMR3A |
| COVID-19 | DKK1 |
| COVID-19 | OXER1 |
| COVID-19 | FAM24B |
| COVID-19 | CRLF1 |
| COVID-19 | PDIA6 |
| COVID-19 | PLA2G12B |
| COVID-19 | FGF7 |
| COVID-19 | ZP4 |
| COVID-19 | BAMBI |
| COVID-19 | GKN2 |
| COVID-19 | IGFBPL1 |
| COVID-19 | MMP7 |
| COVID-19 | MANSC4 |
| COVID-19 | APOA4 |
| COVID-19 | SUSD6 |
| COVID-19 | CELA1 |
| COVID-19 | IGLL1 |
| COVID-19 | IL9 |
| COVID-19 | MADCAM1 |
| COVID-19 | NPBW1 |
| COVID-19 | HAVCR1 |
| COVID-19 | ITPRIPL1 |
| COVID-19 | SOST |
| COVID-19 | LHFPL1 |
| COVID-19 | SDC3 |
| COVID-19 | SEMG1 |
| COVID-19 | C1QB |
| COVID-19 | ASIP |
| COVID-19 | CCL18 |
| COVID-19 | LHFPL5 |
| COVID-19 | IGFL2 |
| COVID-19 | FGFRL1 |
| COVID-19 | EFNB2 |
| COVID-19 | C2orf66 |
| COVID-19 | MFAP3 |
| COVID-19 | C6orf15 |
| COVID-19 | OPN4 |
| COVID-19 | NOV |
| COVID-19 | GNS |
| COVID-19 | FKBP14 |
| COVID-19 | CELA2B |
| COVID-19 | C9 |
| COVID-19 | VWC2L |
| COVID-19 | BMPR2 |
| COVID-19 | CSH2 |
| COVID-19 | IL1RAP |
| COVID-19 | C1QTNF2 |
| COVID-19 | SLC10A4 |
| COVID-19 | IL16 |
| COVID-19 | LRIT3 |
| COVID-19 | GRN |
| COVID-19 | NIPAL4 |
| COVID-19 | GNRH1 |
| COVID-19 | ATP4B |
| COVID-19 | APLP2 |
| COVID-19 | TMEM123 |
| COVID-19 | IL3 |
| COVID-19 | PDGFA |
| COVID-19 | EVI2B |
| COVID-19 | NGFR |
| COVID-19 | PROK1 |
| COVID-19 | SOSTDC1 |
| COVID-19 | FLJ36131 |
| COVID-19 | EREG |
| COVID-19 | TNFRSF9 |
| COVID-19 | LYG1 |
| COVID-19 | SLCO4C1 |
| COVID-19 | GUCA2A |
| COVID-19 | FAM19A5 |
| COVID-19 | IL21 |
| COVID-19 | FCMR |
| COVID-19 | CADM2 |
| COVID-19 | CSF3 |
| COVID-19 | CA11 |
| COVID-19 | NTRK2 |
| COVID-19 | CRELD2 |
| COVID-19 | GPR120 |
| COVID-19 | C9orf135 |
| COVID-19 | SLC1A5 |
| COVID-19 | SYCN |
| COVID-19 | COL9A3 |
| COVID-19 | ADRA1D |
| COVID-19 | GLB1 |
| COVID-19 | SV2C |
| COVID-19 | DKFZp686O24166 |
| COVID-19 | PRSS3P2 |
| COVID-19 | KIRREL3 |
| COVID-19 | VSTM2A |
| COVID-19 | GCG |
| COVID-19 | SERPINE2 |
| COVID-19 | EDA2R |
| COVID-19 | CPAMD8 |
| COVID-19 | SCN3B |
| COVID-19 | OXT |
| COVID-19 | CD3E |
| COVID-19 | INSL3 |
| COVID-19 | CALY |
| COVID-19 | GHSR |
| COVID-19 | SCGB1D1 |
| COVID-19 | C6 |
| COVID-19 | CLDN2 |
| COVID-19 | MUC7 |
| COVID-19 | KISS1 |
| COVID-19 | ULBP2 |
| COVID-19 | CLDN7 |
| COVID-19 | IGFBP2 |
| COVID-19 | EFNB3 |
| COVID-19 | NXPH1 |
| COVID-19 | GHRHR |
| COVID-19 | LILRA4 |
| COVID-19 | OTOL1 |
| COVID-19 | EFNB1 |
| COVID-19 | FGFBP3 |
| COVID-19 | GPR63 |
| COVID-19 | PRRG4 |
| COVID-19 | MUCL1 |
| COVID-19 | XCL1 |
| COVID-19 | TMEM120A |
| COVID-19 | TMEM108 |
| COVID-19 | IL1F5 |
| COVID-19 | MSMP |
| COVID-19 | CXCL12 |
| COVID-19 | GNPTG |
| COVID-19 | SDC4 |
| COVID-19 | FZD9 |
| COVID-19 | CCL4L1 |
| COVID-19 | GPRC6A |
| COVID-19 | GPR156 |
| COVID-19 | ITIH3 |
| COVID-19 | RAMP2 |
| COVID-19 | TNFRSF11A |
| COVID-19 | DKK2 |
| COVID-19 | SPINK13 |
| COVID-19 | SDCBP |
| COVID-19 | CD8B2 |
| COVID-19 | CTSG |
| COVID-19 | CST2 |
| COVID-19 | EDDM3B |
| COVID-19 | CLTRN |
| COVID-19 | PLA2G10 |
| COVID-19 | DCN |
| COVID-19 | DAG1 |
| COVID-19 | CXCL16 |
| COVID-19 | CCRL2 |
| COVID-19 | DEFB108B |
| COVID-19 | MRGPRF |
| COVID-19 | FCRL3 |
| COVID-19 | NPS |
| COVID-19 | OBP2A |
| COVID-19 | ACKR2 |
| COVID-19 | GRM2 |
| COVID-19 | FAM174A |
| COVID-19 | MSR1 |
| COVID-19 | NOG |
| COVID-19 | TMEM102 |
| COVID-19 | LAIR1 |
| COVID-19 | IL22RA2 |
| COVID-19 | SPACA3 |
| COVID-19 | WIF1 |
| COVID-19 | F13B |
| COVID-19 | LRTM1 |
| COVID-19 | ERVH48-1 |
| COVID-19 | CCL2 |
| COVID-19 | TFF1 |
| COVID-19 | ADM2 |
| COVID-19 | IFITM10 |
| COVID-19 | HSD11B1L |
| COVID-19 | AXL |
| COVID-19 | FMR1NB |
| COVID-19 | C6orf25 |
| COVID-19 | OPN3 |
| COVID-19 | MUC13 |
| COVID-19 | CCL28 |
| COVID-19 | CCL26 |
| COVID-19 | PTN |
| COVID-19 | SLC39A8 |
| COVID-19 | FGF21 |
| COVID-19 | TIMD4 |
| COVID-19 | NPTX2 |
| COVID-19 | IL17RD |
| COVID-19 | PAPLN |
| COVID-19 | TMEM219 |
| COVID-19 | CYB5D2 |
| COVID-19 | IL1B |
| COVID-19 | FSTL1 |
| COVID-19 | PTPRJ |
| COVID-19 | NPY1R |
| COVID-19 | CLDN18 |
| COVID-19 | FLT3LG |
| COVID-19 | C17orf99 |
| COVID-19 | SLC6A5 |
| COVID-19 | AIMP1 |
| COVID-19 | TNFRSF8 |
| COVID-19 | CD248 |
| COVID-19 | TM9SF3 |
| COVID-19 | FCGR2C |
| COVID-19 | MPZL3 |
| COVID-19 | OSTN |
| COVID-19 | SPARCL1 |
| COVID-19 | TMPRSS11D |
| COVID-19 | KLK7 |
| COVID-19 | GDPD3 |
| COVID-19 | IL34 |
| COVID-19 | BTNL8 |
| COVID-19 | ASTL |
| COVID-19 | CLDN19 |
| COVID-19 | SCG5 |
| COVID-19 | PSAP |
| COVID-19 | PRRG3 |
| COVID-19 | PLA2G12A |
| COVID-19 | LCN1 |
| COVID-19 | LRRTM2 |
| COVID-19 | FAM3D |
| COVID-19 | PTGS2 |
| COVID-19 | FCRLB |
| COVID-19 | CST8 |
| COVID-19 | ANGPTL5 |
| COVID-19 | OPRK1 |
| COVID-19 | APOD |
| COVID-19 | ADM |
| COVID-19 | CLU |
| COVID-19 | PANX3 |
| COVID-19 | SLC52A3 |
| COVID-19 | VASN |
| COVID-19 | CMKLR1 |
| COVID-19 | BGLAP |
| COVID-19 | IL4 |
| COVID-19 | IL18BP |
| COVID-19 | ACVRL1 |
| COVID-19 | FLRT3 |
| COVID-19 | FAM234A |
| COVID-19 | CPVL |
| COVID-19 | GPR3 |
| COVID-19 | LMBRD2 |
| COVID-19 | TMEM169 |
| COVID-19 | LRRC8B |
| COVID-19 | INSL6 |
| COVID-19 | PDCD1 |
| COVID-19 | EMC10 |
| COVID-19 | IL18RAP |
| COVID-19 | NRN1 |
| COVID-19 | TRABD2A |
| COVID-19 | SSBP3-AS1 |
| COVID-19 | IL17C |
| COVID-19 | LGALS1 |
| COVID-19 | MDK |
| COVID-19 | WFDC1 |
| COVID-19 | NRN1L |
| COVID-19 | TNFRSF1B |
| COVID-19 | HNRNPA2B1 |
| COVID-19 | DKKL1 |
| COVID-19 | NTSR1 |
| COVID-19 | IL32 |
| COVID-19 | FAM24A |
| COVID-19 | SGCA |
| COVID-19 | IL1RN |
| COVID-19 | LY6D |
| COVID-19 | HSD17B7 |
| COVID-19 | SCG3 |
| COVID-19 | TNFRSF4 |
| COVID-19 | CCL22 |
| COVID-19 | XK |
| COVID-19 | RETN |
| COVID-19 | GALP |
| COVID-19 | FGL2 |
| COVID-19 | PDGFB |
| COVID-19 | CTF1 |
| COVID-19 | C8G |
| COVID-19 | EBI3 |
| COVID-19 | EDIL3 |
| COVID-19 | TRABD2B |
| COVID-19 | GP5 |
| COVID-19 | CLEC2B |
| COVID-19 | SEMA6C |
| COVID-19 | CLDN9 |
| COVID-19 | CSN3 |
| COVID-19 | TRH |
| COVID-19 | CCL25 |
| COVID-19 | APOE |
| COVID-19 | IER3 |
| COVID-19 | DHRS7C |
| COVID-19 | C19orf18 |
| COVID-19 | MCHR1 |
| COVID-19 | CHRDL2 |
| COVID-19 | FGF18 |
| COVID-19 | PINLYP |
| COVID-19 | MFAP2 |
| COVID-19 | C11orf44 |
| COVID-19 | CXCL17 |
| COVID-19 | ART1 |
| COVID-19 | LILRB4 |
| COVID-19 | DUOXA2 |
| COVID-19 | CSN1S1 |
| COVID-19 | PEBP4 |
| COVID-19 | RTN4RL1 |
| COVID-19 | SCGB2A2 |
| COVID-19 | TGFBR3L |
| COVID-19 | UCMA |
| COVID-19 | RAET1E |
| COVID-19 | PKD2L1 |
| COVID-19 | ACVR1B |
| COVID-19 | AVPR1A |
| COVID-19 | HEPACAM2 |
| COVID-19 | P4HB |
| COVID-19 | AJAP1 |
| COVID-19 | MOG |
| COVID-19 | EPHA4 |
| COVID-19 | BAGE3 |
| COVID-19 | CPA6 |
| COVID-19 | FSTL3 |
| COVID-19 | ARTN |
| COVID-19 | LRRN4 |
| COVID-19 | BRINP3 |
| COVID-19 | EPOR |
| COVID-19 | NRG1 |
| COVID-19 | MEGF9 |
| COVID-19 | MFSD2A |
| COVID-19 | SERPINA13P |
| COVID-19 | CLDN10 |
| COVID-19 | SCG2 |
| COVID-19 | ENDOD1 |
| COVID-19 | TMEFF1 |
| COVID-19 | F12 |
| COVID-19 | NUCB1 |
| COVID-19 | CEACAM19 |
| COVID-19 | B2M |
| COVID-19 | FETUB |
| COVID-19 | UNQ5830/PRO19650/PRO19816 |
| COVID-19 | DNASE1L2 |
| COVID-19 | CLEC-6 |
| COVID-19 | IL20RB |
| COVID-19 | CHRNA9 |
| COVID-19 | APOC2 |
| COVID-19 | SLC1A4 |
| COVID-19 | MC5R |
| COVID-19 | COLQ |
| COVID-19 | IMPG2 |
| COVID-19 | VTCN1 |
| COVID-19 | DEFB126 |
| COVID-19 | TMEM41A |
| COVID-19 | SDC1 |
| COVID-19 | IL15 |
| COVID-19 | BPIFA3 |
| COVID-19 | LTBR |
| COVID-19 | CELA3B |
| COVID-19 | MPEG1 |
| COVID-19 | ADAMTS16 |
| COVID-19 | S1PR3 |
| COVID-19 | GPR37L1 |
| COVID-19 | LAS2 |
| COVID-19 | SNCA |
| COVID-19 | SLC6A11 |
| COVID-19 | LYPD6B |
| COVID-19 | FLJ46089 |
| COVID-19 | CXCL11 |
| COVID-19 | FAM3A |
| COVID-19 | NINJ2 |
| COVID-19 | HBEGF |
| COVID-19 | C9orf47 |
| COVID-19 | CST6 |
| COVID-19 | CRTAC1 |
| COVID-19 | CD14 |
| COVID-19 | LAG3 |
| COVID-19 | LILRB2 |
| COVID-19 | SLC22A31 |
| COVID-19 | HS3ST1 |
| COVID-19 | GIF |
| COVID-19 | NLGN4X |
| COVID-19 | NOTCH2NL |
| COVID-19 | MFGE8 |
| COVID-19 | RXFP3 |
| COVID-19 | LCAT |
| COVID-19 | TRPC3 |
| COVID-19 | MARCO |
| COVID-19 | IGLL5 |
| COVID-19 | GKN1 |
| COVID-19 | CST7 |
| COVID-19 | FMOD |
| DIL | CXCL1 |
| DIL | TNF |
| DIL | TSLP |
| DM | CD81 |
| MG | CXCL2 |
| MG | PDGFB |
| MC | REG4 |
| MG | CCL22 |
| MG | CCL2 |
| MM | PLA2G2E |
| MM | SPX |
| MM | KCNK1 |
| MM | TNFRSF21 |
| MM | CLDN19 |
| MM | MMP7 |
| MM | NGRN |
| MM | PSORS1C2 |
| MM | FGFBP3 |
| MM | VEGFB |
| MM | LOC644613 |
| MM | C9 |
| MM | COLEC12 |
| MM | SLC38A4 |
| MM | SOST |
| MM | SLC41A2 |
| MM | MOG |
| MM | DNASE2 |
| MM | FMR1NB |
| MM | ODAPH |
| MM | LY6H |
| MM | OPN4 |
| MM | PRRT3 |
| MM | CCL18 |
| MM | TMEM41A |
| MM | APOC3 |
| MM | LGALS1 |
| MM | SSPN |
| MM | IL21 |
| MM | ACRV1 |
| MM | TFF2 |
| MM | AGER |
| MM | DKK1 |
| MM | CST9L |
| MM | EPHA5 |
| MM | PDIA6 |
| MM | DHRS4L2 |
| MM | MZB1 |
| MM | EVI2B |
| MM | C19orf18 |
| MM | SPOCK1 |
| MM | SCN3B |
| MM | CCL11 |
| MM | HCRTR2 |
| MM | MFSD2A |
| MM | IFNA17 |
| MM | LILRB1 |
| MM | SHISA5 |
| MM | GNRH2 |
| MM | COL8A1 |
| MM | TGFA |
| MM | ACP5 |
| MM | SMR3A |
| MM | PSAPL1 |
| MM | ZG16B |
| MM | GYPA |
| MM | IGLL5 |
| MM | CCL22 |
| MM | MANSC4 |
| MM | DNAJC3 |
| MM | TNFRSF8 |
| MM | ARTN |
| MM | NEGR1 |
| MM | CHRNA9 |
| MM | APOO |
| MM | UNQ6190/PRO20217 |
| MM | CST6 |
| MM | CD164L2 |
| MM | ASTN2 |
| MM | KAL1 |
| MM | TRPC3 |
| MM | IGFBP6 |
| MM | MLN |
| MM | IL15RA |
| MM | PPT1 |
| MM | FGF1 |
| MM | PRRG3 |
| MM | IFNA5 |
| MM | C9orf47 |
| MM | FAM3A |
| MM | LCN12 |
| MM | IFNL2 |
| MM | SECTM1 |
| MM | PMCH |
| MM | BMPR2 |
| MM | FAM19A5 |
| MM | PNLIPRP1 |
| MM | IL13RA1 |
| MM | LCN2 |
| MM | LAIR2 |
| MM | ERVK13-1 |
| MM | SLPI |
| MM | OPTC |
| MM | SPN |
| MM | CXCL17 |
| MM | CASQ1 |
| MM | TMEM108 |
| MM | MCFD2 |
| MM | IL19 |
| MM | SLC6A5 |
| MM | POMC |
| MM | ACVRL1 |
| MM | IL5 |
| MM | PRL |
| MM | OVGP1 |
| MM | LCN15 |
| MM | ITPRIPL1 |
| MM | TMEM91 |
| MM | FCGR2C |
| MM | CHGA |
| MM | TIMD4 |
| MM | RBP4 |
| MM | LYG2 |
| MM | OBP2A |
| MM | KIR3DL3 |
| MM | PTHLH |
| MM | CCL8 |
| MM | AMELX |
| MM | CST4 |
| MM | GNLY |
| MM | KCNMB3 |
| MM | IFNW1 |
| MM | WFDC9 |
| MM | CLDN2 |
| MM | KCT2 |
| MM | CPXM2 |
| MM | BCAM |
| MM | RAMP2 |
| MM | ERVK-7 |
| MM | NHLRC3 |
| MM | OS9 |
| MM | DKK2 |
| MM | IL2RA |
| MM | SPINK8 |
| MM | SYNDIG1L |
| MM | SPINK9 |
| MM | DPT |
| MM | AXL |
| MM | SPINK1 |
| MM | BTN1A1 |
| MM | SLC2A2 |
| MM | SLC24A3 |
| MM | DRAXIN |
| MM | ERVK-24 |
| MM | TNFRSF4 |
| MM | CST5 |
| MM | IER3 |
| MM | SLC22A25 |
| MM | CLCC1 |
| MM | TNFRSF1B |
| MM | FP248 |
| MM | LYSMD4 |
| MM | AGRP |
| MM | ADAMTS16 |
| MM | DEFB126 |
| MM | ECM1 |
| MM | IL16 |
| MM | INSL6 |
| MM | XCL2 |
| MM | ENDOU |
| MM | CST8 |
| MM | UGT2A1 |
| MM | FAM174A |
| MM | RCN1 |
| MM | UGT1A1 |
| MM | RTN4RL1 |
| MM | C11orf94 |
| MM | FAM187B |
| MM | APOE |
| MM | BTC |
| MM | LHFPL1 |
| MM | PRLR |
| MM | FGFRL1 |
| MM | CCL15 |
| MM | MPZL3 |
| MM | PPBP |
| MM | PDCD1 |
| MM | SPINK4 |
| MM | RTBDN |
| MM | CD99L2 |
| MM | PGA4 |
| MM | HSPA13 |
| MM | CNTN2 |
| MM | TMED1 |
| MM | IL1B |
| MM | WFDC12 |
| MM | SDF2L1 |
| MM | IL1F9 |
| MM | IGFBP5 |
| MM | TNFRSF12A |
| MM | MICB |
| MM | S100A13 |
| MM | RNASE8 |
| MM | FAM19A2 |
| MM | IMPG1 |
| MM | SERPINE1 |
| MM | CTSA |
| MM | NPPC |
| MM | PLA2G1B |
| MM | OBP2B |
| MM | CCL16 |
| MM | IL13 |
| MM | EREG |
| MM | KLK8 |
| MM | IL6 |
| MM | TNF |
| MM | C1QTNF2 |
| MM | KLK14 |
| MM | PTPRR |
| MM | ADM2 |
| MM | CCL24 |
| MM | NCR3 |
| MM | NETO1 |
| MM | C5orf64 |
| MM | GP6 |
| MM | MIA2 |
| MM | FGF17 |
| MM | TREML4 |
| MM | SOSTDC1 |
| MM | COL9A3 |
| MM | FCER1A |
| MM | ENSP00000320207 |
| MM | IGFBP3 |
| MM | C6orf15 |
| MM | PROK1 |
| MM | SLC22A31 |
| MM | CD151 |
| MM | EPYC |
| MM | PROKR2 |
| MM | FKBP9 |
| MM | IL34 |
| MM | MMP1 |
| MM | LAMC1 |
| MM | SRGN |
| MM | ERVK-18 |
| MM | IGSF4B |
| MM | CALY |
| MM | FKBP14 |
| MM | RCN2 |
| MM | IL17BR |
| MM | CALR |
| MM | CLDN3 |
| MM | GPC6 |
| MM | OTOL1 |
| MM | MANF |
| MM | STC2 |
| MM | CSAG1 |
| MM | TNFRSF9 |
| MM | TMEM161A |
| MM | PRH1; |
| MM | TRH |
| MM | CXCL1 |
| MM | FSTL1 |
| MM | TDGF1 |
| MM | PRSS3 |
| MM | PGA3 |
| MM | VSTM2A |
| MM | IGFL2 |
| MM | CRTAC1 |
| MM | F13B |
| MM | CTRB2 |
| MM | UNQ9165/PRO28630 |
| MM | GNRH1 |
| MM | SERPINA3 |
| MM | APP |
| MM | IGFBP2 |
| MM | ITIH3 |
| MM | TM9SF3 |
| MM | CNPY2 |
| MM | IL29 |
| MM | OTOR |
| MM | TM2D2 |
| MM | CSN3 |
| MM | APOH |
| MM | SEMA6A |
| MM | CD14 |
| MM | MUC7 |
| MM | LAS2 |
| MM | C2orf40 |
| MM | TNFRSF5 |
| MM | FGFR2 |
| MM | CXCL3 |
| MM | ADM |
| MM | IL1RAP |
| MM | CSPG5 |
| MM | RARRES2 |
| MM | MIA |
| MM | FKBP2 |
| MM | JCHAIN |
| MM | NINJ1 |
| MM | RCN3 |
| MM | ZP4 |
| MM | MDK |
| MM | LCN1P1 |
| MM | SIGLEC9 |
| MM | COL10A1 |
| MM | SPACA7 |
| MM | SPAG11B |
| MM | XG |
| MM | CLDN18 |
| MM | CCL17 |
| MM | SHISA7 |
| MM | TMEM149 |
| MM | NBL1 |
| MM | GAST |
| MM | OXT |
| MM | SEMA6C |
| MM | CCL28 |
| MM | LRIT3 |
| MM | CHRNB3 |
| MM | CCDC47 |
| MM | SLC2A10 |
| MM | LECT2 |
| MM | CRLF1 |
| MM | PSAP |
| MM | TMEM119 |
| MM | SPACA5 |
| MM | CALU |
| MM | MUCL3 |
| MM | LILRB2 |
| MM | ODAM |
| MM | CLU |
| MM | CD40LG |
| MM | CFHR1 |
| MM | CHGB |
| MM | IL7 |
| MM | XCL1 |
| MM | CPVL |
| MM | SYCN |
| MM | SLC39A8 |
| MM | DCD |
| MM | PLA2G10 |
| MM | IL36B |
| MM | SLC6A2 |
| MM | FAM24B |
| MM | LEP |
| MM | IL9 |
| MM | PTN |
| MM | CCL26 |
| MM | AHSG |
| MM | RNASE10 |
| MM | CD274 |
| MM | KCNV2 |
| MM | FAM3C |
| MM | LY6G6D |
| MM | SPINK13 |
| MM | ASIP |
| MM | LGALS3 |
| MM | CTSW |
| MM | FCAMR |
| MM | CD320 |
| MM | PRRG4 |
| MM | CA4 |
| MM | LILRB6 |
| MM | APLP2 |
| MM | BMPR1A |
| MM | APOA4 |
| MM | TXNDC12 |
| MM | OLR1 |
| MM | CXCL6 |
| MM | CXCL9 |
| MM | OTOS |
| MM | XK |
| MM | PRG3 |
| MM | ANGPTL4 |
| MM | CCL23 |
| MM | PRRT1 |
| MM | ATP4B |
| MM | IL17C |
| MM | CSF2 |
| MM | CCL13 |
| MM | HSD11B1L |
| MM | MICA |
| MM | IGF1 |
| MM | MSMP |
| MM | TGOLN2 |
| MM | ERP27 |
| MM | PTPRN2 |
| MM | KLRK1 |
| MM | LRP11 |
| MM | PIANP |
| MM | LIF |
| MM | S100A8 |
| MM | CSN2 |
| MM | EVAIC |
| MM | IFNA6 |
| MM | PCSK1 |
| MM | LILRB4 |
| MM | QPCT |
| MM | SNORC |
| MM | SHISA6 |
| MM | PRR27 |
| MM | KLRF1 |
| MM | CTSG |
| MM | PDIA3 |
| MM | CNPY4 |
| MM | RSPO4 |
| MM | REG1A |
| MM | PEBP4 |
| MM | CRTAP |
| MM | TGFBR1 |
| MM | VSTM2B |
| MM | CP |
| MM | VPREB1 |
| MM | CD44 |
| MM | IGFBP7 |
| MM | FGF7 |
| MM | ENSP00000381830 |
| MM | SEMG1 |
| MM | IL1A |
| MM | EPO |
| MM | CDH19 |
| MM | IL32 |
| MM | SUMF1 |
| MM | ANTXRL |
| MM | LHFPL5 |
| MM | CCL21 |
| MM | PLVAP |
| MM | CELA1 |
| MM | ICOSLG |
| MM | FGF23 |
| MM | SLC6A11 |
| MM | CLDN1 |
| MM | SFTPB |
| MM | NTS |
| MM | REG4 |
| MM | IGLL1 |
| MM | CSF3 |
| MM | CNPY3 |
| MM | NOPE |
| MM | TXN |
| MM | CDSN |
| MM | KLK7 |
| MM | TNFRSF13C |
| MM | RAET1L |
| MM | FAM19A3 |
| MM | LALBA |
| MM | RTN4R |
| MM | CFD |
| MM | PGLYRP1 |
| MM | CRELD2 |
| MM | AMTN |
| MM | CCL7 |
| MM | TMEM102 |
| MM | TNFRSF10B |
| MM | C2orf66 |
| MM | HAVCR1 |
| MM | FAM234A |
| MM | NOV |
| MM | RSPO3 |
| MM | IFNA13 |
| MM | CTLA4 |
| MM | PLAC9 |
| MM | UGT2B28 |
| MM | IL28B |
| MM | TOR1B |
| MM | INSL3 |
| MM | APOA1 |
| MM | CFHR2 |
| MM | FCGR2A |
| MM | IGF2 |
| MM | AMBN |
| MM | ASIC5 |
| MM | NTRK2 |
| MM | HNRNPA2B1 |
| MM | PRELP |
| MM | CILP2 |
| MM | EPHA4 |
| MM | KAZALD1 |
| MM | FAM168B |
| MM | CD248 |
| MM | COL14A1 |
| MM | VTN |
| MM | CELA3A |
| MM | PTPRD |
| MM | CELA3B |
| MM | DKK3 |
| MM | CREG2 |
| MM | ANGPTL5 |
| MM | MUCL1 |
| MM | SLC15A1 |
| MM | GREM2 |
| MM | WFDC3 |
| MM | PRR4 |
| MM | VSIG4 |
| MM | FAM19A4 |
| MM | CST7 |
| MM | TEX46 |
| MM | TFF1 |
| MM | FCMR |
| MM | CST1 |
| MM | CGREF1 |
| MM | AIMP1 |
| MM | IL4 |
| MM | SERPINI1 |
| MM | PRAP1 |
| MM | PGC |
| MM | GZMA |
| MM | CXCL11 |
| MM | SDC4 |
| MM | CXCL5 |
| MM | PANX3 |
| MM | CCL20 |
| MM | BPIFC |
| MM | TGFBR3L |
| MM | SNCA |
| MM | IL22RA2 |
| MM | ARSJ |
| MM | SFRP4 |
| MM | TREML1 |
| MM | LYPD6B |
| MM | CCL1 |
| MM | HRC |
| MM | CLTRN |
| MM | FZD4 |
| MM | LRRC8C |
| MM | GH1 |
| MM | IHH |
| MM | IL10RB |
| MM | IGFBP1 |
| MM | IGDCC3 |
| MM | VEGFA |
| MM | SPOCK2 |
| MM | FGF16 |
| MM | SLC39A14 |
| MM | BST2 |
| MM | SCG2 |
| MM | MFAP2 |
| MM | CT83 |
| MM | TMEM95 |
| MM | ABHD12 |
| MM | CLN5 |
| MM | SCGB1A1 |
| MM | HSD17B13 |
| MM | SPACA3 |
| MM | BTNL8 |
| MM | SLC22A9 |
| MM | SLC2A13 |
| MM | MPO |
| MM | TTYH2 |
| MM | TMEM169 |
| MM | CD72 |
| MM | TRABD2B |
| MM | SCG5 |
| MM | SERPINI2 |
| MM | SPP2 |
| MM | S100A7 |
| MM | KRTDAP |
| MM | CST2 |
| MM | CREG1 |
| MM | TSPAN2 |
| MM | NRN1 |
| MM | VSIG2 |
| MM | MEGF9 |
| MM | RNF43 |
| MM | CLDN8 |
| MM | ENH1 |
| MM | SMOC1 |
| MM | LRRN4CL |
| MM | PDGFA |
| MM | PLA2G12B |
| MM | PTTG1IP |
| MM | FAM24A |
| MM | FKBP10 |
| MM | SLC6A13 |
| MM | SLC10A4 |
| MM | GFRA2 |
| MM | SLURP1 |
| MM | OLFM1 |
| MM | BTLA |
| MM | ATP6AP2 |
| MM | SCGB2A2 |
| MM | PILRB |
| MM | SLC22A4 |
| MM | EXOC3-AS1 |
| MM | ART1 |
| MM | MUC5AC |
| MM | CHAD |
| MM | DKKL1 |
| MM | SLC8B1 |
| MM | TSLP |
| MM | SCGB1C2 |
| MM | PDGFB |
| MM | C1QL1 |
| MM | TM4SF6 |
| MM | FRZB |
| MM | TMEFF1 |
| MM | IL17B |
| MM | DAG1 |
| MM | COLQ |
| MM | PLAT |
| MM | TNFRSF6B |
| MM | CLDN4 |
| MM | TREM2 |
| MM | SUSD6 |
| MM | VSTM2L |
| MM | NFASC |
| MM | COMT |
| MM | MSR1 |
| MM | LSR |
| MM | CER1 |
| MM | AZU1 |
| MM | CCK |
| MM | PLA2G2A |
| MM | SMOC2 |
| MM | CXCL13 |
| MM | CRTAM |
| MM | GKN1 |
| MM | NRXN3 |
| MM | DHRS7C |
| MM | CHRDL2 |
| MM | HTR3D |
| MM | TRPC4 |
| NMO | CXCL2 |
| NMO | CXCL3 |
| NMO | IGFBPL1 |
| NMO | CCL22 |
| NMO | IL1F9 |
| NMO | LY6G6D |
| NSCLC | CCL17 |
| NSCLC | CCL24 |
| NSCLC | CXCL1 |
| NSCLC | CXCL3 |
| NSCLC | EDIL3 |
| NSCLC | IFNA13 |
| NSCLC | IFNA14 |
| NSCLC | IFNA17 |
| NSCLC | IFNA2 |
| NSCLC | IFNA5 |
| NSCLC | IFNA6 |
| NSCLC | IFNA8 |
| NSCLC | IFNL2 |
| NSCLC | IFNW1 |
| NSCLC | IL28B |
| NSCLC | IL34 |
| NSCLC | MADCAM1 |
| NSCLC | PDGFB |
| NSCLC | REG1A |
| NSCLC | SDC1 |
| NSCLC | BTN1A1 |
| NSCLC | C6 |
| NSCLC | CD207 |
| NSCLC | CD3D |
| NSCLC | CDH19 |
| NSCLC | COLEC12 |
| NSCLC | EREG |
| NSCLC | FGF23 |
| NSCLC | FGF7 |
| NSCLC | FGFBP3 |
| NSCLC | IGFBPL1 |
| NSCLC | IL15RA |
| NSCLC | IL17F |
| NSCLC | IL1RAP |
| NSCLC | IL22RA2 |
| NSCLC | IL4 |
| NSCLC | IL4R |
| NSCLC | ITGA5 |
| NSCLC | LAG3 |
| NSCLC | LRRC4 |
| NSCLC | MPZL3 |
| NSCLC | NOTCH2NL |
| NSCLC | NTRK3 |
| NSCLC | REG4 |
| NSCLC | SCARA3 |
| NSCLC | STIM2 |
| NSCLC | TNFRSF10C |
| NSCLC | TNFRSF19L |
| NSCLC | TREML1 |
| PANDAS | LRP11 |
| Sarcoidosis | CX3CL1 |
| Sarcoidosis | EPYC |
| Sarcoidosis | PGLYRP1 |
| SLE | CXCL3 |
| SLE | IFNA17 |
| SLE | CXCL1 |
| SLE | LOC644613 |
| SLE | IFNA6 |
| SLE | SV2C |
| SLE | TMEM102 |
| SLE | PDCD1LG2 |
| SLE | SLC29A4 |
| SLE | IL1A |
| SLE | C5orf64 |
| SLE | IFNW1 |
| SLE | SCGB1D1 |
| SLE | EPYC |
| SLE | CNPY2 |
| SLE | CCL4L1 |
| SLE | SPINK9 |
| SLE | TNF |
| SLE | KIRREL3 |
| SLE | IFNA8 |
| SLE | IFNA14 |
| SLE | VEGFB |
| SLE | TMEM108 |
| SLE | IFNA5 |
| SLE | ACVR2B |
| SLE | OBP2B |
| SLE | MCFD2 |
| SLE | DPT |
| SLE | SPACA7 |
| SLE | IFNA13 |
| SLE | FKBP14 |
| SLE | LACRT |
| SLE | IL6 |
| SLE | FAM19A3 |
| SLE | IFNL2 |
| SLE | ERP27 |
| SLE | TMEM149 |
| SLE | PRH1; |
| SLE | ZG16B |
| SLE | IFNA2 |
| SLE | RAET1E |
| SLE | CCDC47 |
| SLE | MUC21 |
| SLE | CCL22 |
| SLE | CGREF1 |
| SLE | TEPP |
| SLE | FAM19A2 |
| SLE | SPOCK1 |
| SLE | SRGN |
| SLE | SHISA7 |
| SLE | CCL17 |
| SLE | RNASE10 |
| SLE | FGF21 |
| SLE | APOA4 |
| SLE | NGFR |
| SLE | KCNV2 |
| SLE | AGER |
| SLE | FGFRL1 |
| SLE | LGR6 |
| SLE | CCL8 |
| SLE | CD44 |
| SLE | ITIH3 |
| SLE | CST8 |
| SLE | SSPN |
| SLE | CELA1 |
| SLE | IL4 |
| SLE | RCN3 |
| SLE | PRRG4 |
| SLE | MFAP5 |
| SLE | CSPG5 |
| SLE | VTCN1 |
| SLE | PLA2G2E |
| SLE | LY6H |
| SLE | GYPC |
| SLE | SLC41A2 |
| SLE | DRAXIN |
| SLE | CSHL1 |
| SLE | LAIR2 |
| SLE | IGFBP2 |
| SLE | CD248 |
| SLE | RGMB |
| SLE | TGOLN2 |
| SLE | CSAG1 |
| SLE | ACP4 |
| SLE | CALU |
| SLE | BTNL8 |
| SLE | SOSTDC1 |
| SLE | LYSMD4 |
| SLE | LCN2 |
| SLE | SCGB1C2 |
| SLE | CST4 |
| SLE | IGF1 |
| SLE | PRRT1 |
| SLE | CHRNA5 |
| SLE | ANTXRL |
| SLE | TNFRSF6 |
| SLE | CD300LG |
| SLE | SERPINE1 |
| SLE | OLFM1 |
| SLE | PLA2G10 |
| SLE | CD300E |
| SLE | CDH19 |
| SLE | RAMP2 |
| SLE | ATP4B |
| SLE | PTPRR |
| SLE | SFN |
| SLE | HCRTR2 |
| SLE | ACRV1 |
| SLE | FAM3A |
| SLE | ACVR1B |
| SLE | FGF23 |
| SLE | IL15RA |
| SLE | IGFBP7 |
| SLE | LHFPL1 |
| SLE | IL28B |
| SLE | VIT |
| SLE | IER3 |
| SLE | C2orf40 |
| SLE | PLVAP |
| SLE | LECT2 |
| SLE | DAG1 |
| SLE | SPINK6 |
| SLE | SLC2A12 |
| SLE | IGLL1 |
| SLE | TFF2 |
| SLE | ASIP |
| SLE | IL16 |
| SLE | EDIL3 |
| SLE | CCL13 |
| SLE | RCN1 |
| SLE | CSH2 |
| SLE | IL33 |
| SLE | LILRB4 |
| SLE | SPESP1 |
| SLE | PDGFB |
| SLE | PTHLH |
| SLE | C9orf47 |
| SLE | CHRDL2 |
| SLE | ART3 |
| SLE | CPVL |
| SLE | CCL15 |
| SSC | SERPINE1 |
| SSC | LEP |
| SSC | LECT2 |
| SSC | OTOR |
| SSC | CASQ1 |
| SSC | CST6 |
| SSC | INSL3 |
| SSC | SPACA3 |
| SSC | AMTN |
| SSC | ZG16B |
| SSC | LOC644613 |
| SSC | PGA4 |
| SSC | LYSMD4 |
| SSC | SRGN |
| SSC | CDH19 |
| SSC | SHISA7 |
| SSC | FAM19A3 |
| SSC | HAVCR1 |
| SSC | BAMBI |
| SSC | MSMP |
| SSC | SPACA7 |
| SSC | PTHLH |
| SSC | PLA2G12B |
| SSC | CXCL3 |
| SSC | CST4 |
| SSC | DKK3 |
| SSC | PIANP |
| SSC | PRG3 |
| SSC | BTC |
| SSC | CCL17 |
| SSC | XCL1 |
| SSC | LMBRD2 |
| SSC | LALBA |
| SSC | TGFA |
| SSC | IL29 |
| SSC | EVI2B |
| SSC | SLPI |
| SSC | CLCC1 |
| SSC | RNASE10 |
| SSC | FGFBP3 |
| SSC | FAM168B |
| SSC | PGLYRP1 |
| SSC | ANGPTL4 |
| SSC | CLU |
| SSC | AGER |
| SSC | TMEM108 |
| SSC | C1QTNF2 |
| SSC | TMEM119 |
| SSC | CCL8 |
| SSC | ODAPH |
| SSC | CNPY3 |
| SSC | MZB1 |
| SSC | CYTL1 |
| SSC | PRH1 |
| SSC | SLC2A10 |
| SSC | PRRG1 |
| SSC | CSPG5 |
| SSC | DRAXIN |
| SSC | PRR27 |
| SSC | DKK1 |
| SSC | NTRK2 |
| SSC | IFNA13 |
| SSC | PDCD1 |
| SSC | FAM19A2 |
| SSC | IFNW1 |
| SSC | RCN1 |
| SSC | CFD |
| SSC | CRELD2 |
| SSC | CCL18 |
| SSC | CD14 |
| SSC | BTN1A1 |
| SSC | PTPRR |
| SSC | TMEM91 |
| SSC | VSIG2 |
| SSC | CCL13 |
| SSC | C2orf40 |
| SSC | VEGFB |
| SSC | REG4 |
| SSC | TXNDC12 |
| SSC | ACVR2B |
| SSC | ODAM |
| SSC | CST5 |
| SSC | PI3 |
| SSC | TMEM149 |
| SSC | TEPP |
| SSC | KCNV2 |
| SSC | PLA2G2E |
| SSC | AIMP1 |
| SSC | IGFBP5 |
| SSC | ASIP |
| SSC | PGC |
| SSC | TM9SF3 |
| SSC | AMELX |
| SSC | CSN2 |
| SSC | CPXM2 |
| SSC | PRSS3 |
| SSC | FAM3A |
| SSC | LILRA3 |
| SSC | CSAG1 |
| SSC | RTBDN |
| SSC | CELA1 |
| SSC | ANTXRL |
| SSC | PLA2G10 |
| SSC | KCT2 |
| SSC | APOH |
| SSC | NENF |
| SSC | NPPC |
| SSC | LY6H |
| SSC | FGF1 |
| SSC | SLC1A1 |
| SSC | IFNL2 |
| SSC | HSPA13 |
| SSC | C6orf15 |
| SSC | FLJ37218 |
| SSC | CCL7 |
| SSC | APOA4 |
| SSC | FSTL1 |
| SSC | IGFBP1 |
| SSC | FCGR2A |
| SSC | SMR3A |
| SSC | IFITM10 |
| SSC | MSLN |
| SSC | PRAP1 |
| SSC | EPO |
| SSC | PLVAP |
| SSC | PROK1 |
| SSC | TSLP |
| SSC | MIA |
| SSC | APP |
| SSC | OBP2A |
| SSC | RTN4RL1 |
| SSC | PRRT3 |
| SSC | APOA1 |
| SSC | FGF7 |
| SSC | TMED1 |
| SSC | LGALS3 |
| SSC | JCHAIN |
| SSC | PRRG3 |
| SSC | IGF1 |
| SSC | ACRV1 |
| SSC | SLC38A4 |
| SSC | FKBP11 |
| SSC | ITPRIPL1 |
| SSC | PLAC9 |
| SSC | TFF2 |
| SSC | WFDC13 |
| SSC | LCN1 |
| SSC | LYG1 |
| SSC | LAIR2 |
| SSC | TNFRSF8 |
| SSC | SOSTDC1 |
| SSC | VSTM2A |
| SSC | IGFBP7 |
| SSC | PSORS1C2 |
| SSC | FGF23 |
| SSC | RSPO3 |
| SSC | S100A9 |
| SSC | CXCL9 |
| SSC | TGOLN2 |
| SSC | ACP5 |
| SSC | MANF |
| SSC | AMBN |
| SSC | PSAPL1 |
| SSC | WFDC10A |
| SSC | PPT1 |
| SSC | MANSC4 |
| SSC | CD248 |
| SSC | NGRN |
| SSC | PSAP |
| SSC | LILRB2 |
| SSC | SCGB2A2 |
| SSC | IGFBPL1 |
| SSC | SV2C |
| SSC | CXCL6 |
| SSC | CD300E |
| SSC | RCN3 |
| SSC | IGFBP3 |
| SSC | RTN4R |
| SSC | PRRT1 |
| SSC | ACVR2A |
| SSC | LCN2 |
| SSC | HCRTR2 |
| SSC | CELA3A |
| SSC | ADM2 |
| SSC | LRIT3 |
| SSC | MIA2 |
| SSC | TNFRSF17 |
| SSC | SPN |
| SSC | SLC6A5 |
| SSC | WFDC1 |
| SSC | LILRB4 |
| SSC | CTSG |
| SSC | CXCL11 |
| SSC | KLK7 |
| SSC | CST8 |
| SSC | NOPE |
| SSC | GAST |
| SSC | ASTN2 |
| SSC | MCFD2 |
| SSC | CCL22 |
| SSC | OTOL1 |
| SSC | SYCN |
| SSC | CCL2 |
| SSC | SOST |
| SSC | PTN |
| SSC | TACSTD2 |
| SSC | IL21 |
| SSC | IGLL1 |
| SSC | MMP7 |
| SSC | APLP2 |
| SSC | SSBP3_AS1 |
| SSC | CST7 |
| SSC | SSPN |
| SSC | HS3ST1 |
| SSC | GP6 |
| SSC | RNASE8 |
| SSC | ACVR1B |
| SSC | PDIA3 |
| SSC | IL15RA |
| SSC | PTPRN2 |
| SSC | IL28B |
| SSC | PMCH |
| SSC | PVRL2 |
| SSC | WIF1 |
| SSC | EREG |
| SSC | EDIL3 |
| SSC | CDSN |
| SSC | REG1A |
| SSC | PTH |
| SSC | LHFPL1 |
| SSC | TRABD2B |
| SSC | TIGIT |
| SSC | KISS1 |
| SSC | CXCL17 |
| SSC | SPOCK2 |
| SSC | CTF1 |
| SSC | CD55 |
| SSC | DEFB108B |
| SSC | IL17C |
| SSC | GPHB5 |
| SSC | PRLR |
| SSC | NLGN4Y |
| SSC | SPACA5 |
| SSC | FGF17 |
| SSC | C9 |
| SSC | CHRDL2 |
| SSC | PF4V1 |
| SSC | RAMP2 |
| SSC | CCL26 |
| SSC | CD151 |
| SSC | TRPC5 |
| SSC | MMP1 |
| SSC | PRRG4 |
| SSC | ART3 |
| SSC | HEPACAM2 |
| SSC | SDF2L1 |
| SSC | IGFBP2 |
| SSC | AXL |
| SSC | SCN3B |
| SSC | EPHA5 |
| SSC | IL1RAP |
| SSC | ATP6AP2 |
| SSC | CCL20 |
| SSC | GNRH1 |
| SSC | SEMG1 |
| SSC | APOE |
| SSC | FGFRL1 |
| SSC | IBSP |
| SSC | TEX264 |
| SSC | CCBE1 |
| SSC | BCAM |
| SSC | LRRC8C |
| SSC | DKK2 |
| SSC | EPHA4 |
| SSC | SFRP4 |
| SSC | SYNDIG1L |
| SSC | FAM19A5 |
| SSC | LYG2 |
| SSC | FAM3C |
| SSC | TUSC5 |
| SSC | MDK |
| SSC | FGF16 |
| SSC | MFGE8 |
| SSC | PRELP |
| SSC | COL10A1 |
| SSC | IGF2 |
| SSC | CSN3 |
| SSC | CLDN18 |
| SSC | PDIA6 |
| SSC | CHAD |
| SSC | TNFRSF21 |
| SSC | C6orf120 |
| SSC | COL9A3 |
| SSC | PDGFB |
| SSC | TOR1B |
| SSC | LHFPL5 |
| SSC | UNQ9165_PRO28630 |
| SSC | CCL15 |
| SSC | BMPR1A |
| SSC | FGFR2 |
| SSC | DGAT2L7P |
| SSC | SERPINA13P |
| SSC | FCAMR |
| SSC | XCL2 |
| SSC | TMEM9B |
| SSC | RNF167 |
| SSC | LCN15 |
| SSC | TREML1 |
| SSC | FGF21 |
| SSC | SLC22A31 |
| SSC | IL20RB |
| SSC | CCL11 |
| SSC | STC2 |
| SSC | FKBP14 |
| SUSAC | CCL24 |
| SUSAC | SDC4 |
| SUSAC | TREML1 |
| SUSAC | VSIG4 |
| Malaria | LCN15 |
| Malaria | IL21 |
| Malaria | LEP |
| Malaria | FKBP7 |
| Malaria | CCL11 |
| Malaria | BMPR2 |
| Malaria | SCGB2A2 |
| Malaria | GZMK |
| Malaria | MSMP |
| Malaria | DCD |
| Malaria | SPARC |
| Malaria | COL9A3 |
| Malaria | FLRT3 |
| Malaria | TNFRSF10B |
| Malaria | FZD4 |
| Malaria | TSPAN13 |
| Malaria | HTRA3 |
| Malaria | PCSK1 |
| Malaria | LYPD6B |
| Malaria | CPE |
| Malaria | GFRAL |
| Malaria | TGOLN2 |
| Malaria | PRLR |
| Malaria | TNFRSF21 |
| Malaria | TSPAN2 |
| Malaria | AMTN |
| Malaria | F12 |
| Malaria | SLC1A1 |
| Malaria | MPZL3 |
| Malaria | F13B |
| Malaria | C6orf120 |
| Malaria | PRAP1 |
| Malaria | IGFBP6 |
| Malaria | FGL2 |
| Malaria | SPX |
| Malaria | GPC6 |
| Malaria | INSL3 |
| Malaria | CYTL1 |
| Malaria | TM4SF6 |
| Malaria | SGCA |
| Malaria | C9orf135 |
| Malaria | CD300A |
| Malaria | CTF1 |
| Malaria | OPN4 |
| Malaria | SLC22A31 |
| Malaria | ZP4 |
| Malaria | IL21R |
| Malaria | ADM |
| Malaria | AXL |
| Malaria | EPHA5 |
| Malaria | IL17A |
| Malaria | PTH |
| Malaria | TNFRSF17 |
| Malaria | SHISA6 |
| Malaria | FGF17 |
| Malaria | GNRH1 |
| Malaria | SDF2L1 |
| Malaria | CNPY4 |
| Malaria | SLC6A9 |
| Malaria | NPR3 |
| Malaria | SIGLEC10 |
| Malaria | IL13 |
| Malaria | SFTPA2 |
| Malaria | GDPD3 |
| Malaria | CD164L2 |
| Malaria | KLK2 |
| Malaria | ENSP00000381830 |
| Malaria | AKR1B10 |
| Malaria | KLK3 |
| Malaria | FCER1A |
| Malaria | SNORC |
| Malaria | CSHL1 |
| Malaria | CSH2 |
| Malaria | CSN3 |
| Malaria | SLC1A4 |
| Malaria | HEPACAM2 |
| Malaria | INS |
| Malaria | GP6 |
| Malaria | RNASE8 |
| Malaria | SLAMF9 |
| Malaria | DPT |
| Malaria | MINPP1 |
| Malaria | FGFR3 |
| Malaria | C2orf66 |
| Malaria | IMPG1 |
| Malaria | NENF |
| Malaria | DKK3 |
| Malaria | NOV |
| Malaria | SERPINI2 |
| Malaria | IFNA6 |
| Malaria | COLEC12 |
| Malaria | CALR |
| Malaria | PRRG1 |
| Malaria | GSN |
| Malaria | SLC10A4 |
| Malaria | CD99 |
| Malaria | FSTL1 |
| Malaria | IL16 |
| Malaria | TRH |
| Malaria | SLC6A14 |
| Malaria | GLB1 |
| Malaria | CCL20 |
| Malaria | ARTN |
| Malaria | SPP2 |
| Malaria | LINC00305 |
| Malaria | LAS2 |
| Malaria | S100A13 |
| Malaria | MZB1 |
| Malaria | RETN |
| Malaria | FAM172A |
| Malaria | CD99L2 |
| Malaria | CD151 |
| Malaria | SDF4 |
| Malaria | CEACAM19 |
| Malaria | CHGB |
| Malaria | SLC8B1 |
| Malaria | CDNF |
| Malaria | BCAM |
| Malaria | TSPAN9 |
| Malaria | ENDOD1 |
| Malaria | EMC10 |
| Malaria | OS9 |
| Malaria | TMEM169 |
| Malaria | IL22 |
| Malaria | NBL1 |
| Malaria | IL1RN |
| Malaria | SMOC2 |
| Malaria | PRRG3 |
| Malaria | LRIT3 |
| Malaria | KCT2 |
| Malaria | XG |
| Malaria | IGF1 |
| Malaria | GAST |
| Malaria | CGREF1 |
| Malaria | RAMP2 |
| Malaria | PRRG4 |
| Malaria | CDSN |
| Malaria | C11orf94 |
| Malaria | OTOL1 |
| Malaria | IBSP |
| Malaria | LGALS3 |
| Malaria | LYSMD4 |
| Malaria | SYCN |
| Malaria | JCHAIN |
| Malaria | CST8 |
| Malaria | PRRT1 |
| Malaria | CCL15 |
| Malaria | SSPN |
| Malaria | APOO |
| Malaria | CST5 |
| Malaria | SPINK1 |
| Malaria | HCRTR2 |
| Malaria | PRRT3 |
| Malaria | PSORS1C2 |
| Malaria | RTBDN |
| Malaria | ACRV1 |
| Malaria | FKBP14 |
| Malaria | SPINK4 |
| Malaria | IGFBP1 |
| Malaria | PLA2G2E |
| Malaria | OBP2A |
| Malaria | CCL8 |
| Malaria | VEGFB |
| Malaria | TGFA |
| Malaria | COL10A1 |
| Malaria | IFNW1 |
| Malaria | RNASE10 |
| Malaria | PRH1; |
| Malaria | CDH19 |
| Malaria | CPXM2 |
| Malaria | CSPG5 |
| Malaria | RCN3 |
| Malaria | IFNA13 |
| Malaria | IGFBP2 |
| Malaria | PLA2G10 |
| Malaria | SRGN |
| Malaria | EPYC |
| Malaria | CXCL1 |
| Malaria | CNPY2 |
| Malaria | MCFD2 |
| Malaria | ANGPTL4 |
| Malaria | SPACA7 |
| Malaria | SLC2A10 |
| Malaria | RTN4R |
| Malaria | CXCL3 |
| Malaria | CCDC47 |
| Malaria | CST4 |
| Malaria | CELA1 |
| Malaria | LALBA |
| Malaria | PTPRR |
| Malaria | OBP2B |
| Malaria | TXNDC12 |
| Malaria | PTN |
| Malaria | ZG16B |
| Malaria | PRSS3 |
| Malaria | CNPY3 |
| Malaria | PTHLH |
| Malaria | PGLYRP1 |
| Malaria | KLK7 |
| Malaria | CCL13 |
| Malaria | FAM19A3 |
| Malaria | KLK8 |
| Malaria | SERPINA3 |
| Malaria | HCTR1 |
| Malaria | DRD5 |
| Malaria | GPR37L1 |
| Malaria | BDKBR1 |
| Malaria | NPY2R |
| Malaria | SCTR |
| Malaria | ADCYAP1R1 |
| Malaria | GPR19 |
| Malaria | S1PR3 |
| Malaria | NMBR |
| Malaria | CCR4 |
| Malaria | GPR17 |
| Malaria | CNR1 |
| Malaria | OPRK1 |
| Malaria | CYSLTR2 |
| Malaria | P2RY10 |
| Malaria | HTR1B |
| Malaria | OPRM1 |
| Malaria | RXFP3 |
| Malaria | OXER1 |
| Malaria | CXCR3 |
| Malaria | HTR2B |
| Malaria | GPR1 |
| Malaria | NPBW1 |
| Malaria | VSTM2A |
| Malaria | LY6G6D |
| Malaria | SLC41A2 |
| Malaria | MOG |
| Malaria | RNASE9 |
| Malaria | IGLL5 |
| Malaria | CHGA |
| Malaria | TREML1 |
| Malaria | GHRHR |
| Malaria | XK |
| Malaria | KITLG |
| Malaria | WFDC10A |
| Malaria | TMEM108 |
| Malaria | OTOR |
| Malaria | GPR63 |
| Malaria | PLGRKT |
| Malaria | CTSG |
| Malaria | SLC6A5 |
| Malaria | CSAG1 |
| Malaria | FZD9 |
| Malaria | CMKLR1 |
| Malaria | FKBP2 |
| Malaria | ITIH3 |
| Malaria | LILRA4 |
| Malaria | TNFRSF12A |
| Malaria | CXCL13 |
| Malaria | PPT1 |
| Malaria | CXCL17 |
| Malaria | ODAM |
| Malaria | IL1RAP |
| Malaria | SLC38A4 |
| Malaria | ACKR1 |
| Malaria | CADM2 |
| Malaria | PAPLN |
| Malaria | GPR37 |
| Malaria | SLC38A2 |
| Malaria | TMEM59 |
| Malaria | RAET1L |
| Malaria | SPINK8 |
| Malaria | TRABD2B |
| Malaria | FGF23 |
| Malaria | TMEM91 |
| Malaria | SV2C |
| Malaria | REG1A |
| KT | SPOCK1 |
| KT | CD99L2 |
| KT | ACRV1 |
| KT | SPINK4 |
| KT | MCFD2 |
| KT | CD80 |
| KT | IL2RA |
| KT | LOC644613 |
| KT | AGRP |
| KT | SHISA7 |
| KT | RCN2 |
| KT | ACKR1 |
| KT | IFNG |
| KT | SCGB3A1 |
| KT | CCL16 |
| KT | IL29 |
| KT | OBP2B |
| KT | CXCL3 |
| KT | CCDC47 |
| KT | SSPN |
| KT | EPYC |
| KT | SPACA3 |
| KT | MRGPRF |
| KT | KLK8 |
| KT | MUCL3 |
| KT | IL9 |
| KT | IFNL2 |
| KT | IGFBP1 |
| KT | CALU |
| KT | MZB1 |
| KT | CCL22 |
| KT | TNFRSF21 |
| KT | SPACA7 |
| KT | LYG2 |
| KT | TNFRSF5 |
| KT | ANGPTL4 |
| KT | ENDOU |
| KT | PTPRR |
| KT | CSPG5 |
| KT | SPINK9 |
| KT | IL7 |
| KT | FLJ37218 |
| KT | DKK3 |
| KT | ZG16B |
| KT | SERPINE1 |
| KT | SLPI |
| KT | CD274 |
| KT | FAM19A2 |
| KT | VSIG2 |
| KT | CD40LG |
| KT | EDDM3B |
| KT | HCRTR2 |
| KT | FGFR2 |
| KT | EXOC3-AS1 |
| KT | IGFBP2 |
| KT | SERPINA3 |
| KT | CXCL1 |
| KT | OTOR |
| KT | TSPAN9 |
| KT | CNPY3 |
| KT | PRR27 |
| KT | RCN3 |
| KT | CNPY2 |
| KT | BTC |
| KT | ADRB3 |
| KT | IGFBP5 |
| KT | NPY1R |
| KT | TMEM102 |
| KT | LALBA |
| KT | CXCL2 |
| KT | CCL13 |
| KT | OTOL1 |
| KT | IL1A |
| KT | APOO |
| KT | LGALS3 |
| KT | LECT2 |
| KT | CDH19 |
| KT | RTN4R |
| KT | RETN |
| KT | CSF2 |
| KT | APOH |
| KT | MICA |
| KT | GPR6 |
| KT | IL4 |
| KT | CRLF1 |
| KT | LAIR2 |
| KT | NPY2R |
| KT | LYSMD4 |
| KT | DCD |
| KT | TXNDC12 |
| KT | GP6 |
| KT | NOV |
| KT | DRAXIN |
| KT | CCR10 |
| KT | PILRA |
| KT | GPR1 |
| KT | OPRL1 |
| KT | FAM168B |
| KT | PRLR |
| KT | CFD |
| KT | IBSP |
| KT | PTPRN2 |
| KT | ERP27 |
| KT | BTN1A1 |
| KT | PDCD1 |
| KT | SV2C |
| KT | CSN2 |
| KT | NINJ1 |
| KT | TMEM91 |
| KT | SLC1A1 |
| KT | ADCYAP1 |
| KT | SEMG2 |
| KT | APOA1 |
| KT | MPO |
| KT | VEGFB |
| KT | IL34 |
| KT | IFNA17 |
| KT | S100A13 |
| KT | AVPR1A |
| KT | CCL17 |
| KT | AMTN |
| KT | IL17RD |
| KT | DKK1 |
| KT | PSORS1C2 |
| KT | SSTR2 |
| KT | SYCN |
| KT | GPR37 |
| KT | ANTXRL |
| KT | AGER |
| KT | PGLYRP1 |
| KT | WFDC12 |
| KT | IMPG1 |
| KT | GNRH1 |
| KT | SLC2A12 |
| KT | FKBP2 |
| KT | ULBP1 |
| KT | TMEM119 |
| KT | PRSS3 |
| KT | MIA2 |
| KT | SLC2A2 |
| KT | C5orf64 |
| KT | TFPI2 |
| KT | PCSK1 |
| KT | PRH1; |
| KT | IGFBP7 |
| KT | UNQ6190/PRO20217 |
| KT | CELA1 |
| KT | OSTN |
| KT | RARRES2 |
| KT | AZGP1 |
| KT | TGFA |
| KT | IL6 |
| KT | FMR1NB |
| KT | REG1B |
| KT | CXCL12 |
| KT | IL28B |
| KT | JCHAIN |
| KT | CES3 |
| KT | FAM19A3 |
| KT | FAM174A |
| KT | CCL4L1 |
| KT | PLA2G2E |
| KT | COL10A1 |
| KT | ITPRIPL1 |
| KT | PPBP |
| KT | MANF |
| KT | TMEM149 |
| KT | PRRG4 |
| KT | GFRA2 |
| KT | CA11 |
| KT | TLR1 |
| KT | CCL21 |
| KT | REG4 |
| KT | PRG3 |
| KT | IFNA13 |
| KT | SLC22A25 |
| KT | CCL7 |
| KT | ATP6AP2 |
| KT | BRICD5 |
| KT | GAST |
| KT | KAL1 |
| KT | TMEM108 |
| KT | IL16 |
| KT | GPR182 |
| KT | TNFRSF6 |
| KT | TSLP |
| KT | APOA4 |
| KT | SIRPA |
| KT | FCER1A |
| KT | PLBD2 |
| KT | KCNV2 |
| KT | NXPH1 |
| KT | BCAM |
| KT | IFNA6 |
| KT | SPESP1 |
| KT | NENF |
| KT | PLA2G10 |
| KT | VSTM2A |
| KT | GPR19 |
| KT | NOG |
| KT | CD300E |
| KT | CST5 |
| KT | MMP7 |
| KT | HAVCR1 |
| KT | CST4 |
| KT | THBD |
| KT | MLN |
| KT | TRABD2A |
| KT | ATP4B |
| KT | PIANP |
| KT | GNLY |
| KT | CCKAR |
| KT | GPR63 |
| KT | ICAM2 |
| KT | LYPD6B |
| KT | TMEM120A |
| KT | DHRS4L2 |
| KT | OTOS |
| KT | RCN1 |
| KT | B2M |
| KT | CCL24 |
| KT | IFNA2 |
| KT | IFNA14 |
| KT | BMPR2 |
| KT | SRGN |
| KT | FCGR2A |
| KT | ITIH3 |
| KT | CPXM2 |
| KT | ACP5 |
| KT | KAZALD1 |
| KT | MIA |
| KT | FGF1 |
| KT | LRRC4B |
| KT | CCL26 |
| KT | C2orf40 |
| KT | PLVAP |
| KT | SOSTDC1 |
| KT | CGREF1 |
| KT | TNFRSF12A |
| KT | CLCC1 |
| KT | SMR3A |
| KT | LY6G6D |
| KT | CCL18 |
| KT | CCL2 |
| KT | RTN4RL2 |
| KT | C10orf54 |
| KT | FAM24B |
| KT | FGF23 |
| KT | RSPO3 |
| KT | GPR156 |
| KT | TGOLN2 |
| KT | XG |
| KT | UNQ9165/PRO28630 |
| KT | FKBP14 |
| KT | GPRC6A |
| KT | C6orf15 |
| KT | CREG2 |
| KT | PTHLH |
| KT | ASIP |
| KT | GPR25 |
| KT | GPR17 |
| KT | HCTR1 |
| KT | SLC38A4 |
| KT | SLC8B1 |
| KT | IL15RA |
| KT | SLC2A10 |
| KT | NPBW1 |
| KT | PAEP |
| KT | DKK2 |
| KT | CADM2 |
| KT | CCL15 |
| KT | CXCR3 |
| KT | ADRA1D |
| KT | IFNA5 |
| KT | KIRREL3 |
| KT | BMPR1A |
| KT | TNFRSF17 |
| KT | MFSD2A |
| KT | C12orf49 |
| KT | FCGR2C |
| KT | COL9A3 |
| KT | SPINK7 |
| KT | WFDC1 |
| KT | ADM |
| KT | SOST |
| KT | RXFP3 |
| KT | TM4SF6 |
| KT | IGFBP3 |
| KT | NETO1 |
| KT | FGF7 |
| KT | LPA4 |
| KT | SPINK1 |
| KT | TMED1 |
| KT | ADM2 |
| KT | RAET1L |
| KT | S1PR4 |
| KT | C2orf66 |
| KT | CST6 |
| KT | SERPINI1 |
| KT | IFITM10 |
| KT | SEMG1 |
| KT | SCG3 |
| KT | SCG5 |
| KT | IL17BR |
| KT | ANGPTL5 |
| KT | CSAG1 |
| KT | REG1A |
| KT | IGFBP6 |
| KT | GPR83 |
| KT | INSL3 |
| KT | PRRG1 |
| KT | CD248 |
| KT | EFNB3 |
| KT | IL21 |
| KT | NOPE |
| KT | APOC3 |
| KT | NPPC |
| KT | JTB |
| KT | SELL |
| KT | UNC5B |
| KT | WFDC13 |
| KT | APLP2 |
| KT | LYPD1 |
| KT | C17orf99 |
| KT | MADCAM1 |
| KT | FZD9 |
| KT | CST1 |
| KT | IL32 |
| KT | PGA3 |
| KT | ADAMTS16 |
| KT | PSAPL1 |
| KT | IL1F5 |
| KT | P4HB |
| KT | CXCL11 |
| KT | SLC20A1 |
| KT | SPX |
| KT | SLC10A4 |
| KT | TMEM41A |
| KT | LRFN2 |
| KT | ULBP2 |
| KT | LAG3 |
| KT | EPCAM |
| KT | OSM |
| KT | SLC39A8 |
| KT | FGFRL1 |
| KT | GPR22 |
| KT | CP |
| KT | AMELX |
| KT | MUCL1 |
| KT | FSTL1 |
| KT | GZMM |
| KT | GSN |
| KT | SLC6A5 |
| KT | LCN1 |
| KT | PRL |
| KT | CXCL9 |
| KT | 229E-S1 |
| KT | F13B |
| KT | CPVL |
| KT | TFF2 |
| KT | SPINK13 |
| KT | SNORC |
| KT | STC2 |
| KT | LIFR |
| KT | OS9 |
| KT | HRC |
| KT | SMOC2 |
| KT | FGFBP3 |
| KT | CRTAP |
| KT | SGCB |
| KT | TOR1B |
| KT | C6 |
| KT | GALP |
| KT | SDC1 |
| KT | PDGFA |
| KT | OXTR |
| KT | KLK7 |
| KT | RNASE8 |
| KT | CYTL1 |
| KT | SPINK8 |
| KT | HRH3 |
| KT | CALY |
| KT | LCN15 |
| KT | APP |
| KT | TRPC3 |
| KT | AVP |
| KT | RNF167 |
| KT | GPR77 |
| KT | IGF1 |
| KT | CXCR5 |
| KT | PGA4 |
| KT | CLDN9 |
| KT | OXER1 |
| KT | CTSG |
| KT | FGF17 |
| KT | GPR3 |
| KT | COV2-S1 |
| KT | EDIL3 |
| KT | AZU1 |
| KT | NPTX2 |
| KT | LRRC8C |
| KT | DEFB126 |
| KT | CXCR1 |
| KT | PMCH |
| KT | CCL11 |
| KT | MOG |
| KT | TNFRSF6B |
| KT | PDGFB |
| KT | TFF1 |
| KT | BTNL8 |
| KT | CHGA |
| KT | NTRK2 |
| KT | PTN |
| KT | ACKR2 |
| KT | SERPINE2 |
| KT | C9 |
| KT | MCP |
| KT | CMKLR1 |
| KT | C6orf25 |
| KT | OBP2A |
| KT | SLC22A8 |
| KT | NGFR |
| KT | CT83 |
| KT | CCL8 |
| KT | IL6R |
| KT | PLGRKT |
| KT | ART1 |
| KT | CXCL13 |
| KT | HNRNPA2B1 |
| KT | CD14 |
| KT | LHFPL6 |
| KT | FAM20A |
| KT | NOTCH2NL |
| KT | ISM2 |
| KT | MUC7 |
| KT | LGALS1 |
| KT | PLAC9 |
| KT | FAM187B |
| KT | FGF19 |
| KT | FAM3D |
| KT | ODAPH |
| KT | KCNK1 |
| KT | LRIT3 |
| KT | RTN4RL1 |
| KT | SLC22A4 |
| KT | FAM19A4 |
| KT | PRRT3 |
| KT | F2R |
| KT | F12 |
| KT | PKD2L1 |
| KT | OPRM1 |
| KT | VSTM2B |
| KT | KLRF1 |
| KT | MC5R |
| KT | CCL1 |
| KT | EREG |
| KT | PLA2G15 |
| KT | CLDN4 |
| KT | LHFPL1 |
| KT | CDSN |
| KT | APOE |
| KT | TNF |
| KT | OPRK1 |
| KT | PDIA6 |
| KT | NTNG2 |
| KT | TRH |
| KT | FAM24A |
| KT | OPN4 |
| KT | TIMP1 |
| KT | CD99 |
| KT | CSN3 |
| KT | AIMP1 |
| KT | XK |
| KT | SLC6A11 |
| KT | IGFBPL1 |
| KT | HAPLN2 |
| KT | ALPI |
| KT | FCMR |
| KT | CSHL1 |
| KT | PRAP1 |
| KT | COL26A1 |
| KT | APLP1 |
| KT | RAMP2 |
| KT | LYPD2 |
| KT | TMEM219 |
| KT | CASQ1 |
| KT | NAPSA |
| KT | COL8A1 |
| KT | FRZB |
| KT | DEFB116 |
| KT | DLL3 |
| KT | KCNMB4 |
| KT | S100A8 |
| KT | COMT |
| KT | ANGPT4 |
| KT | C1QL1 |
| KT | GRM5 |
| KT | KLRK1 |
| KT | VTCN1 |
| KT | MARCO |
| KT | RNASE10 |
| KT | FCN2 |
| KT | IL13 |
| KT | WFDC8 |
| KT | CCL20 |
| KT | CD300A |
| KT | IL1RN |
| KT | GGH |
| KT | IL8RB |
| KT | WNT5A |
| KT | MDK |
| KT | CELA3B |
| KT | PSAP |
| KT | IL25 |
| KT | SELE |
| KT | ACVRL1 |
| KT | PAPLN |
| KT | DEAF1 |
| KT | CDNF |
| KT | SDF2L1 |
| KT | PRR4 |
| KT | SHBG |
| KT | IFNA8 |
| KT | FAM3A |
| KT | SPP2 |
| KT | C1QTNF2 |
| KT | TMPRSS2 |
| KT | CXCL17 |
| KT | PRRT1 |
| KT | EDAR |
| KT | LIPF |
| KT | TREM2 |
| KT | FZD7 |
| KT | FCRL6 |
| KT | CLCF1 |
| KT | FAM20C |
| KT | TNFSF9 |
| KT | LRRN4 |
| KT | CELA3A |
| KT | LCN12 |
| KT | CHODL |
| KT | CLEC-6 |
| KT | RNF149 |
| KT | SYNDIG1L |
| KT | ISLR2 |
| KT | EPOR |
| KT | ASTN2 |
| KT | LGI4 |
| KT | INHBE |
| KT | NRG1 |
| KT | FAM19A5 |
| KT | EGFR |
| KT | CLDN12 |
| KT | CD74 |
| KT | PRSS55 |
| KT | PLA2G2C |
| KT | CFP |
| KT | LCAT |
| KT | BPIFA1 |
| KT | CNNM4 |
| KT | THBS3 |
| KT | CRELD2 |
| KT | C9orf47 |
| KT | MANSC4 |
| KT | METTL24 |
| KT | NPY4R |
| KT | SLCO1B1 |
| KT | ALPPL2 |
| KT | TMPRSS3 |
| KT | SPACA4 |
| KT | CDH9 |
| KT | GYPA |
| KT | GLRA1 |
| KT | CX3CL1 |
| KT | OLR1 |
| KT | EFNA5 |
| KT | PRSS22 |
| KT | LRRC21 |
| KT | IER3 |
| KT | PROK1 |
| KT | TREM1 |
| KT | IL6ST |
| KT | DNASE1L1 |
| KT | MMP17 |
| KT | PRSS23 |
| KT | NPNT |
| KT | IL1B |
| KT | MMP9 |
| KT | CA14 |
| KT | NXPH4 |
| KT | GABRR3 |

### Example 3: Diagnostic or Prognostic Autoantigens

TABLE 4
List of Diagnostic or Prognostic Autoantigens
and their Corresponding Diseases or Disorders

| Disease | Target |
|---|---|
| AAV | EDIL3 |
| AAV | LY6H |
| AAV | TREM2 |
| APECED | IFNA6 |
| APECED | IFNW1 |
| APECED | IFNA17 |
| APECED | IFNA14 |
| APECED | LCN1 |
| APECED | GPHB5 |
| APECED | IFNA13 |
| APECED | IFNA8 |
| APECED | IL22RA2 |
| APECED | PRRT3 |
| APECED | IL22 |
| APECED | C5orf64 |
| APECED | CP |
| APECED | IFNA5 |
| APECED | LEG1 |
| APECED | PNLIPRP2 |
| APECED | IL17A |
| APECED | PRG3 |
| APECED | IL17F |
| APECED | IFNA2 |
| APECED | IL5 |
| APECED | SLC2A10 |
| APECED | GIF |
| APECED | PNLIPRP1 |
| APECED | BPIFA1 |
| APECED | PDILT |
| APECED | IFNL2 |
| APECED | PDGFB |
| APECED | CST5 |
| APECED | PNLIP |
| APECED | IGSF4B |
| APECED | TGFA |
| APECED | BPIFA2 |
| APECED | HSPA13 |
| APECED | ODAPH |
| APECED | SPINK4 |
| APECED | IGFBP1 |
| APECED | IL6 |
| APECED | CLCC1 |
| APECED | BTN1A1 |
| APECED | EREG |
| APECED | FAM19A4 |
| APECED | PTPRR |
| APECED | CST6 |
| APECED | RAMP2 |
| APECED | IL28B |
| APECED | TSLP |
| APECED | SPAG11B |
| APECED | CNPY3 |
| APECED | FAM3A |
| APECED | SLC41A2 |
| APECED | FKBP14 |
| APECED | AFP |
| APECED | TM4SF6 |
| APECED | REG1A |
| APECED | PANX3 |
| APECED | PRRG3 |
| APECED | RNASE8 |
| APECED | SMR3A |
| APECED | SPINK1 |
| APECED | PSAP |
| APECED | SERPINE1 |
| APECED | CST4 |
| APECED | PRRG1 |
| APECED | KLK2 |
| APECED | HCRTR2 |
| APECED | LAIR2 |
| APECED | OTOR |
| APECED | TFF2 |
| APECED | MSR1 |
| APECED | CCL7 |
| APECED | ADM2 |
| APECED | OPN4 |
| APECED | PAP |
| APECED | MMP1 |
| APECED | REG4 |
| APECED | PMCH |
| APECED | CLPS |
| APECED | OBP2A |
| APECED | ACRV1 |
| APECED | DEFA5 |
| APECED | ECSCR |
| APECED | LRIT3 |
| APECED | PLA2G10 |
| APECED | TM9SF3 |
| APS | IL6R |
| APS | IFNA13 |
| APS | IFNA14 |
| APS | IFNA17 |
| APS | IFNA2 |
| APS | IFNA5 |
| APS | IFNA6 |
| APS | IFNA8 |
| APS | IL6R |
| CIDP | CXCL1 |
| CIDP | CXCL2 |
| CIDP | CXCL3 |
| CIDP | PDGFB |
| CIDP | TMEM149 |
| CIDP | CD74 |
| CIDP | CXCL13 |
| COVID-19 | APOO |
| COVID-19 | OPRL1 |
| COVID-19 | IFNA14 |
| COVID-19 | MIA2 |
| COVID-19 | FKBP2 |
| COVID-19 | GPR1 |
| COVID-19 | IL29 |
| COVID-19 | PTPRR |
| COVID-19 | RCN2 |
| COVID-19 | IFNA13 |
| COVID-19 | IFNW1 |
| COVID-19 | IL1A |
| COVID-19 | TSPAN9 |
| COVID-19 | SHISA7 |
| COVID-19 | IFNA17 |
| COVID-19 | LEP |
| COVID-19 | CALU |
| COVID-19 | SSPN |
| COVID-19 | LPAL2 |
| COVID-19 | OBP2B |
| COVID-19 | CST5 |
| COVID-19 | IL6 |
| COVID-19 | CCDC47 |
| COVID-19 | ACRV1 |
| COVID-19 | PGA3 |
| COVID-19 | LRRC8C |
| COVID-19 | PMCH |
| COVID-19 | GPR6 |
| COVID-19 | CSF2 |
| COVID-19 | RCN3 |
| COVID-19 | LYSMD4 |
| COVID-19 | CD99 |
| COVID-19 | IFNA5 |
| COVID-19 | IFNL2 |
| COVID-19 | CXCL9 |
| COVID-19 | SLC41A2 |
| COVID-19 | EPYC |
| COVID-19 | DUOXA1 |
| COVID-19 | LACRT |
| COVID-19 | CNPY2 |
| COVID-19 | KLK8 |
| COVID-19 | MZB1 |
| COVID-19 | LYG2 |
| COVID-19 | MUCL3 |
| COVID-19 | LALBA |
| COVID-19 | ZG16B |
| COVID-19 | ODAM |
| COVID-19 | PILRA |
| COVID-19 | HRC |
| COVID-19 | PPBP |
| COVID-19 | CSPG5 |
| COVID-19 | PTPRN2 |
| COVID-19 | CST4 |
| COVID-19 | FAM168B |
| COVID-19 | TNFRSF17 |
| COVID-19 | OTOS |
| COVID-19 | SPINK9 |
| COVID-19 | KLRC2 |
| COVID-19 | IFNA8 |
| COVID-19 | TMEM119 |
| COVID-19 | CSAG1 |
| COVID-19 | OTOR |
| COVID-19 | KCT2 |
| COVID-19 | PGA4 |
| COVID-19 | SPINK4 |
| COVID-19 | FCGR2A |
| COVID-19 | CNPY3 |
| COVID-19 | NEGR1 |
| COVID-19 | ERP27 |
| COVID-19 | AGRP |
| COVID-19 | PRR27 |
| COVID-19 | MCFD2 |
| COVID-19 | IGFBP6 |
| COVID-19 | IFNA2 |
| COVID-19 | LGALS3 |
| COVID-19 | SPOCK1 |
| COVID-19 | KCNV2 |
| COVID-19 | HCRTR2 |
| COVID-19 | LECT2 |
| COVID-19 | PLA2G2E |
| COVID-19 | FAM19A3 |
| COVID-19 | SPACA7 |
| COVID-19 | NENF |
| COVID-19 | IL6R |
| COVID-19 | SPX |
| COVID-19 | IGFBP1 |
| COVID-19 | SRGN |
| COVID-19 | LAIR2 |
| COVID-19 | CPXM2 |
| COVID-19 | CCL17 |
| COVID-19 | TUSC5 |
| COVID-19 | LOC644613 |
| COVID-19 | TNFRSF21 |
| COVID-19 | GPR77 |
| COVID-19 | C2orf40 |
| COVID-19 | C5A |
| COVID-19 | IFNA6 |
| COVID-19 | SPP1 |
| COVID-19 | SERPINA3 |
| COVID-19 | OXTR |
| COVID-19 | KLRC1 |
| COVID-19 | SEMG2 |
| COVID-19 | APOH |
| COVID-19 | PRRG1 |
| COVID-19 | BTC |
| COVID-19 | MSLN |
| COVID-19 | FAM19A2 |
| COVID-19 | CXCL1 |
| COVID-19 | PRSS55 |
| COVID-19 | SLCO2B1 |
| COVID-19 | BTN1A1 |
| COVID-19 | COV2-RBD |
| COVID-19 | OS9 |
| COVID-19 | PGLYRP1 |
| COVID-19 | DKK3 |
| COVID-19 | TOR1B |
| COVID-19 | CST1 |
| COVID-19 | LRRC8D |
| COVID-19 | ACKR1 |
| COVID-19 | COL8A1 |
| COVID-19 | CXCL3 |
| COVID-19 | ODAPH |
| COVID-19 | PIANP |
| COVID-19 | PSORS1C2 |
| COVID-19 | RNASE10 |
| COVID-19 | CXCR7 |
| COVID-19 | PLVAP |
| COVID-19 | CDSN |
| COVID-19 | SDF2L1 |
| COVID-19 | TFF2 |
| COVID-19 | HSPA13 |
| COVID-19 | CXCR5 |
| COVID-19 | C5orf64 |
| COVID-19 | EPO |
| COVID-19 | GNLY |
| COVID-19 | OPRM1 |
| COVID-19 | TGFA |
| COVID-19 | SLC2A10 |
| COVID-19 | CXCL13 |
| COVID-19 | CD99L2 |
| COVID-19 | AGER |
| COVID-19 | CGA |
| COVID-19 | CRTAM |
| COVID-19 | SLC1A1 |
| COVID-19 | CDH19 |
| COVID-19 | GPR25 |
| COVID-19 | CCL8 |
| COVID-19 | SERPINI1 |
| COVID-19 | SPINK8 |
| COVID-19 | SLPI |
| COVID-19 | HRH3 |
| COVID-19 | TMEM149 |
| COVID-19 | CD38 |
| COVID-19 | REG4 |
| COVID-19 | IGFBP5 |
| COVID-19 | FKBP7 |
| COVID-19 | GRM5 |
| COVID-19 | CXCR3 |
| COVID-19 | PTHLH |
| COVID-19 | LY6K |
| COVID-19 | PLAC9 |
| COVID-19 | LPL |
| COVID-19 | CCKAR |
| COVID-19 | RTN4R |
| COVID-19 | GYPA |
| COVID-19 | TMED1 |
| COVID-19 | DRAXIN |
| COVID-19 | CCL13 |
| COVID-19 | LRRC8A |
| COVID-19 | ANGPTL4 |
| COVID-19 | NPPC |
| COVID-19 | IL22 |
| COVID-19 | CCL21 |
| COVID-19 | RCN1 |
| COVID-19 | CD74 |
| COVID-19 | FGF17 |
| COVID-19 | PAEP |
| COVID-19 | CNPY4 |
| COVID-19 | APOC3 |
| COVID-19 | SPINK1 |
| COVID-19 | AZGP1 |
| COVID-19 | STC2 |
| COVID-19 | S1PR4 |
| COVID-19 | IBSP |
| COVID-19 | CEACAM18 |
| COVID-19 | SLC38A4 |
| COVID-19 | CSN2 |
| COVID-19 | VSIG2 |
| COVID-19 | ENSP00000381830 |
| COVID-19 | CSHL1 |
| COVID-19 | CASQ1 |
| COVID-19 | XG |
| COVID-19 | ENDOU |
| COVID-19 | RAET1L |
| COVID-19 | COL10A1 |
| COVID-19 | PTH |
| COVID-19 | SLC15A1 |
| COVID-19 | SLC6A2 |
| COVID-19 | PRRT1 |
| COVID-19 | CLCC1 |
| COVID-19 | F2R |
| COVID-19 | JTB |
| COVID-19 | TGOLN2 |
| COVID-19 | CCL16 |
| COVID-19 | MIA |
| COVID-19 | TNF |
| COVID-19 | TMEM91 |
| COVID-19 | RTBDN |
| COVID-19 | MPL |
| COVID-19 | RSPO1 |
| COVID-19 | RSPO3 |
| COVID-19 | PRSS3 |
| COVID-19 | GPR17 |
| COVID-19 | CCR9 |
| COVID-19 | GP6 |
| COVID-19 | PRH1; |
| COVID-19 | EQTN |
| COVID-19 | RNF43 |
| COVID-19 | SPN |
| COVID-19 | IGSF4B |
| COVID-19 | CFD |
| COVID-19 | SPACA5 |
| COVID-19 | CHGA |
| COVID-19 | UNQ6190/PRO20217 |
| COVID-19 | APOA1 |
| COVID-19 | PRG3 |
| COVID-19 | SLC2A2 |
| COVID-19 | CCL11 |
| COVID-19 | TSLP |
| COVID-19 | SMOC2 |
| COVID-19 | HTR5 |
| COVID-19 | PRAP1 |
| COVID-19 | LY6H |
| COVID-19 | IMPG1 |
| COVID-19 | TNFRSF12A |
| COVID-19 | SSTR2 |
| COVID-19 | IGFBP3 |
| COVID-19 | PRLR |
| COVID-19 | PRR4 |
| COVID-19 | IL13 |
| COVID-19 | HCTR1 |
| COVID-19 | IGF1 |
| COVID-19 | CD300E |
| COVID-19 | LINC00305 |
| COVID-19 | SPESP1 |
| COVID-19 | FRZB |
| COVID-19 | IL28B |
| COVID-19 | MMP9 |
| COVID-19 | GAST |
| COVID-19 | FGF1 |
| COVID-19 | IL15RA |
| COVID-19 | CCR10 |
| COVID-19 | VEGFB |
| COVID-19 | SERPINE1 |
| COVID-19 | EXOC3-AS1 |
| COVID-19 | PRRT3 |
| COVID-19 | NETO1 |
| COVID-19 | VSTM2B |
| COVID-19 | CCR4 |
| COVID-19 | APP |
| COVID-19 | AMTN |
| COVID-19 | CXCL6 |
| COVID-19 | NINJ1 |
| COVID-19 | KLK9 |
| COVID-19 | SDF4 |
| COVID-19 | CPE |
| COVID-19 | AMELX |
| COVID-19 | DCD |
| COVID-19 | ANTXRL |
| COVID-19 | CCR2 |
| COVID-19 | PCSK1 |
| COVID-19 | QRFP |
| COVID-19 | RGMB |
| COVID-19 | NPY2R |
| COVID-19 | IGFBP7 |
| COVID-19 | SLC2A12 |
| COVID-19 | PPT1 |
| COVID-19 | CCL7 |
| COVID-19 | JCHAIN |
| COVID-19 | ADCYAP1 |
| COVID-19 | PDZD11 |
| COVID-19 | CP |
| COVID-19 | MANF |
| COVID-19 | GZMA |
| COVID-19 | TXNDC12 |
| COVID-19 | PGC |
| COVID-19 | ACVR1 |
| COVID-19 | WFDC13 |
| COVID-19 | SFRP4 |
| COVID-19 | REG1A |
| COVID-19 | GPR37 |
| COVID-19 | NOPE |
| COVID-19 | C11orf94 |
| COVID-19 | SCARA5 |
| COVID-19 | GPR19 |
| COVID-19 | EMC7 |
| COVID-19 | CCL15 |
| COVID-19 | CA4 |
| COVID-19 | RNASE8 |
| COVID-19 | MLN |
| COVID-19 | UNQ9165/PRO28630 |
| COVID-19 | NTRK3 |
| COVID-19 | TREML1 |
| COVID-19 | CDH15 |
| COVID-19 | SMR3A |
| COVID-19 | DKK1 |
| COVID-19 | OXER1 |
| COVID-19 | FAM24B |
| COVID-19 | CRLF1 |
| COVID-19 | PDIA6 |
| COVID-19 | PLA2G12B |
| COVID-19 | FGF7 |
| COVID-19 | ZP4 |
| COVID-19 | BAMBI |
| COVID-19 | GKN2 |
| COVID-19 | IGFBPL1 |
| COVID-19 | MMP7 |
| COVID-19 | MANSC4 |
| COVID-19 | APOA4 |
| COVID-19 | SUSD6 |
| COVID-19 | CELA1 |
| COVID-19 | IGLL1 |
| COVID-19 | IL9 |
| COVID-19 | MADCAM1 |
| COVID-19 | NPBW1 |
| COVID-19 | HAVCR1 |
| COVID-19 | ITPRIPL1 |
| COVID-19 | SOST |
| COVID-19 | LHFPL1 |
| COVID-19 | SDC3 |
| COVID-19 | SEMG1 |
| COVID-19 | C1QB |
| COVID-19 | ASIP |
| COVID-19 | CCL18 |
| COVID-19 | LHFPL5 |
| COVID-19 | IGFL2 |
| COVID-19 | FGFRL1 |
| COVID-19 | EFNB2 |
| COVID-19 | C2orf66 |
| COVID-19 | MFAP3 |
| COVID-19 | C6orf15 |
| COVID-19 | OPN4 |
| COVID-19 | NOV |
| COVID-19 | GNS |
| COVID-19 | FKBP14 |
| COVID-19 | CELA2B |
| COVID-19 | C9 |
| COVID-19 | VWC2L |
| COVID-19 | BMPR2 |
| COVID-19 | CSH2 |
| COVID-19 | IL1RAP |
| COVID-19 | C1QTNF2 |
| COVID-19 | SLC10A4 |
| COVID-19 | IL16 |
| COVID-19 | LRIT3 |
| COVID-19 | GRN |
| COVID-19 | NIPAL4 |
| COVID-19 | GNRH1 |
| COVID-19 | ATP4B |
| COVID-19 | APLP2 |
| COVID-19 | TMEM123 |
| COVID-19 | IL3 |
| COVID-19 | PDGFA |
| COVID-19 | EVI2B |
| COVID-19 | NGFR |
| COVID-19 | PROK1 |
| COVID-19 | SOSTDC1 |
| COVID-19 | FLJ36131 |
| COVID-19 | EREG |
| COVID-19 | TNFRSF9 |
| COVID-19 | LYG1 |
| COVID-19 | SLCO4C1 |
| COVID-19 | GUCA2A |
| COVID-19 | FAM19A5 |
| COVID-19 | IL21 |
| COVID-19 | FCMR |
| COVID-19 | CADM2 |
| COVID-19 | CSF3 |
| COVID-19 | CA11 |
| COVID-19 | NTRK2 |
| COVID-19 | CRELD2 |
| COVID-19 | GPR120 |
| COVID-19 | C9orf135 |
| COVID-19 | SLC1A5 |
| COVID-19 | SYCN |
| COVID-19 | COL9A3 |
| COVID-19 | ADRA1D |
| COVID-19 | GLB1 |
| COVID-19 | SV2C |
| COVID-19 | DKFZp686O24166 |
| COVID-19 | PRSS3P2 |
| COVID-19 | KIRREL3 |
| COVID-19 | VSTM2A |
| COVID-19 | GCG |
| COVID-19 | SERPINE2 |
| COVID-19 | EDA2R |
| COVID-19 | CPAMD8 |
| COVID-19 | SCN3B |
| COVID-19 | OXT |
| COVID-19 | CD3E |
| COVID-19 | INSL3 |
| COVID-19 | CALY |
| COVID-19 | GHSR |
| COVID-19 | SCGB1D1 |
| COVID-19 | C6 |
| COVID-19 | CLDN2 |
| COVID-19 | MUC7 |
| COVID-19 | KISS1 |
| COVID-19 | ULBP2 |
| COVID-19 | CLDN7 |
| COVID-19 | IGFBP2 |
| COVID-19 | EFNB3 |
| COVID-19 | NXPH1 |
| COVID-19 | GHRHR |
| COVID-19 | LILRA4 |
| COVID-19 | OTOL1 |
| COVID-19 | EFNB1 |
| COVID-19 | FGFBP3 |
| COVID-19 | GPR63 |
| COVID-19 | PRRG4 |
| COVID-19 | MUCL1 |
| COVID-19 | XCL1 |
| COVID-19 | TMEM120A |
| COVID-19 | TMEM108 |
| COVID-19 | IL1F5 |
| COVID-19 | MSMP |
| COVID-19 | CXCL12 |
| COVID-19 | GNPTG |
| COVID-19 | SDC4 |
| COVID-19 | FZD9 |
| COVID-19 | CCL4L1 |
| COVID-19 | GPRC6A |
| COVID-19 | GPR156 |
| COVID-19 | ITIH3 |
| COVID-19 | RAMP2 |
| COVID-19 | TNFRSF11A |
| COVID-19 | DKK2 |
| COVID-19 | SPINK13 |
| COVID-19 | SDCBP |
| COVID-19 | CD8B2 |
| COVID-19 | CTSG |
| COVID-19 | CST2 |
| COVID-19 | EDDM3B |
| COVID-19 | CLTRN |
| COVID-19 | PLA2G10 |
| COVID-19 | DCN |
| COVID-19 | DAG1 |
| COVID-19 | CXCL16 |
| COVID-19 | CCRL2 |
| COVID-19 | DEFB108B |
| COVID-19 | MRGPRF |
| COVID-19 | FCRL3 |
| COVID-19 | NPS |
| COVID-19 | OBP2A |
| COVID-19 | ACKR2 |
| COVID-19 | GRM2 |
| COVID-19 | FAM174A |
| COVID-19 | MSR1 |
| COVID-19 | NOG |
| COVID-19 | TMEM102 |
| COVID-19 | LAIR1 |
| COVID-19 | IL22RA2 |
| COVID-19 | SPACA3 |
| COVID-19 | WIF1 |
| COVID-19 | F13B |
| COVID-19 | LRTM1 |
| COVID-19 | ERVH48-1 |
| COVID-19 | CCL2 |
| COVID-19 | TFF1 |
| COVID-19 | ADM2 |
| COVID-19 | IFITM10 |
| COVID-19 | HSD11BIL |
| COVID-19 | AXL |
| COVID-19 | FMR1NB |
| COVID-19 | C6orf25 |
| COVID-19 | OPN3 |
| COVID-19 | MUC13 |
| COVID-19 | CCL28 |
| COVID-19 | CCL26 |
| COVID-19 | PTN |
| COVID-19 | SLC39A8 |
| COVID-19 | FGF21 |
| COVID-19 | TIMD4 |
| COVID-19 | NPTX2 |
| COVID-19 | IL17RD |
| COVID-19 | PAPLN |
| COVID-19 | TMEM219 |
| COVID-19 | CYB5D2 |
| COVID-19 | IL1B |
| COVID-19 | FSTL1 |
| COVID-19 | PTPRJ |
| COVID-19 | NPY1R |
| COVID-19 | CLDN18 |
| COVID-19 | FLT3LG |
| COVID-19 | C17orf99 |
| COVID-19 | SLC6A5 |
| COVID-19 | AIMP1 |
| COVID-19 | TNFRSF8 |
| COVID-19 | CD248 |
| COVID-19 | TM9SF3 |
| COVID-19 | FCGR2C |
| COVID-19 | MPZL3 |
| COVID-19 | OSTN |
| COVID-19 | SPARCL1 |
| COVID-19 | TMPRSS11D |
| COVID-19 | KLK7 |
| COVID-19 | GDPD3 |
| COVID-19 | IL34 |
| COVID-19 | BTNL8 |
| COVID-19 | ASTL |
| COVID-19 | CLDN19 |
| COVID-19 | SCG5 |
| COVID-19 | PSAP |
| COVID-19 | PRRG3 |
| COVID-19 | PLA2G12A |
| COVID-19 | LCN1 |
| COVID-19 | LRRTM2 |
| COVID-19 | FAM3D |
| COVID-19 | PTGS2 |
| COVID-19 | FCRLB |
| COVID-19 | CST8 |
| COVID-19 | ANGPTL5 |
| COVID-19 | OPRK1 |
| COVID-19 | APOD |
| COVID-19 | ADM |
| COVID-19 | CLU |
| COVID-19 | PANX3 |
| COVID-19 | SLC52A3 |
| DIL | CXCL1 |
| DIL | TNF |
| DIL | TSLP |
| DM | CD81 |
| KT | CD99L2 |
| KT | CD80 |
| KT | TNFRSF21 |
| KT | TMEM102 |
| KT | MICA |
| KT | PILRA |
| KT | AGER |
| KT | ULBP1 |
| KT | JCHAIN |
| KT | TLR1 |
| KT | TNFRSF6 |
| KT | SIRPA |
| KT | FCER1A |
| KT | CD300E |
| KT | B2M |
| KT | C10orf54 |
| KT | GPR17 |
| KT | IL15RA |
| KT | TMED1 |
| KT | S1PR4 |
| KT | IFITM10 |
| KT | IL17BR |
| KT | EFNB3 |
| KT | C6 |
| KT | GPR77 |
| KT | IL2RA |
| KT | IFNG |
| KT | IL9 |
| KT | IFNL2 |
| KT | MZB1 |
| KT | IL1A |
| KT | CSF2 |
| KT | IL4 |
| KT | CRLF1 |
| KT | IL34 |
| KT | IFNA17 |
| KT | IL17RD |
| KT | TGFA |
| KT | IL6 |
| KT | IL28B |
| KT | PRG3 |
| KT | IFNA13 |
| KT | IL16 |
| KT | TSLP |
| KT | IFNA6 |
| KT | IFNA2 |
| KT | IFNA14 |
| KT | TNFRSF12A |
| KT | CCL15 |
| KT | IFNA5 |
| KT | TNFRSF17 |
| KT | IL21 |
| KT | C17orf99 |
| KT | IL1F5 |
| KT | OSM |
| KT | GZMM |
| KT | LIFR |
| KT | ACKR1 |
| KT | CCL16 |
| KT | CXCL3 |
| KT | CCL22 |
| KT | CXCL1 |
| KT | CCR10 |
| KT | GPR1 |
| KT | CXCL12 |
| KT | CCL4L1 |
| KT | PPBP |
| KT | CCL26 |
| KT | CCL2 |
| KT | CXCR3 |
| KT | CXCL9 |
| KT | TFF2 |
| KT | CXCR5 |
| KT | ANGPTL4 |
| KT | ADRB3 |
| KT | RETN |
| KT | PRLR |
| KT | ADCYAP1 |
| KT | AVPR1A |
| KT | GNRH1 |
| KT | GAST |
| KT | THBD |
| KT | CCKAR |
| KT | C2orf40 |
| KT | PTHLH |
| KT | NPBW1 |
| KT | RXFP3 |
| KT | ADM2 |
| KT | INSL3 |
| KT | ADM |
| KT | NPPC |
| KT | SPX |
| KT | STC2 |
| KT | OXTR |
| KT | AVP |
| KT | SLC1A1 |
| KT | SLC2A2 |
| KT | SLC22A25 |
| KT | KCNV2 |
| KT | HCTR1 |
| KT | SLC38A4 |
| KT | SLC8B1 |
| KT | SLC2A10 |
| KT | MFSD2A |
| KT | SLC20A1 |
| KT | SLC10A4 |
| KT | SLC6A5 |
| KT | GALP |
| KT | EPYC |
| KT | OTOL1 |
| KT | CDH19 |
| KT | IBSP |
| KT | AMTN |
| KT | PSORS1C2 |
| KT | IMPG1 |
| KT | COL10A1 |
| KT | BCAM |
| KT | ICAM2 |
| KT | SRGN |
| KT | CPXM2 |
| KT | CGREF1 |
| KT | CADM2 |
| KT | COL9A3 |
| KT | CD248 |
| KT | SELL |
| KT | MADCAM1 |
| KT | EPCAM |
| KT | CRTAP |
| KT | SGCB |
| KT | SDC1 |
| KT | LYG2 |
| KT | LGALS3 |
| KT | DCD |
| KT | BTN1A1 |
| KT | MPO |
| KT | PGLYRP1 |
| KT | WFDC12 |
| KT | AZU1 |
| KT | IGFBP1 |
| KT | DKK3 |
| KT | FGFR2 |
| KT | IGFBP2 |
| KT | CNPY2 |
| KT | NOV |
| KT | VEGFB |
| KT | TMEM119 |
| KT | FAM19A3 |
| KT | MANF |
| KT | TMEM149 |
| KT | NENF |
| KT | VSTM2A |
| KT | BMPR2 |
| KT | FGF1 |
| KT | FGF23 |
| KT | RSPO3 |
| KT | BMPR1A |
| KT | TM4SF6 |
| KT | IGFBP3 |
| KT | FGF7 |
| KT | IGFBP6 |
| KT | FZD9 |
| KT | FGFRL1 |
| KT | AMELX |
| KT | FSTL1 |
| KT | SNORC |
| KT | SMOC2 |
| KT | FGFBP3 |
| KT | PDGFA |
| KT | CYTL1 |
| KT | IGF1 |
| KT | FGF17 |
| KT | EDIL3 |
| KT | CNPY3 |
| KT | MCFD2 |
| KT | CCDC47 |
| KT | RCN3 |
| KT | RCN1 |
| KT | TGOLN2 |
| KT | C12orf49 |
| KT | OS9 |
| KT | SHISA7 |
| KT | MRGPRF |
| KT | CSPG5 |
| KT | HCRTR2 |
| KT | OTOR |
| KT | SV2C |
| KT | PRRG4 |
| KT | GFRA2 |
| KT | TMEM108 |
| KT | LRRC4B |
| KT | UNC5B |
| KT | LYPD1 |
| KT | LRFN2 |
| KT | SCGB3A1 |
| KT | OBP2B |
| KT | FLJ37218 |
| KT | VSIG2 |
| KT | EDDM3B |
| KT | EXOC3-AS1 |
| KT | NPY1R |
| KT | APOO |
| KT | GPR6 |
| KT | LYSMD4 |
| KT | OPRL1 |
| KT | PTPRN2 |
| KT | ERP27 |
| KT | NINJ1 |
| KT | TMEM91 |
| KT | S100A13 |
| KT | SSTR2 |
| KT | SYCN |
| KT | ANTXRL |
| KT | SLC2A12 |
| KT | MIA2 |
| KT | C5orf64 |
| KT | REG1B |
| KT | FAM174A |
| KT | ITPRIPL1 |
| KT | REG4 |
| KT | BRICD5 |
| KT | GPR182 |
| KT | NXPH1 |
| KT | NOG |
| KT | MLN |
| KT | GPR63 |
| KT | TMEM120A |
| KT | ACP5 |
| KT | KAZALD1 |
| KT | MIA |
| KT | PLVAP |
| KT | SMR3A |
| KT | RTN4RL2 |
| KT | FAM24B |
| KT | UNQ9165/PRO28630 |
| KT | GPRC6A |
| KT | ASIP |
| KT | GPR25 |
| KT | ADRA1D |
| KT | KIRREL3 |
| KT | SOST |
| KT | LPA4 |
| KT | SCG3 |
| KT | SCG5 |
| KT | REG1A |
| KT | GPR83 |
| KT | PRRG1 |
| KT | JTB |
| KT | CST1 |
| KT | PSAPL1 |
| KT | GPR22 |
| KT | CP |
| KT | GSN |
| KT | LCN1 |
| KT | PRL |
| KT | HRC |
| KT | LCN15 |
| KT | OXER1 |
| KT | NPTX2 |
| KT | APOA1 |
| KT | APOA4 |
| KT | APOC3 |
| KT | F13B |
| KT | SPOCK1 |
| KT | SPINK4 |
| KT | KLK8 |
| KT | PTPRR |
| KT | SERPINE1 |
| KT | LALBA |
| KT | TXNDC12 |
| KT | FKBP2 |
| KT | PRSS3 |
| KT | TFPI2 |
| KT | PCSK1 |
| KT | CELA1 |
| KT | AZGP1 |
| KT | CES3 |
| KT | PLA2G2E |
| KT | ATP6AP2 |
| KT | PLBD2 |
| KT | PLA2G10 |
| KT | CST5 |
| KT | MMP7 |
| KT | CST4 |
| KT | TRABD2A |
| KT | DHRS4L2 |
| KT | ITIH3 |
| KT | FKBP14 |
| KT | SPINK7 |
| KT | WFDC1 |
| KT | SPINK1 |
| KT | CST6 |
| KT | SERPINI1 |
| KT | WFDC13 |
| KT | P4HB |
| KT | TOR1B |
| KT | KLK7 |
| KT | RNASE8 |
| KT | SPINK8 |
| KT | RNF167 |
| KT | CTSG |
| KT | ACRV1 |
| KT | SPACA7 |
| KT | SSPN |
| KT | SPACA3 |
| KT | ZG16B |
| KT | TSPAN9 |
| KT | RTN4R |
| KT | NPY2R |
| KT | GP6 |
| KT | FAM168B |
| KT | CSN2 |
| KT | SEMG2 |
| KT | GPR37 |
| KT | PRH1; |
| KT | OSTN |
| KT | FMR1NB |
| KT | CA11 |
| KT | SPESP1 |
| KT | GPR19 |
| KT | LYPD6B |
| KT | CLCC1 |
| KT | LY6G6D |
| KT | GPR156 |
| KT | XG |
| KT | NETO1 |
| KT | C2orf66 |
| KT | SEMG1 |
| KT | ANGPTL5 |
| KT | CSAG1 |
| KT | MUCL1 |
| KT | HRH3 |
| KT | APP |
| KT | 229E-RBD |
| KT | NL63-RBD |
| KT | COV2-RBD |
| KT | 229E-S1 |
| KT | COV2-S1 |
| KT | LOC644613 |
| KT | AGRP |
| KT | RCN2 |
| KT | IL29 |
| KT | MUCL3 |
| KT | CALU |
| KT | ENDOU |
| KT | SPINK9 |
| KT | SLPI |
| KT | FAM19A2 |
| KT | SERPINA3 |
| KT | PRR27 |
| KT | BTC |
| KT | IGFBP5 |
| KT | CXCL2 |
| KT | CCL13 |
| KT | LECT2 |
| KT | APOH |
| KT | LAIR2 |
| KT | DRAXIN |
| KT | CFD |
| KT | CCL17 |
| KT | DKK1 |
| KT | IGFBP7 |
| KT | UNQ6190/PRO20217 |
| KT | RARRES2 |
| KT | CCL21 |
| KT | CCL7 |
| KT | KAL1 |
| KT | HAVCR1 |
| KT | ATP4B |
| KT | PIANP |
| KT | GNLY |
| KT | OTOS |
| KT | CCL24 |
| KT | FCGR2A |
| KT | SOSTDC1 |
| KT | CCL18 |
| KT | C6orf15 |
| KT | CREG2 |
| KT | DKK2 |
| KT | NOPE |
| KT | APLP2 |
| KT | IL32 |
| KT | PGA3 |
| KT | ADAMTS16 |
| KT | CXCL11 |
| KT | TMEM41A |
| KT | LAG3 |
| KT | SLC39A8 |
| KT | CPVL |
| KT | SPINK13 |
| KT | CALY |
| KT | TRPC3 |
| KT | PGA4 |
| KT | CLDN9 |
| KT | GPR3 |
| Malaria | SPINK8 |
| Malaria | OBP2B |
| Malaria | GPR1 |
| Malaria | MCFD2 |
| Malaria | SDF2L1 |
| Malaria | FKBP2 |
| Malaria | EPYC |
| Malaria | PTPRR |
| Malaria | LGALS3 |
| Malaria | CD99L2 |
| Malaria | HCRTR2 |
| Malaria | TM4SF6 |
| Malaria | CGREF1 |
| Malaria | SSPN |
| Malaria | FZD4 |
| Malaria | SPINK4 |
| Malaria | GPR17 |
| Malaria | SRGN |
| Malaria | PRRG1 |
| Malaria | SLC1A4 |
| Malaria | CCDC47 |
| Malaria | ODAM |
| Malaria | MZB1 |
| Malaria | CSPG5 |
| Malaria | ACKR1 |
| Malaria | C9orf135 |
| Malaria | ZG16B |
| Malaria | KCT2 |
| Malaria | ANGPTL4 |
| Malaria | KLK8 |
| Malaria | DPT |
| Malaria | CD164L2 |
| Malaria | LY6G6D |
| Malaria | COL10A1 |
| Malaria | FAM19A3 |
| Malaria | RCN3 |
| Malaria | KLK3 |
| Malaria | COLEC12 |
| Malaria | DKK3 |
| Malaria | COL9A3 |
| Malaria | CSAG1 |
| Malaria | CNPY4 |
| Malaria | BCAM |
| Malaria | ADM |
| Malaria | ACRV1 |
| Malaria | SLC38A2 |
| Malaria | NBL1 |
| Malaria | TGFA |
| Malaria | CYTL1 |
| Malaria | SPACA7 |
| Malaria | CALR |
| Malaria | SMOC2 |
| Malaria | CSHL1 |
| Malaria | DCD |
| Malaria | IMPG1 |
| Malaria | IL1RN |
| Malaria | RAMP2 |
| Malaria | IGFBP6 |
| Malaria | TNFRSF17 |
| Malaria | SPX |
| Malaria | SERPINA3 |
| Malaria | NPY2R |
| Malaria | GPR19 |
| Malaria | FKBP7 |
| Malaria | CXCL3 |
| Malaria | NOV |
| Malaria | CXCR3 |
| Malaria | CCL15 |
| Malaria | RTBDN |
| Malaria | HEPACAM2 |
| Malaria | CST4 |
| Malaria | LEP |
| Malaria | SNORC |
| Malaria | CHGA |
| Malaria | SLC22A31 |
| Malaria | CCL13 |
| Malaria | OTOL1 |
| Malaria | C11orf94 |
| Malaria | RETN |
| Malaria | PLA2G2E |
| Malaria | PRRG3 |
| Malaria | APOO |
| Malaria | PGLYRP1 |
| Malaria | PRAP1 |
| Malaria | GAST |
| Malaria | TMEM91 |
| Malaria | HTR2B |
| Malaria | SCTR |
| Malaria | CNPY2 |
| Malaria | ZP4 |
| Malaria | CD151 |
| Malaria | SLC6A9 |
| Malaria | TMEM59 |
| Malaria | SERPINI2 |
| Malaria | CYSLTR2 |
| Malaria | SLC8B1 |
| Malaria | TRABD2B |
| Malaria | IGF1 |
| Malaria | S1PR3 |
| Malaria | IBSP |
| Malaria | JCHAIN |
| Malaria | CSH2 |
| Malaria | IL16 |
| Malaria | CELA1 |
| Malaria | NENF |
| Malaria | SGCA |
| Malaria | LINC00305 |
| Malaria | CXCL1 |
| Malaria | CNPY3 |
| Malaria | 229E-RBD |
| Malaria | LAS2 |
| Malaria | LYSMD4 |
| Malaria | PTHLH |
| Malaria | SLC10A4 |
| Malaria | RNASE10 |
| Malaria | KLK2 |
| Malaria | RAET1L |
| Malaria | HCTR1 |
| Malaria | SLC41A2 |
| Malaria | AXL |
| Malaria | CCL20 |
| Malaria | PRSS3 |
| Malaria | GPC6 |
| Malaria | TGOLN2 |
| Malaria | LRIT3 |
| Malaria | EMC10 |
| Malaria | AMTN |
| Malaria | PSORS1C2 |
| Malaria | NPBW1 |
| Malaria | S100A13 |
| Malaria | PCSK1 |
| Malaria | PTH |
| Malaria | INS |
| Malaria | CDNF |
| Malaria | SLC2A10 |
| Malaria | TXNDC12 |
| Malaria | ITIH3 |
| Malaria | LILRA4 |
| Malaria | IL1RAP |
| Malaria | XG |
| Malaria | IL17A |
| Malaria | CST5 |
| Malaria | CPE |
| Malaria | NL63-RBD |
| Malaria | GNRH1 |
| Malaria | CADM2 |
| Malaria | IL21R |
| Malaria | TSPAN13 |
| Malaria | OS9 |
| Malaria | P2RY10 |
| Malaria | SPARC |
| Malaria | PLA2G10 |
| Malaria | FKBP14 |
| Malaria | RXFP3 |
| Malaria | VEGFB |
| Malaria | VSTM2A |
| Malaria | ENSP00000381830 |
| Malaria | IFNA13 |
| Malaria | LYPD6B |
| Malaria | TREML1 |
| Malaria | GDPD3 |
| Malaria | SLC38A4 |
| Malaria | OPRK1 |
| Malaria | SV2C |
| Malaria | CPXM2 |
| Malaria | IGFBP2 |
| Malaria | TMEM169 |
| Malaria | CD300A |
| Malaria | GZMK |
| Malaria | ADCYAP1R1 |
| Malaria | LALBA |
| Malaria | PRH1; |
| Malaria | IFNW1 |
| Malaria | PTN |
| Malaria | OPN4 |
| Malaria | FLRT3 |
| Malaria | TRH |
| Malaria | FGF23 |
| Malaria | NPR3 |
| Malaria | MPZL3 |
| Malaria | TMEM108 |
| Malaria | TNFRSF10B |
| Malaria | SIGLEC10 |
| Malaria | GLB1 |
| Malaria | PRRT1 |
| Malaria | OPRM1 |
| Malaria | AKR1B10 |
| Malaria | KITLG |
| Malaria | OTOR |
| Malaria | CNR1 |
| Malaria | MINPP1 |
| Malaria | SDF4 |
| Malaria | GP6 |
| Malaria | GPR63 |
| Malaria | RNASE8 |
| Malaria | BDKBR1 |
| Malaria | CDH19 |
| Malaria | CCR4 |
| Malaria | SLC6A5 |
| Malaria | IL22 |
| Malaria | SHISA6 |
| Malaria | FZD9 |
| Malaria | GSN |
| Malaria | FCER1A |
| Malaria | IFNA6 |
| Malaria | KLK7 |
| Malaria | CTF1 |
| Malaria | NMBR |
| Malaria | C2orf66 |
| Malaria | TNFRSF12A |
| Malaria | INSL3 |
| Malaria | DRD5 |
| Malaria | SFTPA2 |
| Malaria | GPR37 |
| Malaria | IL13 |
| Malaria | GFRAL |
| Malaria | MOG |
| Malaria | TSPAN2 |
| Malaria | IGFBP1 |
| Malaria | SPINK1 |
| Malaria | PLGRKT |
| Malaria | PAPLN |
| Malaria | SCGB2A2 |
| Malaria | LCN15 |
| Malaria | SLC6A14 |
| Malaria | RNASE9 |
| MG | CXCL2 |
| MG | PDGFB |
| MG | REG4 |
| MG | CCL22 |
| MG | CCL2 |
| MM | CTLA4 |
| MM | RCN2 |
| MM | IL36B |
| MM | TNF |
| MM | CP |
| MM | CALU |
| MM | KLK8 |
| MM | SSPN |
| MM | IL1A |
| MM | TNFRSF9 |
| MM | SERPINA3 |
| MM | CDH19 |
| MM | OBP2B |
| MM | FGFBP3 |
| MM | NEGR1 |
| MM | XCL1 |
| MM | CST5 |
| MM | CNPY2 |
| MM | SRGN |
| MM | SPINK9 |
| MM | TM2D2 |
| MM | HSPA13 |
| MM | AXL |
| MM | FSTL1 |
| MM | MCFD2 |
| MM | ZG16B |
| MM | LEP |
| MM | TMEM108 |
| MM | MUCL3 |
| MM | IL17BR |
| MM | ODAPH |
| MM | CNPY3 |
| MM | FAM168B |
| MM | FAM19A3 |
| MM | IGFL2 |
| MM | DPT |
| MM | CCDC47 |
| MM | CXCL1 |
| MM | COL10A1 |
| MM | SPINK4 |
| MM | WFDC9 |
| MM | CSPG5 |
| MM | ENDOU |
| MM | VEGFB |
| MM | SPINK8 |
| MM | GNLY |
| MM | CRELD2 |
| MM | ERP27 |
| MM | RCN3 |
| MM | TMEM119 |
| MM | LOC644613 |
| MM | AGRP |
| MM | PIANP |
| MM | FAM19A2 |
| MM | IL9 |
| MM | GNRH2 |
| MM | LECT2 |
| MM | GNRH1 |
| MM | CCL17 |
| MM | IL29 |
| MM | KAZALD1 |
| MM | CST4 |
| MM | KCNK1 |
| MM | PANX3 |
| MM | FKBP14 |
| MM | PGA3 |
| MM | IGFBP2 |
| MM | PGLYRP1 |
| MM | NTS |
| MM | OTOL1 |
| MM | SOST |
| MM | SHISA7 |
| MM | CCL13 |
| MM | CGREF1 |
| MM | PRR27 |
| MM | IFNL2 |
| MM | DHRS4L2 |
| MM | LYG2 |
| MM | OTOS |
| MM | UNQ6190/PRO20217 |
| MM | GPC6 |
| MM | TNFRSF21 |
| MM | PSORS1C2 |
| MM | IFNA13 |
| MM | JCHAIN |
| MM | ACP5 |
| MM | TXNDC12 |
| MM | C5orf64 |
| MM | CLCC1 |
| MM | IL10RB |
| MM | FMR1NB |
| MM | SLPI |
| MM | HRC |
| MM | CCL22 |
| MM | CASQ1 |
| MM | CELA1 |
| MM | LCN1P1 |
| MM | ODAM |
| MM | TMED1 |
| MM | REG1A |
| MM | MZB1 |
| MM | ACRV1 |
| MM | IGLL1 |
| MM | HCRTR2 |
| MM | CST8 |
| MM | PLA2G2E |
| MM | BTN1A1 |
| MM | CLDN19 |
| MM | CSAG1 |
| MM | REG4 |
| MM | VEGFA |
| MM | COLEC12 |
| MM | LYSMD4 |
| MM | CCL24 |
| MM | C1QTNF2 |
| MM | PCSK1 |
| MM | PGA4 |
| MM | ITIH3 |
| MM | ICOSLG |
| MM | SDF2L1 |
| MM | LALBA |
| MM | PTPRN2 |
| MM | FGFRL1 |
| MM | SERPINE1 |
| MM | CSN2 |
| MM | BTC |
| MM | ANGPTL4 |
| MM | C2orf40 |
| MM | FCGR2A |
| MM | FGF1 |
| MM | IGSF4B |
| MM | CLTRN |
| MM | ERVK-18 |
| MM | BPIFC |
| MM | LAIR2 |
| MM | IFNW1 |
| MM | APOC3 |
| MM | CCL21 |
| MM | WFDC3 |
| MM | CD274 |
| MM | PTHLH |
| MM | PROKR2 |
| MM | LRRN4CL |
| MM | CA4 |
| MM | TMEM102 |
| MM | SLC41A2 |
| MM | MIA2 |
| MM | CDSN |
| MM | SLC6A13 |
| MM | CLDN2 |
| MM | RNF43 |
| MM | CALR |
| MM | PSAP |
| MM | AMELX |
| MM | RTBDN |
| MM | MICA |
| MM | HAVCR1 |
| MM | PDCD1 |
| MM | C9orf47 |
| MM | DRAXIN |
| MM | OTOR |
| MM | CCL18 |
| MM | PRSS3 |
| MM | IL6 |
| MM | C6orf15 |
| MM | NETO1 |
| MM | TMEM149 |
| MM | AMTN |
| MM | KLK14 |
| MM | RAMP2 |
| MM | SHISA6 |
| MM | TNFRSF12A |
| MM | FAM3A |
| MM | PLA2G10 |
| MM | MFAP2 |
| MM | PMCH |
| MM | CCL23 |
| MM | PRL |
| MM | LCN2 |
| MM | MOG |
| MM | ITPRIPL1 |
| MM | CST2 |
| MM | APOO |
| MM | CFD |
| MM | CTSW |
| MM | GP6 |
| MM | NOV |
| MM | MMP7 |
| MM | CXCL13 |
| MM | EREG |
| MM | NPPC |
| MM | IGFBP6 |
| MM | PRLR |
| MM | EXOC3-AS1 |
| MM | MIA |
| MM | OPN4 |
| MM | KCNV2 |
| MM | IL1F9 |
| MM | INSL3 |
| MM | CXCL6 |
| MM | SMR3A |
| MM | CFHR2 |
| MM | SHISA5 |
| MM | SLC2A2 |
| MM | PRH1; |
| MM | CHRNB3 |
| MM | TNFRSF13C |
| MM | RCN1 |
| MM | CCL15 |
| MM | TMEM91 |
| MM | RNASE10 |
| MM | PTPRR |
| MM | IL15RA |
| MM | CD151 |
| MM | SLC2A10 |
| MM | ERVK-7 |
| MM | PLVAP |
| MM | FKBP10 |
| MM | CCL28 |
| MM | ANTXRL |
| MM | CTRB2 |
| MM | FGF17 |
| MM | APP |
| MM | PNLIPRP1 |
| MM | LILRB6 |
| MM | ATP4B |
| MM | IGFBP5 |
| MM | LGALS3 |
| MM | IFNA17 |
| MM | LRIT3 |
| MM | CCL8 |
| MM | CTSA |
| MM | PRR4 |
| MM | DNAJC3 |
| MM | LCN15 |
| MM | TGOLN2 |
| MM | TSLP |
| MM | TGFA |
| MM | APOA1 |
| MM | CCL7 |
| MM | EVA1C |
| MM | SDC4 |
| MM | CSF2 |
| MM | IL28B |
| MM | ENSP00000381830 |
| MM | PPT1 |
| MM | CRTAM |
| MM | SPN |
| MM | DCD |
| MM | LAS2 |
| MM | CHGB |
| MM | DKK1 |
| MM | IL34 |
| MM | ERVK-24 |
| MM | IL1B |
| MM | LRP11 |
| MM | AIMP1 |
| MM | RSPO4 |
| MM | APOA4 |
| MM | PROK1 |
| MM | RSPO3 |
| MM | FKBP2 |
| MM | SCGB1A1 |
| MM | TM9SF3 |
| MM | MANSC4 |
| MM | CST6 |
| MM | SPACA7 |
| MM | SPACA5 |
| MM | DEFB126 |
| MM | SLC6A2 |
| MM | EPHA5 |
| MM | ASIP |
| MM | CD14 |
| MM | CRLF1 |
| MM | SNORC |
| MM | PRG3 |
| MM | RNASE8 |
| MM | IGF1 |
| MM | MUCL1 |
| MM | CLN5 |
| MM | STC2 |
| MM | SOSTDC1 |
| MM | MMP1 |
| MM | VSTM2A |
| MM | PRRT1 |
| MM | CELA3A |
| MM | PRRG4 |
| MM | C1QL1 |
| MM | CXCL17 |
| MM | IGFBP1 |
| MM | SLC22A31 |
| MM | LHFPL5 |
| MM | SLC6A5 |
| MM | VPREB1 |
| MM | FGF7 |
| MM | OLR1 |
| MM | AGER |
| MM | PRRT3 |
| MM | ATP6AP2 |
| MM | APOH |
| MM | CCL11 |
| MM | S100A13 |
| MM | CPXM2 |
| MM | CD248 |
| MM | FAM24B |
| MM | TDGF1 |
| MM | XG |
| MM | TNFRSF6B |
| MM | KLK7 |
| MM | PGC |
| MM | IGFBP3 |
| MM | IFNA6 |
| MM | SUMF1 |
| MM | FAM19A4 |
| MM | AHSG |
| MM | SMOC2 |
| MM | AMBN |
| MM | IL5 |
| MM | OVGP1 |
| MM | CCL26 |
| MM | EPYC |
| MM | FAM19A5 |
| MM | MSR1 |
| MM | IER3 |
| MM | OS9 |
| MM | XCL2 |
| MM | TRABD2B |
| MM | ADM2 |
| MM | CXCL3 |
| MM | MICB |
| MM | PDIA3 |
| MM | TMEM95 |
| MM | TM4SF6 |
| MM | RTN4R |
| MM | FKBP9 |
| MM | LHFPL1 |
| MM | TFF2 |
| MM | TNFRSF1B |
| MM | SPOCK1 |
| MM | GAST |
| MM | FAM174A |
| MM | CNPY4 |
| MM | C19orf18 |
| MM | TREML1 |
| MM | CLU |
| MM | KAL1 |
| MM | NBL1 |
| MM | TGFBR1 |
| MM | MANF |
| MM | MUC7 |
| MM | KCT2 |
| MM | PRRG3 |
| MM | FGF23 |
| MM | CTSG |
| MM | IL1RAP |
| MM | SCGB2A2 |
| MM | LY6H |
| MM | IHH |
| MM | NRN1 |
| MM | PTN |
| MM | PRAP1 |
| MM | FCMR |
| MM | APLP2 |
| MM | IL21 |
| MM | TNFRSF4 |
| MM | VSIG2 |
| MM | SIGLEC9 |
| MM | TRH |
| MM | SPP2 |
| MM | SPINK13 |
| MM | SEMA6C |
| MM | MEGF9 |
| MM | IL32 |
| MM | IL16 |
| MM | PLAC9 |
| MM | UNQ9165/PRO28630 |
| MM | DNASE2 |
| MM | IGFBP7 |
| MM | COL8A1 |
| MM | HSD11B1L |
| MM | CLDN3 |
| MM | HSD17B13 |
| MM | OBP2A |
| NMO | CXCL2 |
| NMO | CXCL3 |
| NMO | IGFBPL1 |
| NMO | CCL22 |
| NMO | IL1F9 |
| NMO | LY6G6D |
| NSCLC | CCL17 |
| NSCLC | CCL24 |
| NSCLC | CXCL1 |
| NSCLC | CXCL3 |
| NSCLC | EDIL3 |
| NSCLC | IFNA13 |
| NSCLC | IFNA14 |
| NSCLC | IFNA17 |
| NSCLC | IFNA2 |
| NSCLC | IFNA5 |
| NSCLC | IFNA6 |
| NSCLC | IFNA8 |
| NSCLC | IFNL2 |
| NSCLC | IFNW1 |
| NSCLC | IL28B |
| NSCLC | IL34 |
| NSCLC | MADCAM1 |
| NSCLC | PDGFB |
| NSCLC | REG1A |
| NSCLC | SDC1 |
| NSCLC | BTN1A1 |
| NSCLC | C6 |
| NSCLC | CD207 |
| NSCLC | CD3D |
| NSCLC | CDH19 |
| NSCLC | COLEC12 |
| NSCLC | EREG |
| NSCLC | FGF23 |
| NSCLC | FGF7 |
| NSCLC | FGFBP3 |
| NSCLC | IGFBPL1 |
| NSCLC | IL15RA |
| NSCLC | IL17F |
| NSCLC | IL1RAP |
| NSCLC | IL22RA2 |
| NSCLC | IL4 |
| NSCLC | IL4R |
| NSCLC | ITGA5 |
| NSCLC | LAG3 |
| NSCLC | LRRC4 |
| NSCLC | MPZL3 |
| NSCLC | NOTCH2NL |
| NSCLC | NTRK3 |
| NSCLC | REG4 |
| NSCLC | SCARA3 |
| NSCLC | STIM2 |
| NSCLC | TNFRSF10C |
| NSCLC | TNFRSF19L |
| NSCLC | TREML1 |
| PANDAS | LRP11 |
| Sarcoidosis | CX3CL1 |
| Sarcoidosis | EPYC |
| Sarcoidosis | PGLYRP1 |
| SLE | CXCL3 |
| SLE | IFNA17 |
| SLE | CXCL1 |
| SLE | LOC644613 |
| SLE | IFNA6 |
| SLE | SV2C |
| SLE | TMEM102 |
| SLE | PDCD1LG2 |
| SLE | SLC29A4 |
| SLE | IL1A |
| SLE | C5orf64 |
| SLE | IFNW1 |
| SLE | SCGB1D1 |
| SLE | EPYC |
| SLE | CNPY2 |
| SLE | CCL4L1 |
| SLE | SPINK9 |
| SLE | TNF |
| SLE | KIRREL3 |
| SLE | IFNA8 |
| SLE | IFNA14 |
| SLE | VEGFB |
| SLE | TMEM108 |
| SLE | IFNA5 |
| SLE | ACVR2B |
| SLE | OBP2B |
| SLE | MCFD2 |
| SLE | DPT |
| SLE | SPACA7 |
| SLE | IFNA13 |
| SLE | FKBP14 |
| SLE | LACRT |
| SLE | IL6 |
| SLE | FAM19A3 |
| SLE | IFNL2 |
| SLE | ERP27 |
| SLE | TMEM149 |
| SLE | PRH1; |
| SLE | ZG16B |
| SLE | IFNA2 |
| SLE | RAET1E |
| SLE | CCDC47 |
| SLE | MUC21 |
| SLE | CCL22 |
| SLE | CGREF1 |
| SLE | TEPP |
| SLE | FAM19A2 |
| SLE | SPOCK1 |
| SLE | SRGN |
| SLE | SHISA7 |
| SLE | CCL17 |
| SLE | RNASE10 |
| SLE | FGF21 |
| SLE | APOA4 |
| SLE | NGFR |
| SLE | KCNV2 |
| SLE | AGER |
| SLE | FGFRL1 |
| SLE | LGR6 |
| SLE | CCL8 |
| SLE | CD44 |
| SLE | ITIH3 |
| SLE | CST8 |
| SLE | SSPN |
| SLE | CELA1 |
| SLE | IL4 |
| SLE | RCN3 |
| SLE | PRRG4 |
| SLE | MFAP5 |
| SLE | CSPG5 |
| SLE | VTCN1 |
| SLE | PLA2G2E |
| SLE | LY6H |
| SLE | GYPC |
| SLE | SLC41A2 |
| SLE | DRAXIN |
| SLE | CSHL1 |
| SLE | LAIR2 |
| SLE | IGFBP2 |
| SLE | CD248 |
| SLE | RGMB |
| SLE | TGOLN2 |
| SLE | CSAG1 |
| SLE | ACP4 |
| SLE | CALU |
| SLE | BTNL8 |
| SLE | SOSTDC1 |
| SLE | LYSMD4 |
| SLE | LCN2 |
| SLE | SCGB1C2 |
| SLE | CST4 |
| SLE | IGF1 |
| SLE | PRRT1 |
| SLE | CHRNA5 |
| SLE | ANTXRL |
| SLE | TNFRSF6 |
| SLE | CD300LG |
| SLE | SERPINE1 |
| SLE | OLFM1 |
| SLE | PLA2G10 |
| SLE | CD300E |
| SLE | CDH19 |
| SLE | RAMP2 |
| SLE | ATP4B |
| SLE | PTPRR |
| SLE | SFN |
| SLE | HCRTR2 |
| SLE | ACRV1 |
| SLE | FAM3A |
| SLE | ACVR1B |
| SLE | FGF23 |
| SLE | IL15RA |
| SLE | IGFBP7 |
| SLE | LHFPL1 |
| SLE | IL28B |
| SLE | VIT |
| SLE | IER3 |
| SLE | C2orf40 |
| SLE | PLVAP |
| SLE | LECT2 |
| SLE | DAG1 |
| SLE | SPINK6 |
| SLE | SLC2A12 |
| SLE | IGLL1 |
| SLE | TFF2 |
| SLE | ASIP |
| SLE | IL16 |
| SLE | EDIL3 |
| SLE | CCL13 |
| SLE | RCN1 |
| SLE | CSH2 |
| SLE | IL33 |
| SLE | LILRB4 |
| SLE | SPESP1 |
| SLE | PDGFB |
| SLE | PTHLH |
| SLE | C9orf47 |
| SLE | CHRDL2 |
| SLE | ART3 |
| SLE | CPVL |
| SLE | CCL15 |
| SLE | CFD |
| SLE | MFSD2A |
| SLE | RTN4RL1 |
| SLE | ADM2 |
| SLE | APOO |
| SLE | CTSG |
| SLE | PMCH |
| SLE | DKK2 |
| SLE | CARTPT |
| SLE | BTC |
| SLE | IL18RAP |
| SLE | LRIT3 |
| SLE | LHFPL5 |
| SLE | SPN |
| SLE | FAM19A5 |
| SLE | IL6R |
| SLE | SDC1 |
| SLE | IL20RB |
| SLE | CXCL9 |
| SLE | RNASE8 |
| SLE | LILRB2 |
| SLE | CDSN |
| SS | CXCL1 |
| SS | CXCL3 |
| SS | PDCD1LG2 |
| SSC | KLK10 |
| SSC | RCN2 |
| SSC | IGFBP6 |
| SSC | SERPINA3 |
| SSC | SPOCK1 |
| SSC | SPINK9 |
| SSC | AGRP |
| SSC | CCL21 |
| SSC | CSF2 |
| SSC | CALU |
| SSC | ENDOU |
| SSC | CXCL1 |
| SSC | NEGR1 |
| SSC | C5orf64 |
| SSC | CCDC47 |
| SSC | IL1A |
| SSC | EPYC |
| SSC | GNLY |
| SSC | PGA3 |
| SSC | UNQ6190_PRO20217 |
| SSC | CCL4L1 |
| SSC | OBP2B |
| SSC | KLK8 |
| SSC | OTOS |
| SSC | CNPY2 |
| SSC | ERP27 |
| SSC | CP |
| SSC | MUCL3 |
| SSC | RAET1L |
| SSC | ULBP2 |
| SSC | TM2D2 |
| SSC | SLC2A2 |
| SSC | IL6 |
| SSC | SERPINE1 |
| SSC | LEP |
| SSC | LECT2 |
| SSC | OTOR |
| SSC | CASQ1 |
| SSC | CST6 |
| SSC | INSL3 |
| SSC | SPACA3 |
| SSC | AMTN |
| SSC | ZG16B |
| SSC | LOC644613 |
| SSC | PGA4 |
| SSC | LYSMD4 |
| SSC | SRGN |
| SSC | CDH19 |
| SSC | SHISA7 |
| SSC | FAM19A3 |
| SSC | HAVCR1 |
| SSC | BAMBI |
| SSC | MSMP |
| SSC | SPACA7 |
| SSC | PTHLH |
| SSC | PLA2G12B |
| SSC | CXCL3 |
| SSC | CST4 |
| SSC | DKK3 |
| SSC | PIANP |
| SSC | PRG3 |
| SSC | BTC |
| SSC | CCL17 |
| SSC | XCL1 |
| SSC | LMBRD2 |
| SSC | LALBA |
| SSC | TGFA |
| SSC | IL29 |
| SSC | EVI2B |
| SSC | SLPI |
| SSC | CLCC1 |
| SSC | RNASE10 |
| SSC | FGFBP3 |
| SSC | FAM168B |
| SSC | PGLYRP1 |
| SSC | ANGPTL4 |
| SSC | CLU |
| SSC | AGER |
| SSC | TMEM108 |
| SSC | C1QTNF2 |
| SSC | TMEM119 |
| SSC | CCL8 |
| SSC | ODAPH |
| SSC | CNPY3 |
| SSC | MZB1 |
| SSC | CYTL1 |
| SSC | PRH1 |
| SSC | SLC2A10 |
| SSC | PRRG1 |
| SSC | CSPG5 |
| SSC | DRAXIN |
| SSC | PRR27 |
| SSC | DKK1 |
| SSC | NTRK2 |
| SSC | IFNA13 |
| SSC | PDCD1 |
| SSC | FAM19A2 |
| SSC | IFNW1 |
| SSC | RCN1 |
| SSC | CFD |
| SSC | CRELD2 |
| SSC | CCL18 |
| SSC | CD14 |
| SSC | BTN1A1 |
| SSC | PTPRR |
| SSC | TMEM91 |
| SSC | VSIG2 |
| SSC | CCL13 |
| SSC | C2orf40 |
| SSC | VEGFB |
| SSC | REG4 |
| SSC | TXNDC12 |
| SSC | ACVR2B |
| SSC | ODAM |
| SSC | CST5 |
| SSC | PI3 |
| SSC | TMEM149 |
| SSC | TEPP |
| SSC | KCNV2 |
| SSC | PLA2G2E |
| SSC | AIMP1 |
| SSC | IGFBP5 |
| SSC | ASIP |
| SSC | PGC |
| SSC | TM9SF3 |
| SSC | AMELX |
| SSC | CSN2 |
| SSC | CPXM2 |
| SSC | PRSS3 |
| SSC | FAM3A |
| SSC | LILRA3 |
| SSC | CSAG1 |
| SSC | RTBDN |
| SSC | CELA1 |
| SSC | ANTXRL |
| SSC | PLA2G10 |
| SSC | KCT2 |
| SSC | APOH |
| SSC | NENF |
| SSC | NPPC |
| SSC | LY6H |
| SSC | FGF1 |
| SSC | SLC1A1 |
| SSC | IFNL2 |
| SSC | HSPA13 |
| SSC | C6orf15 |
| SSC | FLJ37218 |
| SSC | CCL7 |
| SSC | APOA4 |
| SSC | FSTL1 |
| SSC | IGFBP1 |
| SSC | FCGR2A |
| SSC | SMR3A |
| SSC | IFITM10 |
| SSC | MSLN |
| SSC | PRAP1 |
| SSC | EPO |
| SSC | PLVAP |
| SSC | PROK1 |
| SSC | TSLP |
| SSC | MIA |
| SSC | APP |
| SSC | OBP2A |
| SSC | RTN4RL1 |
| SSC | PRRT3 |
| SSC | APOA1 |
| SSC | FGF7 |
| SSC | TMED1 |
| SSC | LGALS3 |
| SSC | JCHAIN |
| SSC | PRRG3 |
| SSC | IGF1 |
| SSC | ACRV1 |
| SSC | SLC38A4 |
| SSC | FKBP11 |
| SSC | ITPRIPL1 |
| SSC | PLAC9 |
| SSC | TFF2 |
| SSC | WFDC13 |
| SSC | LCN1 |
| SSC | LYG1 |
| SSC | LAIR2 |
| SSC | TNFRSF8 |
| SSC | SOSTDC1 |
| SSC | VSTM2A |
| SSC | IGFBP7 |
| SSC | PSORS1C2 |
| SSC | FGF23 |
| SSC | RSPO3 |
| SSC | S100A9 |
| SSC | CXCL9 |
| SSC | TGOLN2 |
| SSC | ACP5 |
| SSC | MANF |
| SSC | AMBN |
| SSC | PSAPL1 |
| SSC | WFDC10A |
| SSC | PPT1 |
| SSC | MANSC4 |
| SSC | CD248 |
| SSC | NGRN |
| SSC | PSAP |
| SSC | LILRB2 |
| SSC | SCGB2A2 |
| SSC | IGFBPL1 |
| SSC | SV2C |
| SSC | CXCL6 |
| SSC | CD300E |
| SSC | RCN3 |
| SSC | IGFBP3 |
| SSC | RTN4R |
| SSC | PRRT1 |
| SSC | ACVR2A |
| SSC | LCN2 |
| SSC | HCRTR2 |
| SSC | CELA3A |
| SSC | ADM2 |
| SSC | LRIT3 |
| SSC | MIA2 |
| SSC | TNFRSF17 |
| SSC | SPN |
| SSC | SLC6A5 |
| SSC | WFDC1 |
| SSC | LILRB4 |
| SSC | CTSG |
| SSC | CXCL11 |
| SSC | KLK7 |
| SSC | CST8 |
| SSC | NOPE |
| SSC | GAST |
| SSC | ASTN2 |
| SSC | MCFD2 |
| SSC | CCL22 |
| SSC | OTOL1 |
| SSC | SYCN |
| SSC | CCL2 |
| SSC | SOST |
| SSC | PTN |
| SSC | TACSTD2 |
| SSC | IL21 |
| SSC | IGLL1 |
| SSC | MMP7 |
| SSC | APLP2 |
| SSC | SSBP3_AS1 |
| SSC | CST7 |
| SSC | SSPN |
| SUSAC | CCL24 |
| SUSAC | SDC4 |
| SUSAC | TREML1 |
| SUSAC | VSIG4 |

TABLE 5
Therapeutic Autoantigens and Corresponding Disease or Disorder

| Disease | Target |
|---|---|
| APECED | IL22RA2 |
| CLE | TYRO3 |
| CLE | CD300E |
| COVID-19 | IL13 |
| COVID-19 | IL18RAP |
| COVID-19 | TNFRSF8 |
| COVID-19 | CCR10 |
| COVID-19 | CD74 |
| COVID-19 | TNFRSF17 |
| COVID-19 | CCR9 |
| COVID-19 | CRTAM |
| COVID-19 | C6 |
| DM | CD81 |
| GN | IL34 |
| KT | IGFBP1 |
| KT | IL15RA |
| KT | NXPH1 |
| KT | CST5 |
| KT | C6 |
| MG | CCL22 |
| MG | CCL2 |
| MM | PSORS1C2 |
| MM | LHFPL1 |
| MM | PTPRR |
| MM | ZG16B |
| MM | IGF1 |
| MM | IGLL1 |
| MM | LRIT3 |
| MM | VEGFB |
| NSCLC | CCL22 |
| NMO | CCL22 |
| NMO | IL1F9 |
| NSCLC | FGF23 |
| NSCLC | FGF7 |
| NSCLC | EREG |
| NSCLC | CXCL1 |
| NSCLC | CXCL2 |
| NSCLC | CXCL3 |
| NSCLC | VEGFB |
| NSCLC | IL1A |
| NSCLC | LAG3 |
| NSCLC | IFNA13 |
| NSCLC | IFNA14 |
| NSCLC | IFNA17 |
| NSCLC | IFNA2 |
| NSCLC | IFNA5 |
| NSCLC | IFNA6 |
| NSCLC | IFNA8 |
| NSCLC | IFNW1 |
| NSCLC | IL34 |
| NSCLC | IL22RA2 |
| SLE | PDCD1LG2 |
| SLE | LIF |
| SLE | IFNA13 |
| SLE | IFNA14 |
| SLE | IFNA17 |
| SLE | IFNA2 |
| SLE | IFNA5 |
| SLE | IFNA6 |
| SLE | IFNA8 |
| SLE | IFNB1 |
| SLE | IFNL2 |
| SLE | IFNW1 |
| SLE | IL6 |
| SLE | IL6R |
| SLE | IL33 |
| SLE | IL34 |
| SLE | IL16 |
| SLE | IL19 |
| SLE | IL20RB |
| SLE | IL18RAP |
| SLE | MADCAM1 |
| SLE | TNF |
| SLE | TRAILR4 |
| SLE | TYRO3 |
| SLE | CD44 |
| SLE | CD300E |
| SLE | CXCL1 |
| SLE | CXCL2 |
| SLE | CXCL3 |
| SLE | VEGFB |
| SLE | IL1A |
| SLE | LILRB2 |
| SLE | LILRB4 |
| SS | PDCD1LG2 |
| NSCLC | IGFBPL1 |

TABLE 6
Autoantigen Specific Therapies

| Disease | Target |
|---|---|
| COVID-19 | IFITM10 |
| COVID-19 | IFNA13 |
| COVID-19 | IFNA14 |
| COVID-19 | IFNA17 |
| COVID-19 | IFNA2 |
| COVID-19 | IFNA5 |
| COVID-19 | IFNA6 |
| COVID-19 | IFNA8 |
| COVID-19 | IFNW1 |
| COVID-19 | KLRC1 |
| COVID-19 | KLRC2 |
| COVID-19 | KLRC3 |
| COVID-19 | CCR2 |
| COVID-19 | CD38 |
| COVID-19 | C5A |
| COVID-19 | CCR4 |
| COVID-19 | CD3E |
| COVID-19 | TNFRSF9 |
| COVID-19 | ADCYAP1 |
| COVID-19 | CGA |
| COVID-19 | HCTR2 |
| COVID-19 | AZGP1 |
| COVID-19 | SLC41A2 |
| COVID-19 | LAIR1 |
| KT | IFITM10 |
| KT | IL4 |
| KT | EXOC3-AS1 |
| KT | IFNA13 |
| KT | CD99L2 |
| KT | OSTN |
| KT | SYCN |
| KT | LYG2 |
| KT | BTN1A1 |
| MM | IFNA13 |
| MM | OBP2B |
| MM | TMEM108 |
| MM | CELA1 |
| MM | OTOL1 |
| MM | ATP4B |
| MM | ICOSLG |
| MM | REG1A |
| MM | CCL24 |
| MM | TMEM91 |
| MM | LALBA |
| MM | ITPRIPL1 |
| MM | LCN2 |
| MM | BTN1A1 |
| MM | OS9 |
| MM | FGF17 |
| NSCLC | IFNL2 |
| NSCLC | VSTM2A |
| NSCLC | PDGFB |
| SLE | TMEM102 |
| SLE | CCL8 |
| SLE | CCL4L1 |
| SLE | ACVR2B |
| SLE | FGF21 |
| SLE | IGFBP2 |
| SLE | RGMB |
| SLE | ACVR1B |
| SLE | ACRV1 |
| SLE | SCGB1D1 |
| SLE | TFF2 |
| SLE | SFN |
| SLE | ANTXRL |
| SLE | SLC41A2 |
| SLE | CD248 |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of identifying at least one polypeptide which binds to at least one antibody, wherein the method comprises:
(a) contacting a library of display cells or particles with a sample comprising at least one antibody, wherein the library of display cells comprises a plurality of cells or particles wherein together the plurality of cells or particles comprises nucleic acid molecules for expression of a plurality of extracellular proteins, secreted proteins or a combination thereof;
wherein each cell or particle of the plurality of cells or particles comprises a barcoded nucleic acid molecule, wherein each nucleic acid molecule comprises
i) a nucleotide sequence encoding a polypeptide of interest for display on the surface of the cell or particle; and
ii) a unique nucleotide barcode sequence;
(b) isolating one or more antibody-bound cell or particle;
(c) isolating at least one barcoded nucleic acid molecule from at least one cell or particle of step (b); and
(d) identifying the barcoded nucleic acid molecule, thereby identifying the associated encoded polypeptide as an antigen for binding by at least one antibody in the sample.

2. The method of claim 1, wherein the method of isolating one or more antibody-bound cell or particle comprises high-throughput magnetic separation.

3. The method of claim 1, wherein the method further comprises the step of:
(b′) expanding the one or more isolated antibody-bound cell or particle.

4. The method of claim 1, wherein the method of identifying the barcoded nucleic acid molecule comprises at least one selected from the group consisting of amplifying the barcoded nucleic acid molecule and sequencing the barcoded nucleic acid molecule.

5. The method of claim 1, comprising:
in step (b), isolating multiple antibody bound cells,
in step (c), isolating the barcoded nucleic acid molecules from the cells of step (b), and
in step (d), sequencing the isolated barcoded nucleic acid molecules, and identifying the associated encoded polypeptide as an antigen for binding by the antibody based on an enrichment of the number of reads of the associated barcode in the sequencing data as compared to a threshold level.

6. The method of claim 3, wherein the threshold level is selected from the group consisting of a predetermined threshold level, a statistically determined threshold, and a threshold level determined using z-scores.

7. The method of claim 1, wherein the library of display cells or particles comprises a library of barcoded nucleic acid molecules encoding at least one selected from an extracellular domain of a protein, an extracellular protein, and a secreted protein.

8. The method of claim 7, wherein the library of barcoded nucleic acid molecules comprises a plurality of nucleic acid molecules which together encode the human exoproteome.

9. The method of claim 7, wherein the library of barcoded nucleic acid molecules comprises at least one nucleic acid molecule encoding at least one polypeptide sequence selected from SEQ ID NO:1-3092.

10. The method of claim 7, wherein the library of barcoded nucleic acid molecules comprises a plurality of nucleic acid molecules which together encode each of SEQ ID NO:1-3092.

11. The method of claim 7, wherein the library of barcoded nucleic acid molecules comprises at least one nucleic acid molecule comprising a nucleotide sequence selected from SEQ ID NO:3093-6185.

12. The method of claim 7, wherein the library of barcoded nucleic acid molecules comprises a plurality of nucleic acid molecules which together comprise each of SEQ ID NO:3093-6185.

13. The method of claim 1, wherein the sample comprises a biological sample selected from the group consisting of a body fluid, blood, serum, plasma, cerebrospinal fluid, tissue, and any combination thereof.

14. The method of claim 1, wherein the sample comprises at least one antibody purified from a biological sample selected from the group consisting of a body fluid, blood, serum, plasma, cerebrospinal fluid, tissue, and any combination thereof.

15. The method of claim 14, wherein at least one antibody is purified from a biological sample by at least one selected from the group consisting of:
(a) affinity purification for a specific antibody isotype of interest, and
(b) contacting the sample with a control cell or particle comprising an empty expression plasmid.

16. The method of claim 1, wherein the sample is from a subject diagnosed as having a disease or disorder, and whereby the antigen for binding by at least one antibody is a disease-associated antigen.

17. The method of claim 1, wherein the antibody is an autoantibody.

18. The method of claim 1, wherein the antibody is associated with an autoimmune disease or disorder, cancer, inflammatory disease or disorder, metabolic disease or disorder, neurodegenerative disease or disorder, organ tissue rejection, organ transplant rejection, or any combination thereof.

19. A method of preventing or treating a disease or disorder in a subject in need thereof; the method comprising administering a therapeutic agent to the subject, wherein the therapeutic agent comprises an agent for modifying the level or reactivity of at least one antibody which interacts with at least one antigen selected from the group consisting of the antigens as set forth in SEQ ID NO:1-3092.

20. The method of claim 19, wherein the antigen is identified as a target for at least one antibody according to the method of claim 1.

21. The method of claim 19, wherein the at least one antigen is selected from the group consisting of an antigen as set forth in Table 3, and further wherein the disease or disorder is the disease or disorder associated with the antigen as set forth in Table 3.

22. The method of claim 21, wherein the therapeutic agent comprises an agent for decreasing the level or reactivity of at least one antibody with at least one disease-associated antigen selected from the group consisting of the antigens as set forth in Table 3.

23. The method of claim 19, wherein the at least one antigen is selected from the group consisting of an antigen as set forth in Table 6, and further wherein the disease or disorder is the disease or disorder associated with the antigen as set forth in Table 6.

23. The method of claim 19, wherein the therapeutic agent comprises a therapeutically effective amount of at least agent that reduces or eliminates at least one antibody.

24. The method of claim 23, wherein the therapeutic agent comprises a composition comprising an antigen selected from the group consisting of an antigen as set forth in SEQ ID NO:1-3092 linked to a domain for endocytosis and degradation.

25. The method of claim 23, wherein the therapeutic agent comprises a composition comprising an antigen selected from the group consisting of an antigen as set forth in Table 6 linked to a domain for endocytosis and degradation.

26. The method of claim 24, wherein the domain for endocytosis and degradation comprises an asialoglycoprotein receptor binding domain.

27. The method of claim 23, wherein the agent that reduces or eliminates at least one antibody comprises a molecule for targeting and destruction of at least one antibody-expressing cell.

28. The method of claim 27, wherein the agent comprises a chimeric antigen receptor (CAR) T cell expressing an antigen selected from the group consisting of an antigen as set forth in SEQ ID NO:1-3092, or a fragment thereof.

29. The method of claim 28, wherein the CAR T cell expresses an antigen selected from the group consisting of an antigen as set forth in Table 6.

30. The method of claim 19, wherein the therapeutic agent comprises an agent for increasing the level or reactivity of at least one antibody with at least one disease-associated antigen selected from the group consisting of the antigens as set forth in Table 3.

31. The method of claim 30, wherein the at least one antigen is selected from the group consisting of an antigen as set forth in Table 5, and further wherein the disease or disorder is the disease or disorder associated with the antigen as set forth in Table 5.

32. The method of claim 30, wherein the therapeutic agent comprises a therapeutically effective amount of at least one antibody, or fragment thereof, wherein the antibody specifically binds to a disease-associated antigen.

33. The method of claim 19, wherein the disease or disorder is selected from the group consisting of an autoimmune disease or disorder, cancer, inflammatory disease or disorder, metabolic disease or disorder, neurodegenerative disease or disorder, organ tissue rejection, organ transplant rejection, or any combination thereof.

34. The method of claim 19, wherein the disease or disorder is selected from the group consisting of antineutrophil cytoplasmic antibody (ANCA)-associated vasculitis, autoimmune polyendocrinopathy candidiasis ecto-dermal dystrophy, antiphospholipid antibody syndrome, chronic inflammatory demyelinating polyradiculoneuropathy, cutaneous lupus erythematosus, COVID-19, drug-induced lupus, dermatomyositis, glomerulonephritis, a disease or disorder associated with kidney transplant, malaria, mixed connective tissue disease, myasthenia gravis, malignant melanoma, neuromyelitis optica, non-small cell lung cancer, pediatric autoimmune neuropsychiatric disorders associated with streptococcal infections, systemic lupus erythematosus, sjogren's syndrome, scleroderma, susac syndrome, undifferentiated connective tissue disease, and any combination thereof.

35. A method of diagnosing, assessing the prognosis, or assessing the effectiveness of treatment of a disease or disorder in a subject in need thereof; the method comprising assessing the level or reactivity of at least one antibody which interacts with at least one antigen selected from the group consisting of an antigen as set forth in SEQ ID NO:1-3092.

36. The method of claim 35, wherein the at least one antigen is selected from the group consisting of an antigen as set forth in Table 3, and further wherein the disease or disorder is the disease or disorder associated with the antigen as set forth in Table 3.

37. The method of claim 35, wherein the at least one antigen is selected from the group consisting of an antigen as set forth in Table 4, and further wherein the disease or disorder is the disease or disorder associated with the antigen as set forth in Table 4.

38. The method of claim 35, wherein the disease or disorder is selected from the group consisting of an autoimmune disease or disorder, cancer, inflammatory disease or disorder, metabolic disease or disorder, neurodegenerative disease or disorder, organ tissue rejection, organ transplant rejection, or any combination thereof.

39. The method of claim 35, wherein the disease or disorder is selected from the group consisting of antineutrophil cytoplasmic antibody (ANCA)-associated vasculitis, autoimmune polyendocrinopathy candidiasis ecto-dermal dystrophy, antiphospholipid antibody syndrome, chronic inflammatory demyelinating polyradiculoneuropathy, cutaneous lupus erythematosus, COVID-19, drug-induced lupus, dermatomyositis, glomerulonephritis, a disease or disorder associated with kidney transplant, malaria, mixed connective tissue disease, myasthenia gravis, malignant melanoma, neuromyelitis optica, non-small cell lung cancer, pediatric autoimmune neuropsychiatric disorders associated with streptococcal infections, systemic lupus erythematosus, sjogren's syndrome, scleroderma, susac syndrome, undifferentiated connective tissue disease, and any combination thereof.

40. A composition comprising an antigen selected from the group consisting of an antigen as set forth in SEQ ID NO:1-3092, or a fragment thereof, linked to a domain for endocytosis, degradation, or a combination thereof.

41. The composition of claim 40, wherein the composition comprises an antigen selected from the group consisting of an antigen as set forth in Table 6 linked to a domain for endocytosis, degradation, or a combination thereof.

42. The composition of claim 40, wherein the domain for endocytosis, degradation, or a combination thereof comprises an asialoglycoprotein receptor binding domain.

43. A composition for targeting and destruction of at least one antibody-expressing cell comprising an antigen selected from the group consisting of an antigen as set forth in SEQ ID NO:1-3092, or a fragment thereof.

44. The composition of claim 43, wherein the agent comprises a chimeric antigen receptor (CAR) T cell expressing an antigen as set forth in SEQ ID NO:1-3092, or a fragment thereof.

45. The composition of claim 44, wherein the CAR T cell expresses an antigen selected from the group consisting of an antigen as set forth in Table 6.
